(19) United States
(12) Patent Application Publication
(10) Pub. No.: US 2020/0216392 A1
(43) Pub. Date: Jul. 9, 2020

(54) A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Bitnari KIM, Gyeonggi-do (KR); Sang-Hee CHO, Gyeonggi-do (KR); Kyung-Hoon CHOI, Gyeonggi-do (KR)

(21) Appl. No.: 16/631,186

(22) PCT Filed: Jul. 25, 2018
(86) PCT No.: PCT/KR2018/008434

§ 371 Date: Jan. 15, 2020

(30) Foreign Application Priority Data
Jul. 26, 2017 (KR) 10-2017-0094827
Jul. 18, 2018 (KR) 10-2018-0083455

(51) Int. Cl. C07D 209/82; H01L 51/00; C07D 409/14; C07D 405/14; C07D 403/14; H01L 51/50

(52) U.S. Cl. CPC C07D 209/82; H01L 51/0052; H01L 51/0072; H01L 51/5024; C07D 409/14; C07D 405/14; C07D 403/14; H01L 51/006

(57) ABSTRACT

The present disclosure relates to a plurality of host materials comprising a compound represented by formula 1 and a compound represented by formula 2, and an organic electroluminescent device comprising the same. The organic electroluminescent device according to the present disclosure can exhibit excellent lifespan characteristic, while maintaining low driving voltage and high luminous efficiency, by comprising a plurality of host compounds in a specific combination.
wherein the substituents are as defined in the specification.

### TECHNICAL FIELD

The present disclosure relates to a plurality of host materials and an organic electroluminescent device comprising the same.

### BACKGROUND ART

An electroluminescent device (EL device) is a self-light-emitting device which has advantages in that it provides a wide viewing angle, a great contrast ratio, and a fast response time. The first organic EL device was developed by Eastman Kodak in 1987, by using small aromatic diamine molecules, and aluminum complexes as materials for forming a light-emitting layer [Appl. Phys. Lett. 51, 913, 1987].

An organic electroluminescent device (OLED) is a device changing electrical energy to light by applying electricity to an organic electroluminescent material, and generally has a structure comprising an anode, a cathode, and an organic layer between the anode and the cathode. The organic layer of an organic EL device may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc. The materials used for the organic layer are categorized by their functions in a hole injection material, a hole transport material, a hole auxiliary material, a light-emitting auxiliary material, an electron blocking material, a light-emitting material (including host and dopant materials), an electron buffer material, a hole blocking material, an electron transport material, an electron injection material, etc. In the organic EL device, due to an application of a voltage, holes are injected from the anode to the light-emitting layer, electrons are injected from the cathode to the light-emitting layer, and excitons of high energies are formed by a recombination of the holes and the electrons. By this energy, organic luminescent compounds reach an excited state, and light emission occurs by emitting light from energy due to the excited state of the organic luminescent compounds returning to a ground state.

The most important factor determining luminous efficiency in an organic EL device is light-emitting materials. A light-emitting material must have high quantum efficiency, high electron and hole mobility, and the formed light-emitting material layer must be uniform and stable. Light-emitting materials are categorized into blue, green, and red light-emitting materials dependent on the color of the light emission, and additionally yellow or orange light-emitting materials. In addition, light-emitting materials can also be categorized into host and dopant materials according to their functions. Recently, the development of an organic EL device providing high efficiency and long lifespan is an urgent issue. In particular, considering EL characteristic requirements for a middle or large-sized panel of OLED, materials showing better characteristics than conventional ones must be urgently developed. The host material, which acts as a solvent in a solid state and transfers energy, needs to have high purity and a molecular weight appropriate for vacuum deposition. Furthermore, the host material needs to have high glass transition temperature and high thermal degradation temperature to achieve thermal stability, high electro-chemical stability to achieve long lifespan, ease of forming an amorphous thin film, good adhesion to materials of adjacent layers, and non-migration to other layers.

A light-emitting material can be used as a combination of a host and a dopant to improve color purity, luminous efficiency, and stability. Generally, a device having excellent EL characteristics has a structure comprising a light-emitting layer formed by doping a dopant to a host. Since host materials greatly influence the efficiency and lifespan of the EL device when using a dopant/host material system as a light-emitting material, their selection is important.

Korean Patent Application Laying-Open No. 2011-0066766 discloses an organic EL device using a benzothienocarbazole derivative fused with a benzene ring as a host material. Further, Korean Patent Application Laying-Open No. 2016-0149994 discloses an organic EL device using a compound, in which an arylamine is bonded directly or via a linker to a carbazole, as a host material. However, said references do not specifically disclose an organic EL device using a compound, in which an arylamine is bonded directly or via a linker to a carbazole, and a benzothieno carbazole, benzofurano carbazole, indolocarbazole, or indenocarbazole derivative fused with a benzene ring, as a plurality of host materials. In addition, the organic EL devices disclosed in said references still need to be improved in terms of driving voltage, current efficiency, and operational lifespan.

### DISCLOSURE OF THE INVENTION

### Problems to be Solved

The objective of the present disclosure is to provide an organic electroluminescent device having long lifespan, while maintaining low driving voltage and/or high luminous efficiency.

### Solution to Problems

As a result of intensive studies, the present inventors found that the objective above can be achieved by a plurality of host materials comprising at least one first host compound and at least one second host compound, wherein the first host compound is represented by the following formula 1, and the second host compound is represented by the following formula 2:

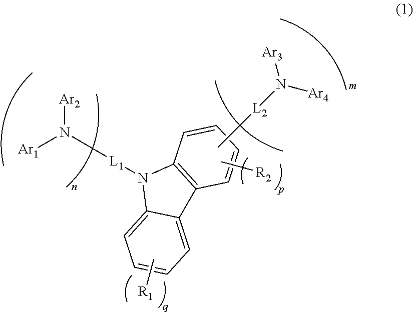

wherein

Ar₁ to Ar₄ each independently represent a substituted or unsubstituted (C6-C30)aryl; or Ar₁ and Ar₂, and Ar₃ and Ar₄ may be linked to each other to form a substituted or unsubstituted (3- to 30-membered) ring;

L₁ represents a single bond, or a substituted or unsubstituted (C6-C30)aryl(ene);

L₂ represents a single bond, or a substituted or unsubstituted (C6-C30)arylene;

if Ar₁ or Ar₂ represents a substituted or unsubstituted (C6-C30)aryl, and L₁ represents a substituted or unsubstituted (C6-C30)arylene, Ar₁ or Ar₂ and L₁ may be linked via a single bond to form a substituted or unsubstituted (3- to 30-membered) ring;

if Ar₃ or Ar₄ represents a substituted or unsubstituted (C6-C30)aryl, and L₂ represents a substituted or unsubstituted (C6-C30)arylene, Ar₃ or Ar₄ and L₂ may be linked via a single bond to form a substituted or unsubstituted (3- to 30-membered) ring;

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring;

m and n each independently represent an integer of 0 to 2, with a proviso that at least one of m and n is 1 or more; and

p and q each independently represent an integer of 1 to 4, in which if p and q represent an integer of 2 or more, each of R₁ and R₂ may be the same or different;

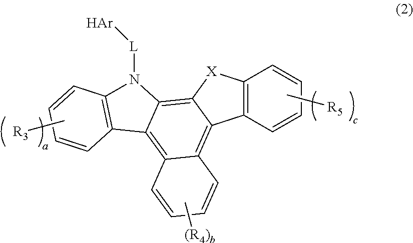

wherein

X represents —NR₁₁—, —CR₁₂R₁₃—, —O—, or —S—;

HAr represents a substituted or unsubstituted (3- to 30-membered)heteroaryl;

L represents a single bond, a substituted or unsubstituted (C6-C30)arylene, or a substituted or unsubstituted (3- to 30-membered)heteroarylene;

R₁₁ to R₁₃ each independently represent a substituted or unsubstituted (C1-C30)alkyl, a substituted or unsubstituted (C6-C30)aryl, or a substituted or unsubstituted (3- to 30-membered)heteroaryl;

R₃ to R₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring; and

a, b, and c each independently represent an integer of 1 to 4, in which if a, b, and c represent an integer of 2 or more, each of R₃, R₄, and R₅ may be the same or different.

### Effects of the Invention

According to the present disclosure, an organic electroluminescent device having long lifespan while maintaining low driving voltage and/or high luminous efficiency is provided, and a display device or a lighting device using the organic electroluminescent device can be manufactured.

### BRIEF DESCRIPTION OF THE FIGURES

FIG. 1 illustrates current efficiency versus luminance of organic electroluminescent devices produced in Comparative Example 1 and Device Example 2.

### EMBODIMENTS OF THE INVENTION

Hereinafter, the present disclosure will be described in detail. However, the following description is intended to explain the invention, and is not meant in any way to restrict the scope of the invention.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (a host material or a dopant material), an electron buffer material, a hole blocking material, an electron transport material, or an electron injection material.

The term “a plurality of organic electroluminescent materials” in the present disclosure means an organic electroluminescent material as a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. At least two compounds may be comprised in the same layer or different layers, and may be mixture-evaporated or co-evaporated, or may be individually evaporated.

The term “a plurality of host materials” in the present disclosure means an organic electroluminescent material as a combination of at least two host materials. It may mean both a material before being comprised in an organic electroluminescent device (for example, before vapor deposition) and a material after being comprised in an organic electroluminescent device (for example, after vapor deposition). A plurality of host materials of the present disclosure may be comprised in any light-emitting layer constituting an organic electroluminescent device. At least two compounds comprised in a plurality of host materials may be comprised together in one light-emitting layer or may respectively be comprised in different light-emitting layers. If at least two host materials are comprised in one layer, for example, they may be mixture-evaporated to form a layer, or may be separately co-evaporated at the same time to form a layer.

The benzothieno carbazole, benzofurano carbazole, indolocarbazole, or indenocarbazole derivative fused with a benzene ring, which corresponds to formula 2, inherently has high electronegativity and an electron-enriched group, and is rigid as a fused structure, and thus intermolecular transition is easy. In addition, if the intermolecular stacking is enhanced, the horizontal molecular orientation is more easily achieved, thereby realizing fast current characteristic. Thus, using the limited structures of triazine, quinazoline, quinoxaline, pyrimidine derivatives, etc., as host materials, light-emitting devices having a relatively low driving voltage, excellent luminous efficiency such as current efficiency and power efficiency, and capable of realizing high color purity have been provided. However, since said compounds have relatively strong electron current characteristic, excitons generated in an organic electroluminescent device are extremely formed between a hole transport layer and a light-emitting layer, resulting in exciton quenching or triplet-polaron quenching. Thus, improvement of efficiency and lifespan still has been required.

To solve the above problems, the compound represented by formula 1, in which carbazole or fused carbazole is substituted with an amine having strong hole current characteristic, is used as a first host, and the fused cabazole group material having strong electron current characteristic is used as a second host. The combination of said two compounds may be used as a host material of a light-emitting layer to provide an organic electroluminescent device having high efficiency and long lifespan together with low driving voltage.

Generally, external quantum efficiency (Nₑₓₜ) of an organic electroluminescent device means the number of photons emitted outside compared to the number of charges injected, and the definition is as follows:

Nₑₓₜ=Nᵢₙₜ*Nₒᵤₜ=γ*NₑₓØₚ*Nₒᵤₜ

Herein, Nₑₓₜ is the external quantum efficiency, Nᵢₙₜ is the internal quantum efficiency, and Nₒᵤₜ is the emission rate outside the device to the internally produced light. In addition, γ is the combining rate of holes and electrons, Nₑₓ is a producing rate of the excitons, and Øₚ is the PL quantum efficiency.

If the carbazole group material fused with a hetero group, etc. used as the second host is used alone in a light-emitting layer, a charge-balance factor corresponding to γ may be reduced due to relatively fast electron current characteristic. However, in the combination of organic electroluminescent compounds according to the present disclosure, the insufficient hole current characteristic is compensated by an appropriate charge balance through the first host compound and the factor corresponding to γ is improved, which may contribute to an enhancement of the organic electroluminescent device performance. Further, the interfacial characteristic is improved by releasing the excitons extremely formed between the hole transport layer and the light-emitting layer to the light emitting layer/electron transport zone's side. Thus, an organic electroluminescent device of a relatively low driving voltage, excellent luminous efficiency such as current efficiency and power efficiency, and capable of realizing high color purity may be provided.

Hereinafter, a plurality of host materials comprising the organic electroluminescent compounds represented by formulas 1 and 2 will be described in more detail.

The compound of formula 1 can be represented by formula 1-1 or 1-2:

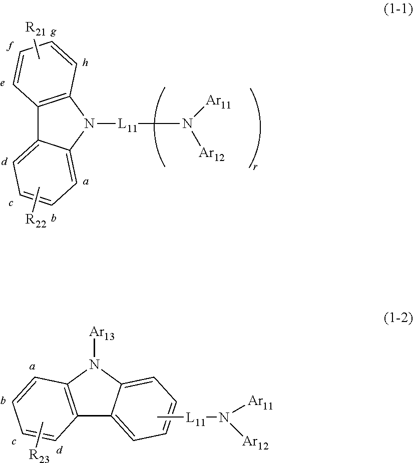

wherein

Ar₁₁ to Ar₁₃ each independently represent a substituted or unsubstituted (C6-C30)aryl; or Ar₁₁ and Ar₁₂ may be linked to each other to form a substituted or unsubstituted (3- to 30-membered) ring;

L₁₁ represents a single bond, or a substituted or unsubstituted (C6-C30)arylene;

if Ar₁₁ or Ar₁₂ represents a substituted or unsubstituted (C6-C30)aryl, and L₁₁ represents a substituted or unsubstituted (C6-C30)arylene, Ar₁₁ or Ar₁₂ and L₁₁ may be linked via a single bond to form a substituted or unsubstituted (3- to 30-membered) ring;

at least one of a and b, b and c, c and d, e and f, f and g, or g and h in formulas 1-1 and 1-2 and two * positions of the following formula 1-a, 1-b, or 1-c may be fused to form a ring:

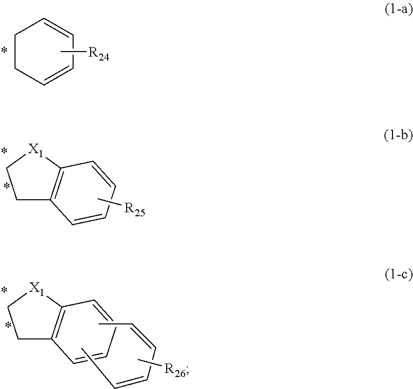

X₁ represents NR₃₁, O, S, or CR₃₂R₃₃;

R₃₁ to R₃₃ each independently represent a substituted or unsubstituted (C1-C30)alkyl, a substituted or unsubstituted (C6-C30)aryl, or a substituted or unsubstituted (3- to 30-membered)heteroaryl;

R₂₁ to R₂₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; and

r represents 1 or 2.

In formula 1, Ar₁ to Ar₄ each independently represent a substituted or unsubstituted (C6-C30)aryl; or Ar₁ and Ar₂, and Ar₃ and Ar₄ may be linked to each other to form a substituted or unsubstituted (3- to 30-membered) ring. In an embodiment of the present disclosure, Ar₁ to Ar₄ each independently represent a substituted or unsubstituted (C6-C25)aryl. In another embodiment of the present disclosure, Ar₁ to Ar₄ each independently represent a (C6-C25)aryl unsubstituted or substituted with a (C1-C6)alkyl, a (5- to 15-membered)heteroaryl, or a tri(C6-C12)arylsilyl. Specifically, Ar₁ to Ar₄ may each independently represent phenyl, naphthyl, biphenyl, terphenyl, naphthyl phenyl, phenanthrenylphenyl, dimethylfluorenyl, diphenylfluorenyl, dimethylbezofluorenyl, phenyl substituted with dibenzofuranyl, phenyl substituted with dibenzothiophenyl, phenyl substituted with triphenylsilyl, etc.

In formula 1, L₁ represents a single bond, or a substituted or unsubstituted (C6-C30)aryl(ene) (if n is 0, L₁ is an aryl, and if n is 1 or more, L₁ is an arylene). In one embodiment of the present disclosure, L₁ represents a substituted or unsubstituted (C6-C25)aryl(ene). In another embodiment of the present disclosure, L₁ represents a (C6-C25)aryl(ene) unsubstituted or substituted with a (C1-C6)alkyl, a (5- to 15-membered)heteroaryl, or a tri(C6-C12)arylsilyl. Specifically, L₁ may represent phenyl(ene), naphthyl(ene), biphenyl(ene), terphenyl(ene), naphthylphenyl(ene), phenylnaphthyl(ene), dimethylfluorenyl(ene), diphenylfluorenyl(ene), phenyl(ene) substituted with dibenzofuranyl, phenyl(ene) substituted with dibenzothiophenyl, phenyl(ene) substituted with triphenylsilyl, etc.

In formula 1, L₂ represents a single bond, or a substituted or unsubstituted (C6-C30)arylene. In one embodiment of the present disclosure, L₂ represents a substituted or unsubstituted (C6-C20)arylene. In another embodiment of the present disclosure, L₂ represents an unsubstituted (C6-C20)arylene. Specifically, L₂ may represent phenylene, biphenylene, terphenylene, etc.

In formula 1, if Ar₁ or Ar₂ represents a substituted or unsubstituted (C6-C30)aryl, and L₁ represents a substituted or unsubstituted (C6-C30)arylene, Ar₁ or Ar₂ and L₁ may be linked via a single bond to form a substituted or unsubstituted (3- to 30-membered) ring. In one embodiment of the present disclosure, if Ar₁ or Ar₂ represents a phenyl and L₁ represents a phenylene, Ar₁ or Ar₂ and L₁ may be linked via a single bond to form a carbazole ring.

In formula 1, if Ar₃ or Ar₄ represents a substituted or unsubstituted (C6-C30)aryl, and L₂ represents a substituted or unsubstituted (C6-C30)arylene, Ar₃ or Ar₄ and L₂ may be linked via a single bond to form a substituted or unsubstituted (3- to 30-membered) ring. In one embodiment of the present disclosure, if Ar₃ or Ar₄ represents an phenyl and L₂ represents an phenylene, Ar₃ or Ar₄ and L₂ may be linked via a single bond to form a carbazole ring.

In formula 1, R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring. In one embodiment of the present disclosure, R₁ and R₂ each independently represent hydrogen, a substituted or unsubstituted (C6-C12)aryl, or a substituted or unsubstituted (5- to 15-membered)heteroaryl; or may be linked to an adjacent substituent to form a substituted or unsubstituted, mono- or polycyclic (5- to 15-membered) ring. In another embodiment of the present disclosure, R₁ and R₂ each independently represent hydrogen, an unsubstituted (C6-C12)aryl, or an unsubstituted (5- to 15-membered)heteroaryl; or may be linked to an adjacent substituent to form a mono- or polycyclic (5- to 15-membered) ring unsubstituted or substituted with a (C1-C6)alkyl or an (C6-C12)aryl. Specifically, R₁ and R₂ may each independently represent hydrogen, phenyl, dibenzofuranyl, dibenzothiophenyl, etc.; or may be linked to an adjacent substituent to form a benzene ring, a dimethylindene ring, a benzofuran ring, a benzothiophene ring, a naphthothiophene ring, a phenylindole ring, or a phenylbenzindole ring.

In formula 1, m and n each independently represent an integer of 0 to 2, with a proviso that at least one of m and n is 1 or more. In one embodiment of the present disclosure, if m is 0, n is 1 or 2, and if m is 1, n is 0.

In formula 1, p and q each independently represent an integer of 1 to 4, in which if p and q represent an integer of 2 or more, each of R₁ and R₂ may be the same or different.

In formula 2, X represents —NR₁₁—, —CR₁₂R₁₃—, —O—, or −S—.

In formula 2, HAr represents a substituted or unsubstituted (3- to 30-membered)heteroaryl. In one embodiment of the present disclosure, HAr represents a substituted or unsubstituted, nitrogen-containing (3- to 30-membered)heteroaryl. In another embodiment of the present disclosure, HAr represents a substituted or unsubstituted, nitrogen-containing (5- to 20-membered)heteroaryl. In a further embodiment of the present disclosure, HAr represents a nitrogen-containing (5- to 20-membered)heteroaryl unsubstituted or substituted with a (C6-C20)aryl, a (C1-C6)alkyl(C6-C20)aryl, or (C6-C12)aryl(5- to 15-membered)heteroaryl. Specifically, HAr represents a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, a substituted or unsubstituted pyridazinyl, a substituted or unsubstituted benzoimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted quinoxalinyl, a substituted or unsubstituted naphthyridyl, a substituted or unsubstituted phenanthridinyl, a substituted or unsubstituted benzofuranopyrimidinyl, a substituted or unsubstituted benzothiophenopyrimidinyl, a substituted or unsubstituted benzoquinazolinyl, a substituted or unsubstituted benzoquinoxalinyl, a substituted or unsubstituted pyridoquinoxalinyl, a substituted or unsubstituted pyrazinoquinoxalinyl, a substituted or unsubstituted dibenzoquinoxalinyl, or a substituted or unsubstituted pyridobenzoquinoxalinyl, in which these may be further substituted with at least one substituent selected from the group consisting of phenyl, naphthyl, biphenyl, naphthylphenyl, methylphenyl, dimethylfluorenyl, dibenzofuranyl, di benzothiophenyl, and phenylcarbazolyl.

In formula 2, L represents a single bond, a substituted or unsubstituted (C6-C30)arylene, or a substituted or unsubstituted (3- to 30-membered)heteroarylene. In one embodiment of the present disclosure, L represents a single bond, or a substituted or unsubstituted (C6-C12)arylene. In another embodiment of the present disclosure, L represents a single bond, or an unsubstituted (C6-C12)arylene. Specifically, L may represent a single bond, phenylene, or naphthylene.

In formula 2, R₁₁ to R₁₃ each independently represent a substituted or unsubstituted (C1-C30)alkyl, a substituted or unsubstituted (C6-C30)aryl, or a substituted or unsubstituted (3- to 30-membered)heteroaryl. In one embodiment of the present disclosure, R₁₁ represents a substituted or unsubstituted (C6-C12)aryl, and R₁₂ and R₁₃ each independently represent a substituted or unsubstituted (C1-C6)alkyl. In another embodiment of the present disclosure, R₁₁ represents an unsubstituted (C6-C12)aryl, and R₁₂ and R₁₃ each independently represent an unsubstituted (C1-C6)alkyl. Specifically, R₁₁ may represent phenyl, and R₁₂ and R₁₃ may each independently represent methyl.

In formula 2, R₃ to R₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring. In one embodiment of the present disclosure, R₃ to R₅ each independently represent hydrogen.

In formula 2, a, b, and c each independently represent an integer of 1 to 4, in which if a, b, and c represent an integer of 2 or more, each of R₃, R₄, and R₅ may be the same or different.

In the formulas of the present disclosure, if a substituent is linked to an adjacent substituent to form a substituted or unsubstituted (3- to 30-membered) ring, the ring may be a mono- or polycyclic, alicyclic or aromatic ring, or the combination thereof, in which the formed ring may contain at least one heteroatom selected from nitrogen, oxygen, and sulfur.

In the formulas of the present disclosure, the heteroaryl(ene) may each independently contain at least one heteroatom selected from B, N, O, S, Si, and P. In addition, the heteroatom may be substituted with at least one substituent selected from the group consisting of deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Herein, “(C1-C30)alkyl” is meant to be a linear or branched alkyl having 1 to 30 carbon atoms constituting the chain, in which the number of carbon atoms is preferably 1 to 20, more preferably 1 to 10, and includes methyl, ethyl, n-propyl, isopropyl, n-butyl, isobutyl, tert-butyl, etc. “(C2-C30)alkenyl” is meant to be a linear or branched alkenyl having 2 to 30 carbon atoms constituting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is meant to be a linear or branched alkynyl having 2 to 30 carbon atoms constituting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7, and includes cyclopropyl, cyclobutyl, cyclopentyl, cyclohexyl, etc. “(3- to 7-membered)heterocycloalkyl” is meant to be a cycloalkyl having 3 to 7, preferably 5 to 7, ring backbone atoms, including at least one heteroatom selected from the group consisting of B, N, O, S, P(═O), Si, and P, and preferably O, S, and N, and includes tetrahydrofuran, pyrrolidine, thiolan, tetrahydropyran, etc. “(C6-C30)aryl(ene)” is meant to be a monocyclic or fused ring radical derived from an aromatic hydrocarbon having 6 to 30 ring backbone carbon atoms, in which the number of the ring backbone carbon atoms is preferably 6 to 25, and more preferably 6 to 20, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, phenanthrenylphenyl, anthracenyl, indenyl, triphenylenyl, pyrenyl, tetracenyl, perylenyl, chrysenyl, naphthacenyl, fluoranthenyl, etc. “(3- to 30-membered)heteroaryl(ene)” is meant to be an aryl group having 3 to 30 ring backbone atoms, including at least one, preferably 1 to 4 heteroatoms selected from the group consisting of B, N, O, S, Si, and P. The above heteroaryl may be a monocyclic ring, or a fused ring condensed with at least one benzene ring; may be partially saturated; may be one formed by linking at least one heteroaryl or aryl group to a heteroaryl group via a single bond. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, naphthothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, quinoxalinyl, naphthyridyl, carbazolyl, phenoxazinyl, phenanthridinyl, benzodioxolyl, benzofuranopyrimidinyl, benzothiophenopyrimidinyl, benzoquinazolinyl, benzoquinoxalinyl, pyridoquinoxalinyl, pyrazinoquinoxalinyl, dibenzoquinoxalinyl, and pyridobenzoquinoxalinyl. “Nitrogen-containing (5- to 30-membered)heteroaryl” is meant to be an aryl group having 5 to 30 ring backbone atoms, including at least one heteroatom of N, in which the number of the ring backbone atoms is preferably 5 to 20, and more preferably 5 to 15, and the number of heteroatoms is preferably 1 to 4. The above nitrogen-containing heteroaryl may be a monocyclic ring, or a fused ring condensed with at least one benzene ring; may be partially saturated; may be one formed by linking at least one heteroaryl or aryl group to a heteroaryl group via a single bond. The above nitrogen-containing heteroaryl may include a monocyclic ring-type heteroaryl such as pyrrolyl, imidazolyl, pyrazolyl, triazinyl, tetrazinyl, triazolyl, tetrazolyl, pyridyl, pyrazinyl, pyrimidinyl, and pyridazinyl, and a fused ring-type heteroaryl such as benzoimidazolyl, isoindolyl, indolyl, indazolyl, benzothiadiazolyl, quinolyl, isoquinolyl, cinnolinyl, quinazolinyl, quinoxalinyl, naphthyridyl, carbazolyl, benzocarbazolyl, dibenzocarbazolyl, phenanthridinyl, benzofuranopyrimidinyl, benzothiophenopyrimidinyl, benzoquinazolinyl, benzoquinoxalinyl, pyridoquinoxalinyl, pyrazinoquinoxalinyl, dibenzoquinoxalinyl, and pyridobenzoquinoxalinyl. “Halogen” includes F, Cl, Br, and I.

Herein, “substituted” in the expression “substituted or unsubstituted” means that a hydrogen atom in a certain functional group is replaced with another atom or functional group, i.e., a substituent. In formulas 1, 2, 1-1, and 1-2, the substituents of the substituted alkyl, the substituted alkoxy, the substituted cycloalkyl, the substituted aryl(ene), the substituted heteroaryl(ene), the substituted trialkylsilyl, the substituted triarylsilyl, the substituted dialkylarylsilyl, the substituted alkyldiarylsilyl, the substituted mono- or di-alkylamino, the substituted mono- or di-arylamino, the substituted alkylarylamino, and the substituted ring in Ar₁ to Ar₄, Ar₁₁ to Ar₁₃, HAr, R₁ to R₅, R₁₁ to R₁₃, R₂₁ to R₂₆, R₃₁ to R₃₃, L, L₁, L₂, and L₁₁ each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (3- to 7-membered)heterocycloalkyl, a (C6-C30)aryloxy, a (C6-C30)arylthio, a (3- to 30-membered)heteroaryl unsubstituted or substituted with a (C6-C30)aryl, a (C6-C30)aryl unsubstituted or substituted with a (3- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl; preferably at least one selected from the group consisting of a (C1-C6)alkyl, a (5- to 15-membered)heteroaryl unsubstituted or substituted with a (C6-C12)aryl, a (C6-C20)aryl, a tri(C6-C12)arylsilyl, and a (C1-C6)alkyl(C6-C20)aryl. Specifically, said substituent may be methyl, phenyl, biphenyl, phenanthrenyl, naphthylphenyl, methylphenyl, dimethylfluorenyl, triphenylsilyl, dibenzofuranyl, dibenzothiophenyl, or phenylcarbazolyl.

The compound represented by formula 1 includes the following compounds, but is not limited thereto:

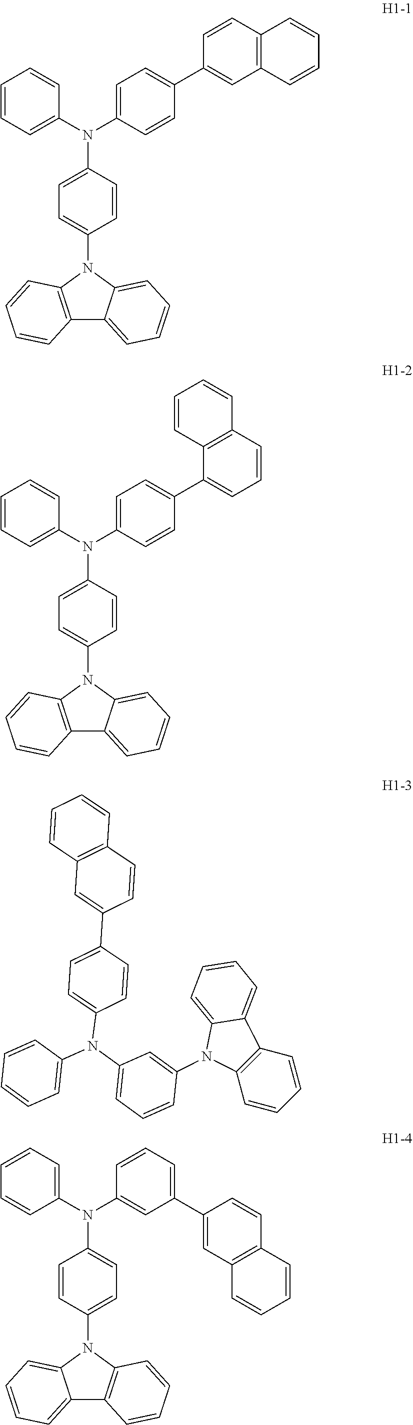

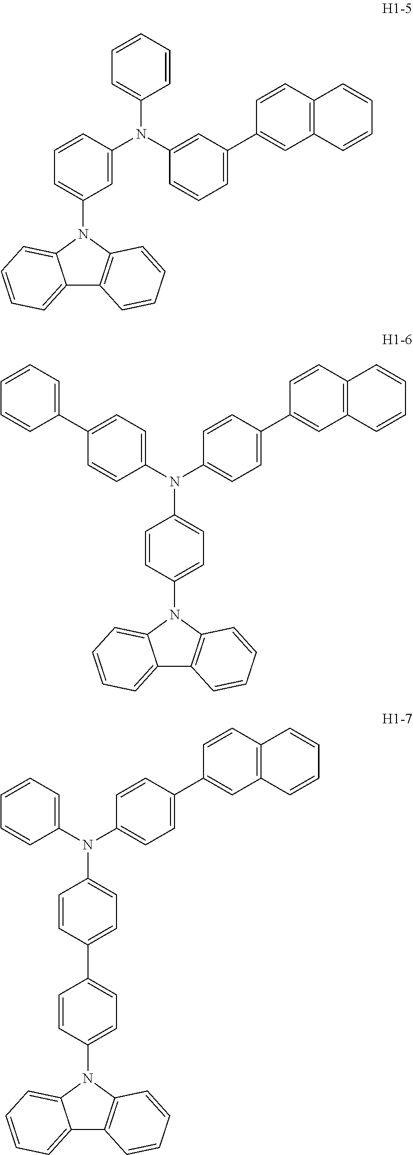

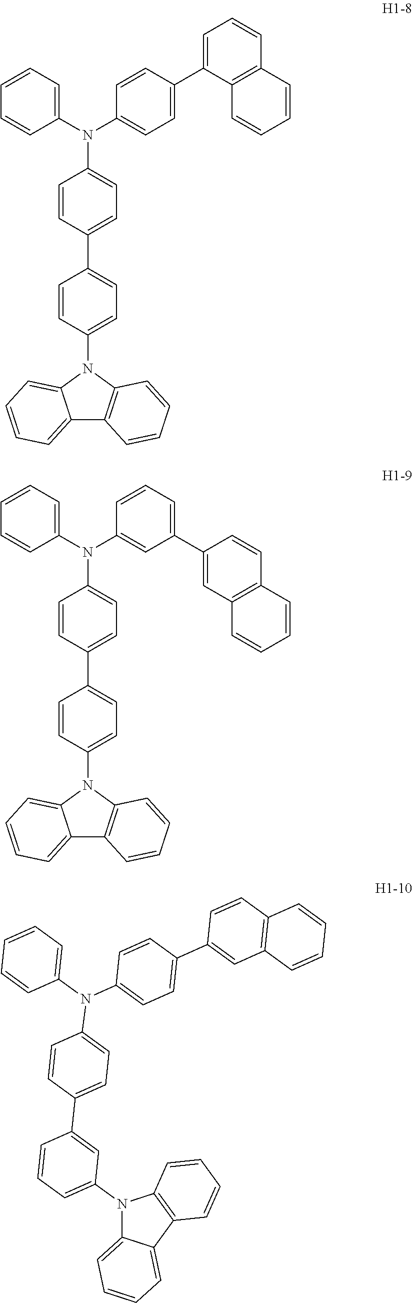

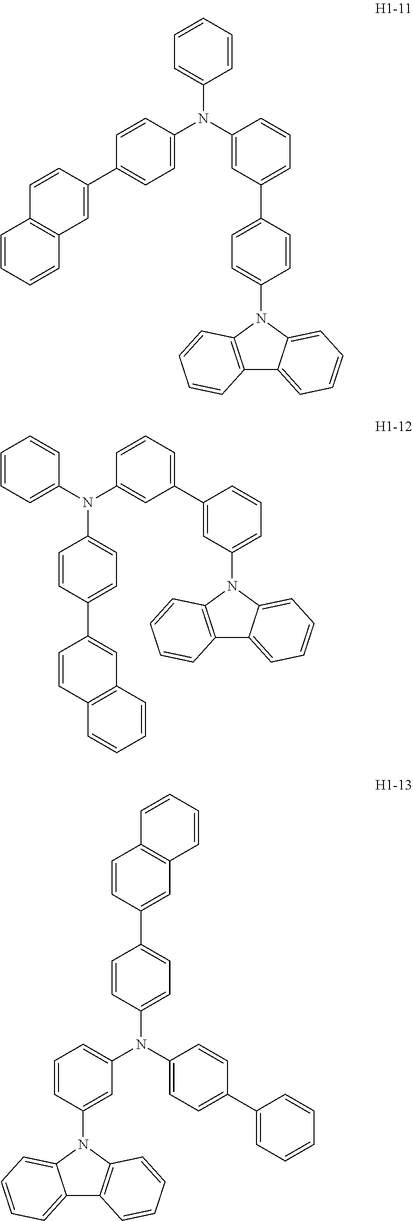 [SMILES: c1ccc(N(c2ccc(-c3ccc4ccccc4c3)cc2)c2cccc(-c3ccc(-n4c5ccccc5c5ccccc54)cc3)c2)cc1 ; c1ccc(N(c2ccc(-c3ccc4ccccc4c3)cc2)c2cccc(-c3cccc(-n4c5ccccc5c5ccccc54)c3)c2)cc1 ; c1ccc(-c2ccc(N(c3ccc(-c4ccc5ccccc5c4)cc3)c3cccc(-n4c5ccccc5c5ccccc54)c3)cc2)cc1]

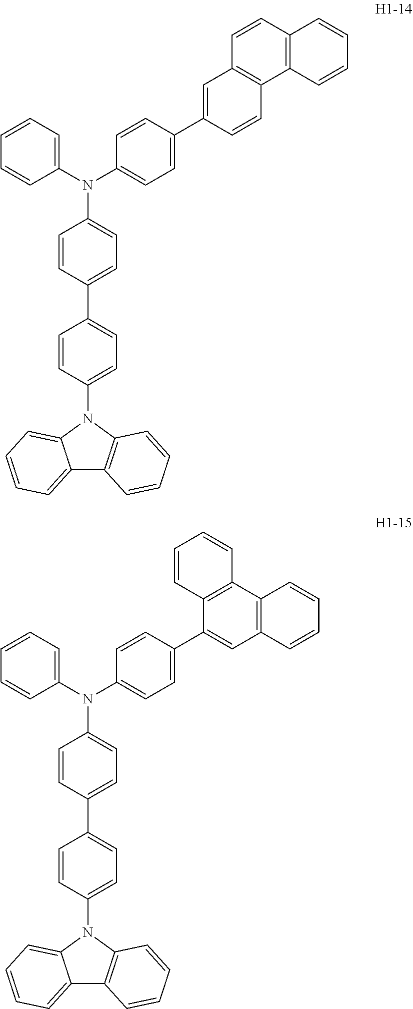 [SMILES: c1ccc(N(c2ccc(-c3ccc(-n4c5ccccc5c5ccccc54)cc3)cc2)c2ccc(-c3ccc4c(ccc5ccccc54)c3)cc2)cc1 ; c1ccc(N(c2ccc(-c3ccc(-n4c5ccccc5c5ccccc54)cc3)cc2)c2ccc(-c3cc4ccccc4c4ccccc34)cc2)cc1]

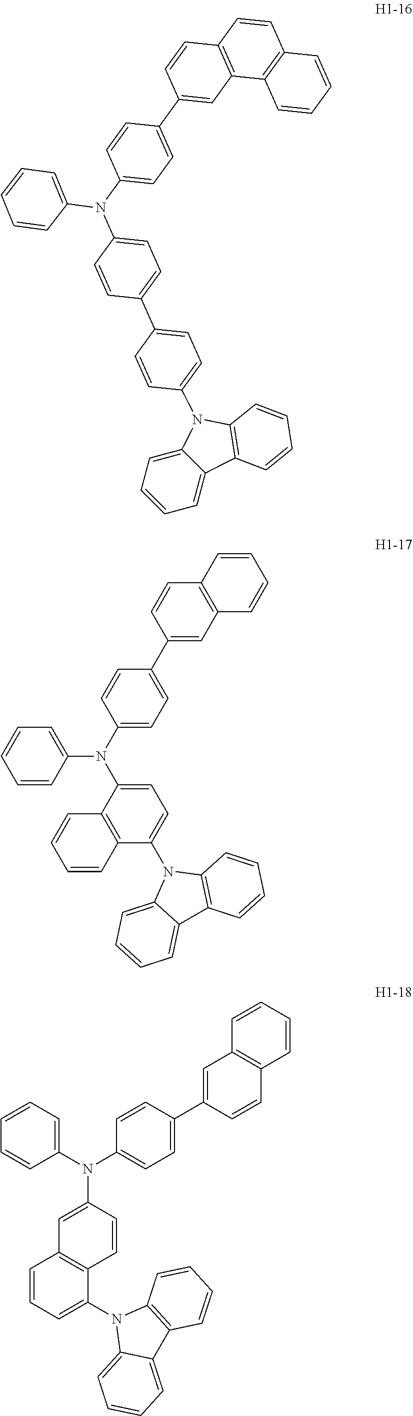 [SMILES: c1ccc(N(c2ccc(-c3ccc(-n4c5ccccc5c5ccccc54)cc3)cc2)c2ccc(-c3ccc4ccc5ccccc5c4c3)cc2)cc1 ; c1ccc(N(c2ccc(-c3ccc4ccccc4c3)cc2)c2ccc(-n3c4ccccc4c4ccccc43)c3ccccc23)cc1 ; c1ccc(N(c2ccc(-c3ccc4ccccc4c3)cc2)c2ccc3c(-n4c5ccccc5c5ccccc54)cccc3c2)cc1]

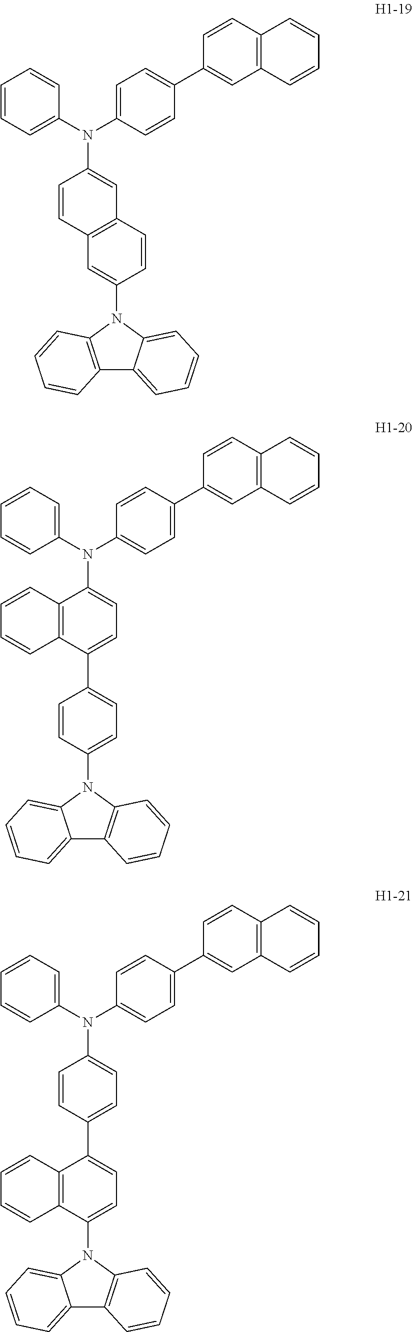 [SMILES: c1ccc(N(c2ccc(-c3ccc4ccccc4c3)cc2)c2ccc3cc(-n4c5ccccc5c5ccccc54)ccc3c2)cc1 ; c1ccc(N(c2ccc(-c3ccc4ccccc4c3)cc2)c2ccc(-c3ccc(-n4c5ccccc5c5ccccc54)cc3)c3ccccc23)cc1 ; c1ccc(N(c2ccc(-c3ccc4ccccc4c3)cc2)c2ccc(-c3ccc(-n4c5ccccc5c5ccccc54)c4ccccc34)cc2)cc1]

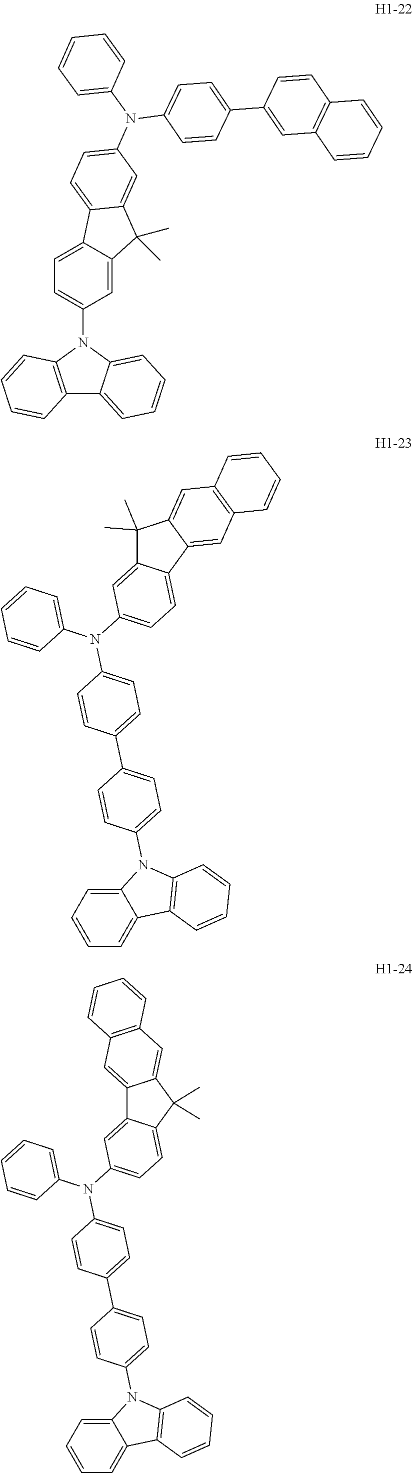

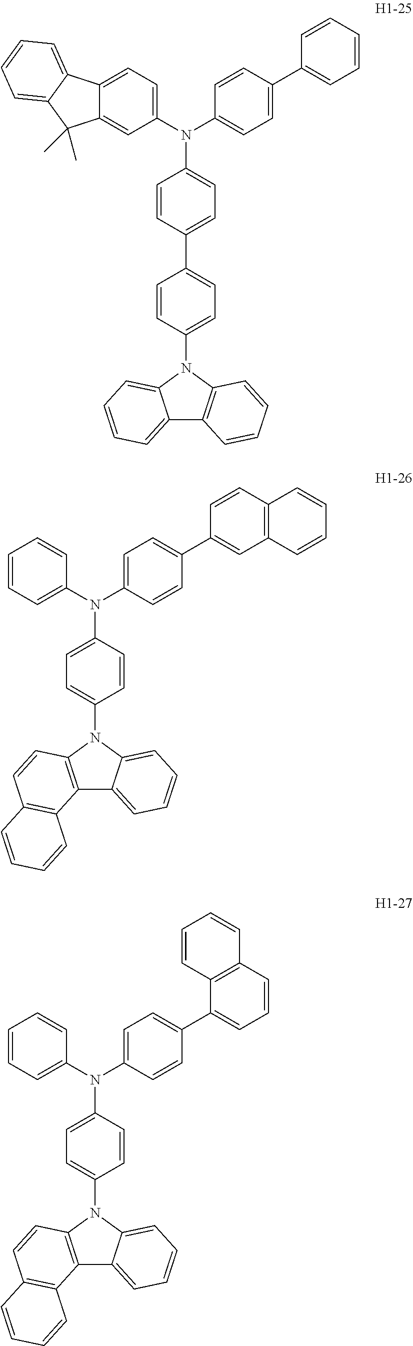

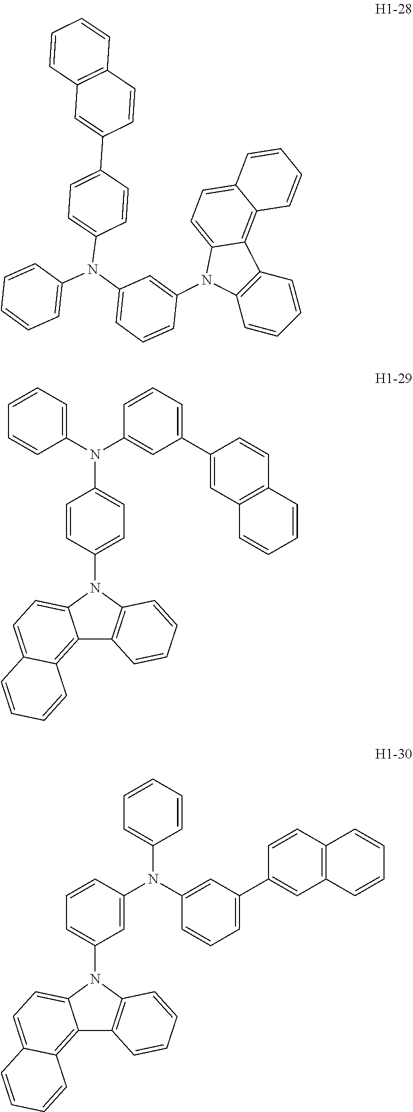 [SMILES: c1ccc(N(c2ccc(-c3ccc4ccccc4c3)cc2)c2cccc(-n3c4ccccc4c4c5ccccc5ccc43)c2)cc1 ; c1ccc(N(c2ccc(-n3c4ccccc4c4c5ccccc5ccc43)cc2)c2cccc(-c3ccc4ccccc4c3)c2)cc1 ; c1ccc(N(c2cccc(-c3ccc4ccccc4c3)c2)c2cccc(-n3c4ccccc4c4c5ccccc5ccc43)c2)cc1]

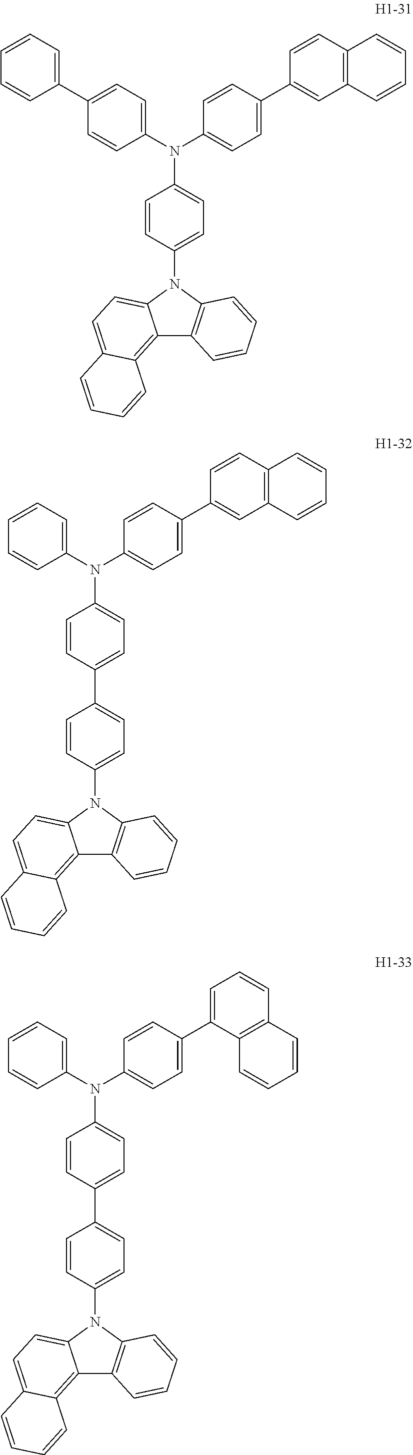 [SMILES: c1ccc(-c2ccc(N(c3ccc(-c4ccc5ccccc5c4)cc3)c3ccc(-n4c5ccccc5c5c6ccccc6ccc54)cc3)cc2)cc1 ; c1ccc(N(c2ccc(-c3ccc(-n4c5ccccc5c5c6ccccc6ccc54)cc3)cc2)c2ccc(-c3ccc4ccccc4c3)cc2)cc1 ; c1ccc(N(c2ccc(-c3ccc(-n4c5ccccc5c5c6ccccc6ccc54)cc3)cc2)c2ccc(-c3cccc4ccccc34)cc2)cc1]

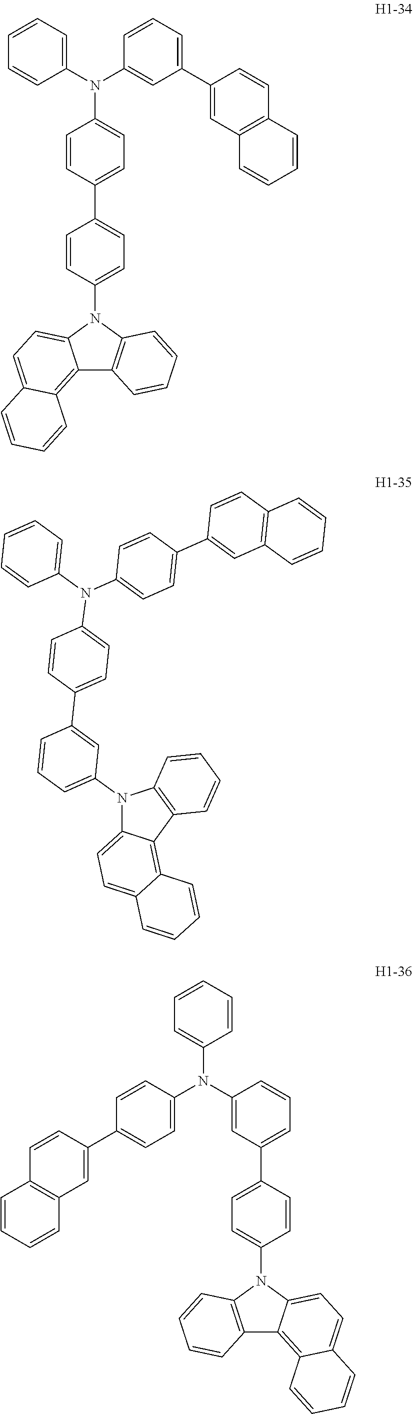 [SMILES: c1ccc(N(c2ccc(-c3ccc(-n4c5ccccc5c5c6ccccc6ccc54)cc3)cc2)c2cccc(-c3ccc4ccccc4c3)c2)cc1 ; c1ccc(N(c2ccc(-c3cccc(-n4c5ccccc5c5c6ccccc6ccc54)c3)cc2)c2ccc(-c3ccc4ccccc4c3)cc2)cc1 ; c1ccc(N(c2ccc(-c3ccc4ccccc4c3)cc2)c2cccc(-c3ccc(-n4c5ccccc5c5c6ccccc6ccc54)cc3)c2)cc1]

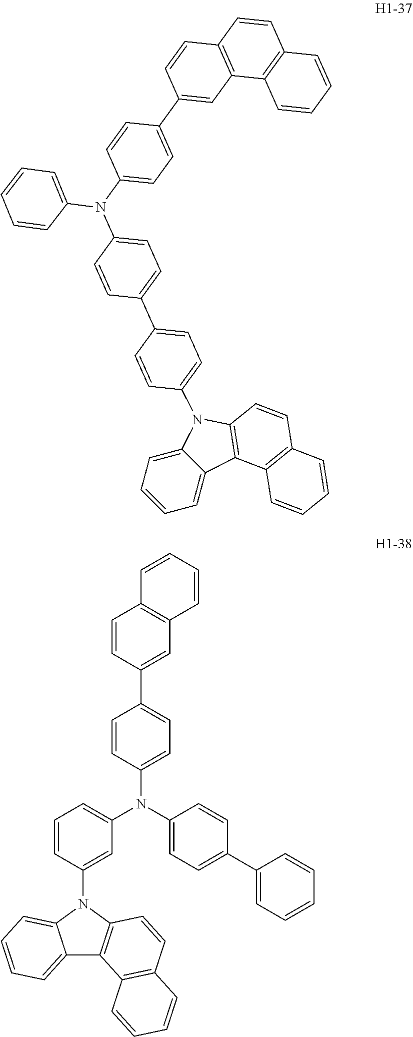

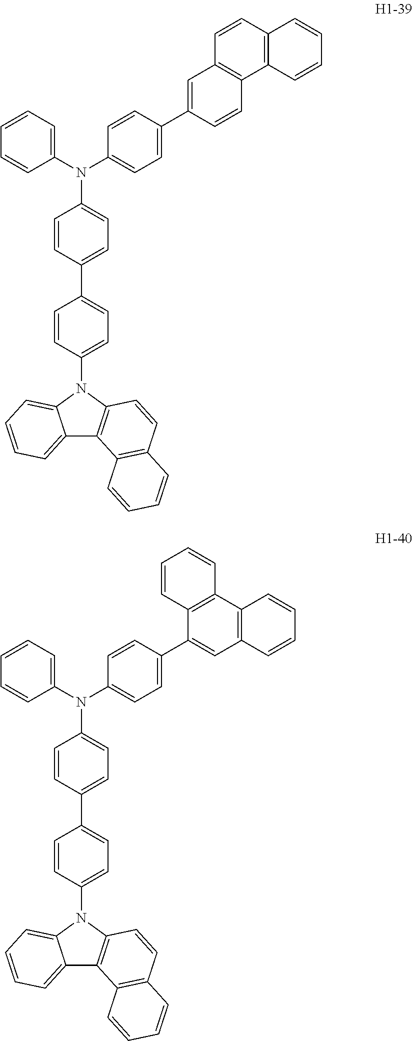 [SMILES: c1ccc(N(c2ccc(-c3ccc(-n4c5ccccc5c5c6ccccc6ccc54)cc3)cc2)c2ccc(-c3ccc4c(ccc5ccccc54)c3)cc2)cc1 ; c1ccc(N(c2ccc(-c3ccc(-n4c5ccccc5c5c6ccccc6ccc54)cc3)cc2)c2ccc(-c3cc4ccccc4c4ccccc34)cc2)cc1]

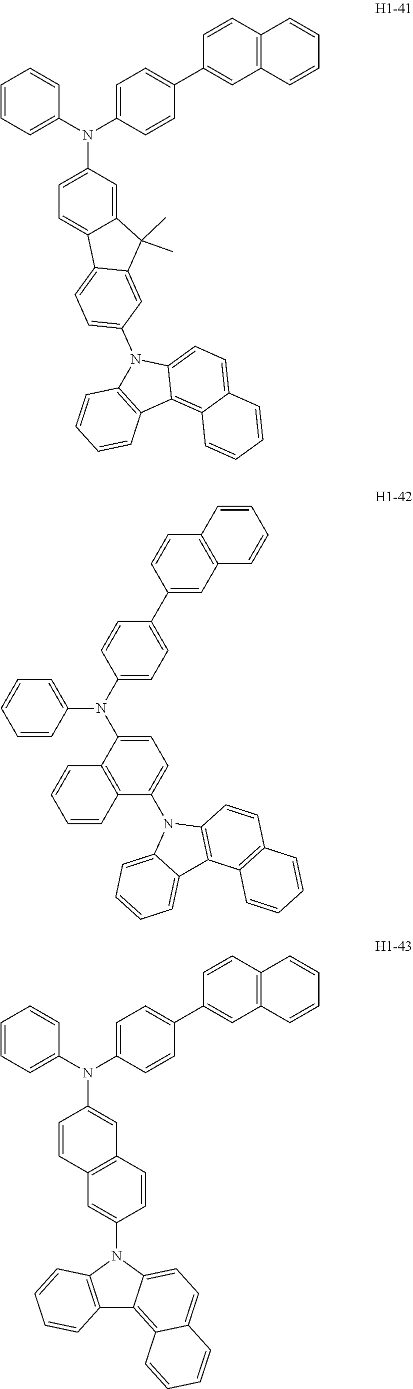

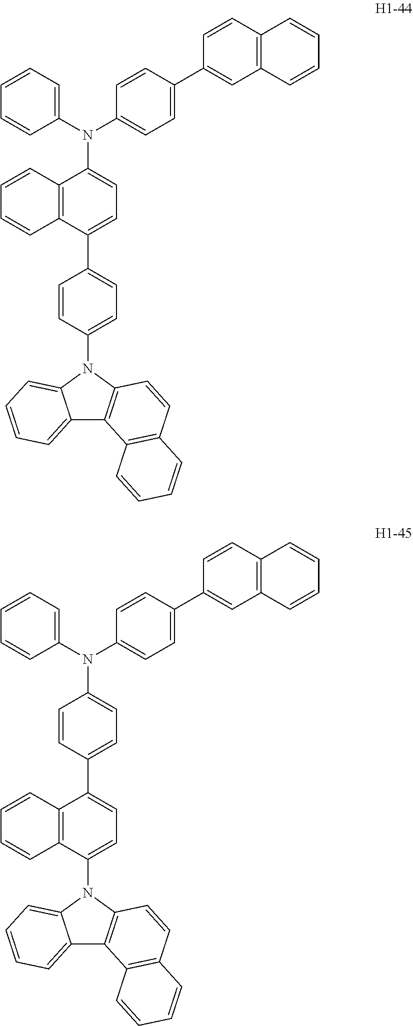 [SMILES: c1ccc(N(c2ccc(-c3ccc4ccccc4c3)cc2)c2ccc(-c3ccc(-n4c5ccccc5c5c6ccccc6ccc54)cc3)c3ccccc23)cc1 ; c1ccc(N(c2ccc(-c3ccc4ccccc4c3)cc2)c2ccc(-c3ccc(-n4c5ccccc5c5c6ccccc6ccc54)c4ccccc34)cc2)cc1]

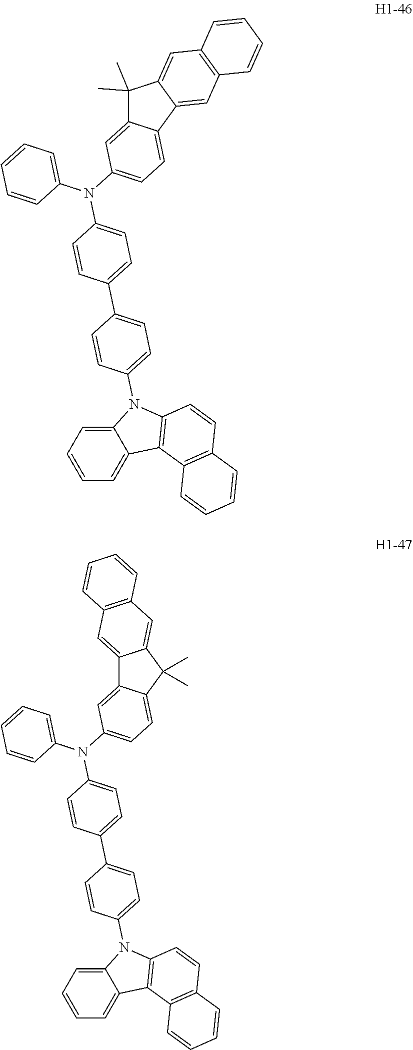 [SMILES: CC1(C)c2cc(N(c3ccccc3)c3ccc(-c4ccc(-n5c6ccccc6c6c7ccccc7ccc65)cc4)cc3)ccc2-c2cc3ccccc3cc21 ; CC1(C)c2ccc(N(c3ccccc3)c3ccc(-c4ccc(-n5c6ccccc6c6c7ccccc7ccc65)cc4)cc3)cc2-c2cc3ccccc3cc21]

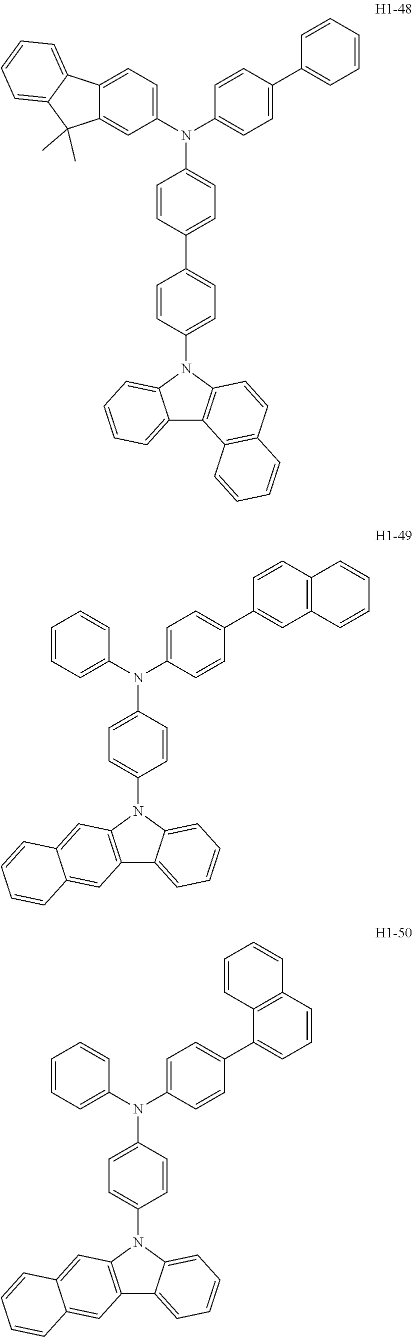 [SMILES: CC1(C)c2ccccc2-c2ccc(N(c3ccc(-c4ccccc4)cc3)c3ccc(-c4ccc(-n5c6ccccc6c6c7ccccc7ccc65)cc4)cc3)cc21 ; c1ccc(N(c2ccc(-c3ccc4ccccc4c3)cc2)c2ccc(-n3c4ccccc4c4cc5ccccc5cc43)cc2)cc1 ; c1ccc(N(c2ccc(-c3cccc4ccccc34)cc2)c2ccc(-n3c4ccccc4c4cc5ccccc5cc43)cc2)cc1]

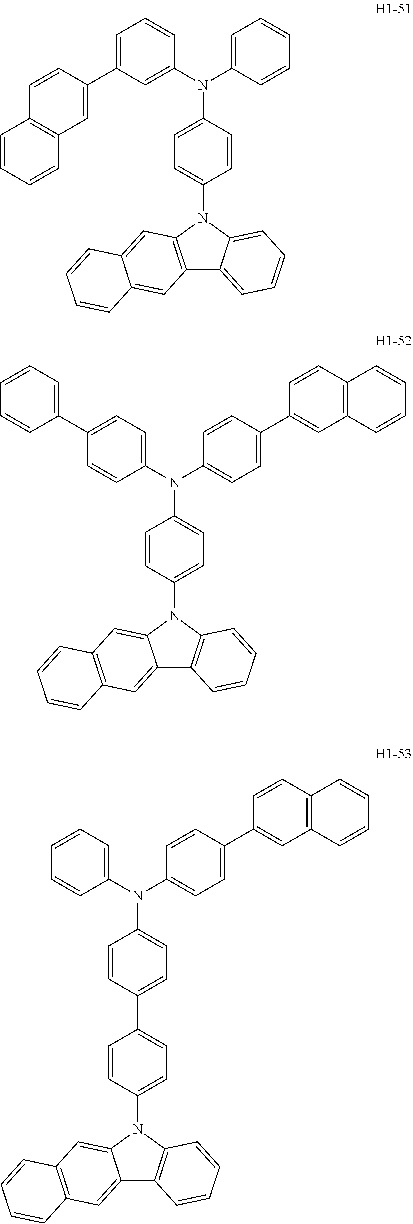 [SMILES: c1ccc(N(c2ccc(-n3c4ccccc4c4cc5ccccc5cc43)cc2)c2cccc(-c3ccc4ccccc4c3)c2)cc1 ; c1ccc(-c2ccc(N(c3ccc(-c4ccc5ccccc5c4)cc3)c3ccc(-n4c5ccccc5c5cc6ccccc6cc54)cc3)cc2)cc1 ; c1ccc(N(c2ccc(-c3ccc(-n4c5ccccc5c5cc6ccccc6cc54)cc3)cc2)c2ccc(-c3ccc4ccccc4c3)cc2)cc1]

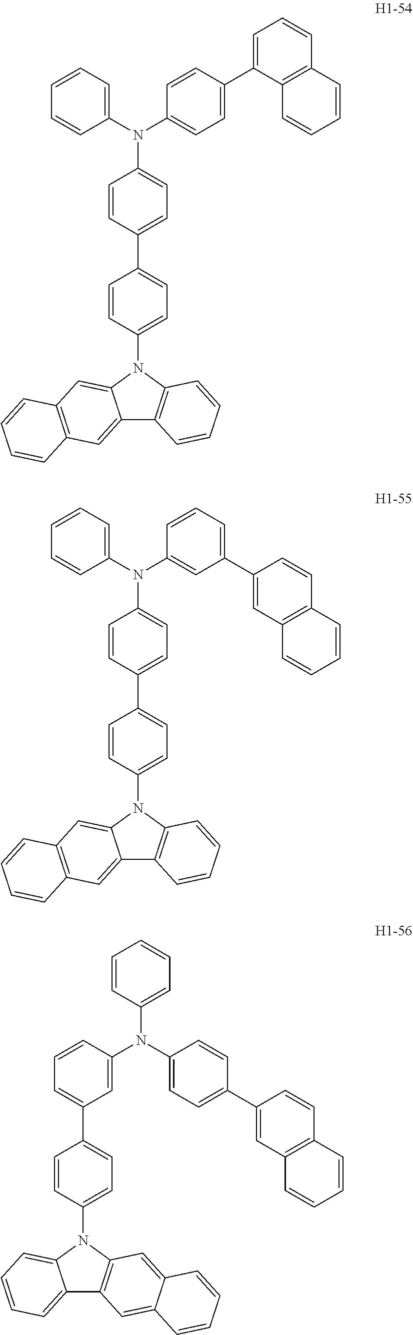 [SMILES: c1ccc(N(c2ccc(-c3ccc(-n4c5ccccc5c5cc6ccccc6cc54)cc3)cc2)c2ccc(-c3cccc4ccccc34)cc2)cc1 ; c1ccc(N(c2ccc(-c3ccc(-n4c5ccccc5c5cc6ccccc6cc54)cc3)cc2)c2cccc(-c3ccc4ccccc4c3)c2)cc1 ; c1ccc(N(c2ccc(-c3ccc4ccccc4c3)cc2)c2cccc(-c3ccc(-n4c5ccccc5c5cc6ccccc6cc54)cc3)c2)cc1]

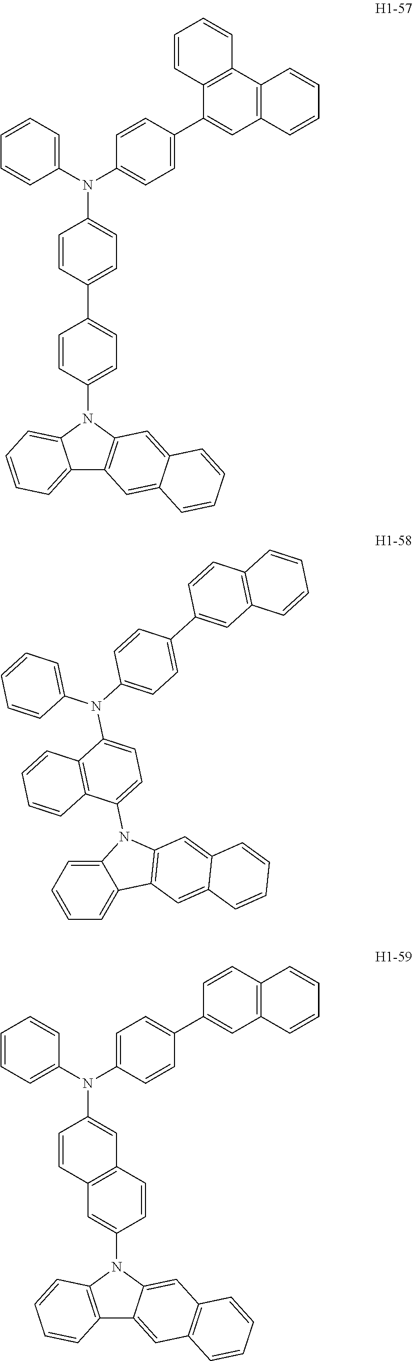 [SMILES: c1ccc(N(c2ccc(-c3ccc(-n4c5ccccc5c5cc6ccccc6cc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccccc4c4cc5ccccc5cc43)c3ccccc23)cc1 ; c1ccc(N(c2ccc(-c3ccc4ccccc4c3)cc2)c2ccc3cc(-n4c5ccccc5c5cc6ccccc6cc54)ccc3c2)cc1]

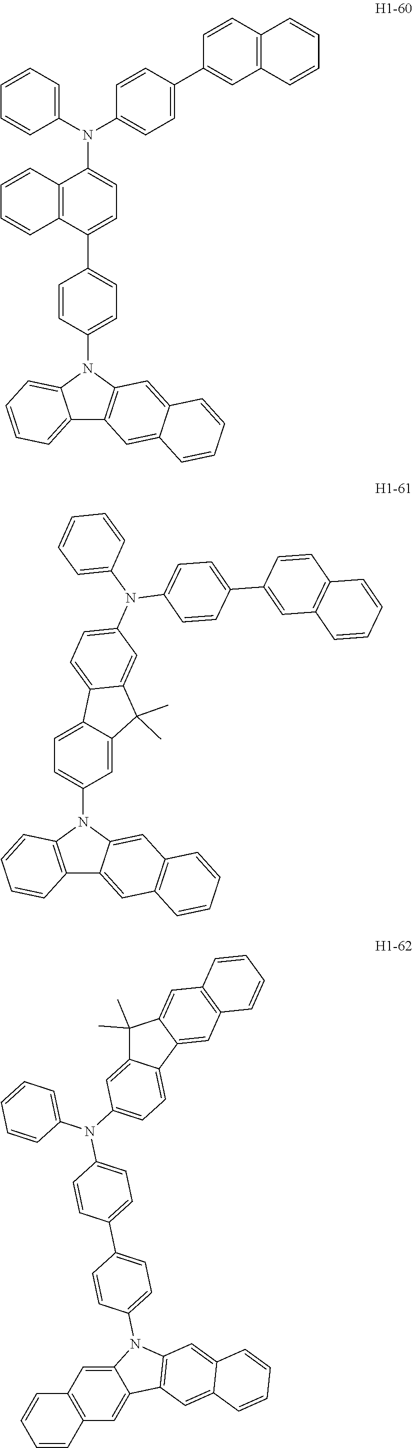 [SMILES: c1ccc(N(c2ccc(-c3ccc4ccccc4c3)cc2)c2ccc(-c3ccc(-n4c5ccccc5c5cc6ccccc6cc54)cc3)c3ccccc23)cc1 ; CC1(C)c2cc(N(c3ccccc3)c3ccc(-c4ccc5ccccc5c4)cc3)ccc2-c2ccc(-n3c4ccccc4c4cc5ccccc5cc43)cc21 ; CC1(C)c2cc(N(c3ccccc3)c3ccc(-c4ccc(-n5c6cc7ccccc7cc6c6cc7ccccc7cc65)cc4)cc3)ccc2-c2cc3ccccc3cc21]

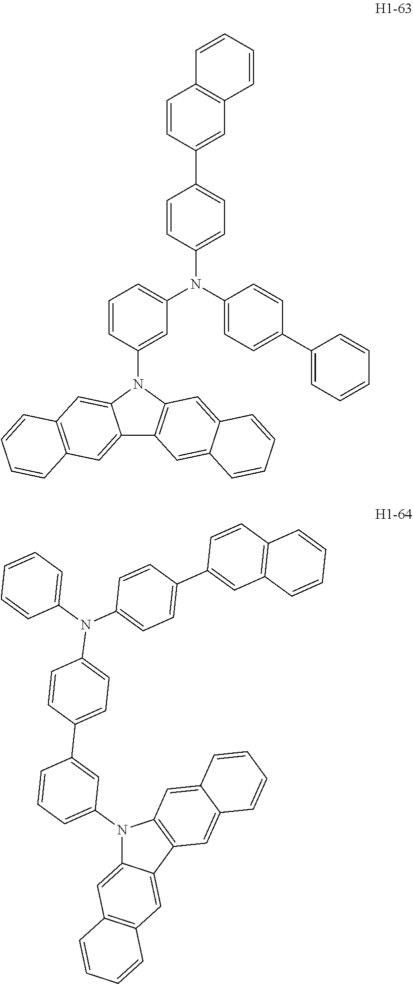 [SMILES: c1ccc(-c2ccc(N(c3ccc(-c4ccc5ccccc5c4)cc3)c3cccc(-n4c5cc6ccccc6cc5c5cc6ccccc6cc54)c3)cc2)cc1 ; c1ccc(N(c2ccc(-c3cccc(-n4c5cc6ccccc6cc5c5cc6ccccc6cc54)c3)cc2)c2ccc(-c3ccc4ccccc4c3)cc2)cc1]

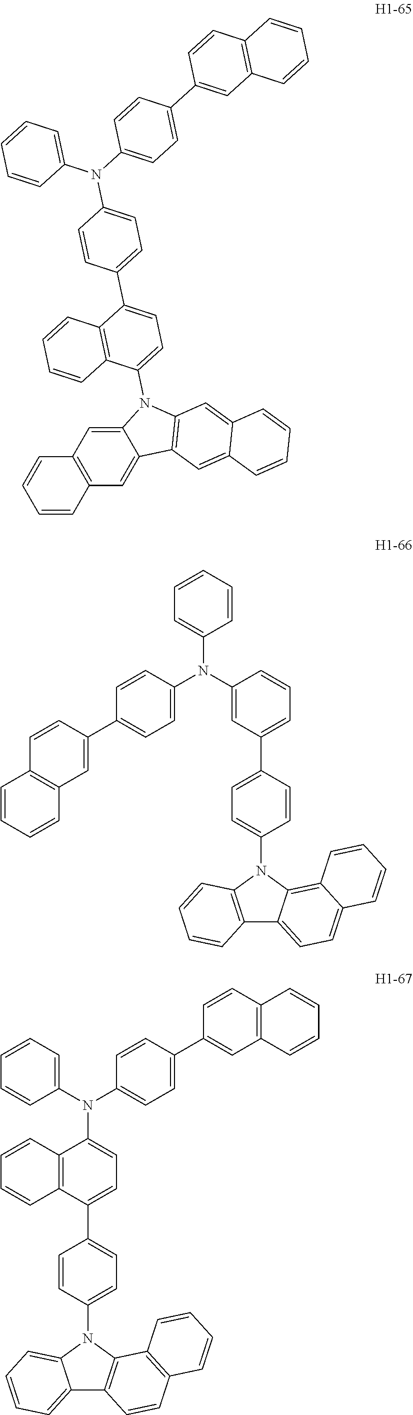

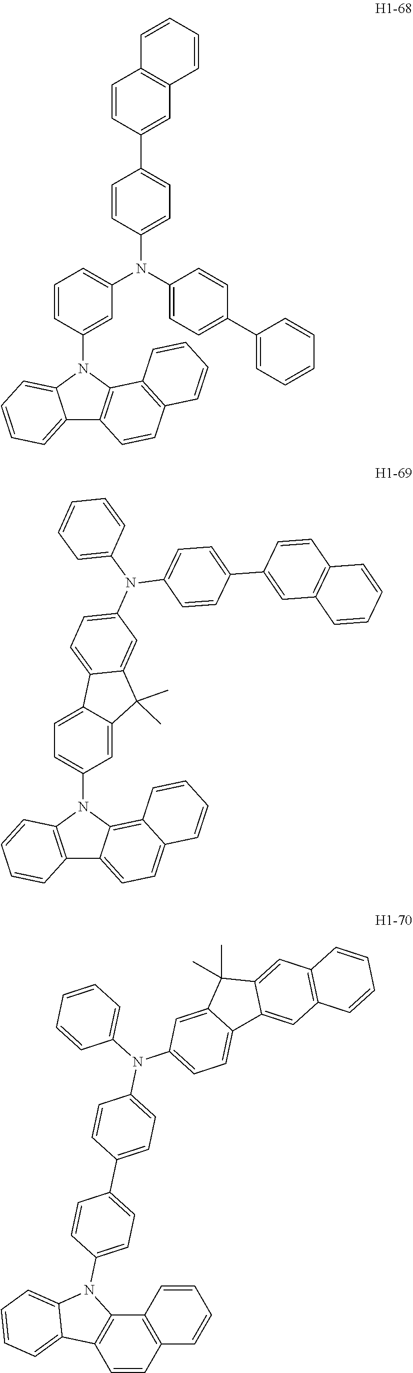 [SMILES: c1ccc(-c2ccc(N(c3ccc(-c4ccc5ccccc5c4)cc3)c3cccc(-n4c5ccccc5c5ccc6ccccc6c54)c3)cc2)cc1 ; CC1(C)c2cc(N(c3ccccc3)c3ccc(-c4ccc5ccccc5c4)cc3)ccc2-c2ccc(-n3c4ccccc4c4ccc5ccccc5c43)cc21 ; CC1(C)c2cc(N(c3ccccc3)c3ccc(-c4ccc(-n5c6ccccc6c6ccc7ccccc7c65)cc4)cc3)ccc2-c2cc3ccccc3cc21]

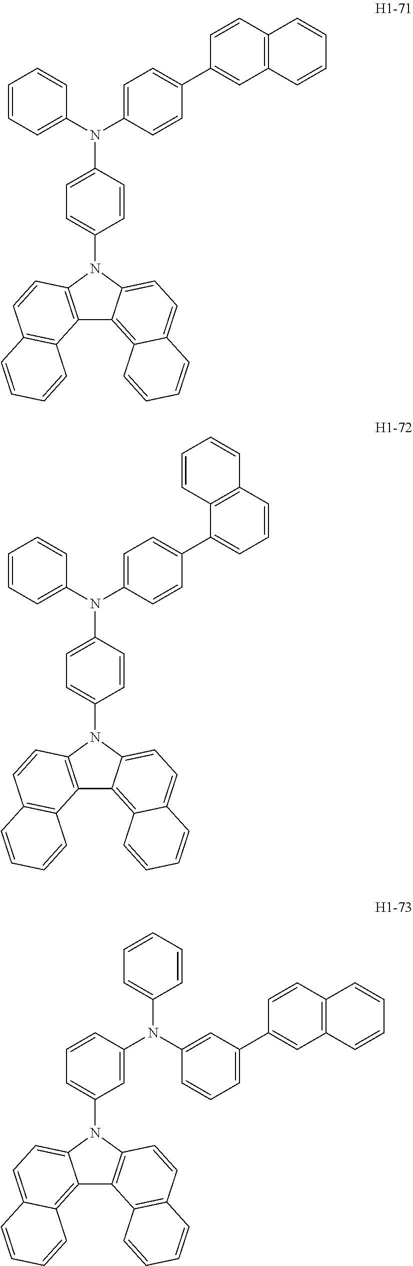 [SMILES: c1ccc(N(c2ccc(-c3ccc4ccccc4c3)cc2)c2ccc(-n3c4ccc5ccccc5c4c4c5ccccc5ccc43)cc2)cc1 ; c1ccc(N(c2ccc(-c3cccc4ccccc34)cc2)c2ccc(-n3c4ccc5ccccc5c4c4c5ccccc5ccc43)cc2)cc1 ; c1ccc(N(c2cccc(-c3ccc4ccccc4c3)c2)c2cccc(-n3c4ccc5ccccc5c4c4c5ccccc5ccc43)c2)cc1]

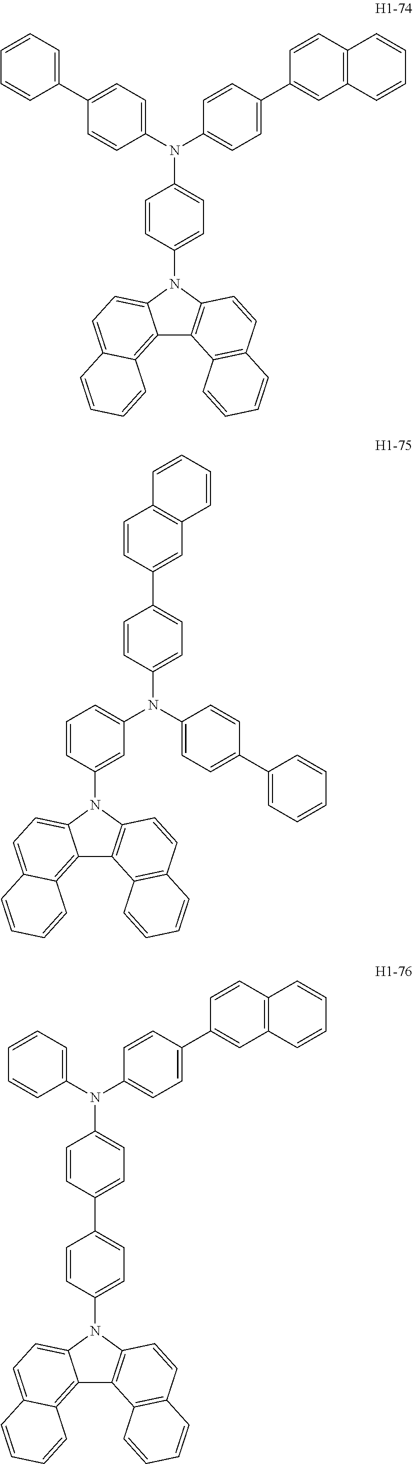 [SMILES: c1ccc(-c2ccc(N(c3ccc(-c4ccc5ccccc5c4)cc3)c3ccc(-n4c5ccc6ccccc6c5c5c6ccccc6ccc54)cc3)cc2)cc1 ; c1ccc(-c2ccc(N(c3ccc(-c4ccc5ccccc5c4)cc3)c3cccc(-n4c5ccc6ccccc6c5c5c6ccccc6ccc54)c3)cc2)cc1 ; c1ccc(N(c2ccc(-c3ccc(-n4c5ccc6ccccc6c5c5c6ccccc6ccc54)cc3)cc2)c2ccc(-c3ccc4ccccc4c3)cc2)cc1]

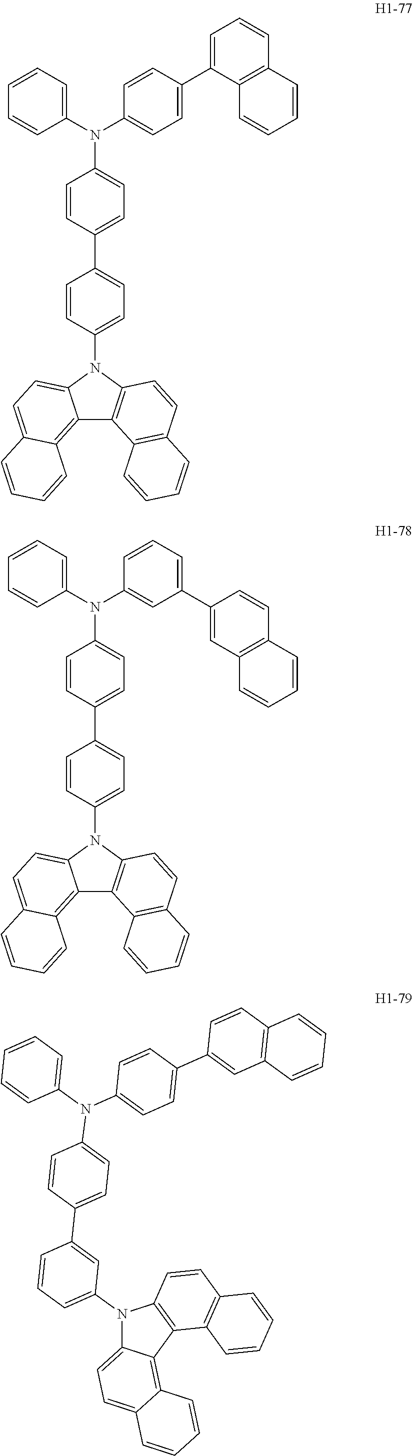

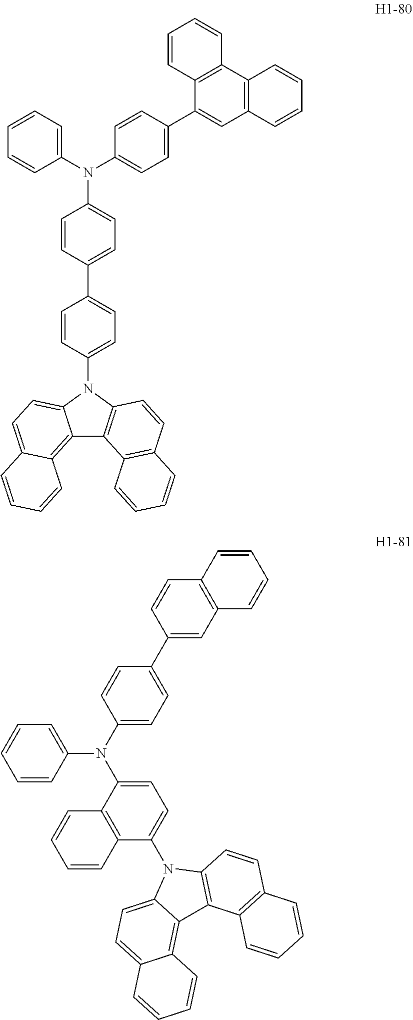 [SMILES: c1ccc(N(c2ccc(-c3ccc(-n4c5ccc6ccccc6c5c5c6ccccc6ccc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccc5ccccc5c4c4c5ccccc5ccc43)c3ccccc23)cc1]

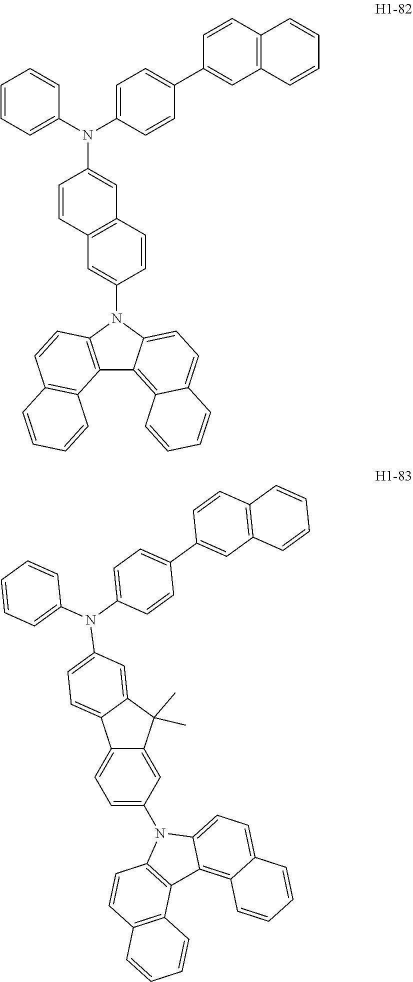 [SMILES: c1ccc(N(c2ccc(-c3ccc4ccccc4c3)cc2)c2ccc3cc(-n4c5ccc6ccccc6c5c5c6ccccc6ccc54)ccc3c2)cc1 ; CC1(C)c2cc(N(c3ccccc3)c3ccc(-c4ccc5ccccc5c4)cc3)ccc2-c2ccc(-n3c4ccc5ccccc5c4c4c5ccccc5ccc43)cc21]

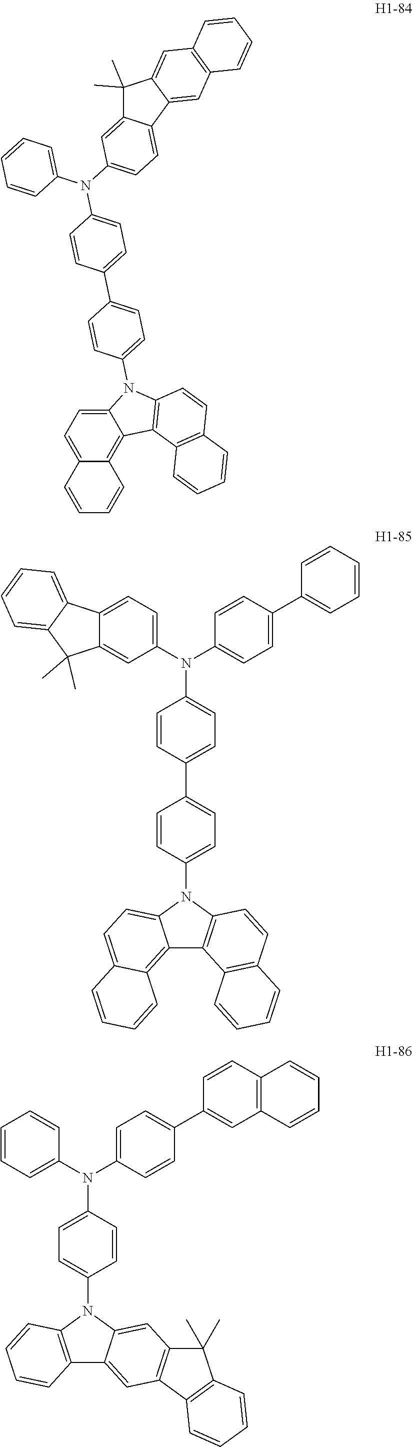

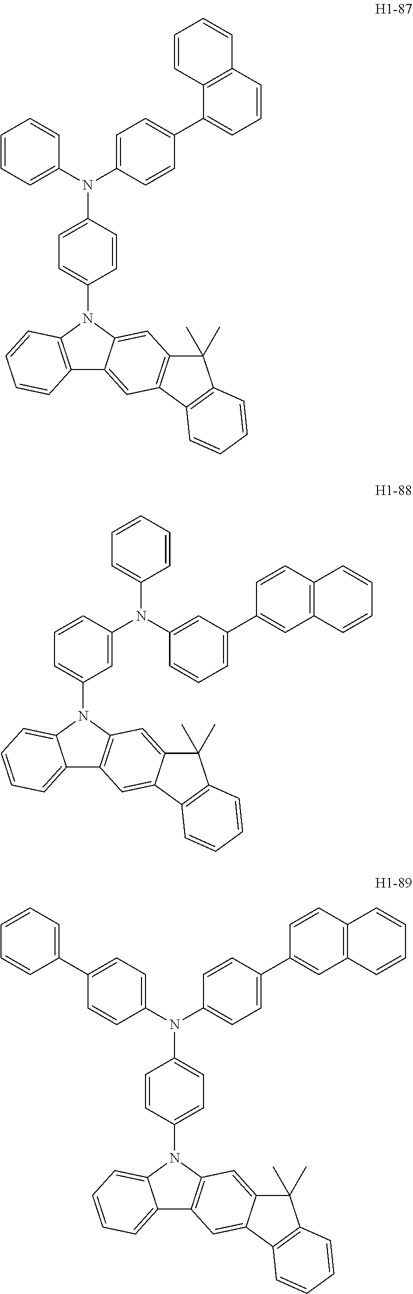 [SMILES: CC1(C)c2ccccc2-c2cc3c4ccccc4n(-c4ccc(N(c5ccccc5)c5ccc(-c6cccc7ccccc67)cc5)cc4)c3cc21 ; CC1(C)c2ccccc2-c2cc3c4ccccc4n(-c4cccc(N(c5ccccc5)c5cccc(-c6ccc7ccccc7c6)c5)c4)c3cc21 ; CC1(C)c2ccccc2-c2cc3c4ccccc4n(-c4ccc(N(c5ccc(-c6ccccc6)cc5)c5ccc(-c6ccc7ccccc7c6)cc5)cc4)c3cc21]

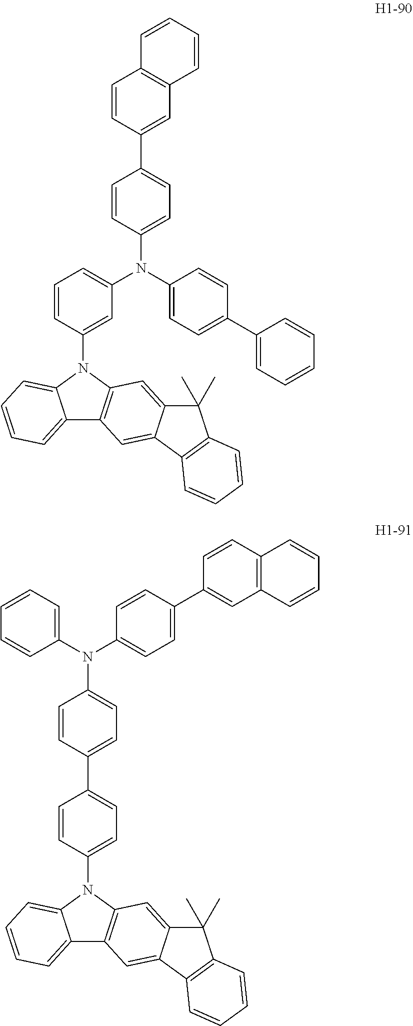

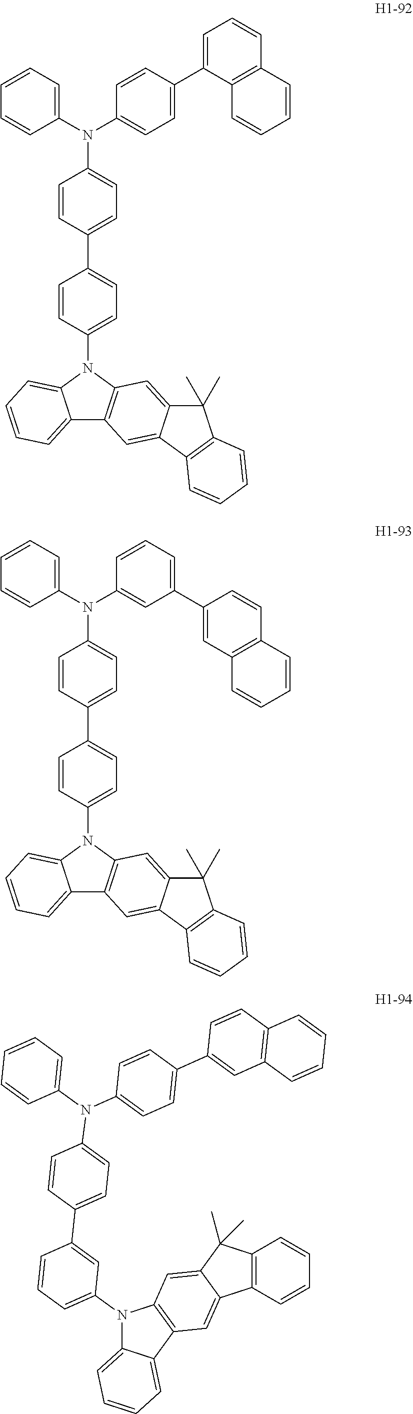

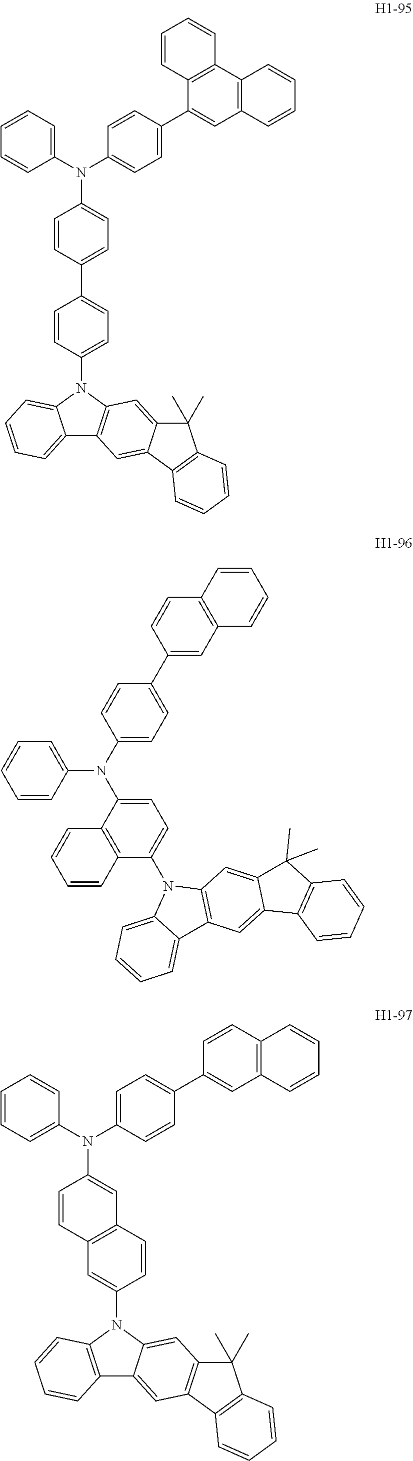 [SMILES: CC1(C)c2ccccc2-c2cc3c4ccccc4n(-c4ccc(-c5ccc(N(c6ccccc6)c6ccc(-c7cc8ccccc8c8ccccc78)cc6)cc5)cc4)c3cc21 ; CC1(C)c2ccccc2-c2cc3c4ccccc4n(-c4ccc(N(c5ccccc5)c5ccc(-c6ccc7ccccc7c6)cc5)c5ccccc45)c3cc21 ; CC1(C)c2ccccc2-c2cc3c4ccccc4n(-c4ccc5cc(N(c6ccccc6)c6ccc(-c7ccc8ccccc8c7)cc6)ccc5c4)c3cc21]

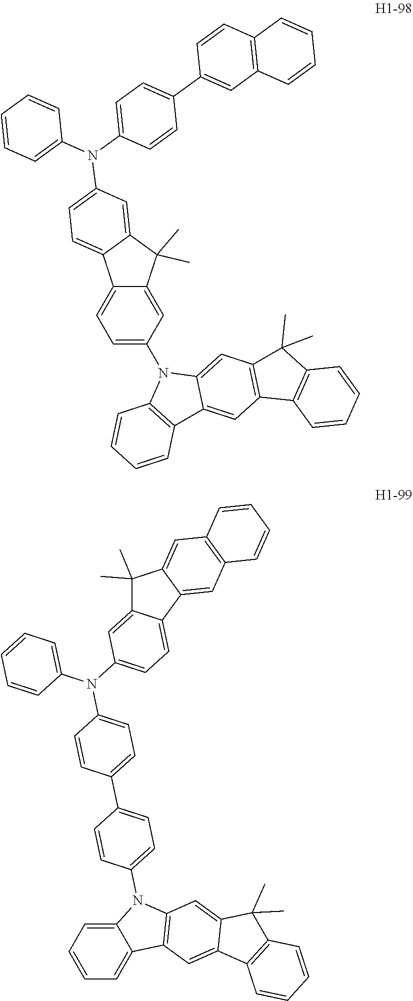 [SMILES: CC1(C)c2cc(N(c3ccccc3)c3ccc(-c4ccc5ccccc5c4)cc3)ccc2-c2ccc(-n3c4ccccc4c4cc5c(cc43)C(C)(C)c3ccccc3-5)cc21 ; CC1(C)c2cc(N(c3ccccc3)c3ccc(-c4ccc(-n5c6ccccc6c6cc7c(cc65)C(C)(C)c5ccccc5-7)cc4)cc3)ccc2-c2cc3ccccc3cc21]

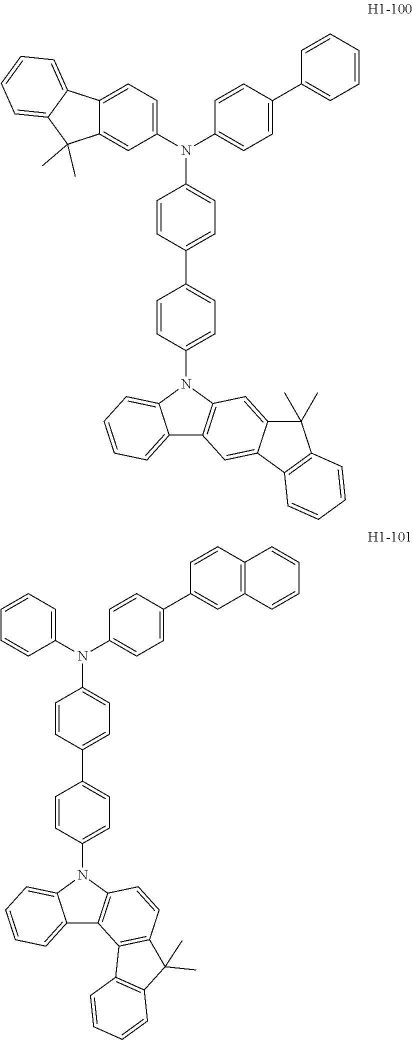

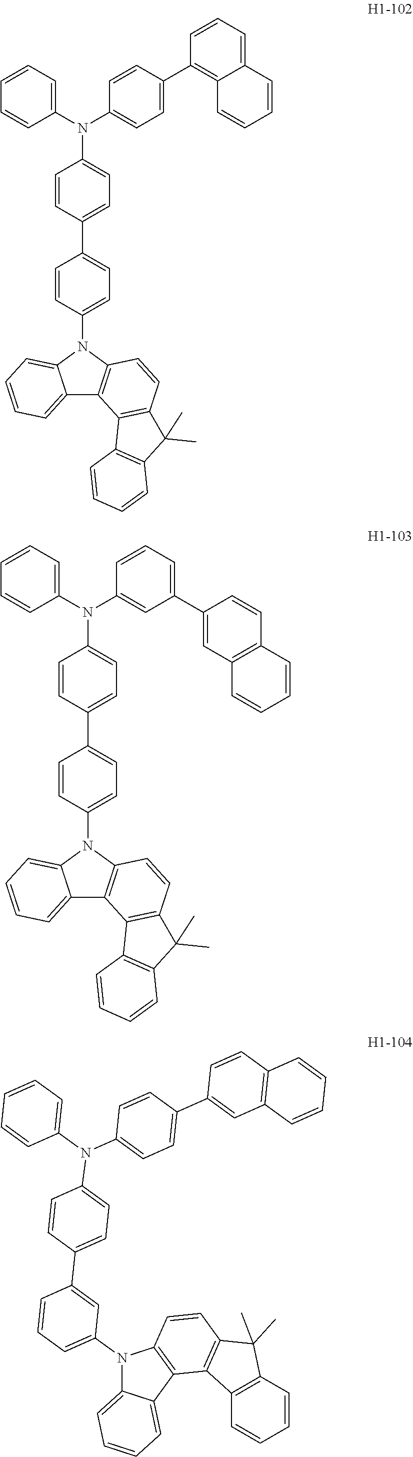

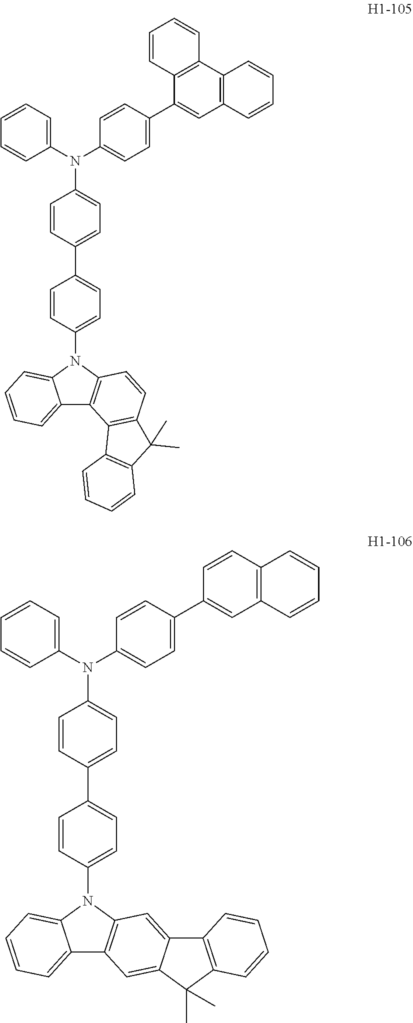 [SMILES: CC1(C)c2ccccc2-c2c1ccc1c2c2ccccc2n1-c1ccc(-c2ccc(N(c3ccccc3)c3ccc(-c4cc5ccccc5c5ccccc45)cc3)cc2)cc1 ; CC1(C)c2ccccc2-c2cc3c(cc21)c1ccccc1n3-c1ccc(-c2ccc(N(c3ccccc3)c3ccc(-c4ccc5ccccc5c4)cc3)cc2)cc1]

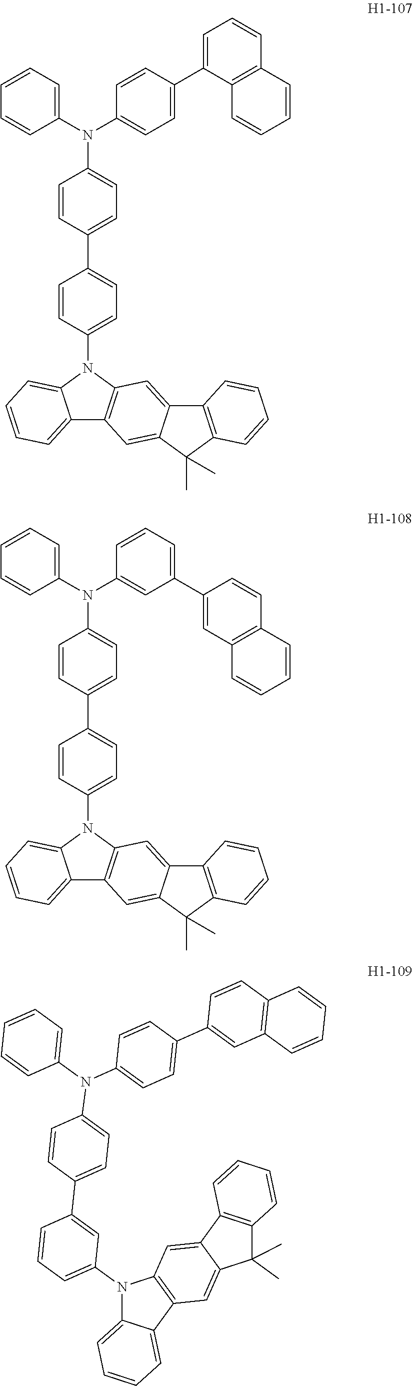 [SMILES: CC1(C)c2ccccc2-c2cc3c(cc21)c1ccccc1n3-c1ccc(-c2ccc(N(c3ccccc3)c3ccc(-c4cccc5ccccc45)cc3)cc2)cc1 ; CC1(C)c2ccccc2-c2cc3c(cc21)c1ccccc1n3-c1ccc(-c2ccc(N(c3ccccc3)c3cccc(-c4ccc5ccccc5c4)c3)cc2)cc1 ; CC1(C)c2ccccc2-c2cc3c(cc21)c1ccccc1n3-c1cccc(-c2ccc(N(c3ccccc3)c3ccc(-c4ccc5ccccc5c4)cc3)cc2)c1]

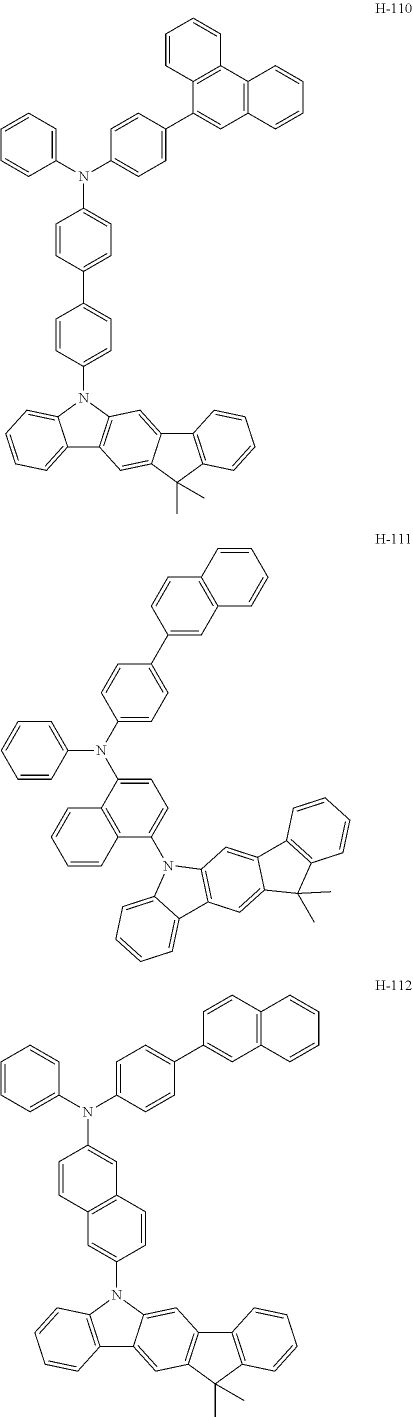 [SMILES: CC1(C)c2ccccc2-c2cc3c(cc21)c1ccccc1n3-c1ccc(-c2ccc(N(c3ccccc3)c3ccc(-c4cc5ccccc5c5ccccc45)cc3)cc2)cc1 ; CC1(C)c2ccccc2-c2cc3c(cc21)c1ccccc1n3-c1ccc(N(c2ccccc2)c2ccc(-c3ccc4ccccc4c3)cc2)c2ccccc12 ; CC1(C)c2ccccc2-c2cc3c(cc21)c1ccccc1n3-c1ccc2cc(N(c3ccccc3)c3ccc(-c4ccc5ccccc5c4)cc3)ccc2c1]

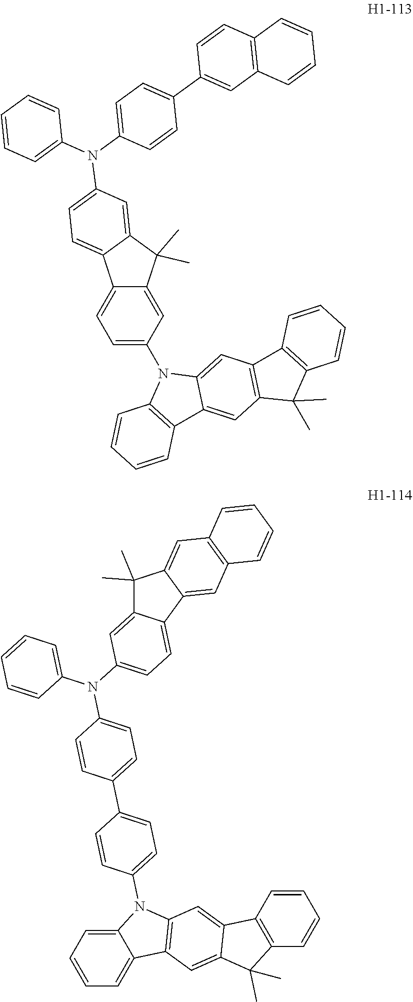 [SMILES: CC1(C)c2cc(N(c3ccccc3)c3ccc(-c4ccc5ccccc5c4)cc3)ccc2-c2ccc(-n3c4ccccc4c4cc5c(cc43)-c3ccccc3C5(C)C)cc21 ; CC1(C)c2cc(N(c3ccccc3)c3ccc(-c4ccc(-n5c6ccccc6c6cc7c(cc65)-c5ccccc5C7(C)C)cc4)cc3)ccc2-c2cc3ccccc3cc21]

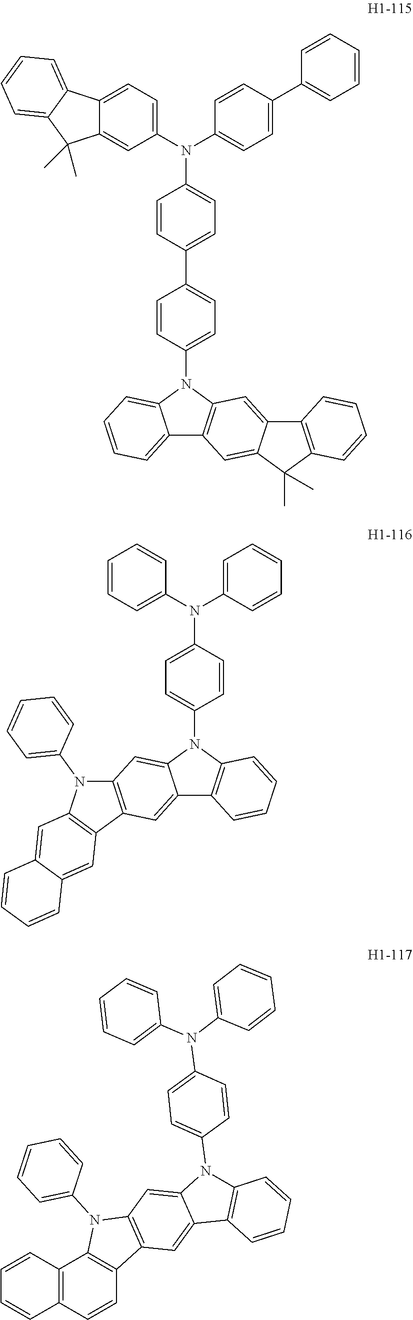 [SMILES: CC1(C)c2ccccc2-c2ccc(N(c3ccc(-c4ccccc4)cc3)c3ccc(-c4ccc(-n5c6ccccc6c6cc7c(cc65)-c5ccccc5C7(C)C)cc4)cc3)cc21 ; c1ccc(N(c2ccccc2)c2ccc(-n3c4ccccc4c4cc5c6cc7ccccc7cc6n(-c6ccccc6)c5cc43)cc2)cc1 ; c1ccc(N(c2ccccc2)c2ccc(-n3c4ccccc4c4cc5c6ccc7ccccc7c6n(-c6ccccc6)c5cc43)cc2)cc1]

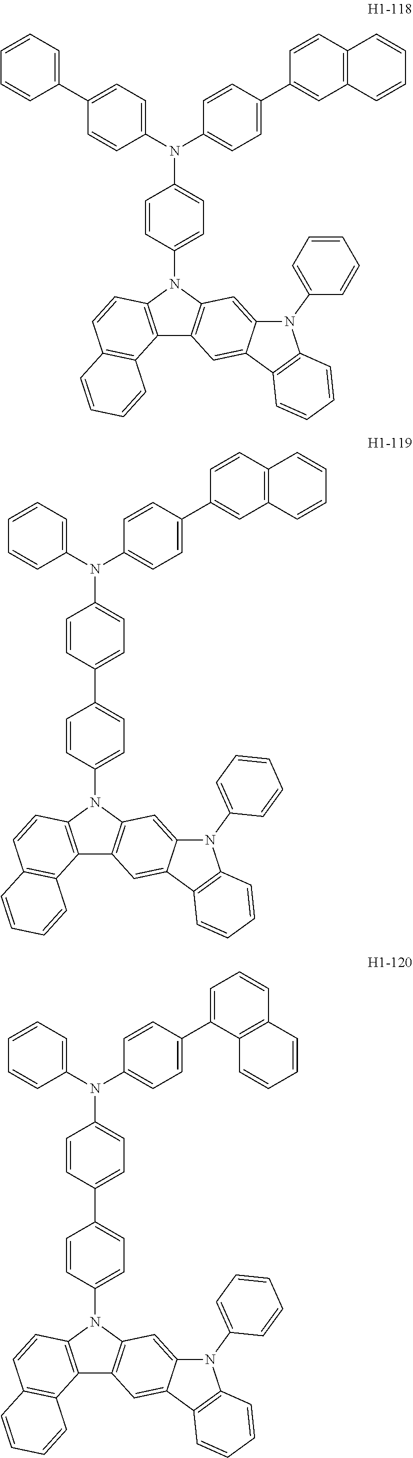

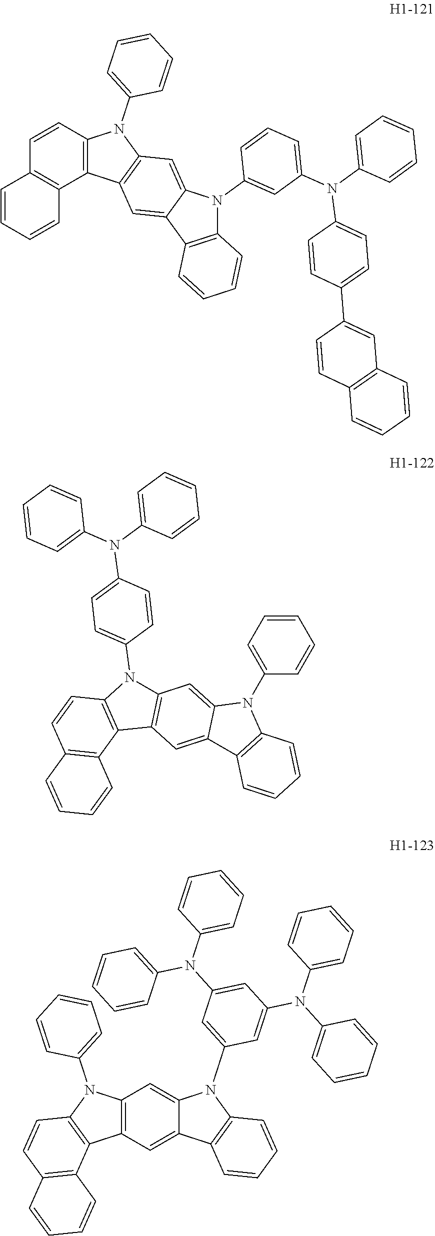 [SMILES: c1ccc(N(c2ccc(-c3ccc4ccccc4c3)cc2)c2cccc(-n3c4ccccc4c4cc5c6c7ccccc7ccc6n(-c6ccccc6)c5cc43)c2)cc1 ; c1ccc(N(c2ccccc2)c2ccc(-n3c4cc5c(cc4c4c6ccccc6ccc43)c3ccccc3n5-c3ccccc3)cc2)cc1 ; c1ccc(N(c2ccccc2)c2cc(N(c3ccccc3)c3ccccc3)cc(-n3c4ccccc4c4cc5c6c7ccccc7ccc6n(-c6ccccc6)c5cc43)c2)cc1]

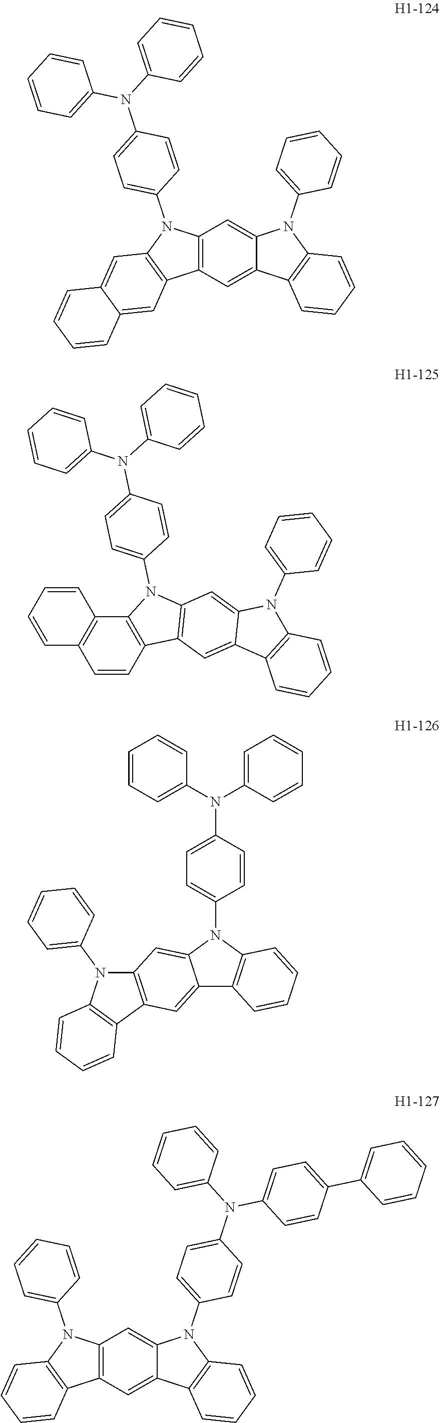

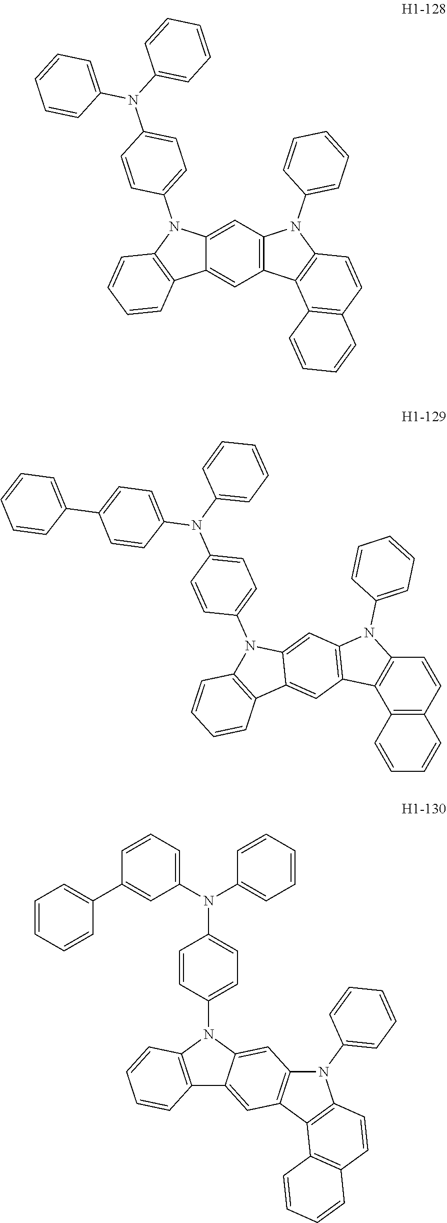 [SMILES: c1ccc(N(c2ccccc2)c2ccc(-n3c4ccccc4c4cc5c6c7ccccc7ccc6n(-c6ccccc6)c5cc43)cc2)cc1 ; c1ccc(-c2ccc(N(c3ccccc3)c3ccc(-n4c5ccccc5c5cc6c7c8ccccc8ccc7n(-c7ccccc7)c6cc54)cc3)cc2)cc1 ; c1ccc(-c2cccc(N(c3ccccc3)c3ccc(-n4c5ccccc5c5cc6c7c8ccccc8ccc7n(-c7ccccc7)c6cc54)cc3)c2)cc1]

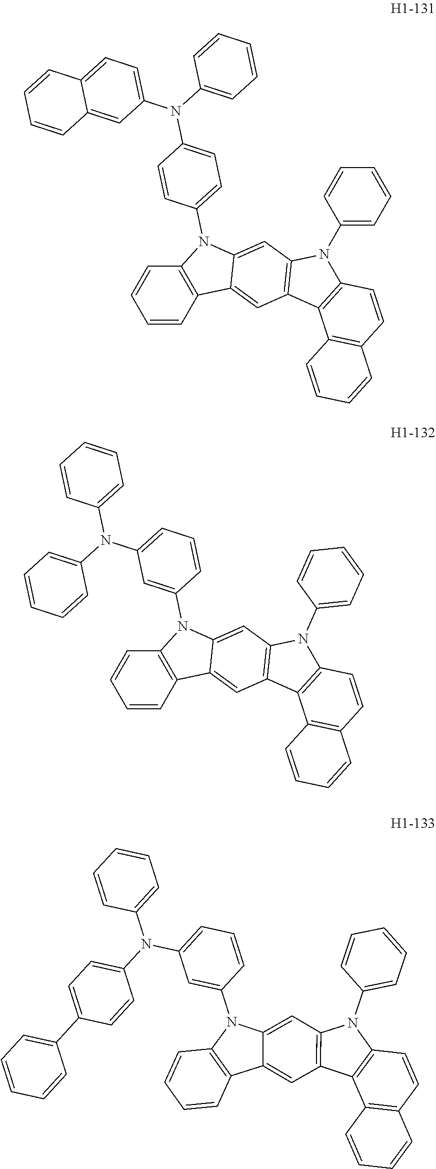 [SMILES: c1ccc(N(c2ccc(-n3c4ccccc4c4cc5c6c7ccccc7ccc6n(-c6ccccc6)c5cc43)cc2)c2ccc3ccccc3c2)cc1 ; c1ccc(N(c2ccccc2)c2cccc(-n3c4ccccc4c4cc5c6c7ccccc7ccc6n(-c6ccccc6)c5cc43)c2)cc1 ; c1ccc(-c2ccc(N(c3ccccc3)c3cccc(-n4c5ccccc5c5cc6c7c8ccccc8ccc7n(-c7ccccc7)c6cc54)c3)cc2)cc1]

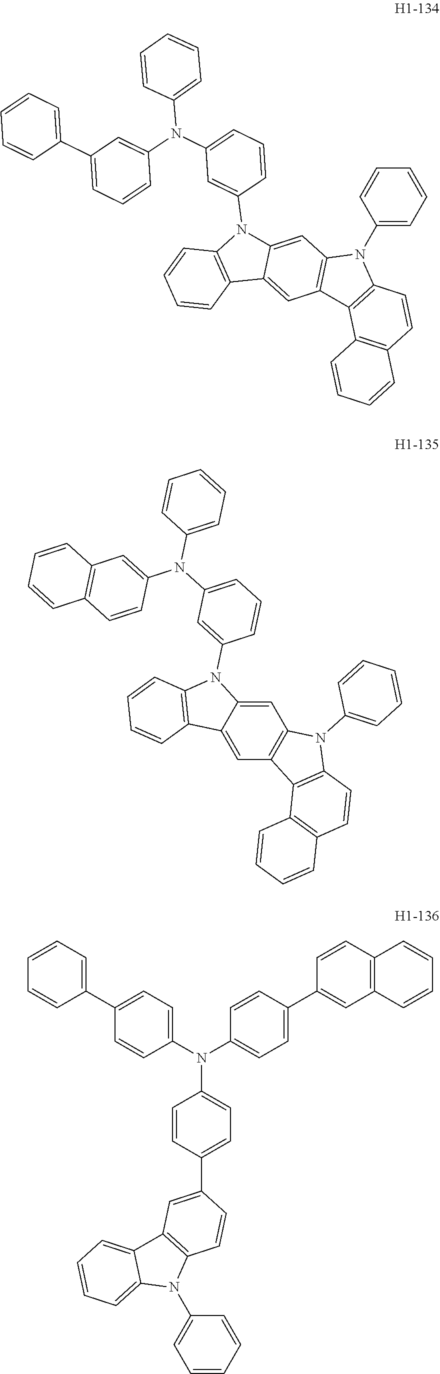 [SMILES: c1ccc(-c2cccc(N(c3ccccc3)c3cccc(-n4c5ccccc5c5cc6c7c8ccccc8ccc7n(-c7ccccc7)c6cc54)c3)c2)cc1 ; c1ccc(N(c2cccc(-n3c4ccccc4c4cc5c6c7ccccc7ccc6n(-c6ccccc6)c5cc43)c2)c2ccc3ccccc3c2)cc1 ; c1ccc(-c2ccc(N(c3ccc(-c4ccc5ccccc5c4)cc3)c3ccc(-c4ccc5c(c4)c4ccccc4n5-c4ccccc4)cc3)cc2)cc1]

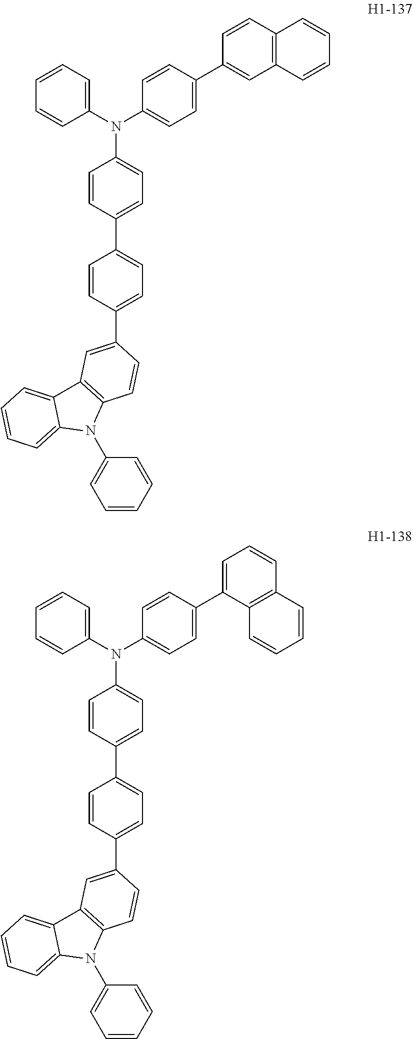 [SMILES: c1ccc(N(c2ccc(-c3ccc(-c4ccc5c(c4)c4ccccc4n5-c4ccccc4)cc3)cc2)c2ccc(-c3ccc4ccccc4c3)cc2)cc1 ; c1ccc(N(c2ccc(-c3ccc(-c4ccc5c(c4)c4ccccc4n5-c4ccccc4)cc3)cc2)c2ccc(-c3cccc4ccccc34)cc2)cc1]

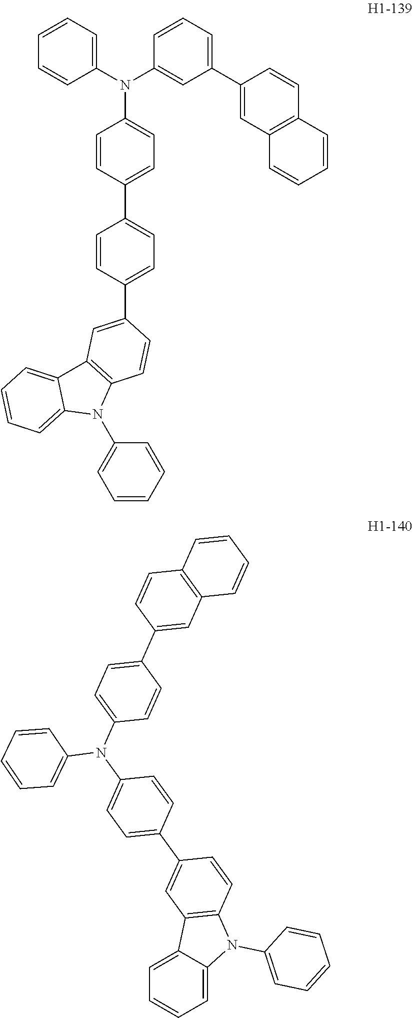 [SMILES: c1ccc(N(c2ccc(-c3ccc(-c4ccc5c(c4)c4ccccc4n5-c4ccccc4)cc3)cc2)c2cccc(-c3ccc4ccccc4c3)c2)cc1 ; c1ccc(N(c2ccc(-c3ccc4ccccc4c3)cc2)c2ccc(-c3ccc4c(c3)c3ccccc3n4-c3ccccc3)cc2)cc1]

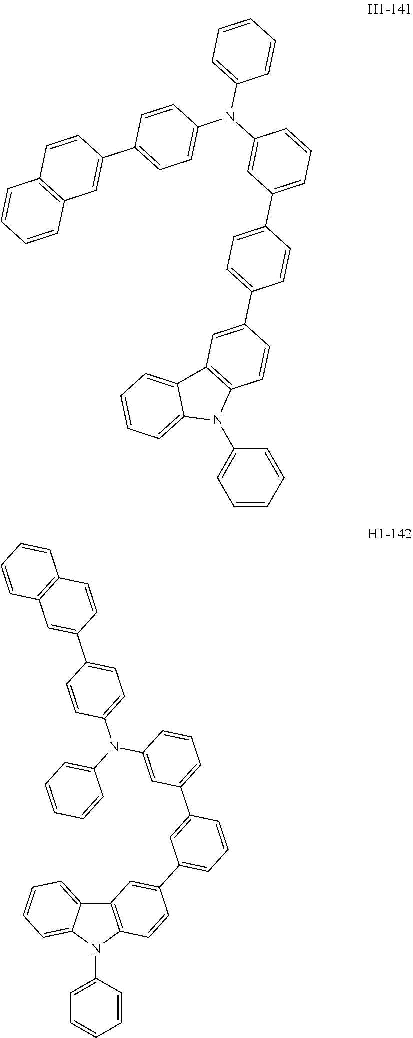 [SMILES: c1ccc(N(c2ccc(-c3ccc4ccccc4c3)cc2)c2cccc(-c3ccc(-c4ccc5c(c4)c4ccccc4n5-c4ccccc4)cc3)c2)cc1 ; c1ccc(N(c2ccc(-c3ccc4ccccc4c3)cc2)c2cccc(-c3cccc(-c4ccc5c(c4)c4ccccc4n5-c4ccccc4)c3)c2)cc1]

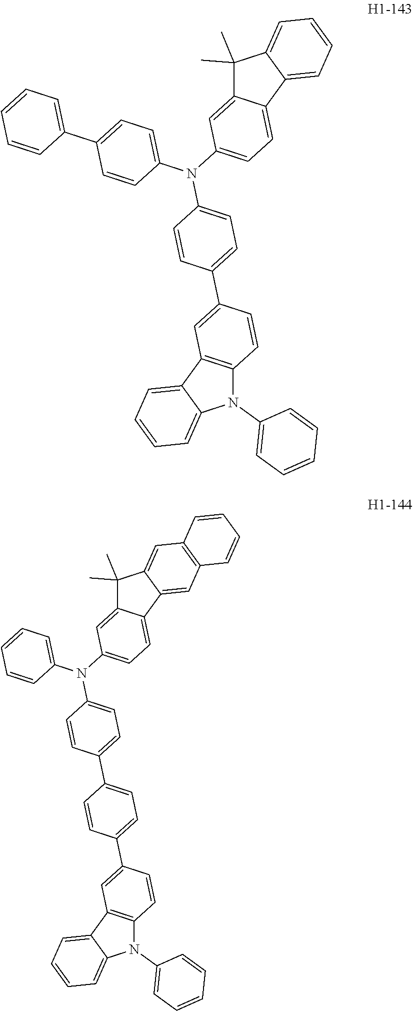 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2cc(N(c3ccccc3)c3ccc(-c4ccc(-c5ccc6c(c5)c5ccccc5n6-c5ccccc5)cc4)cc3)ccc2-c2cc3ccccc3cc21]

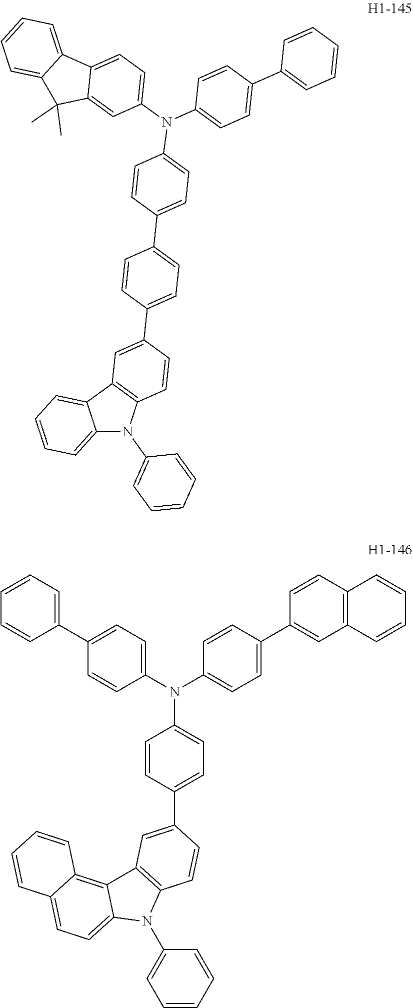 [SMILES: CC1(C)c2ccccc2-c2ccc(N(c3ccc(-c4ccccc4)cc3)c3ccc(-c4ccc(-c5ccc6c(c5)c5ccccc5n6-c5ccccc5)cc4)cc3)cc21 ; c1ccc(-c2ccc(N(c3ccc(-c4ccc5ccccc5c4)cc3)c3ccc(-c4ccc5c(c4)c4c6ccccc6ccc4n5-c4ccccc4)cc3)cc2)cc1]

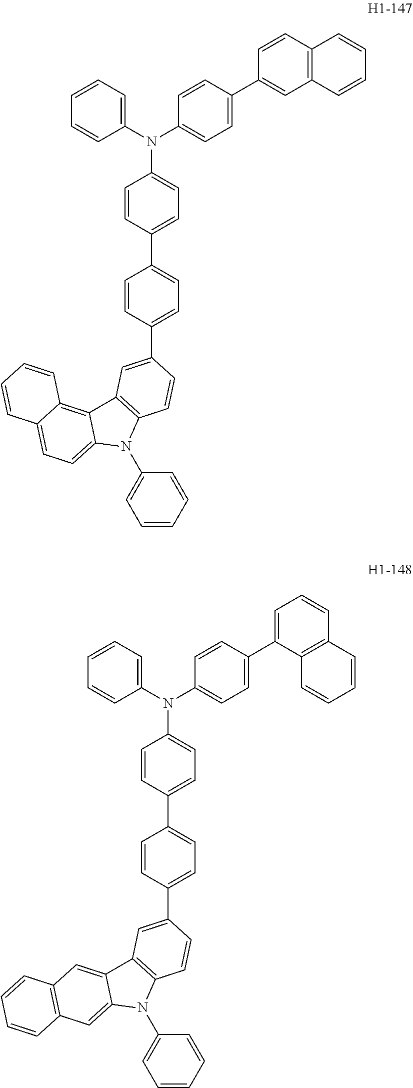 [SMILES: c1ccc(N(c2ccc(-c3ccc(-c4ccc5c(c4)c4c6ccccc6ccc4n5-c4ccccc4)cc3)cc2)c2ccc(-c3ccc4ccccc4c3)cc2)cc1 ; c1ccc(N(c2ccc(-c3ccc(-c4ccc5c(c4)c4cc6ccccc6cc4n5-c4ccccc4)cc3)cc2)c2ccc(-c3cccc4ccccc34)cc2)cc1]

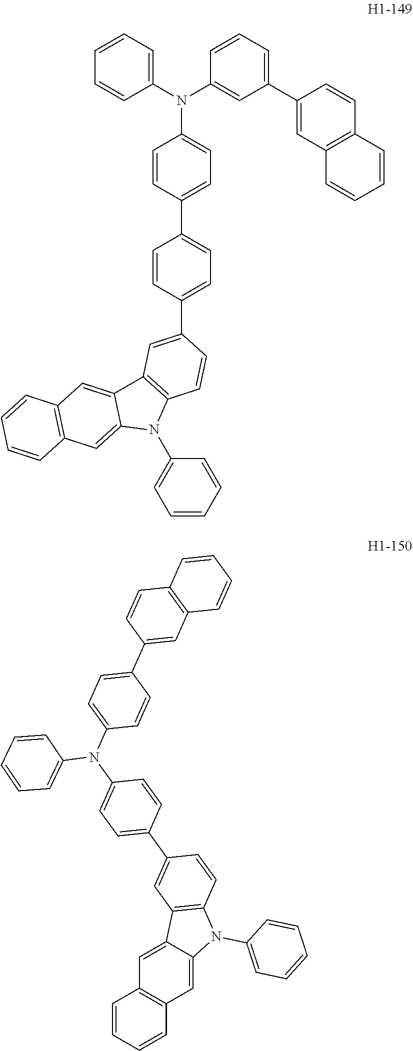

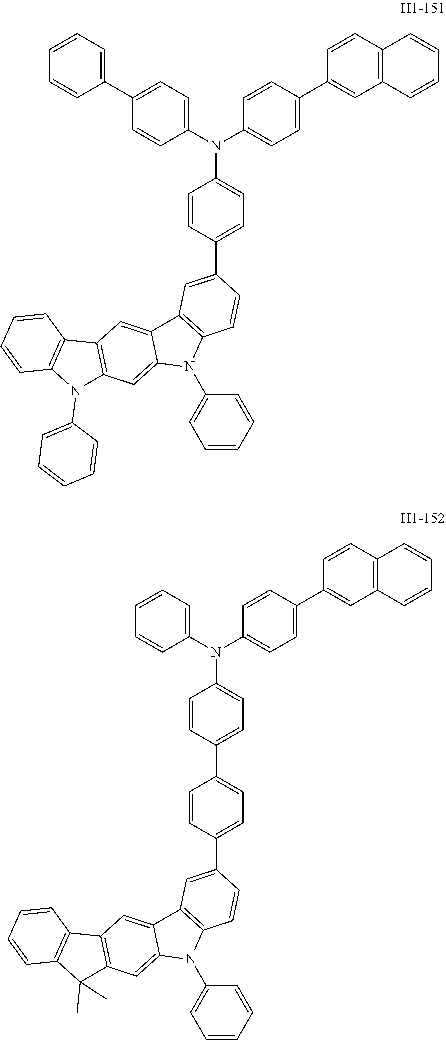 [SMILES: c1ccc(-c2ccc(N(c3ccc(-c4ccc5ccccc5c4)cc3)c3ccc(-c4ccc5c(c4)c4cc6c7ccccc7n(-c7ccccc7)c6cc4n5-c4ccccc4)cc3)cc2)cc1 ; CC1(C)c2ccccc2-c2cc3c4cc(-c5ccc(-c6ccc(N(c7ccccc7)c7ccc(-c8ccc9ccccc9c8)cc7)cc6)cc5)ccc4n(-c4ccccc4)c3cc21]

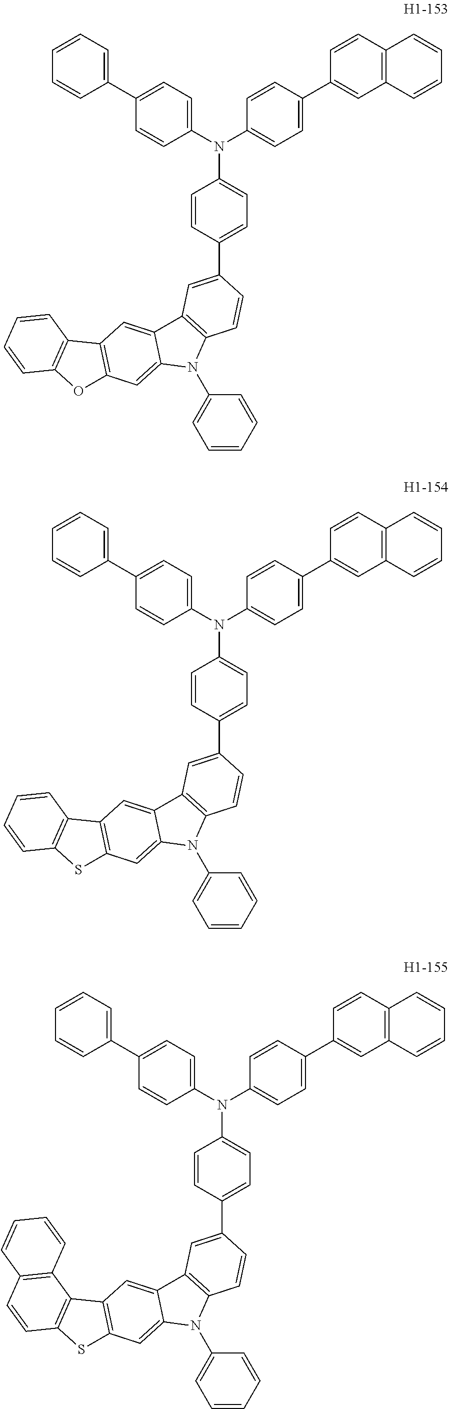 [SMILES: c1ccc(-c2ccc(N(c3ccc(-c4ccc5ccccc5c4)cc3)c3ccc(-c4ccc5c(c4)c4cc6c(cc4n5-c4ccccc4)oc4ccccc46)cc3)cc2)cc1 ; c1ccc(-c2ccc(N(c3ccc(-c4ccc5ccccc5c4)cc3)c3ccc(-c4ccc5c(c4)c4cc6c(cc4n5-c4ccccc4)sc4ccccc46)cc3)cc2)cc1 ; c1ccc(-c2ccc(N(c3ccc(-c4ccc5ccccc5c4)cc3)c3ccc(-c4ccc5c(c4)c4cc6c(cc4n5-c4ccccc4)sc4ccc5ccccc5c46)cc3)cc2)cc1]

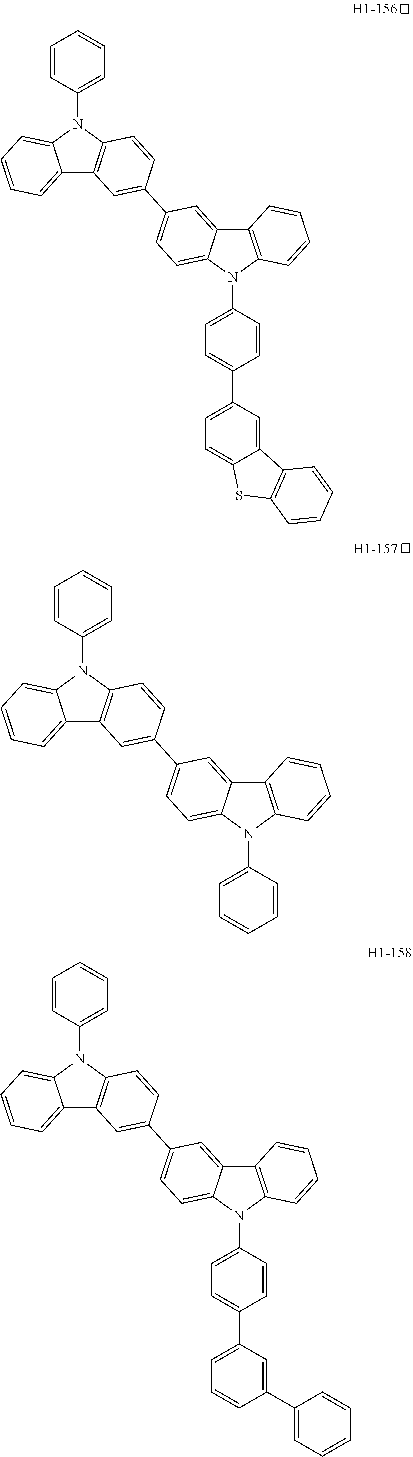 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccccc4)ccc32)cc1 ; c1ccc(-c2cccc(-c3ccc(-n4c5ccccc5c5cc(-c6ccc7c(c6)c6ccccc6n7-c6ccccc6)ccc54)cc3)c2)cc1]

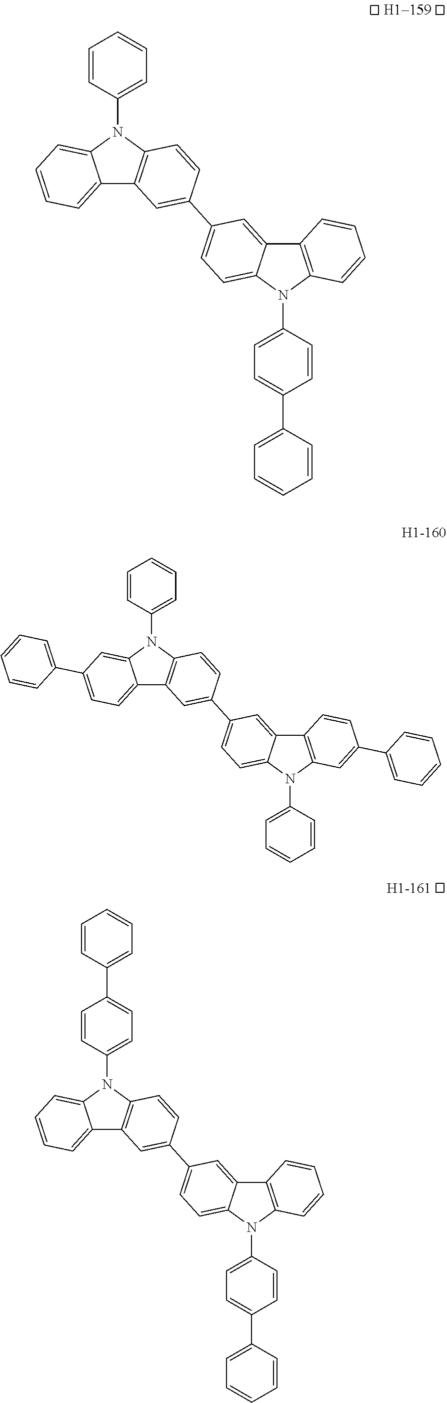 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc3c4cc(-c5ccc6c(c5)c5ccc(-c7ccccc7)cc5n6-c5ccccc5)ccc4n(-c4ccccc4)c3c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

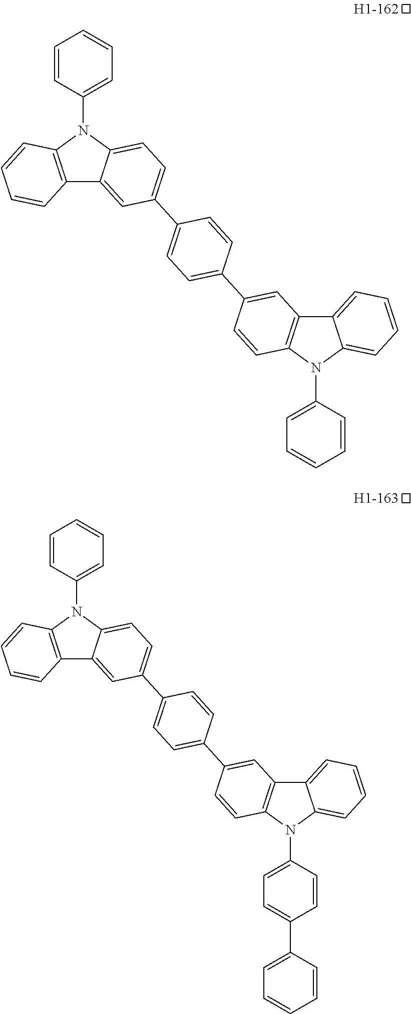 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

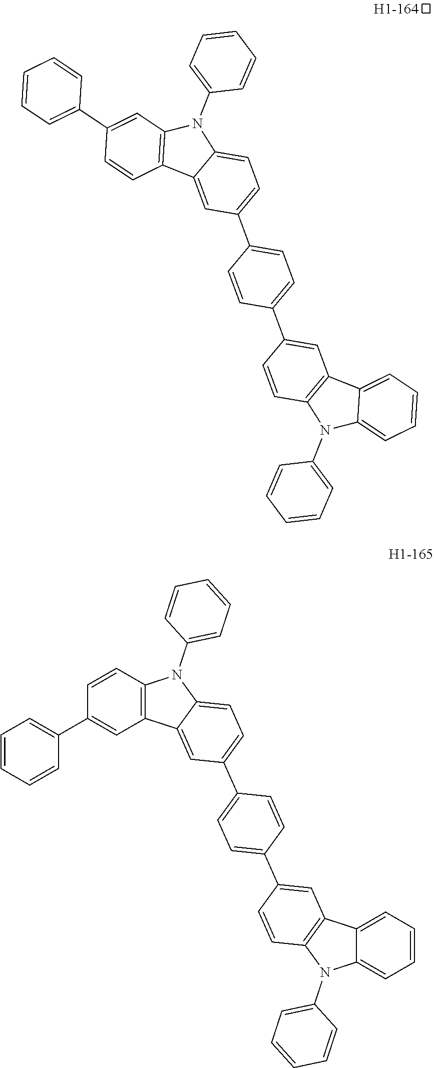 [SMILES: c1ccc(-c2ccc3c4cc(-c5ccc(-c6ccc7c(c6)c6ccccc6n7-c6ccccc6)cc5)ccc4n(-c4ccccc4)c3c2)cc1 ; c1ccc(-c2ccc3c(c2)c2cc(-c4ccc(-c5ccc6c(c5)c5ccccc5n6-c5ccccc5)cc4)ccc2n3-c2ccccc2)cc1]

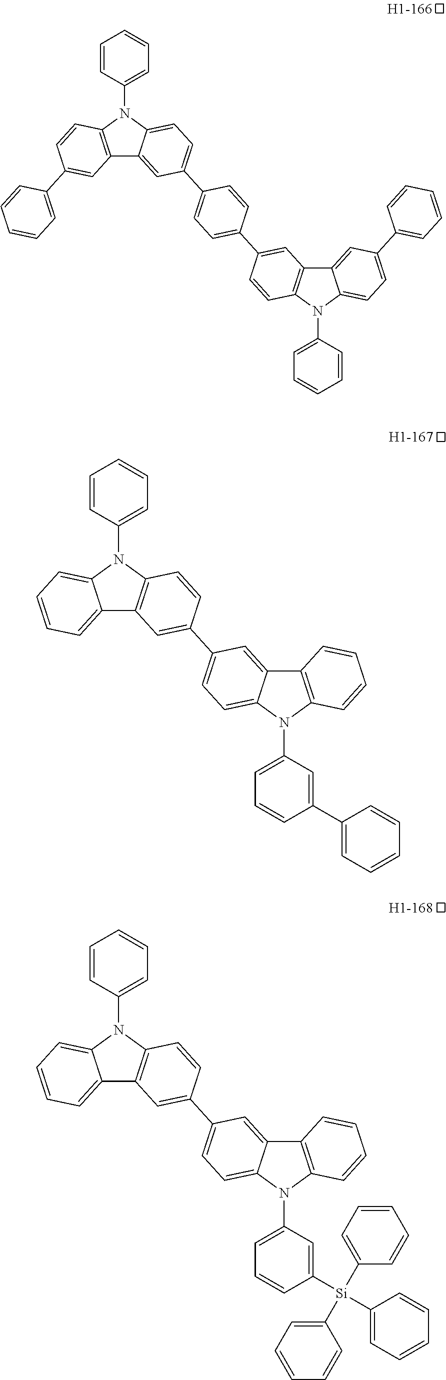 [SMILES: c1ccc(-c2ccc3c(c2)c2cc(-c4ccc(-c5ccc6c(c5)c5cc(-c7ccccc7)ccc5n6-c5ccccc5)cc4)ccc2n3-c2ccccc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

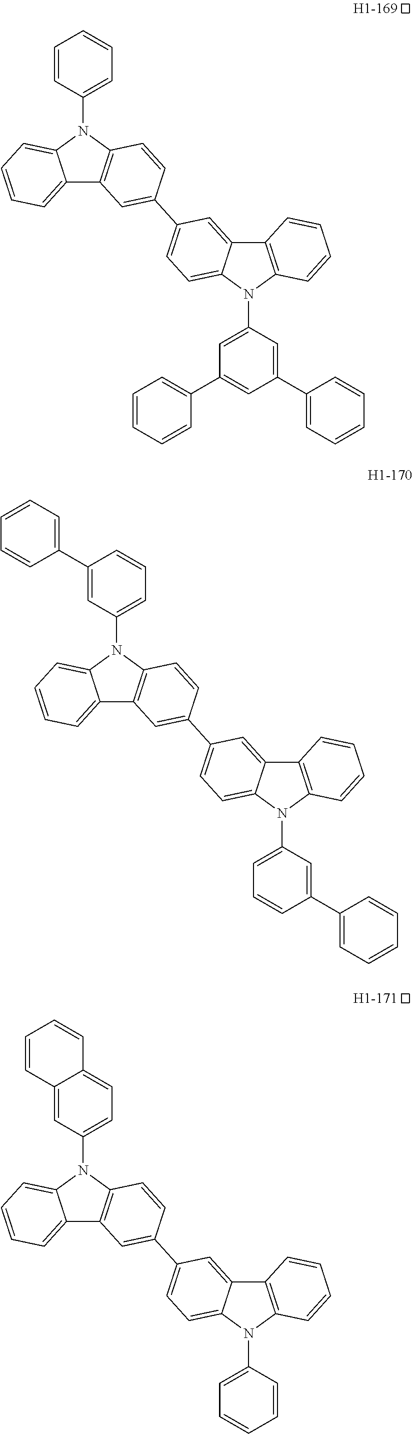 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c(c4)c4ccccc4n5-c4ccc5ccccc5c4)ccc32)cc1]

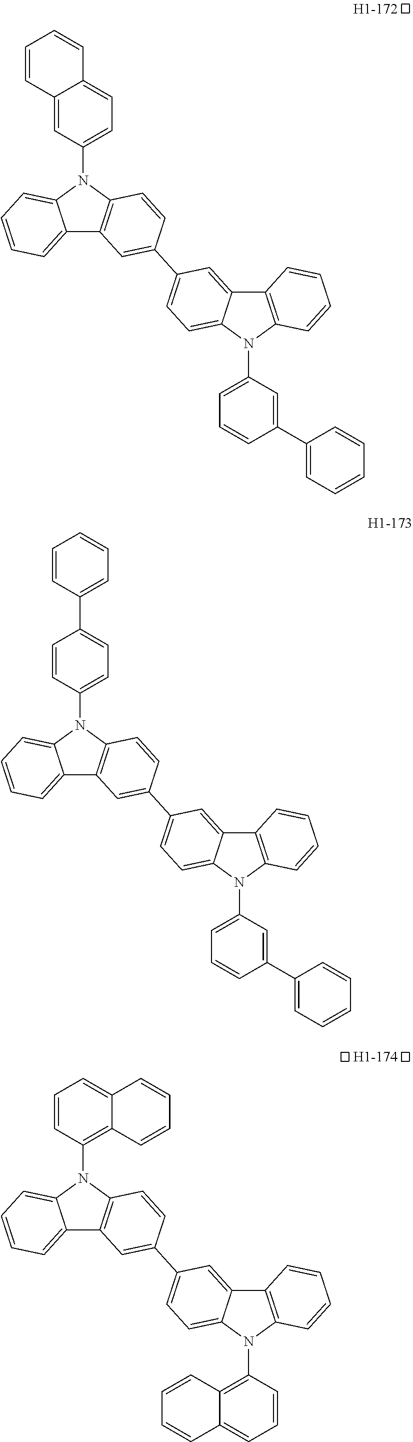 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-n3c4ccccc4c4cc(-c5ccc6c(c5)c5ccccc5n6-c5cccc6ccccc56)ccc43)cccc2c1]

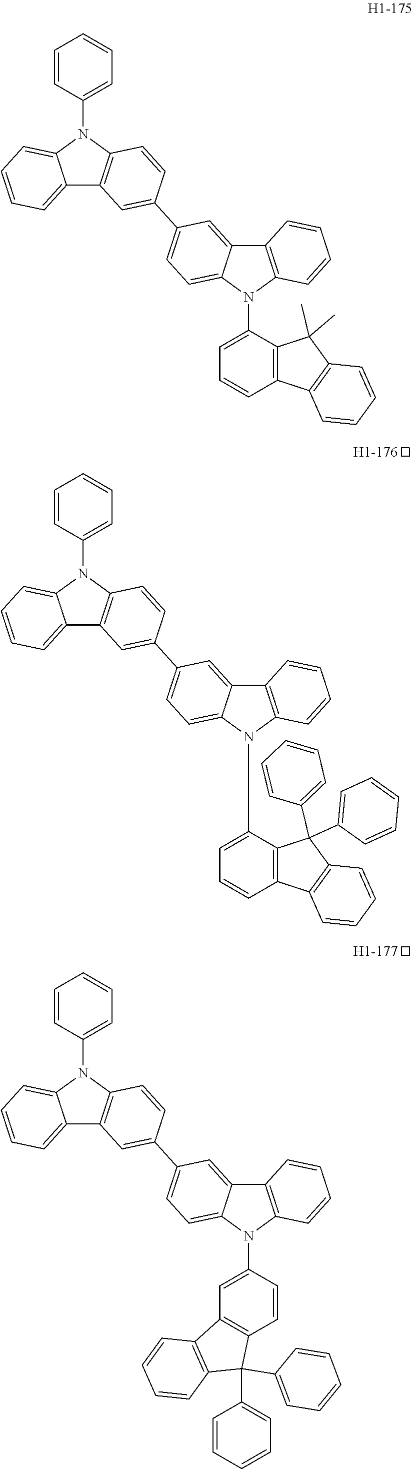 [SMILES: CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1]

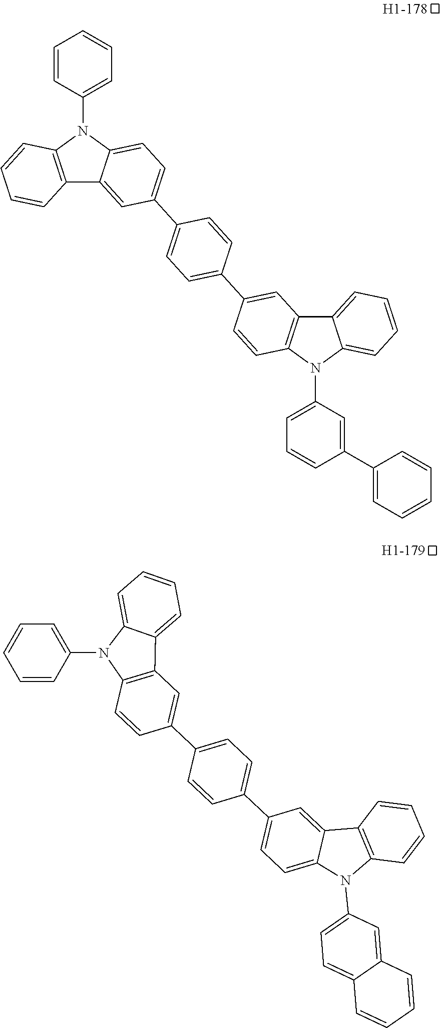 [SMILES: c1ccc(-c2cccc(-n3c4ccccc4c4cc(-c5ccc(-c6ccc7c(c6)c6ccccc6n7-c6ccccc6)cc5)ccc43)c2)cc1 ; c1ccc(-n2c3ccccc3c3cc(-c4ccc(-c5ccc6c(c5)c5ccccc5n6-c5ccc6ccccc6c5)cc4)ccc32)cc1]

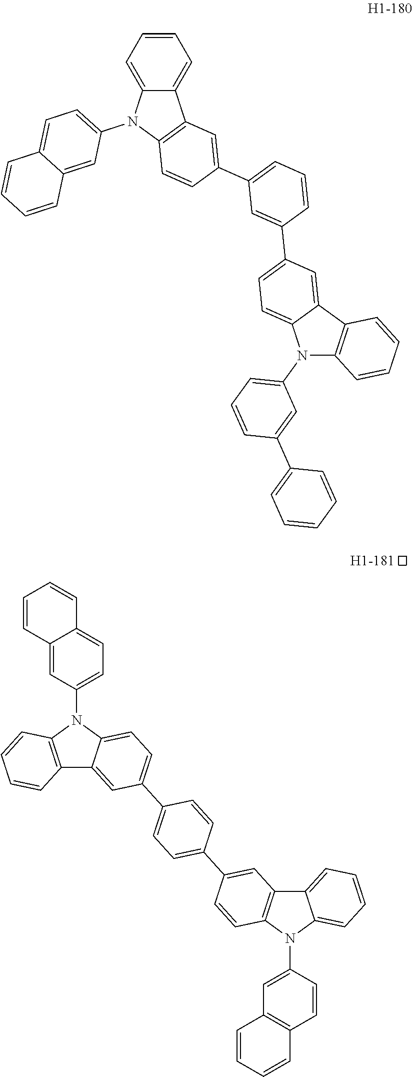 [SMILES: c1ccc(-c2cccc(-n3c4ccccc4c4cc(-c5cccc(-c6ccc7c(c6)c6ccccc6n7-c6ccc7ccccc7c6)c5)ccc43)c2)cc1 ; c1ccc2cc(-n3c4ccccc4c4cc(-c5ccc(-c6ccc7c(c6)c6ccccc6n7-c6ccc7ccccc7c6)cc5)ccc43)ccc2c1]

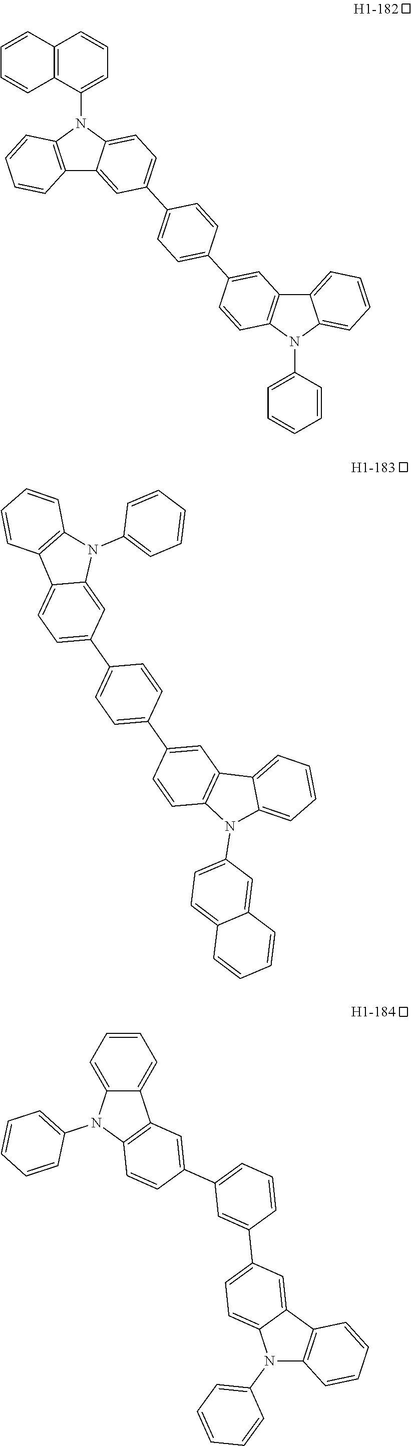 [SMILES: c1ccc(-n2c3ccccc3c3cc(-c4ccc(-c5ccc6c(c5)c5ccccc5n6-c5cccc6ccccc56)cc4)ccc32)cc1 ; c1ccc(-n2c3ccccc3c3ccc(-c4ccc(-c5ccc6c(c5)c5ccccc5n6-c5ccc6ccccc6c5)cc4)cc32)cc1 ; c1ccc(-n2c3ccccc3c3cc(-c4cccc(-c5ccc6c(c5)c5ccccc5n6-c5ccccc5)c4)ccc32)cc1]

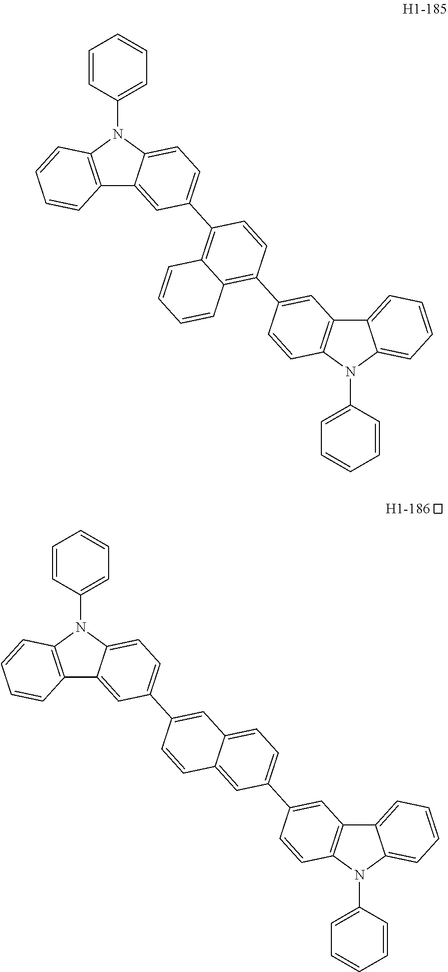 [SMILES: c1ccc(-n2c3ccccc3c3cc(-c4ccc(-c5ccc6c(c5)c5ccccc5n6-c5ccccc5)c5ccccc45)ccc32)cc1 ; c1ccc(-n2c3ccccc3c3cc(-c4ccc5cc(-c6ccc7c(c6)c6ccccc6n7-c6ccccc6)ccc5c4)ccc32)cc1]

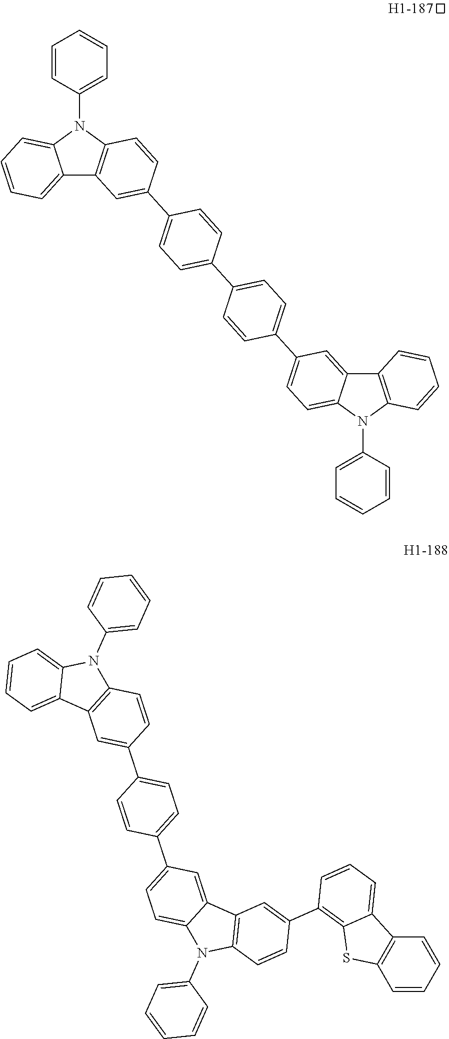 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5cc(-c7cccc8c7sc7ccccc78)ccc5n6-c5ccccc5)cc4)ccc32)cc1]

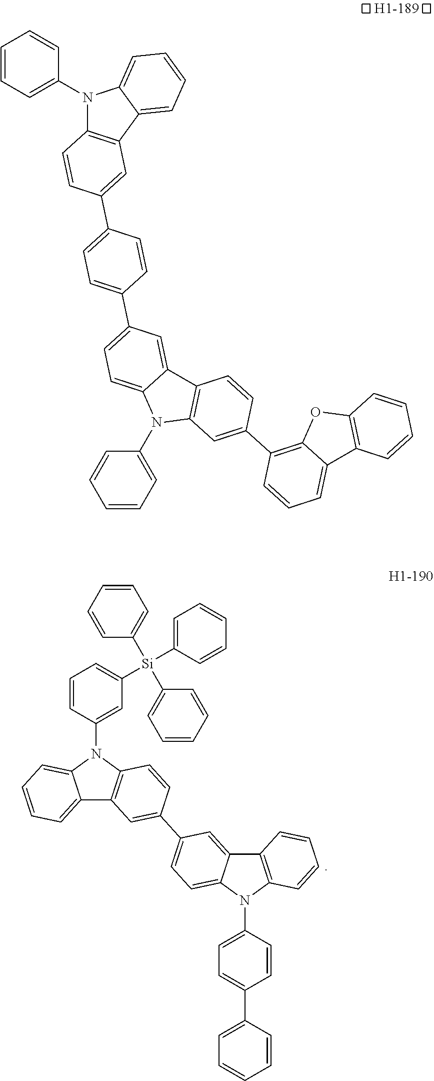 [SMILES: c1ccc(-n2c3ccccc3c3cc(-c4ccc(-c5ccc6c(c5)c5ccc(-c7cccc8c7oc7ccccc78)cc5n6-c5ccccc5)cc4)ccc32)cc1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1]

The compound represented by formula 2 includes the following compounds, but is not limited thereto:

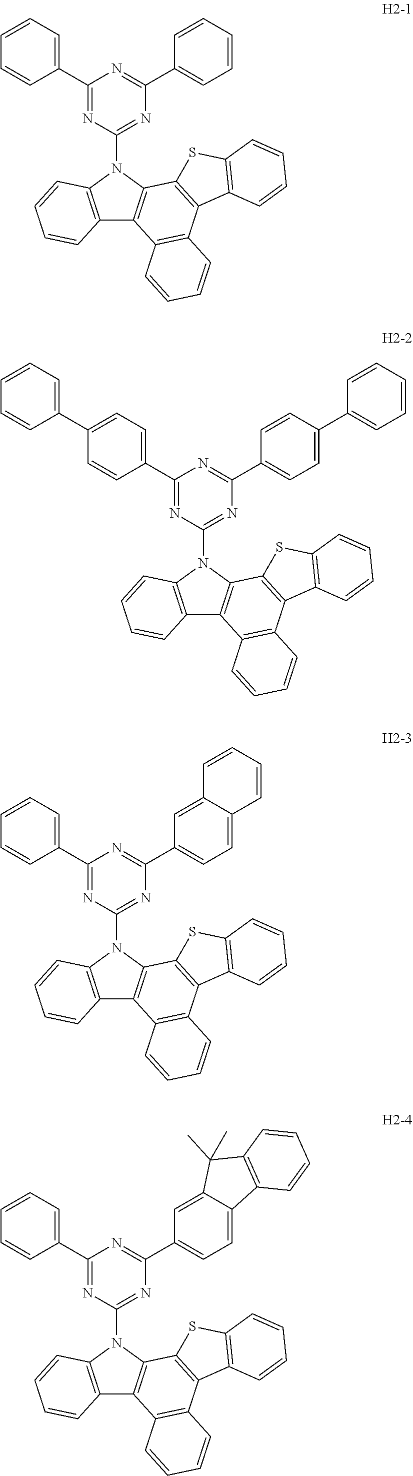 [SMILES: c1ccc(-c2nc(-c3ccccc3)nc(-n3c4ccccc4c4c5ccccc5c5c6ccccc6sc5c43)n2)cc1 ; c1ccc(-c2ccc(-c3nc(-c4ccc(-c5ccccc5)cc4)nc(-n4c5ccccc5c5c6ccccc6c6c7ccccc7sc6c54)n3)cc2)cc1 ; c1ccc(-c2nc(-c3ccc4ccccc4c3)nc(-n3c4ccccc4c4c5ccccc5c5c6ccccc6sc5c43)n2)cc1 ; CC1(C)c2ccccc2-c2ccc(-c3nc(-c4ccccc4)nc(-n4c5ccccc5c5c6ccccc6c6c7ccccc7sc6c54)n3)cc21]

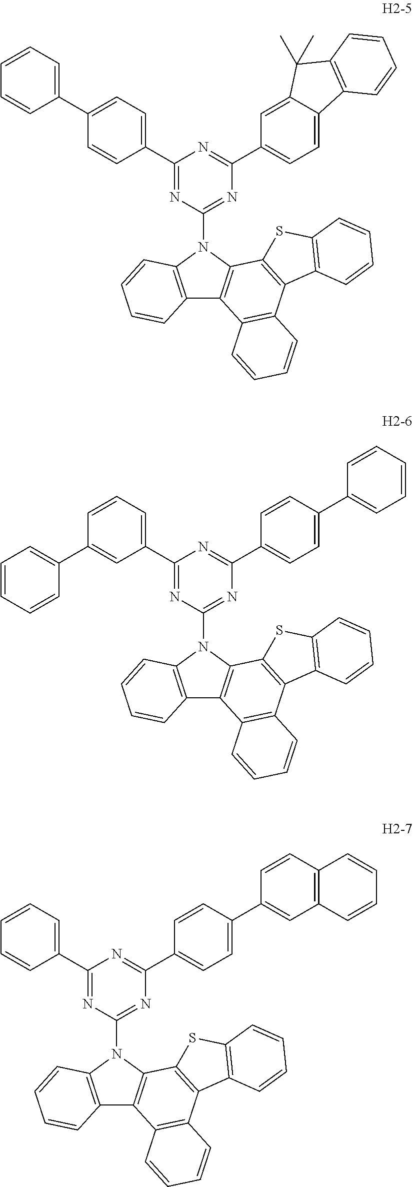 [SMILES: CC1(C)c2ccccc2-c2ccc(-c3nc(-c4ccc(-c5ccccc5)cc4)nc(-n4c5ccccc5c5c6ccccc6c6c7ccccc7sc6c54)n3)cc21 ; c1ccc(-c2ccc(-c3nc(-c4cccc(-c5ccccc5)c4)nc(-n4c5ccccc5c5c6ccccc6c6c7ccccc7sc6c54)n3)cc2)cc1 ; c1ccc(-c2nc(-c3ccc(-c4ccc5ccccc5c4)cc3)nc(-n3c4ccccc4c4c5ccccc5c5c6ccccc6sc5c43)n2)cc1]

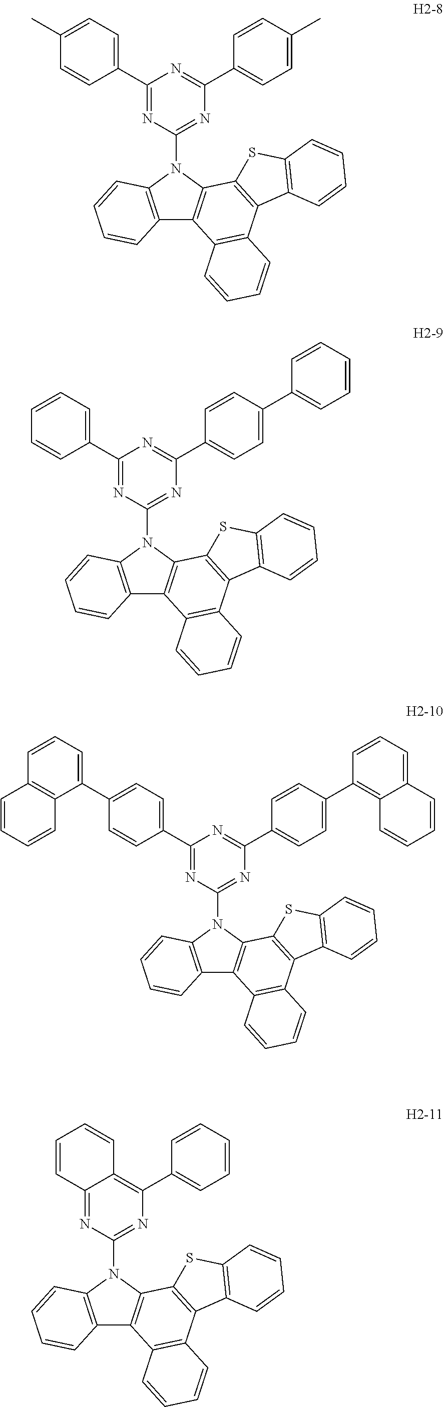 [SMILES: Cc1ccc(-c2nc(-c3ccc(C)cc3)nc(-n3c4ccccc4c4c5ccccc5c5c6ccccc6sc5c43)n2)cc1 ; c1ccc(-c2ccc(-c3nc(-c4ccccc4)nc(-n4c5ccccc5c5c6ccccc6c6c7ccccc7sc6c54)n3)cc2)cc1 ; c1ccc2c(-c3ccc(-c4nc(-c5ccc(-c6cccc7ccccc67)cc5)nc(-n5c6ccccc6c6c7ccccc7c7c8ccccc8sc7c65)n4)cc3)cccc2c1 ; c1ccc(-c2nc(-n3c4ccccc4c4c5ccccc5c5c6ccccc6sc5c43)nc3ccccc23)cc1]

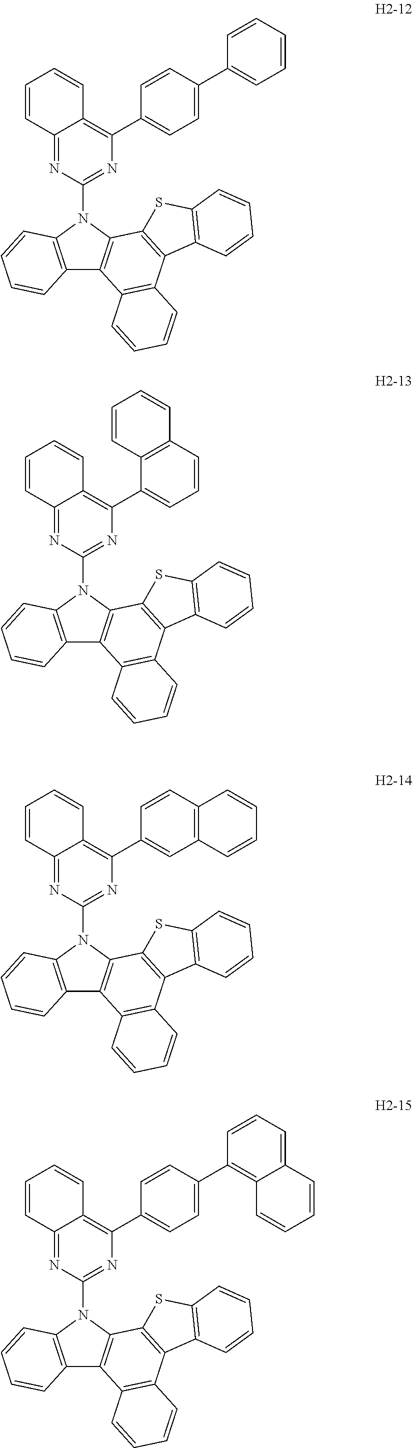

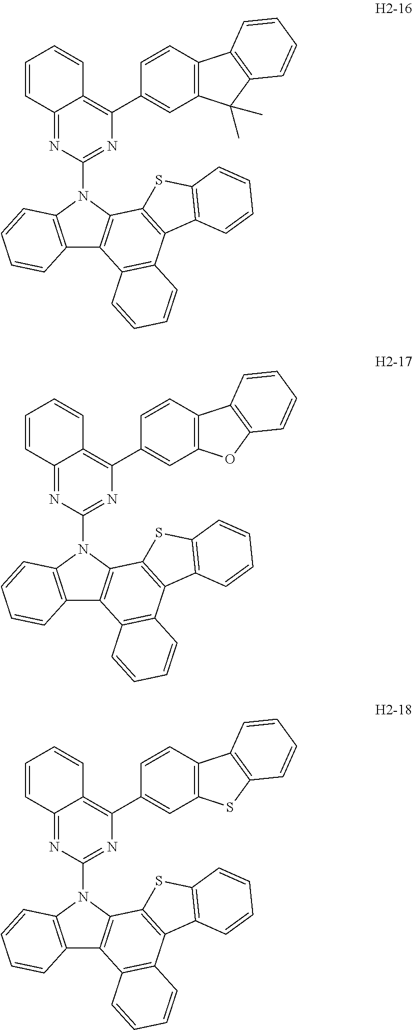 [SMILES: CC1(C)c2ccccc2-c2ccc(-c3nc(-n4c5ccccc5c5c6ccccc6c6c7ccccc7sc6c54)nc4ccccc34)cc21 ; c1ccc2c(-c3ccc4c(c3)oc3ccccc34)nc(-n3c4ccccc4c4c5ccccc5c5c6ccccc6sc5c43)nc2c1 ; c1ccc2c(-c3ccc4c(c3)sc3ccccc34)nc(-n3c4ccccc4c4c5ccccc5c5c6ccccc6sc5c43)nc2c1]

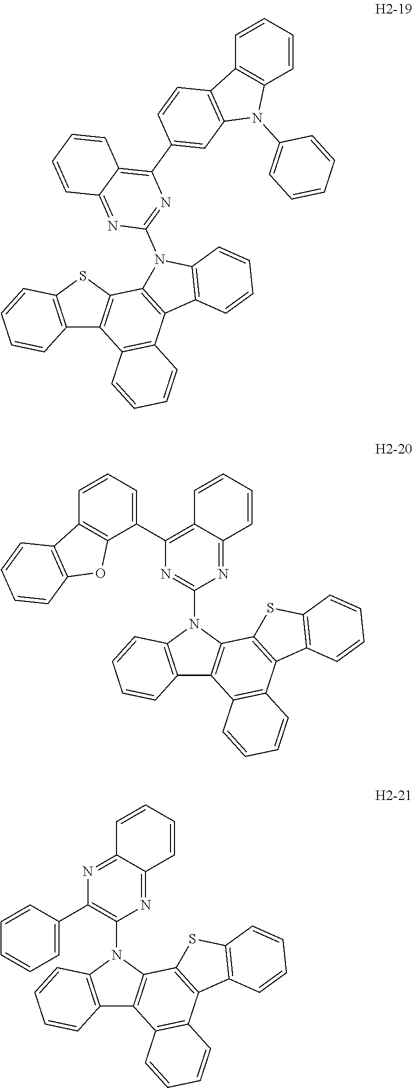 [SMILES: c1ccc(-n2c3ccccc3c3ccc(-c4nc(-n5c6ccccc6c6c7ccccc7c7c8ccccc8sc7c65)nc5ccccc45)cc32)cc1 ; c1ccc2c(-c3cccc4c3oc3ccccc34)nc(-n3c4ccccc4c4c5ccccc5c5c6ccccc6sc5c43)nc2c1 ; c1ccc(-c2nc3ccccc3nc2-n2c3ccccc3c3c4ccccc4c4c5ccccc5sc4c32)cc1]

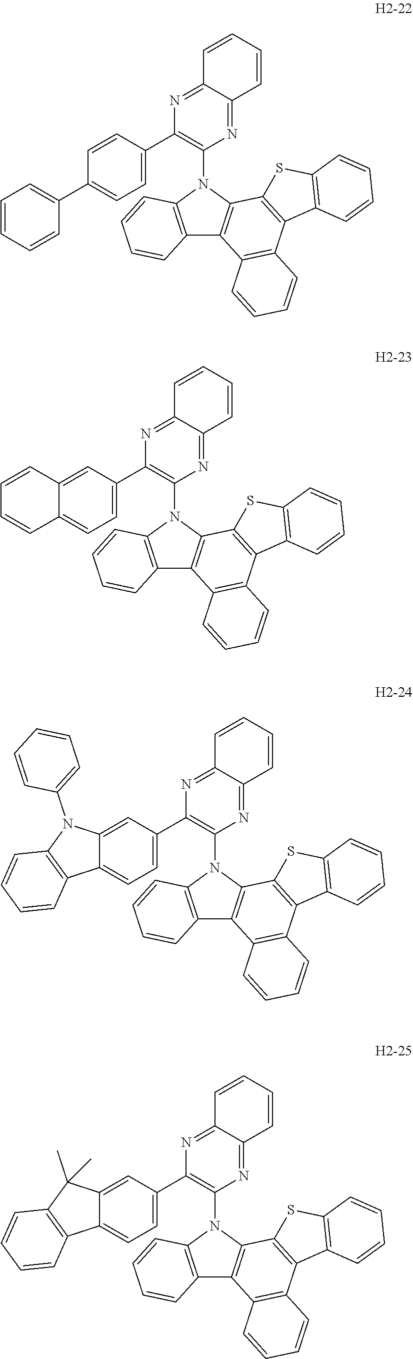 [SMILES: c1ccc(-c2ccc(-c3nc4ccccc4nc3-n3c4ccccc4c4c5ccccc5c5c6ccccc6sc5c43)cc2)cc1 ; c1ccc2cc(-c3nc4ccccc4nc3-n3c4ccccc4c4c5ccccc5c5c6ccccc6sc5c43)ccc2c1 ; c1ccc(-n2c3ccccc3c3ccc(-c4nc5ccccc5nc4-n4c5ccccc5c5c6ccccc6c6c7ccccc7sc6c54)cc32)cc1 ; CC1(C)c2ccccc2-c2ccc(-c3nc4ccccc4nc3-n3c4ccccc4c4c5ccccc5c5c6ccccc6sc5c43)cc21]

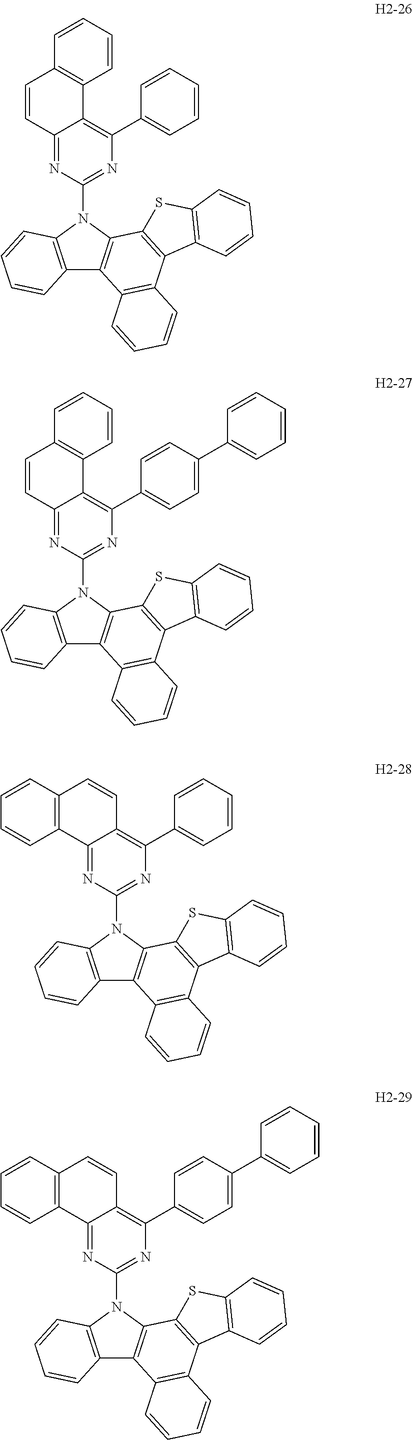 [SMILES: c1ccc(-c2nc(-n3c4ccccc4c4c5ccccc5c5c6ccccc6sc5c43)nc3ccc4ccccc4c23)cc1 ; c1ccc(-c2ccc(-c3nc(-n4c5ccccc5c5c6ccccc6c6c7ccccc7sc6c54)nc4ccc5ccccc5c34)cc2)cc1 ; c1ccc(-c2nc(-n3c4ccccc4c4c5ccccc5c5c6ccccc6sc5c43)nc3c2ccc2ccccc23)cc1 ; c1ccc(-c2ccc(-c3nc(-n4c5ccccc5c5c6ccccc6c6c7ccccc7sc6c54)nc4c3ccc3ccccc34)cc2)cc1]

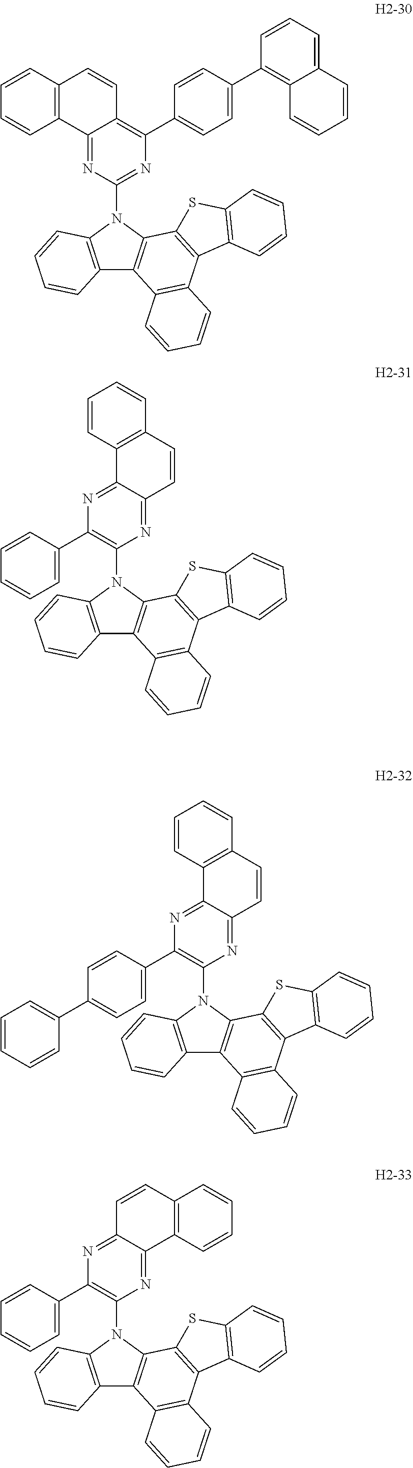 [SMILES: c1ccc2c(-c3ccc(-c4nc(-n5c6ccccc6c6c7ccccc7c7c8ccccc8sc7c65)nc5c4ccc4ccccc45)cc3)cccc2c1 ; c1ccc(-c2nc3c(ccc4ccccc43)nc2-n2c3ccccc3c3c4ccccc4c4c5ccccc5sc4c32)cc1 ; c1ccc(-c2ccc(-c3nc4c(ccc5ccccc54)nc3-n3c4ccccc4c4c5ccccc5c5c6ccccc6sc5c43)cc2)cc1 ; c1ccc(-c2nc3ccc4ccccc4c3nc2-n2c3ccccc3c3c4ccccc4c4c5ccccc5sc4c32)cc1]

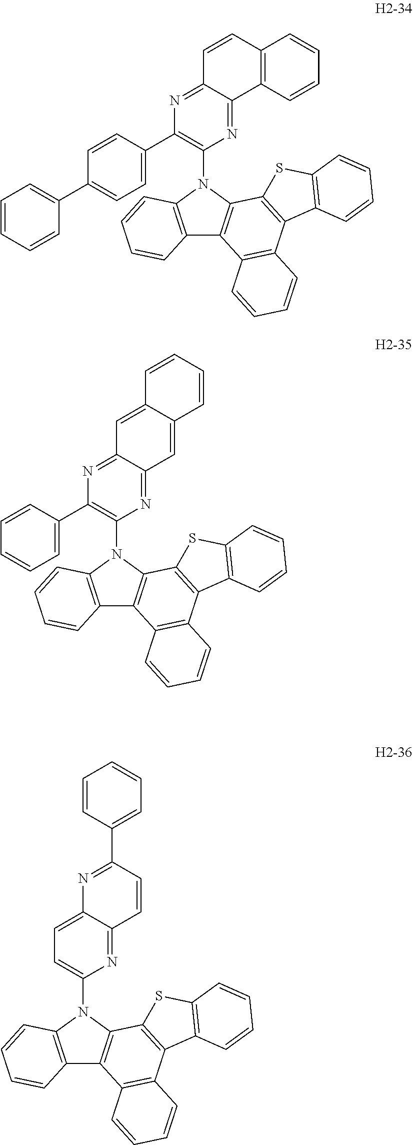 [SMILES: c1ccc(-c2ccc(-c3nc4ccc5ccccc5c4nc3-n3c4ccccc4c4c5ccccc5c5c6ccccc6sc5c43)cc2)cc1 ; c1ccc(-c2nc3cc4ccccc4cc3nc2-n2c3ccccc3c3c4ccccc4c4c5ccccc5sc4c32)cc1 ; c1ccc(-c2ccc3nc(-n4c5ccccc5c5c6ccccc6c6c7ccccc7sc6c54)ccc3n2)cc1]

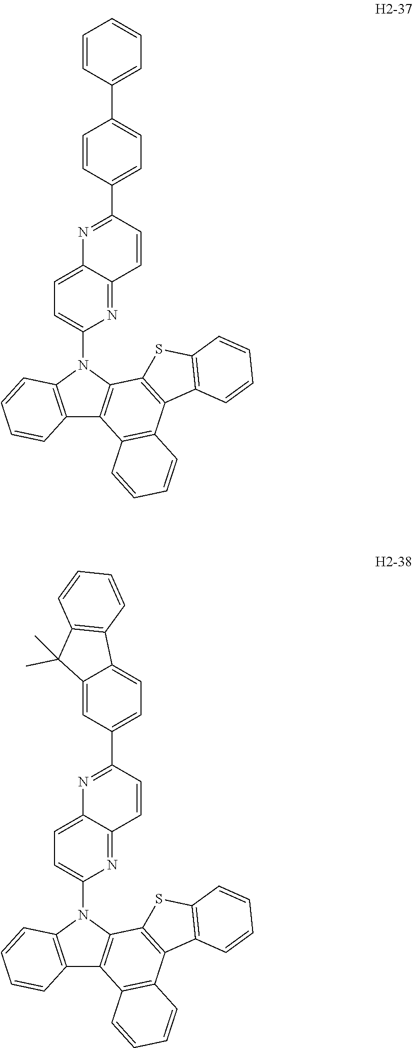 [SMILES: c1ccc(-c2ccc(-c3ccc4nc(-n5c6ccccc6c6c7ccccc7c7c8ccccc8sc7c65)ccc4n3)cc2)cc1 ; CC1(C)c2ccccc2-c2ccc(-c3ccc4nc(-n5c6ccccc6c6c7ccccc7c7c8ccccc8sc7c65)ccc4n3)cc21]

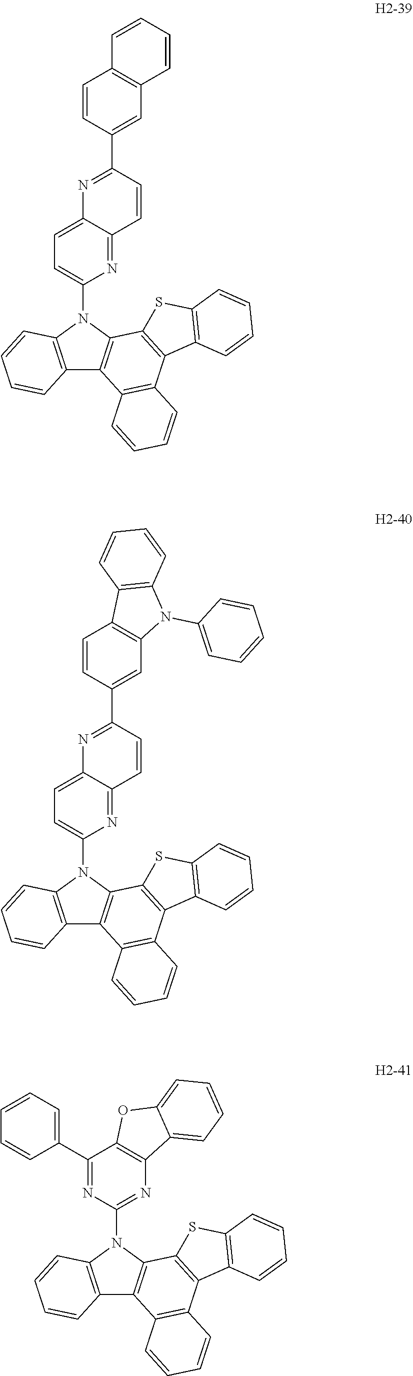 [SMILES: c1ccc2cc(-c3ccc4nc(-n5c6ccccc6c6c7ccccc7c7c8ccccc8sc7c65)ccc4n3)ccc2c1 ; c1ccc(-n2c3ccccc3c3ccc(-c4ccc5nc(-n6c7ccccc7c7c8ccccc8c8c9ccccc9sc8c76)ccc5n4)cc32)cc1 ; c1ccc(-c2nc(-n3c4ccccc4c4c5ccccc5c5c6ccccc6sc5c43)nc3c2oc2ccccc23)cc1]

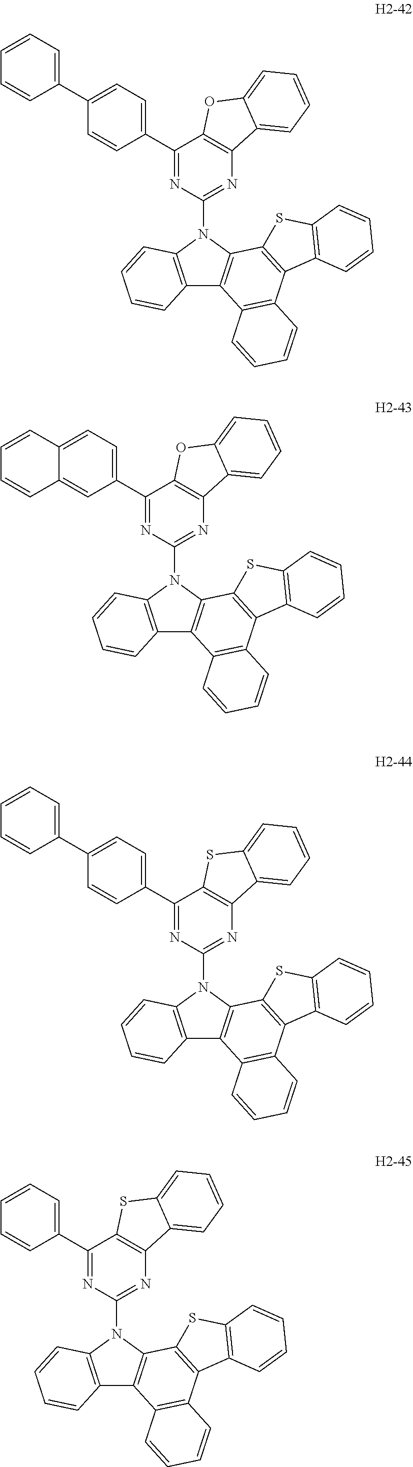 [SMILES: c1ccc(-c2ccc(-c3nc(-n4c5ccccc5c5c6ccccc6c6c7ccccc7sc6c54)nc4c3oc3ccccc34)cc2)cc1 ; c1ccc2cc(-c3nc(-n4c5ccccc5c5c6ccccc6c6c7ccccc7sc6c54)nc4c3oc3ccccc34)ccc2c1 ; c1ccc(-c2ccc(-c3nc(-n4c5ccccc5c5c6ccccc6c6c7ccccc7sc6c54)nc4c3sc3ccccc34)cc2)cc1 ; c1ccc(-c2nc(-n3c4ccccc4c4c5ccccc5c5c6ccccc6sc5c43)nc3c2sc2ccccc23)cc1]

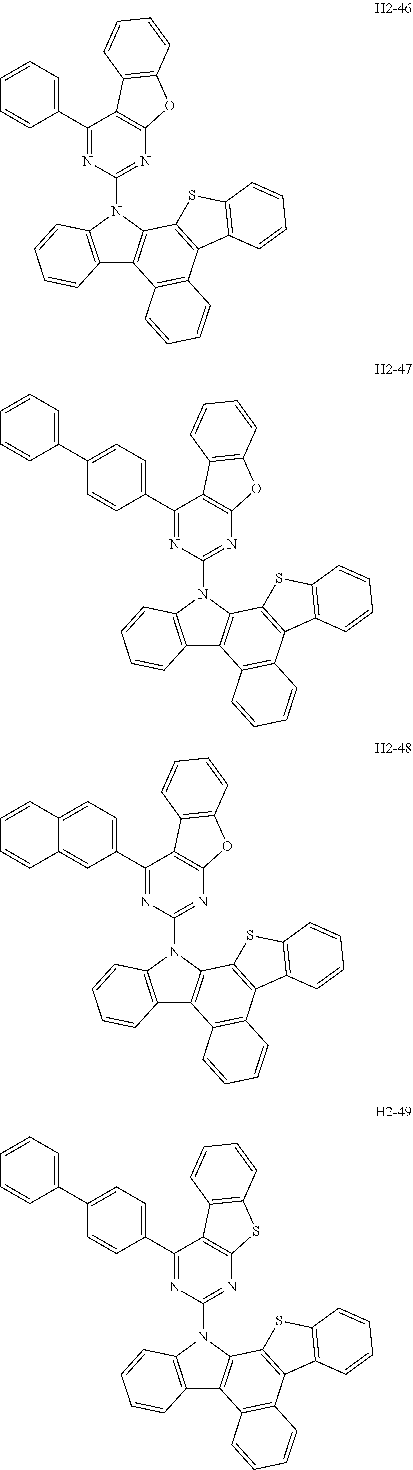 [SMILES: c1ccc(-c2nc(-n3c4ccccc4c4c5ccccc5c5c6ccccc6sc5c43)nc3oc4ccccc4c23)cc1 ; c1ccc(-c2ccc(-c3nc(-n4c5ccccc5c5c6ccccc6c6c7ccccc7sc6c54)nc4oc5ccccc5c34)cc2)cc1 ; c1ccc2cc(-c3nc(-n4c5ccccc5c5c6ccccc6c6c7ccccc7sc6c54)nc4oc5ccccc5c34)ccc2c1 ; c1ccc(-c2ccc(-c3nc(-n4c5ccccc5c5c6ccccc6c6c7ccccc7sc6c54)nc4sc5ccccc5c34)cc2)cc1]

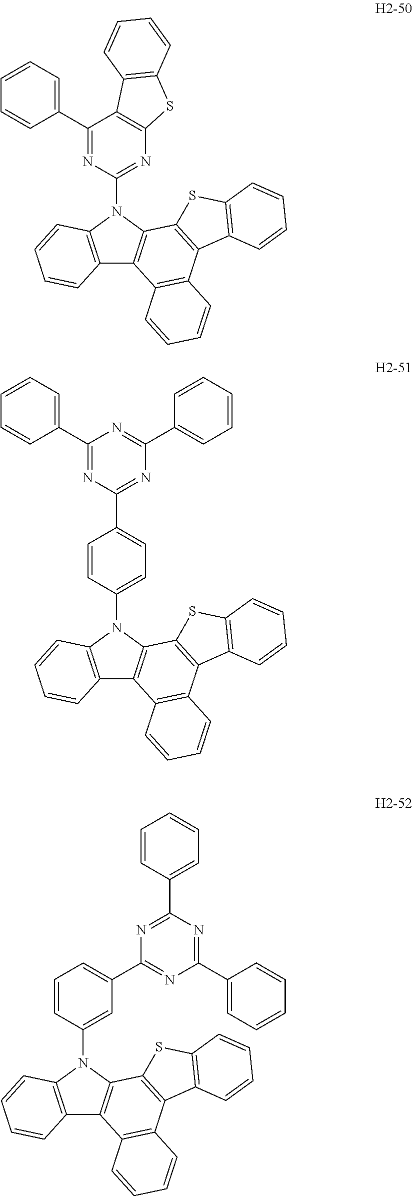

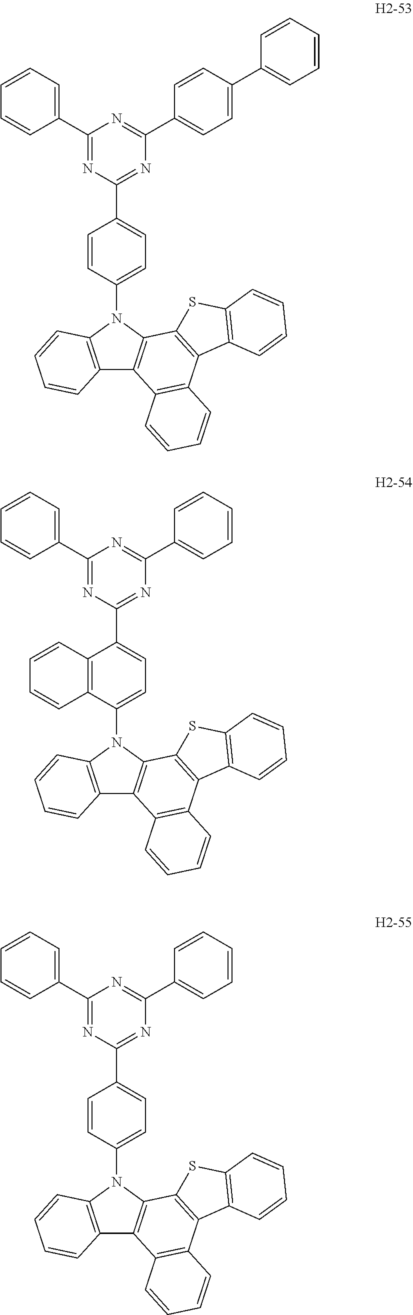 [SMILES: c1ccc(-c2ccc(-c3nc(-c4ccccc4)nc(-c4ccc(-n5c6ccccc6c6c7ccccc7c7c8ccccc8sc7c65)cc4)n3)cc2)cc1 ; c1ccc(-c2nc(-c3ccccc3)nc(-c3ccc(-n4c5ccccc5c5c6ccccc6c6c7ccccc7sc6c54)c4ccccc34)n2)cc1 ; c1ccc(-c2nc(-c3ccccc3)nc(-c3ccc(-n4c5ccccc5c5c6ccccc6c6c7ccccc7sc6c54)cc3)n2)cc1]

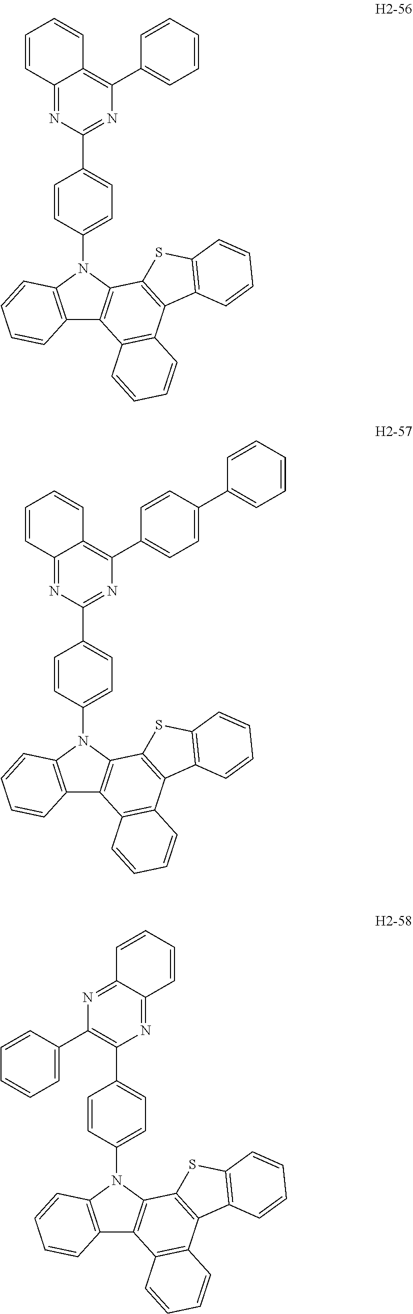 [SMILES: c1ccc(-c2nc(-c3ccc(-n4c5ccccc5c5c6ccccc6c6c7ccccc7sc6c54)cc3)nc3ccccc23)cc1 ; c1ccc(-c2ccc(-c3nc(-c4ccc(-n5c6ccccc6c6c7ccccc7c7c8ccccc8sc7c65)cc4)nc4ccccc34)cc2)cc1 ; c1ccc(-c2nc3ccccc3nc2-c2ccc(-n3c4ccccc4c4c5ccccc5c5c6ccccc6sc5c43)cc2)cc1]

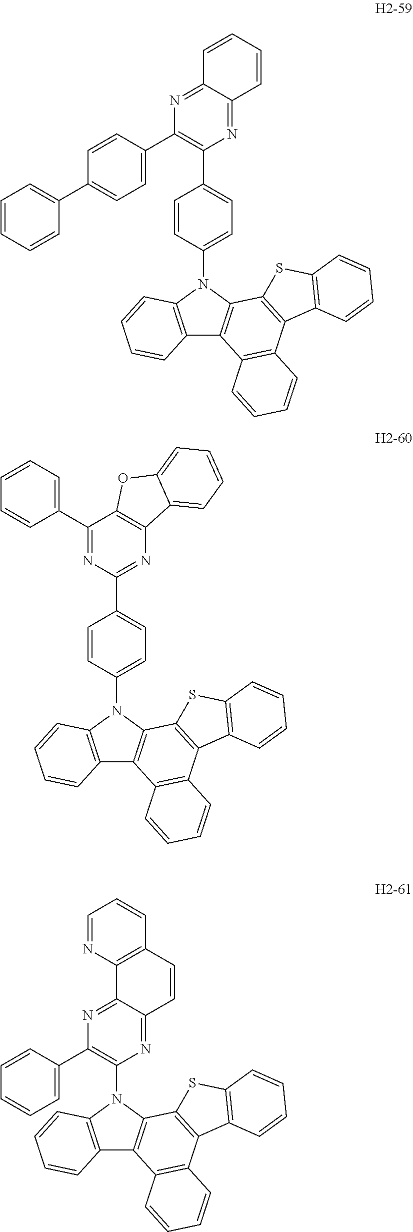 [SMILES: c1ccc(-c2ccc(-c3nc4ccccc4nc3-c3ccc(-n4c5ccccc5c5c6ccccc6c6c7ccccc7sc6c54)cc3)cc2)cc1 ; c1ccc(-c2nc(-c3ccc(-n4c5ccccc5c5c6ccccc6c6c7ccccc7sc6c54)cc3)nc3c2oc2ccccc23)cc1 ; c1ccc(-c2nc3c(ccc4cccnc43)nc2-n2c3ccccc3c3c4ccccc4c4c5ccccc5sc4c32)cc1]

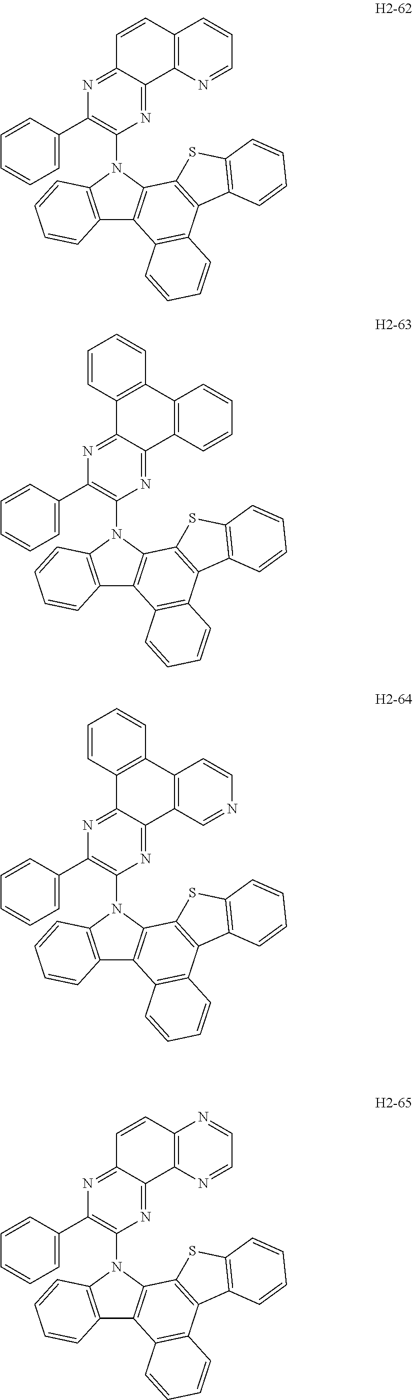 [SMILES: c1ccc(-c2nc3ccc4cccnc4c3nc2-n2c3ccccc3c3c4ccccc4c4c5ccccc5sc4c32)cc1 ; c1ccc(-c2nc3c4ccccc4c4ccccc4c3nc2-n2c3ccccc3c3c4ccccc4c4c5ccccc5sc4c32)cc1 ; c1ccc(-c2nc3c4ccccc4c4ccncc4c3nc2-n2c3ccccc3c3c4ccccc4c4c5ccccc5sc4c32)cc1 ; c1ccc(-c2nc3ccc4nccnc4c3nc2-n2c3ccccc3c3c4ccccc4c4c5ccccc5sc4c32)cc1]

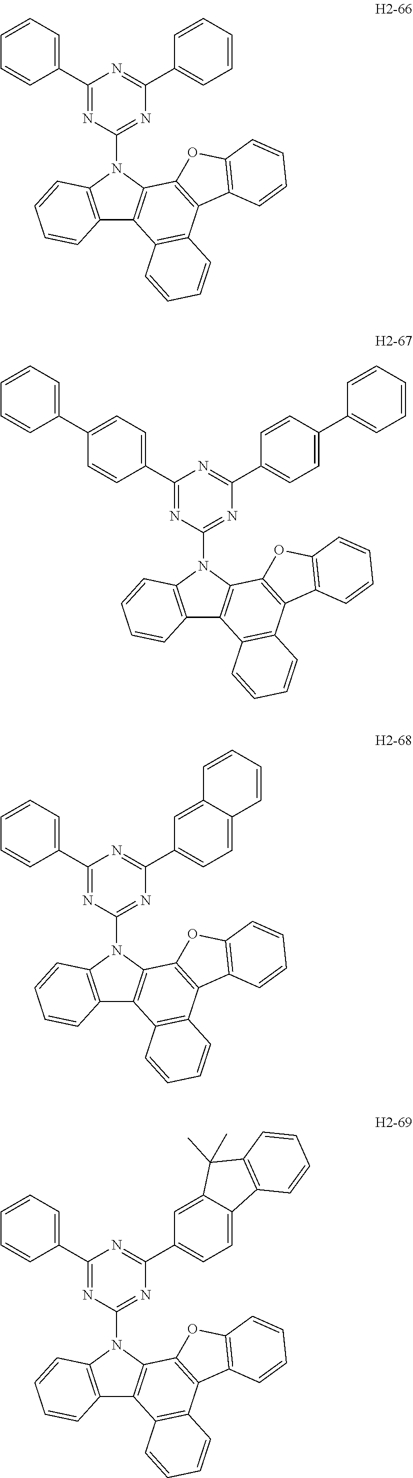 [SMILES: c1ccc(-c2nc(-c3ccccc3)nc(-n3c4ccccc4c4c5ccccc5c5c6ccccc6oc5c43)n2)cc1 ; c1ccc(-c2ccc(-c3nc(-c4ccc(-c5ccccc5)cc4)nc(-n4c5ccccc5c5c6ccccc6c6c7ccccc7oc6c54)n3)cc2)cc1 ; c1ccc(-c2nc(-c3ccc4ccccc4c3)nc(-n3c4ccccc4c4c5ccccc5c5c6ccccc6oc5c43)n2)cc1 ; CC1(C)c2ccccc2-c2ccc(-c3nc(-c4ccccc4)nc(-n4c5ccccc5c5c6ccccc6c6c7ccccc7oc6c54)n3)cc21]

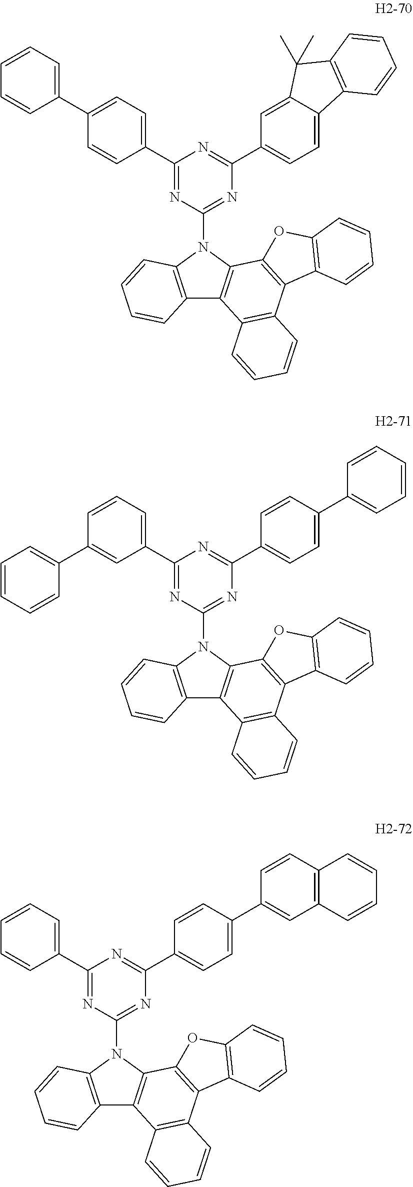 [SMILES: CC1(C)c2ccccc2-c2ccc(-c3nc(-c4ccc(-c5ccccc5)cc4)nc(-n4c5ccccc5c5c6ccccc6c6c7ccccc7oc6c54)n3)cc21 ; c1ccc(-c2ccc(-c3nc(-c4cccc(-c5ccccc5)c4)nc(-n4c5ccccc5c5c6ccccc6c6c7ccccc7oc6c54)n3)cc2)cc1 ; c1ccc(-c2nc(-c3ccc(-c4ccc5ccccc5c4)cc3)nc(-n3c4ccccc4c4c5ccccc5c5c6ccccc6oc5c43)n2)cc1]

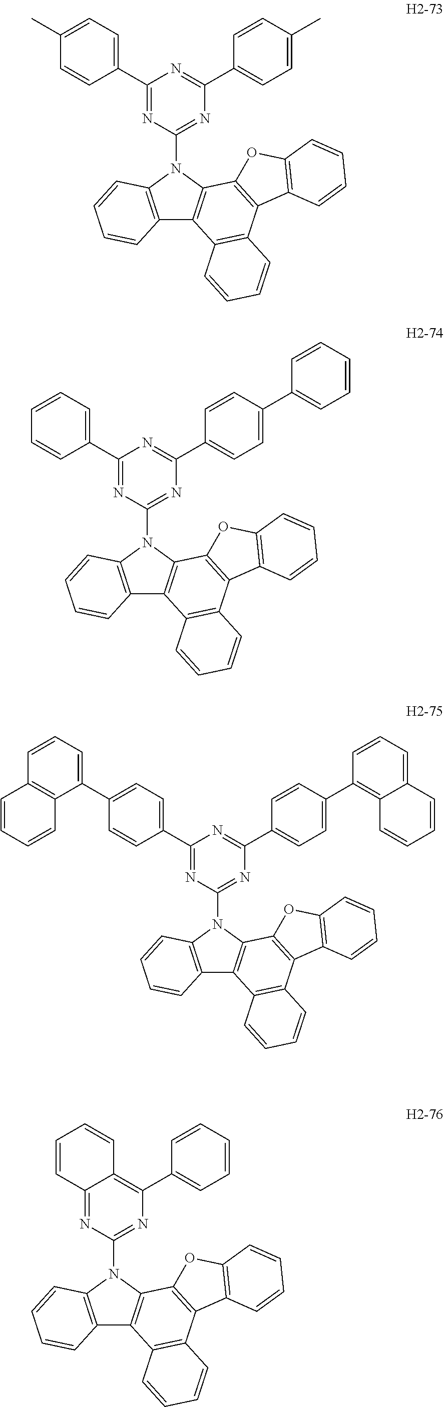 [SMILES: Cc1ccc(-c2nc(-c3ccc(C)cc3)nc(-n3c4ccccc4c4c5ccccc5c5c6ccccc6oc5c43)n2)cc1 ; c1ccc(-c2ccc(-c3nc(-c4ccccc4)nc(-n4c5ccccc5c5c6ccccc6c6c7ccccc7oc6c54)n3)cc2)cc1 ; c1ccc2c(-c3ccc(-c4nc(-c5ccc(-c6cccc7ccccc67)cc5)nc(-n5c6ccccc6c6c7ccccc7c7c8ccccc8oc7c65)n4)cc3)cccc2c1 ; c1ccc(-c2nc(-n3c4ccccc4c4c5ccccc5c5c6ccccc6oc5c43)nc3ccccc23)cc1]

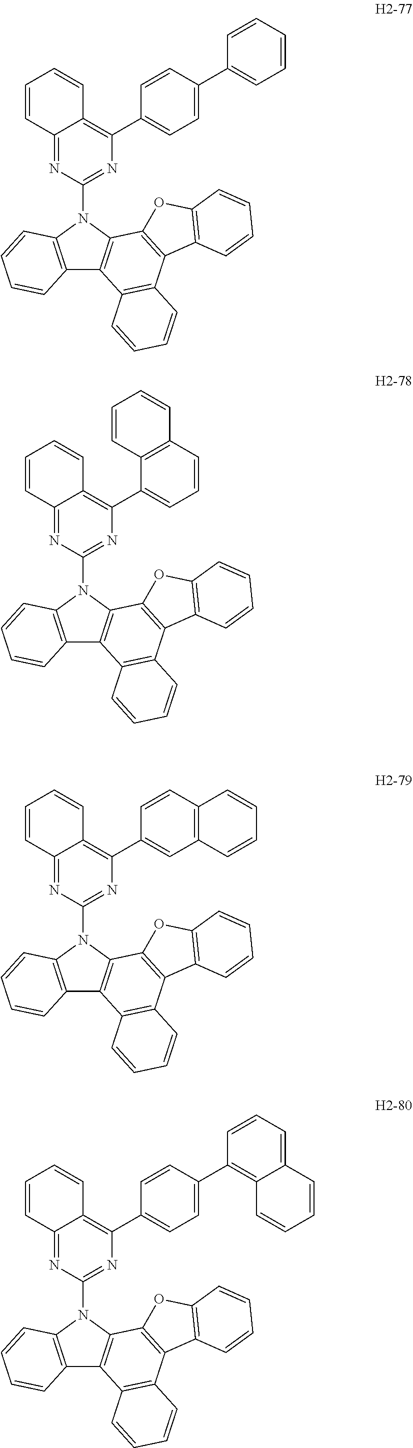

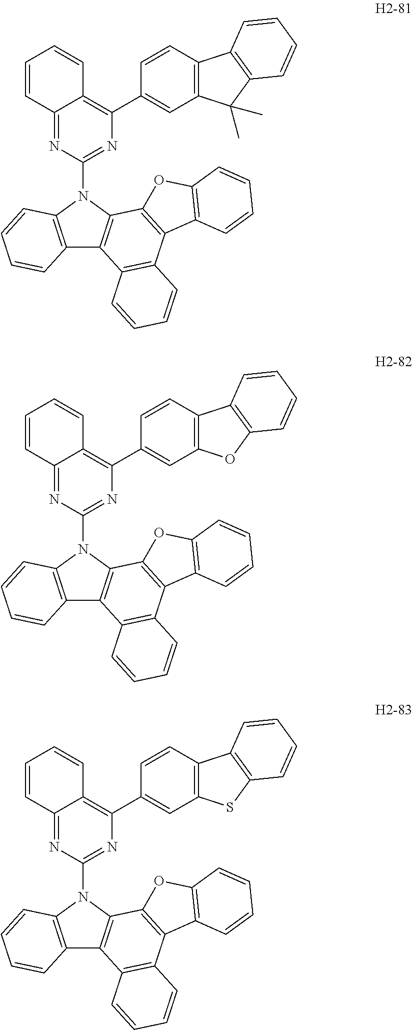 [SMILES: CC1(C)c2ccccc2-c2ccc(-c3nc(-n4c5ccccc5c5c6ccccc6c6c7ccccc7oc6c54)nc4ccccc34)cc21 ; c1ccc2c(-c3ccc4c(c3)oc3ccccc34)nc(-n3c4ccccc4c4c5ccccc5c5c6ccccc6oc5c43)nc2c1 ; c1ccc2c(-c3ccc4c(c3)sc3ccccc34)nc(-n3c4ccccc4c4c5ccccc5c5c6ccccc6oc5c43)nc2c1]

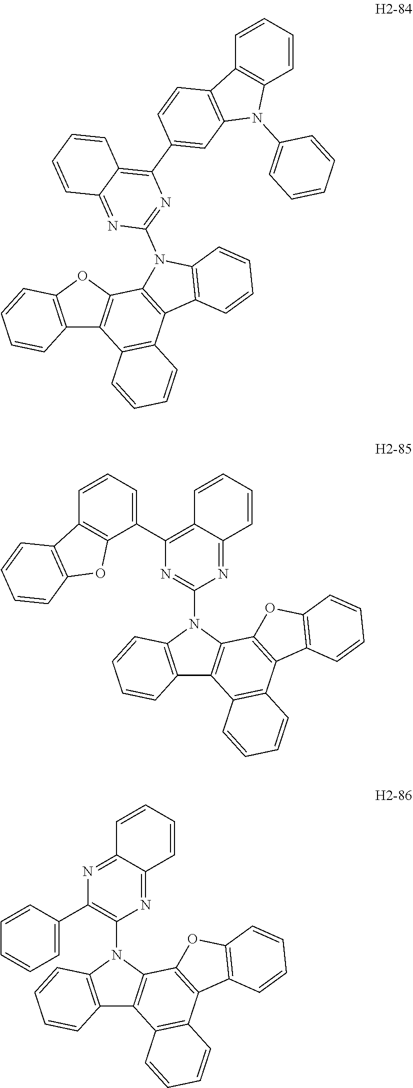 [SMILES: c1ccc(-n2c3ccccc3c3ccc(-c4nc(-n5c6ccccc6c6c7ccccc7c7c8ccccc8oc7c65)nc5ccccc45)cc32)cc1 ; c1ccc2c(-c3cccc4c3oc3ccccc34)nc(-n3c4ccccc4c4c5ccccc5c5c6ccccc6oc5c43)nc2c1 ; c1ccc(-c2nc3ccccc3nc2-n2c3ccccc3c3c4ccccc4c4c5ccccc5oc4c32)cc1]

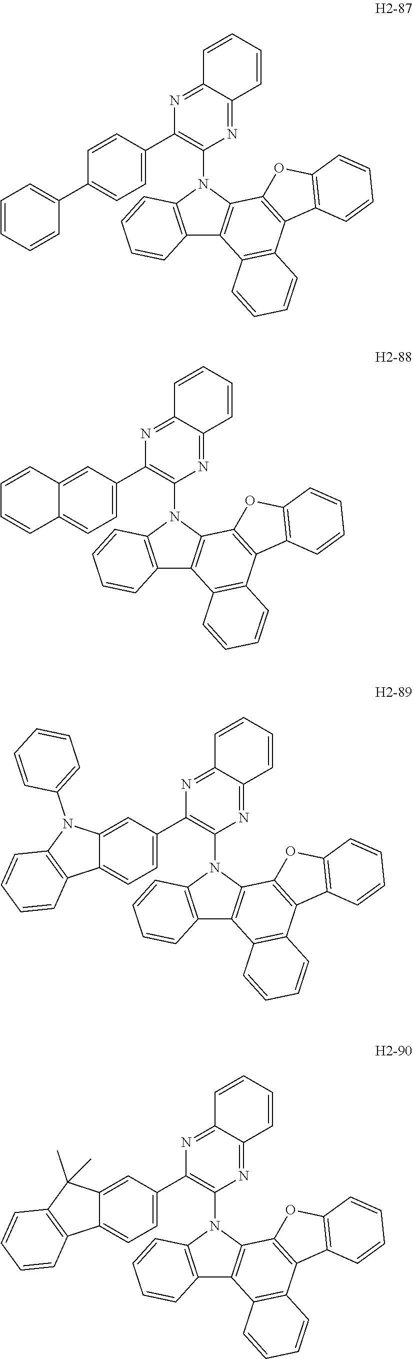 [SMILES: c1ccc(-c2ccc(-c3nc4ccccc4nc3-n3c4ccccc4c4c5ccccc5c5c6ccccc6oc5c43)cc2)cc1 ; c1ccc2cc(-c3nc4ccccc4nc3-n3c4ccccc4c4c5ccccc5c5c6ccccc6oc5c43)ccc2c1 ; c1ccc(-n2c3ccccc3c3ccc(-c4nc5ccccc5nc4-n4c5ccccc5c5c6ccccc6c6c7ccccc7oc6c54)cc32)cc1 ; CC1(C)c2ccccc2-c2ccc(-c3nc4ccccc4nc3-n3c4ccccc4c4c5ccccc5c5c6ccccc6oc5c43)cc21]

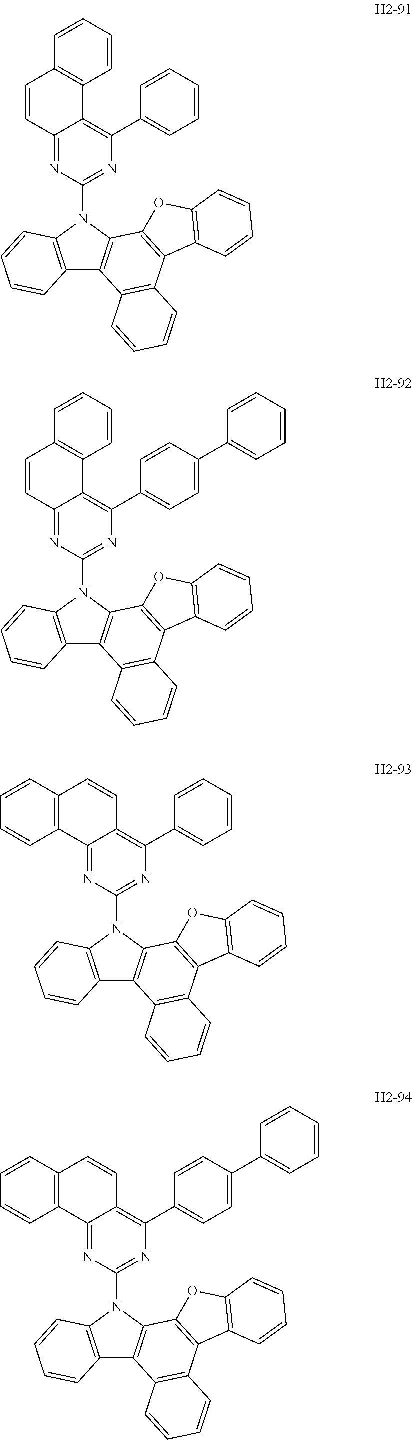 [SMILES: c1ccc(-c2nc(-n3c4ccccc4c4c5ccccc5c5c6ccccc6oc5c43)nc3ccc4ccccc4c23)cc1 ; c1ccc(-c2ccc(-c3nc(-n4c5ccccc5c5c6ccccc6c6c7ccccc7oc6c54)nc4ccc5ccccc5c34)cc2)cc1 ; c1ccc(-c2nc(-n3c4ccccc4c4c5ccccc5c5c6ccccc6oc5c43)nc3c2ccc2ccccc23)cc1 ; c1ccc(-c2ccc(-c3nc(-n4c5ccccc5c5c6ccccc6c6c7ccccc7oc6c54)nc4c3ccc3ccccc34)cc2)cc1]

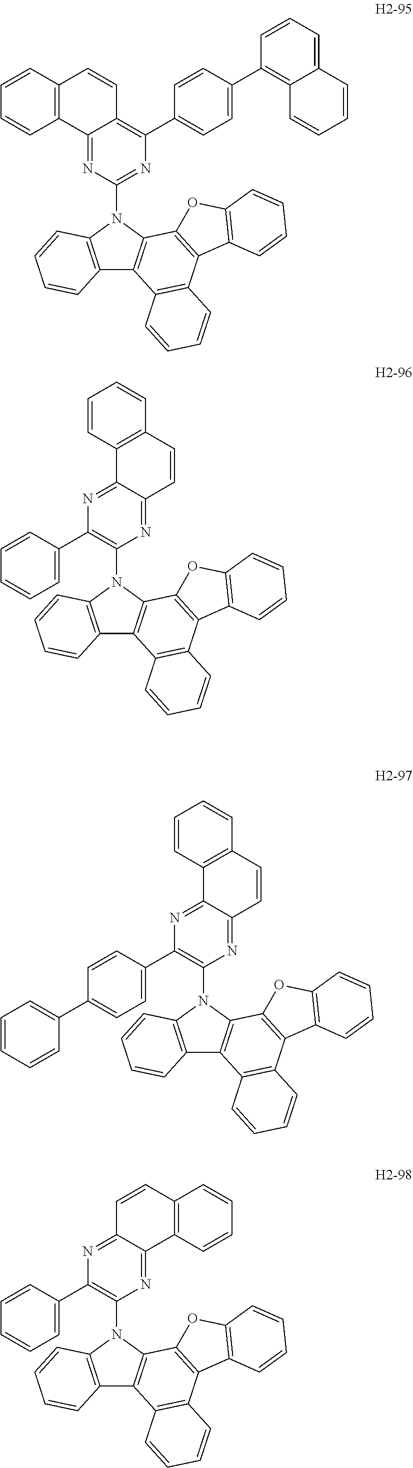 [SMILES: c1ccc2c(-c3ccc(-c4nc(-n5c6ccccc6c6c7ccccc7c7c8ccccc8oc7c65)nc5c4ccc4ccccc45)cc3)cccc2c1 ; c1ccc(-c2nc3c(ccc4ccccc43)nc2-n2c3ccccc3c3c4ccccc4c4c5ccccc5oc4c32)cc1 ; c1ccc(-c2ccc(-c3nc4c(ccc5ccccc54)nc3-n3c4ccccc4c4c5ccccc5c5c6ccccc6oc5c43)cc2)cc1 ; c1ccc(-c2nc3ccc4ccccc4c3nc2-n2c3ccccc3c3c4ccccc4c4c5ccccc5oc4c32)cc1]

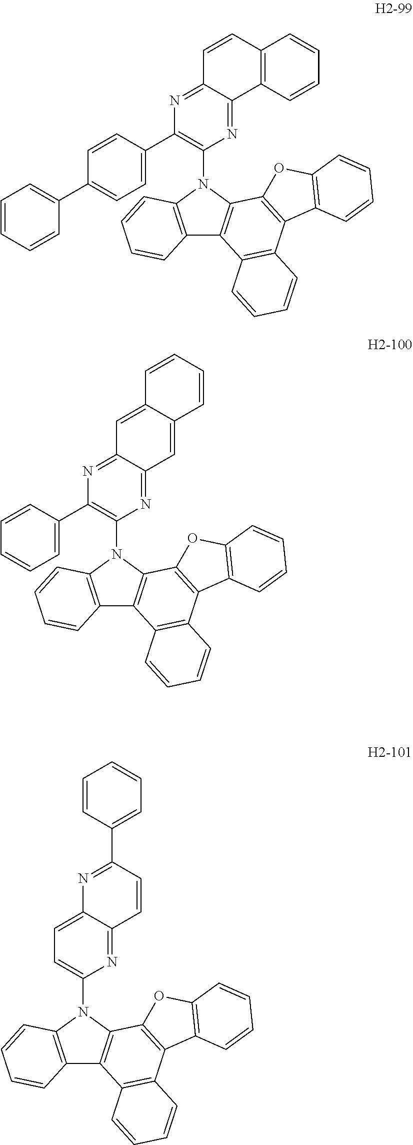 [SMILES: c1ccc(-c2ccc(-c3nc4ccc5ccccc5c4nc3-n3c4ccccc4c4c5ccccc5c5c6ccccc6oc5c43)cc2)cc1 ; c1ccc(-c2nc3cc4ccccc4cc3nc2-n2c3ccccc3c3c4ccccc4c4c5ccccc5oc4c32)cc1 ; c1ccc(-c2ccc3nc(-n4c5ccccc5c5c6ccccc6c6c7ccccc7oc6c54)ccc3n2)cc1]

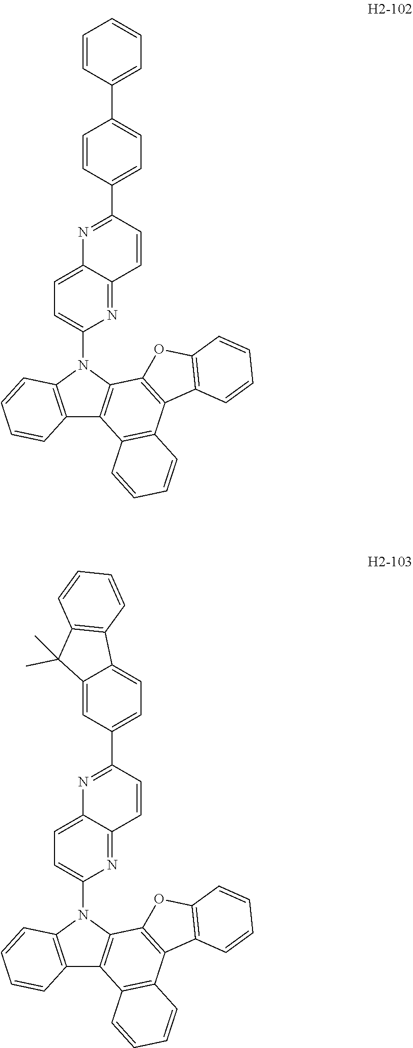 [SMILES: c1ccc(-c2ccc(-c3ccc4nc(-n5c6ccccc6c6c7ccccc7c7c8ccccc8oc7c65)ccc4n3)cc2)cc1 ; CC1(C)c2ccccc2-c2ccc(-c3ccc4nc(-n5c6ccccc6c6c7ccccc7c7c8ccccc8oc7c65)ccc4n3)cc21]

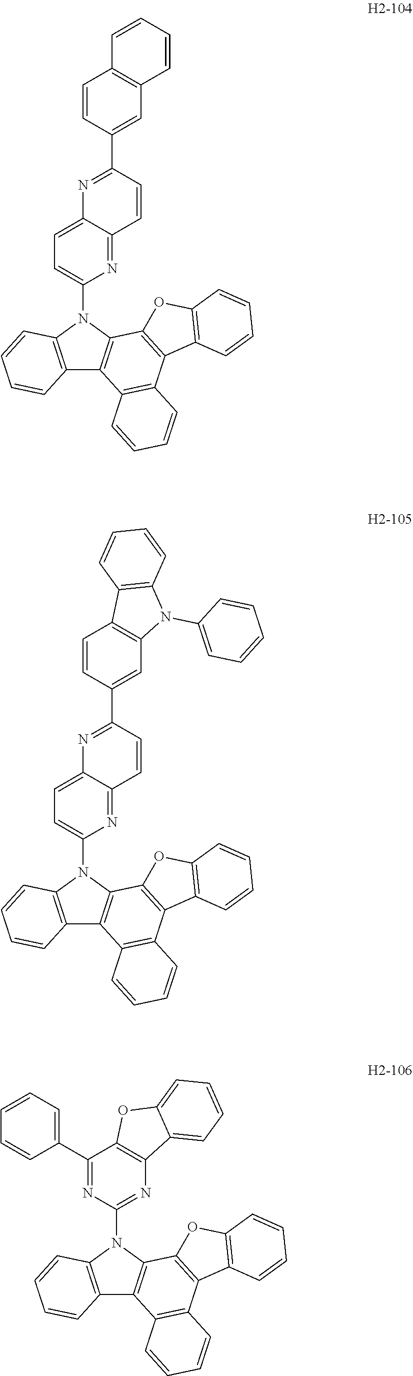 [SMILES: c1ccc2cc(-c3ccc4nc(-n5c6ccccc6c6c7ccccc7c7c8ccccc8oc7c65)ccc4n3)ccc2c1 ; c1ccc(-n2c3ccccc3c3ccc(-c4ccc5nc(-n6c7ccccc7c7c8ccccc8c8c9ccccc9oc8c76)ccc5n4)cc32)cc1 ; c1ccc(-c2nc(-n3c4ccccc4c4c5ccccc5c5c6ccccc6oc5c43)nc3c2oc2ccccc23)cc1]

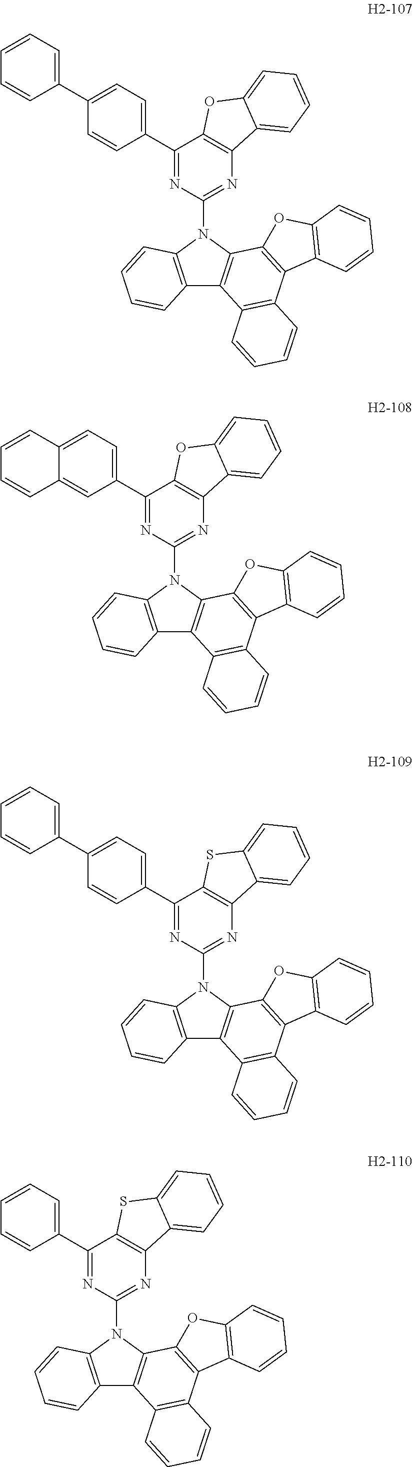 [SMILES: c1ccc(-c2ccc(-c3nc(-n4c5ccccc5c5c6ccccc6c6c7ccccc7oc6c54)nc4c3oc3ccccc34)cc2)cc1 ; c1ccc2cc(-c3nc(-n4c5ccccc5c5c6ccccc6c6c7ccccc7oc6c54)nc4c3oc3ccccc34)ccc2c1 ; c1ccc(-c2ccc(-c3nc(-n4c5ccccc5c5c6ccccc6c6c7ccccc7oc6c54)nc4c3sc3ccccc34)cc2)cc1 ; c1ccc(-c2nc(-n3c4ccccc4c4c5ccccc5c5c6ccccc6oc5c43)nc3c2sc2ccccc23)cc1]

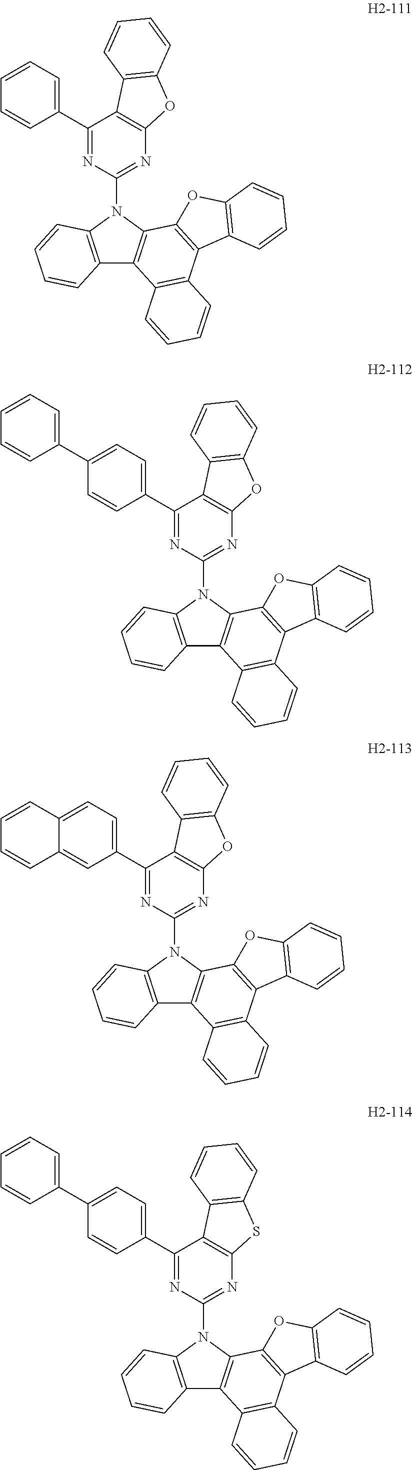 [SMILES: c1ccc(-c2nc(-n3c4ccccc4c4c5ccccc5c5c6ccccc6oc5c43)nc3oc4ccccc4c23)cc1 ; c1ccc(-c2ccc(-c3nc(-n4c5ccccc5c5c6ccccc6c6c7ccccc7oc6c54)nc4oc5ccccc5c34)cc2)cc1 ; c1ccc2cc(-c3nc(-n4c5ccccc5c5c6ccccc6c6c7ccccc7oc6c54)nc4oc5ccccc5c34)ccc2c1 ; c1ccc(-c2ccc(-c3nc(-n4c5ccccc5c5c6ccccc6c6c7ccccc7oc6c54)nc4sc5ccccc5c34)cc2)cc1]

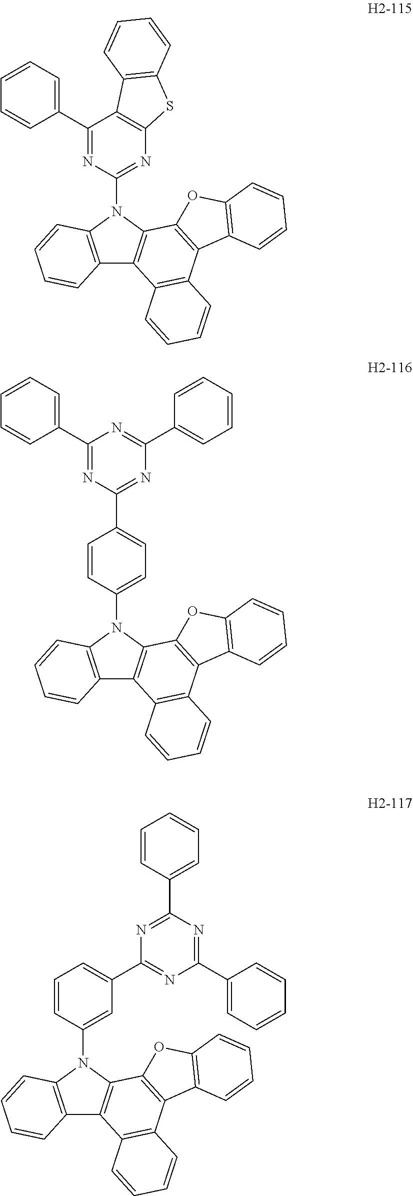

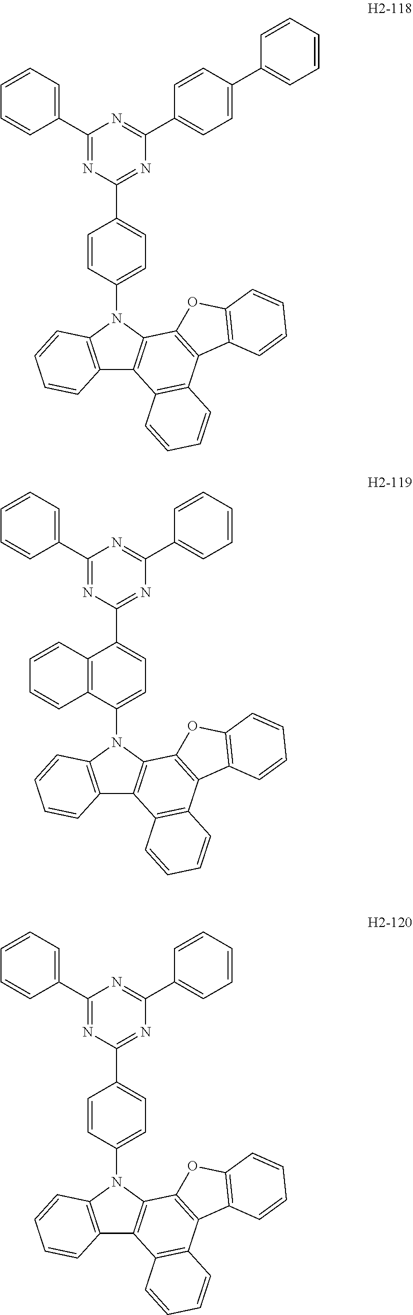 [SMILES: c1ccc(-c2ccc(-c3nc(-c4ccccc4)nc(-c4ccc(-n5c6ccccc6c6c7ccccc7c7c8ccccc8oc7c65)cc4)n3)cc2)cc1 ; c1ccc(-c2nc(-c3ccccc3)nc(-c3ccc(-n4c5ccccc5c5c6ccccc6c6c7ccccc7oc6c54)c4ccccc34)n2)cc1 ; c1ccc(-c2nc(-c3ccccc3)nc(-c3ccc(-n4c5ccccc5c5c6ccccc6c6c7ccccc7oc6c54)cc3)n2)cc1]

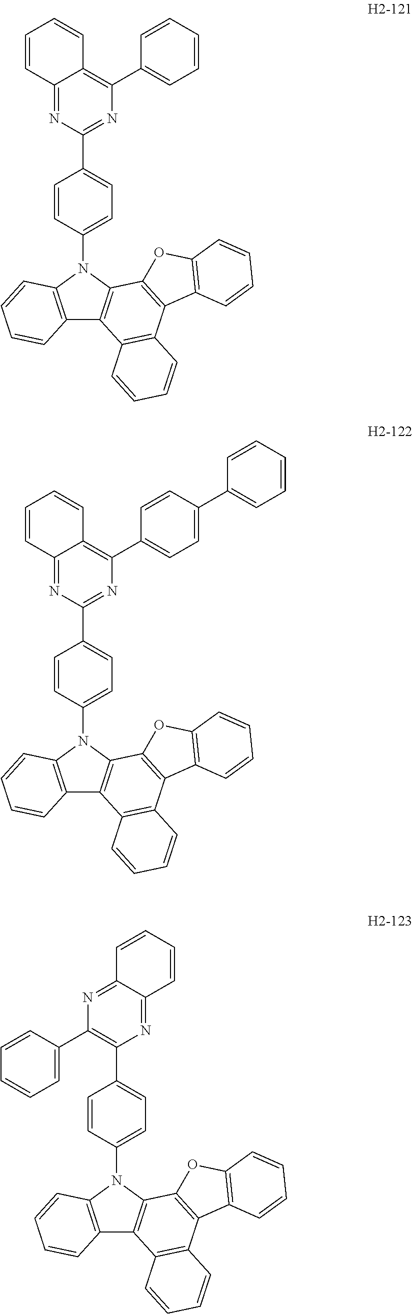 [SMILES: c1ccc(-c2nc(-c3ccc(-n4c5ccccc5c5c6ccccc6c6c7ccccc7oc6c54)cc3)nc3ccccc23)cc1 ; c1ccc(-c2ccc(-c3nc(-c4ccc(-n5c6ccccc6c6c7ccccc7c7c8ccccc8oc7c65)cc4)nc4ccccc34)cc2)cc1 ; c1ccc(-c2nc3ccccc3nc2-c2ccc(-n3c4ccccc4c4c5ccccc5c5c6ccccc6oc5c43)cc2)cc1]

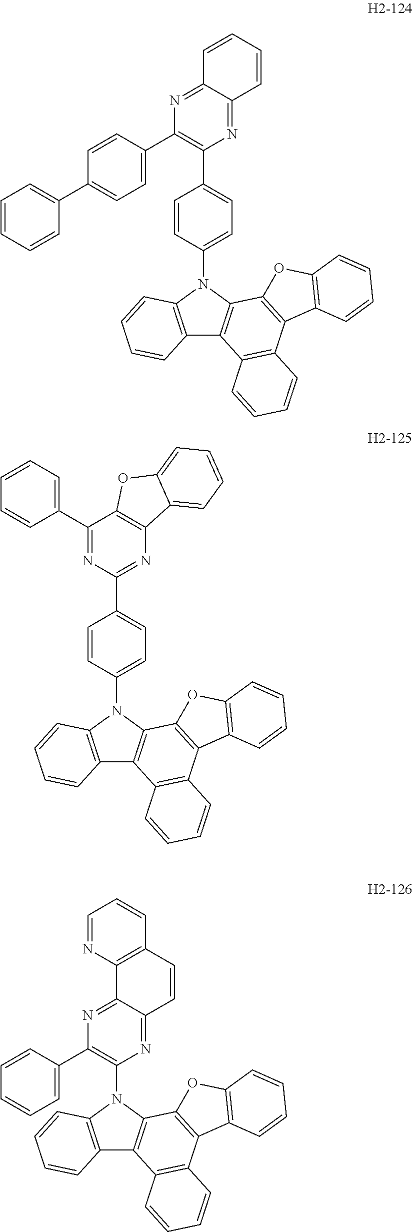 [SMILES: c1ccc(-c2ccc(-c3nc4ccccc4nc3-c3ccc(-n4c5ccccc5c5c6ccccc6c6c7ccccc7oc6c54)cc3)cc2)cc1 ; c1ccc(-c2nc(-c3ccc(-n4c5ccccc5c5c6ccccc6c6c7ccccc7oc6c54)cc3)nc3c2oc2ccccc23)cc1 ; c1ccc(-c2nc3c(ccc4cccnc43)nc2-n2c3ccccc3c3c4ccccc4c4c5ccccc5oc4c32)cc1]

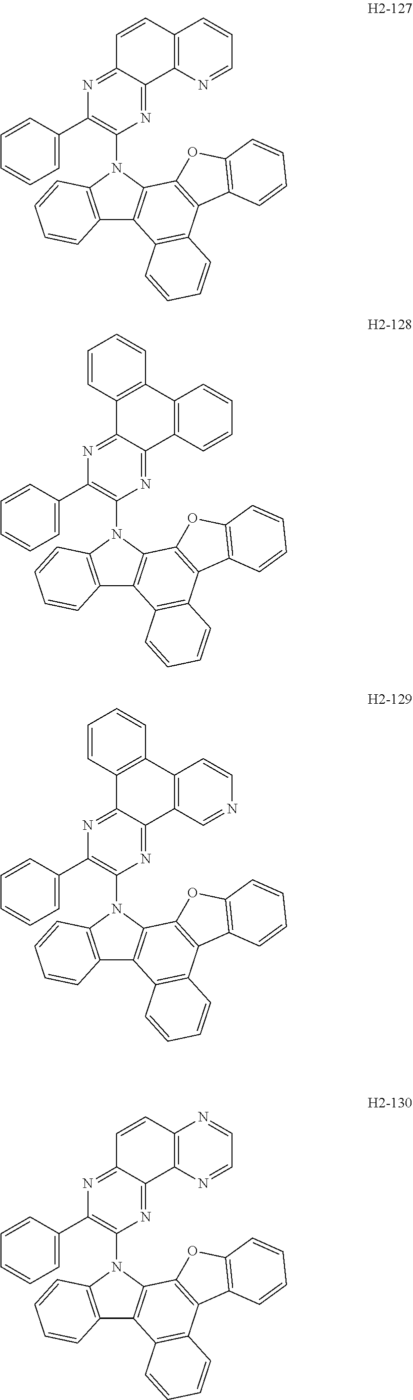 [SMILES: c1ccc(-c2nc3ccc4cccnc4c3nc2-n2c3ccccc3c3c4ccccc4c4c5ccccc5oc4c32)cc1 ; c1ccc(-c2nc3c4ccccc4c4ccccc4c3nc2-n2c3ccccc3c3c4ccccc4c4c5ccccc5oc4c32)cc1 ; c1ccc(-c2nc3c4ccccc4c4ccncc4c3nc2-n2c3ccccc3c3c4ccccc4c4c5ccccc5oc4c32)cc1 ; c1ccc(-c2nc3ccc4nccnc4c3nc2-n2c3ccccc3c3c4ccccc4c4c5ccccc5oc4c32)cc1]

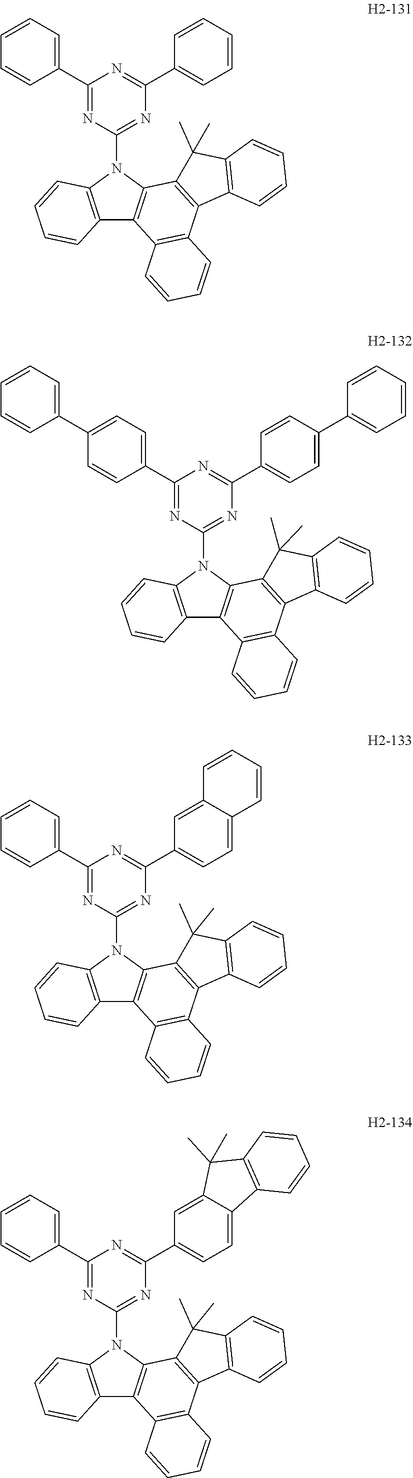 [SMILES: CC1(C)c2ccccc2-c2c1c1c(c3ccccc23)c2ccccc2n1-c1nc(-c2ccccc2)nc(-c2ccccc2)n1 ; CC1(C)c2ccccc2-c2c1c1c(c3ccccc23)c2ccccc2n1-c1nc(-c2ccc(-c3ccccc3)cc2)nc(-c2ccc(-c3ccccc3)cc2)n1 ; CC1(C)c2ccccc2-c2c1c1c(c3ccccc23)c2ccccc2n1-c1nc(-c2ccccc2)nc(-c2ccc3ccccc3c2)n1 ; CC1(C)c2ccccc2-c2ccc(-c3nc(-c4ccccc4)nc(-n4c5ccccc5c5c6ccccc6c6c(c54)C(C)(C)c4ccccc4-6)n3)cc21]

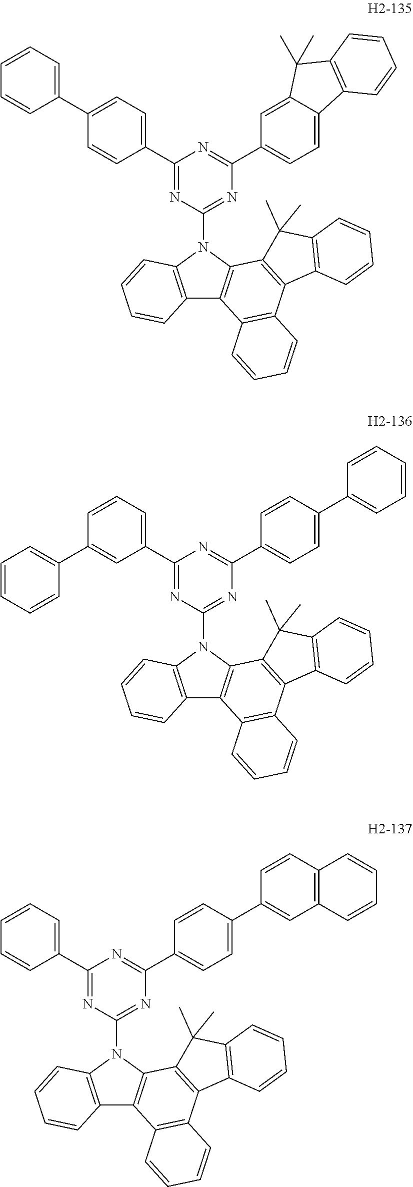 [SMILES: CC1(C)c2ccccc2-c2ccc(-c3nc(-c4ccc(-c5ccccc5)cc4)nc(-n4c5ccccc5c5c6ccccc6c6c(c54)C(C)(C)c4ccccc4-6)n3)cc21 ; CC1(C)c2ccccc2-c2c1c1c(c3ccccc23)c2ccccc2n1-c1nc(-c2ccc(-c3ccccc3)cc2)nc(-c2cccc(-c3ccccc3)c2)n1 ; CC1(C)c2ccccc2-c2c1c1c(c3ccccc23)c2ccccc2n1-c1nc(-c2ccccc2)nc(-c2ccc(-c3ccc4ccccc4c3)cc2)n1]

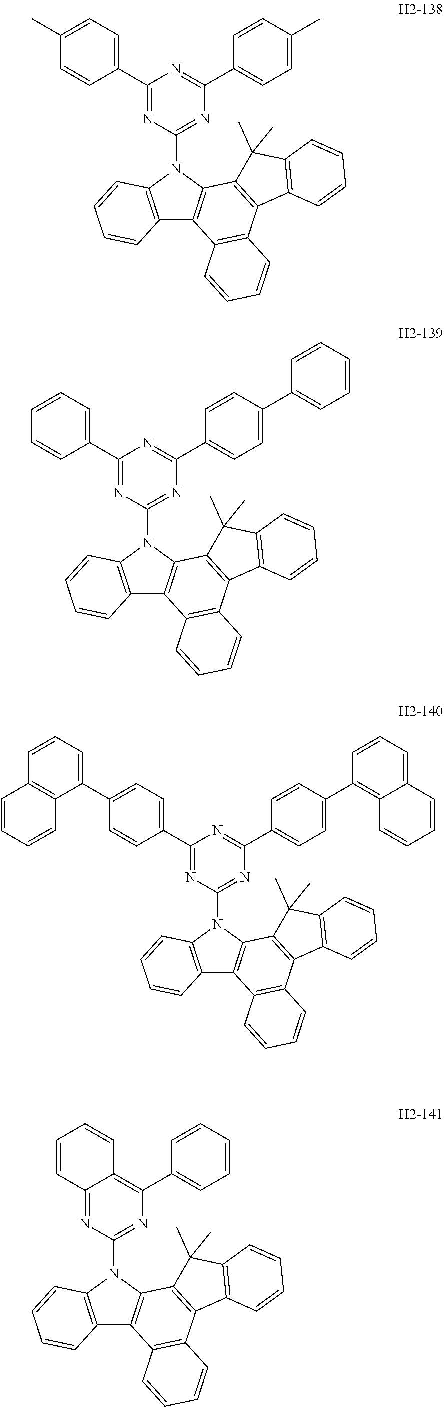 [SMILES: Cc1ccc(-c2nc(-c3ccc(C)cc3)nc(-n3c4ccccc4c4c5ccccc5c5c(c43)C(C)(C)c3ccccc3-5)n2)cc1 ; CC1(C)c2ccccc2-c2c1c1c(c3ccccc23)c2ccccc2n1-c1nc(-c2ccccc2)nc(-c2ccc(-c3ccccc3)cc2)n1 ; CC1(C)c2ccccc2-c2c1c1c(c3ccccc23)c2ccccc2n1-c1nc(-c2ccc(-c3cccc4ccccc34)cc2)nc(-c2ccc(-c3cccc4ccccc34)cc2)n1 ; CC1(C)c2ccccc2-c2c1c1c(c3ccccc23)c2ccccc2n1-c1nc(-c2ccccc2)c2ccccc2n1]

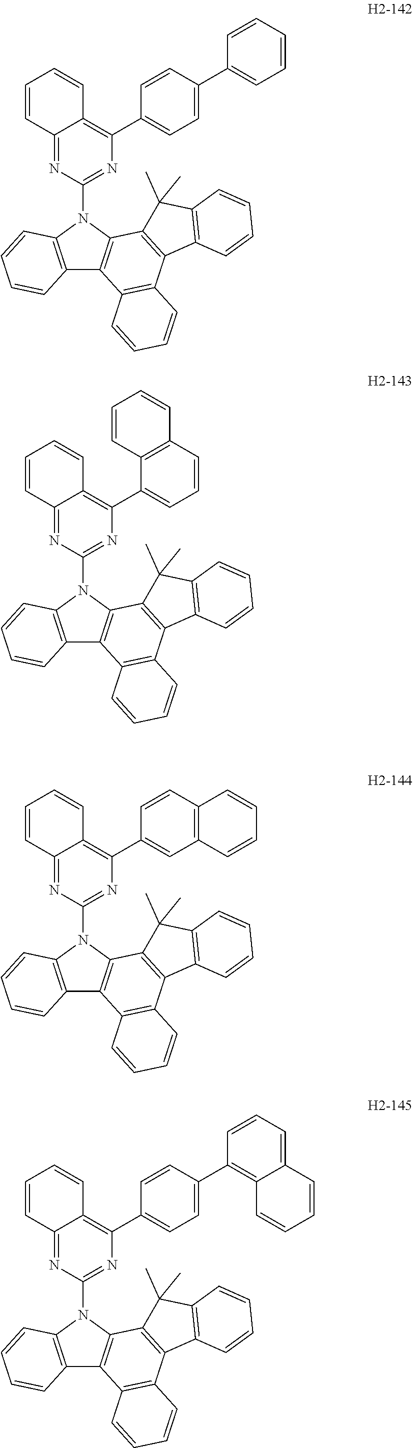

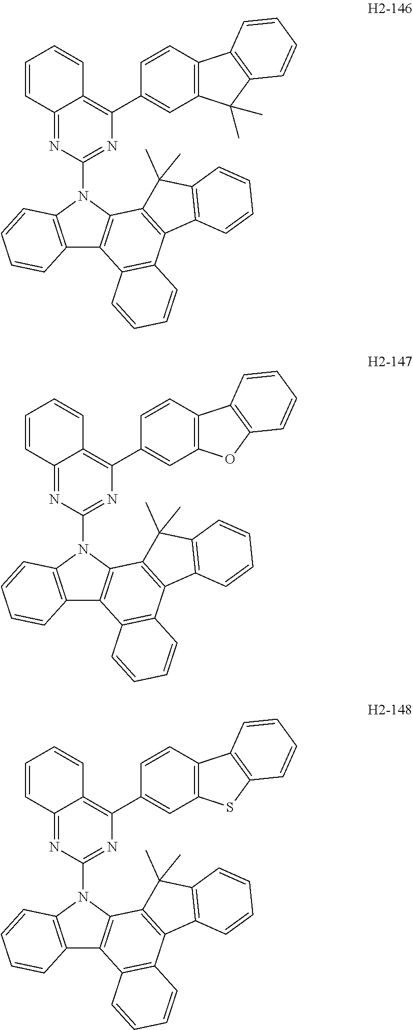 [SMILES: CC1(C)c2ccccc2-c2ccc(-c3nc(-n4c5ccccc5c5c6ccccc6c6c(c54)C(C)(C)c4ccccc4-6)nc4ccccc34)cc21 ; CC1(C)c2ccccc2-c2c1c1c(c3ccccc23)c2ccccc2n1-c1nc(-c2ccc3c(c2)oc2ccccc23)c2ccccc2n1 ; CC1(C)c2ccccc2-c2c1c1c(c3ccccc23)c2ccccc2n1-c1nc(-c2ccc3c(c2)sc2ccccc23)c2ccccc2n1]

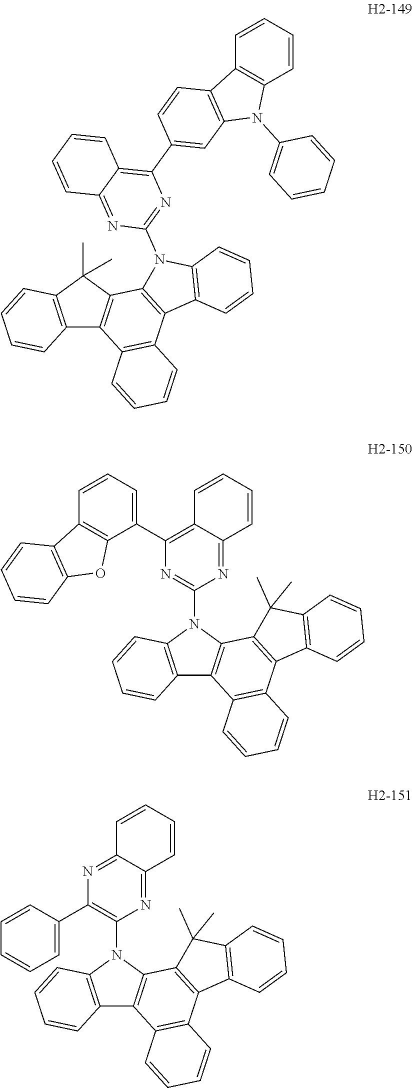 [SMILES: CC1(C)c2ccccc2-c2c1c1c(c3ccccc23)c2ccccc2n1-c1nc(-c2ccc3c4ccccc4n(-c4ccccc4)c3c2)c2ccccc2n1 ; CC1(C)c2ccccc2-c2c1c1c(c3ccccc23)c2ccccc2n1-c1nc(-c2cccc3c2oc2ccccc23)c2ccccc2n1 ; CC1(C)c2ccccc2-c2c1c1c(c3ccccc23)c2ccccc2n1-c1nc2ccccc2nc1-c1ccccc1]

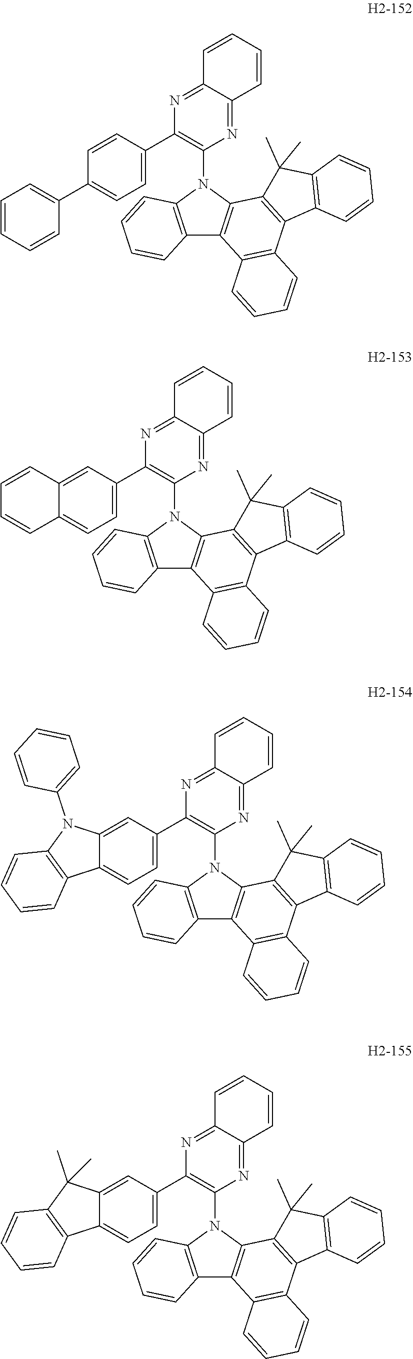 [SMILES: CC1(C)c2ccccc2-c2c1c1c(c3ccccc23)c2ccccc2n1-c1nc2ccccc2nc1-c1ccc(-c2ccccc2)cc1 ; CC1(C)c2ccccc2-c2c1c1c(c3ccccc23)c2ccccc2n1-c1nc2ccccc2nc1-c1ccc2ccccc2c1 ; CC1(C)c2ccccc2-c2c1c1c(c3ccccc23)c2ccccc2n1-c1nc2ccccc2nc1-c1ccc2c3ccccc3n(-c3ccccc3)c2c1 ; CC1(C)c2ccccc2-c2ccc(-c3nc4ccccc4nc3-n3c4ccccc4c4c5ccccc5c5c(c43)C(C)(C)c3ccccc3-5)cc21]

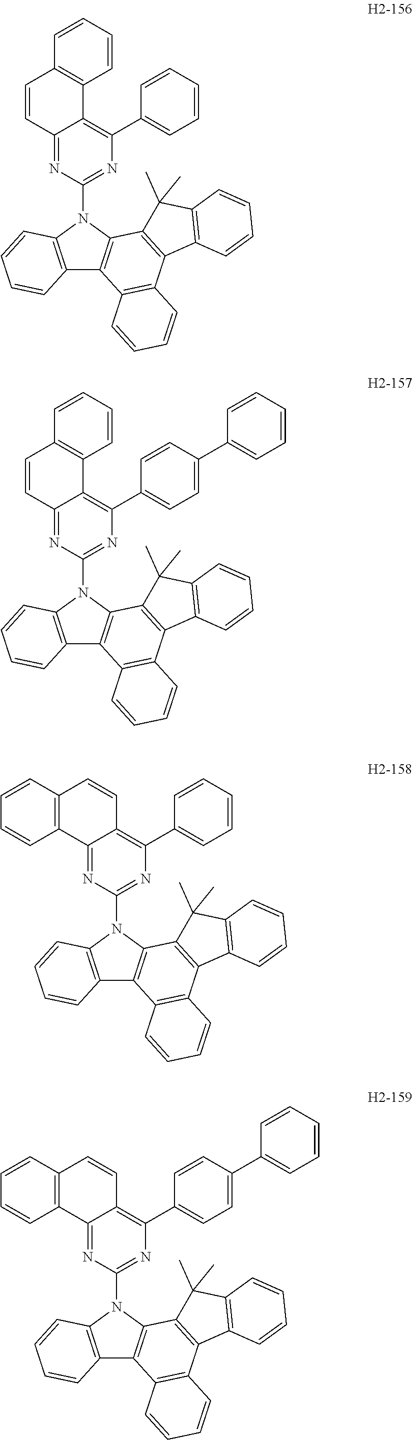 [SMILES: CC1(C)c2ccccc2-c2c1c1c(c3ccccc23)c2ccccc2n1-c1nc(-c2ccccc2)c2c(ccc3ccccc32)n1 ; CC1(C)c2ccccc2-c2c1c1c(c3ccccc23)c2ccccc2n1-c1nc(-c2ccc(-c3ccccc3)cc2)c2c(ccc3ccccc32)n1 ; CC1(C)c2ccccc2-c2c1c1c(c3ccccc23)c2ccccc2n1-c1nc(-c2ccccc2)c2ccc3ccccc3c2n1 ; CC1(C)c2ccccc2-c2c1c1c(c3ccccc23)c2ccccc2n1-c1nc(-c2ccc(-c3ccccc3)cc2)c2ccc3ccccc3c2n1]

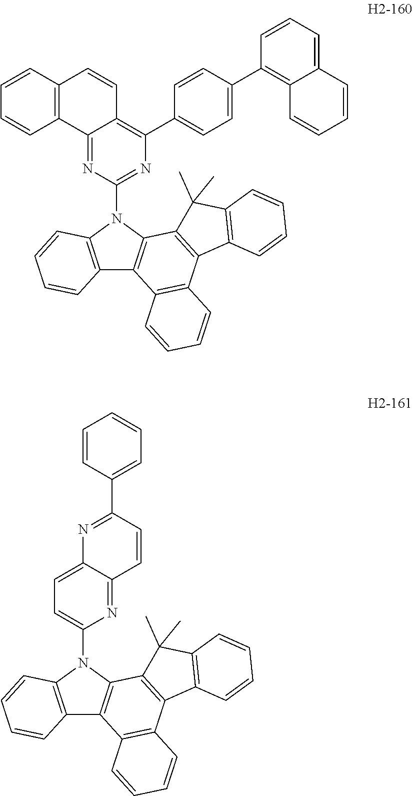 [SMILES: CC1(C)c2ccccc2-c2c1c1c(c3ccccc23)c2ccccc2n1-c1nc(-c2ccc(-c3cccc4ccccc34)cc2)c2ccc3ccccc3c2n1 ; CC1(C)c2ccccc2-c2c1c1c(c3ccccc23)c2ccccc2n1-c1ccc2nc(-c3ccccc3)ccc2n1]

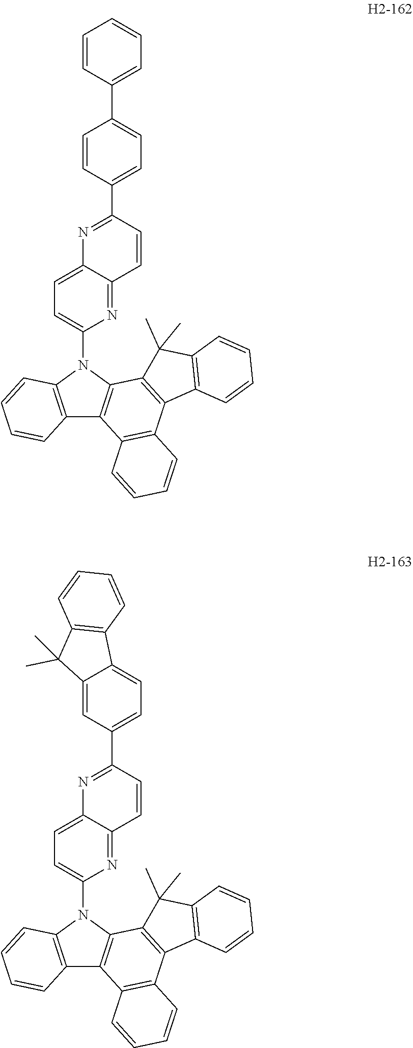 [SMILES: CC1(C)c2ccccc2-c2c1c1c(c3ccccc23)c2ccccc2n1-c1ccc2nc(-c3ccc(-c4ccccc4)cc3)ccc2n1 ; CC1(C)c2ccccc2-c2ccc(-c3ccc4nc(-n5c6ccccc6c6c7ccccc7c7c(c65)C(C)(C)c5ccccc5-7)ccc4n3)cc21]

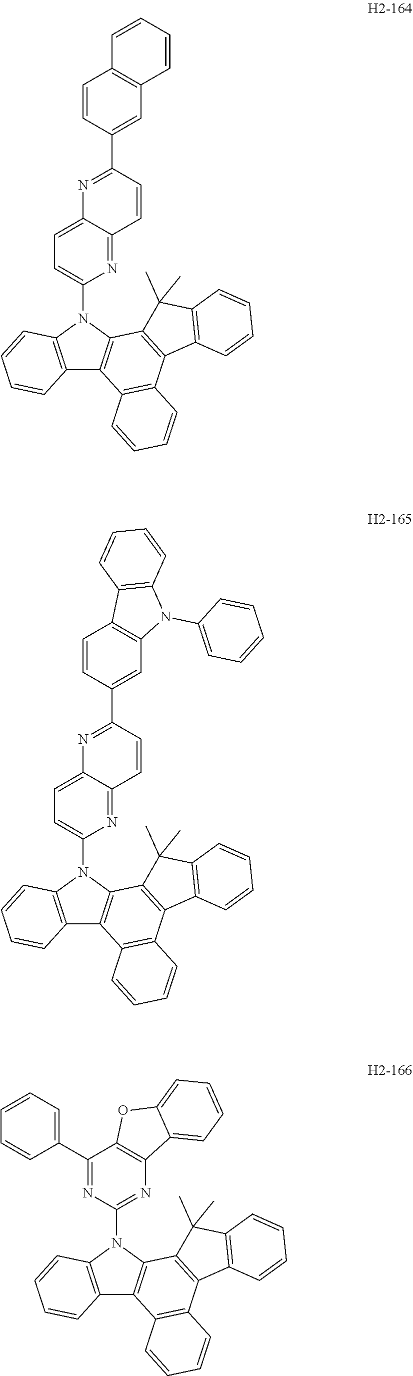 [SMILES: CC1(C)c2ccccc2-c2c1c1c(c3ccccc23)c2ccccc2n1-c1ccc2nc(-c3ccc4ccccc4c3)ccc2n1 ; CC1(C)c2ccccc2-c2c1c1c(c3ccccc23)c2ccccc2n1-c1ccc2nc(-c3ccc4c5ccccc5n(-c5ccccc5)c4c3)ccc2n1 ; CC1(C)c2ccccc2-c2c1c1c(c3ccccc23)c2ccccc2n1-c1nc(-c2ccccc2)c2oc3ccccc3c2n1]

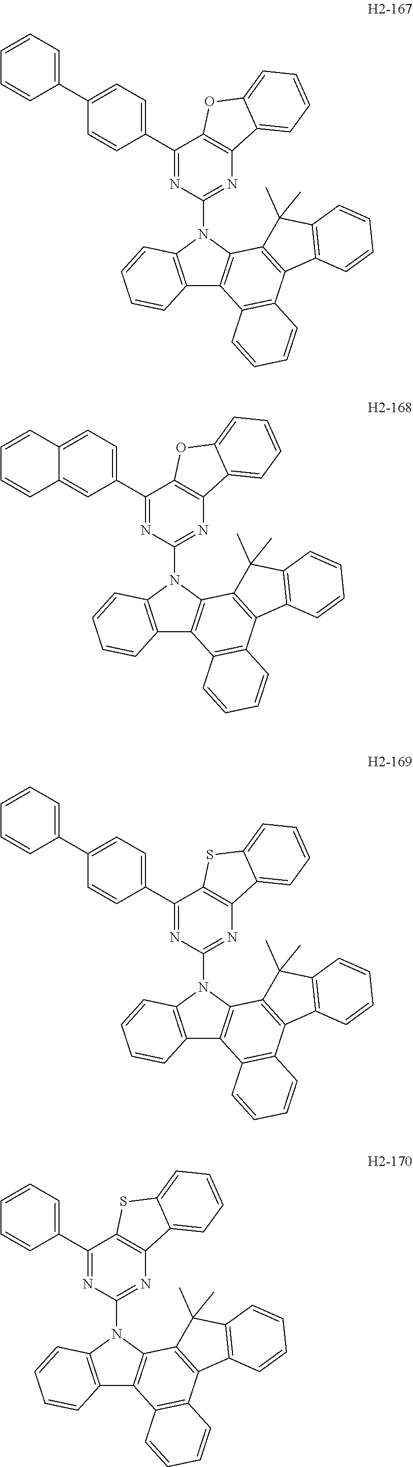 [SMILES: CC1(C)c2ccccc2-c2c1c1c(c3ccccc23)c2ccccc2n1-c1nc(-c2ccc(-c3ccccc3)cc2)c2oc3ccccc3c2n1 ; CC1(C)c2ccccc2-c2c1c1c(c3ccccc23)c2ccccc2n1-c1nc(-c2ccc3ccccc3c2)c2oc3ccccc3c2n1 ; CC1(C)c2ccccc2-c2c1c1c(c3ccccc23)c2ccccc2n1-c1nc(-c2ccc(-c3ccccc3)cc2)c2sc3ccccc3c2n1 ; CC1(C)c2ccccc2-c2c1c1c(c3ccccc23)c2ccccc2n1-c1nc(-c2ccccc2)c2sc3ccccc3c2n1]

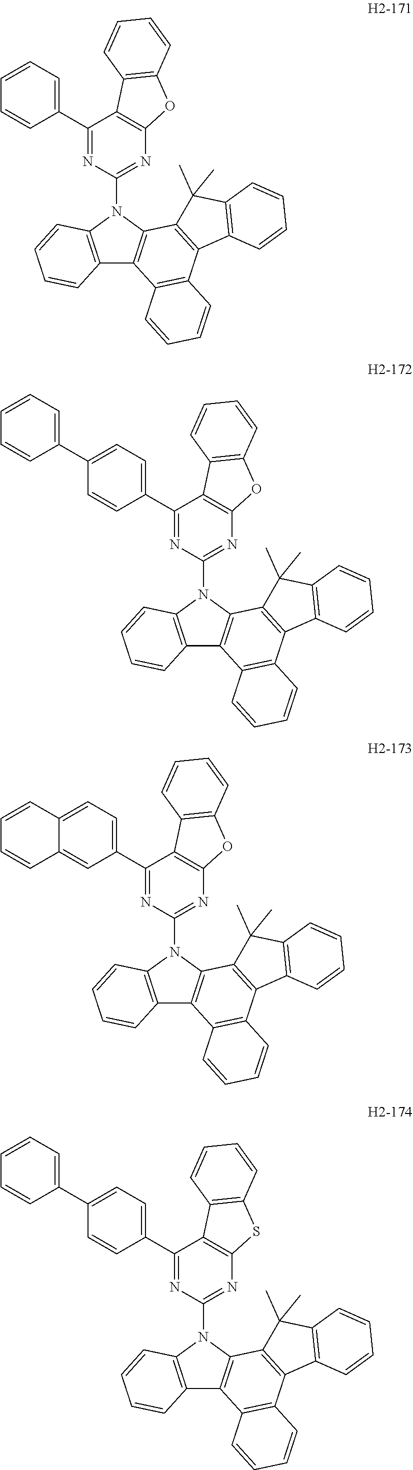 [SMILES: CC1(C)c2ccccc2-c2c1c1c(c3ccccc23)c2ccccc2n1-c1nc(-c2ccccc2)c2c(n1)oc1ccccc12 ; CC1(C)c2ccccc2-c2c1c1c(c3ccccc23)c2ccccc2n1-c1nc(-c2ccc(-c3ccccc3)cc2)c2c(n1)oc1ccccc12 ; CC1(C)c2ccccc2-c2c1c1c(c3ccccc23)c2ccccc2n1-c1nc(-c2ccc3ccccc3c2)c2c(n1)oc1ccccc12 ; CC1(C)c2ccccc2-c2c1c1c(c3ccccc23)c2ccccc2n1-c1nc(-c2ccc(-c3ccccc3)cc2)c2c(n1)sc1ccccc12]

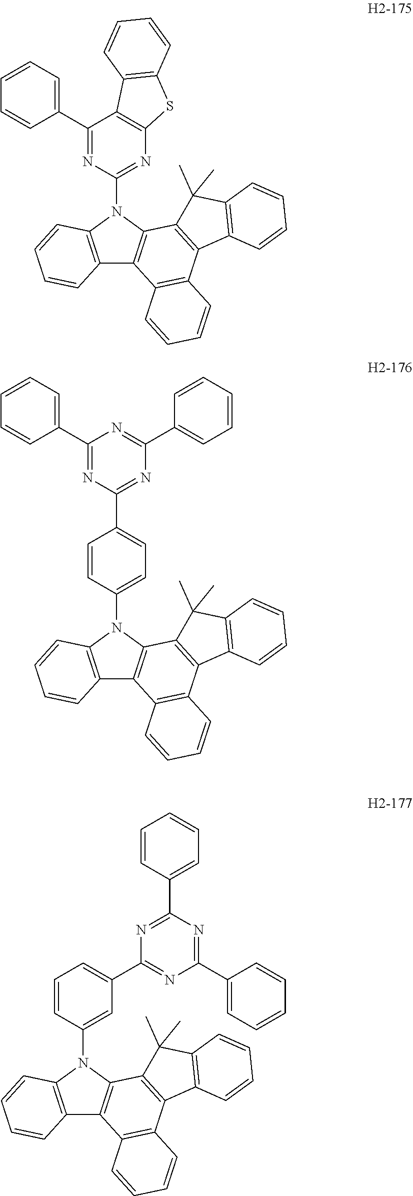

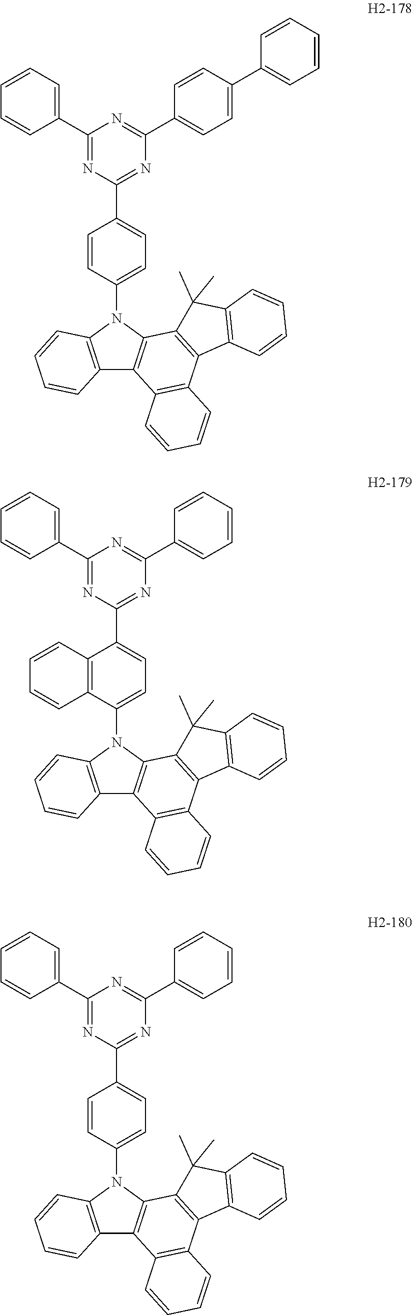 [SMILES: CC1(C)c2ccccc2-c2c1c1c(c3ccccc23)c2ccccc2n1-c1ccc(-c2nc(-c3ccccc3)nc(-c3ccc(-c4ccccc4)cc3)n2)cc1 ; CC1(C)c2ccccc2-c2c1c1c(c3ccccc23)c2ccccc2n1-c1ccc(-c2nc(-c3ccccc3)nc(-c3ccccc3)n2)c2ccccc12 ; CC1(C)c2ccccc2-c2c1c1c(c3ccccc23)c2ccccc2n1-c1ccc(-c2nc(-c3ccccc3)nc(-c3ccccc3)n2)cc1]

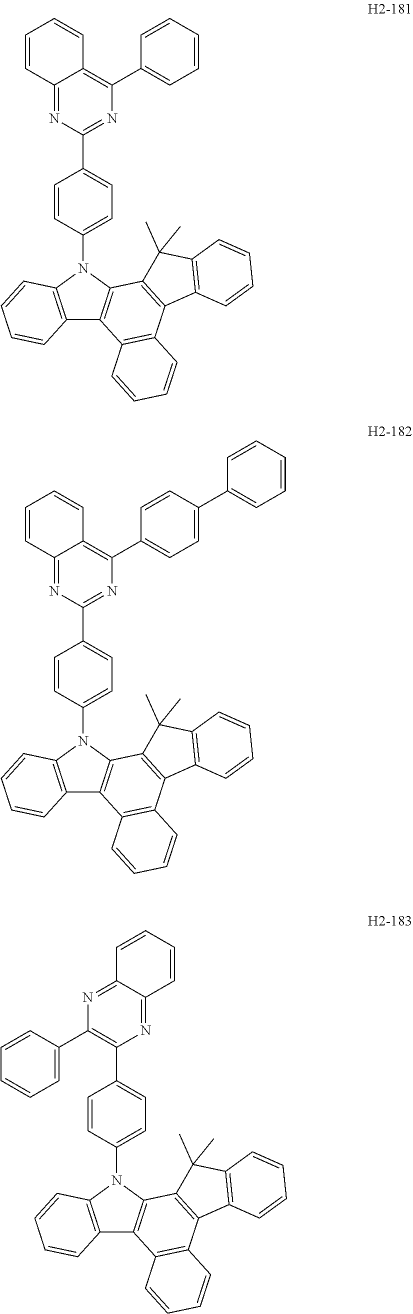 [SMILES: CC1(C)c2ccccc2-c2c1c1c(c3ccccc23)c2ccccc2n1-c1ccc(-c2nc(-c3ccccc3)c3ccccc3n2)cc1 ; CC1(C)c2ccccc2-c2c1c1c(c3ccccc23)c2ccccc2n1-c1ccc(-c2nc(-c3ccc(-c4ccccc4)cc3)c3ccccc3n2)cc1 ; CC1(C)c2ccccc2-c2c1c1c(c3ccccc23)c2ccccc2n1-c1ccc(-c2nc3ccccc3nc2-c2ccccc2)cc1]

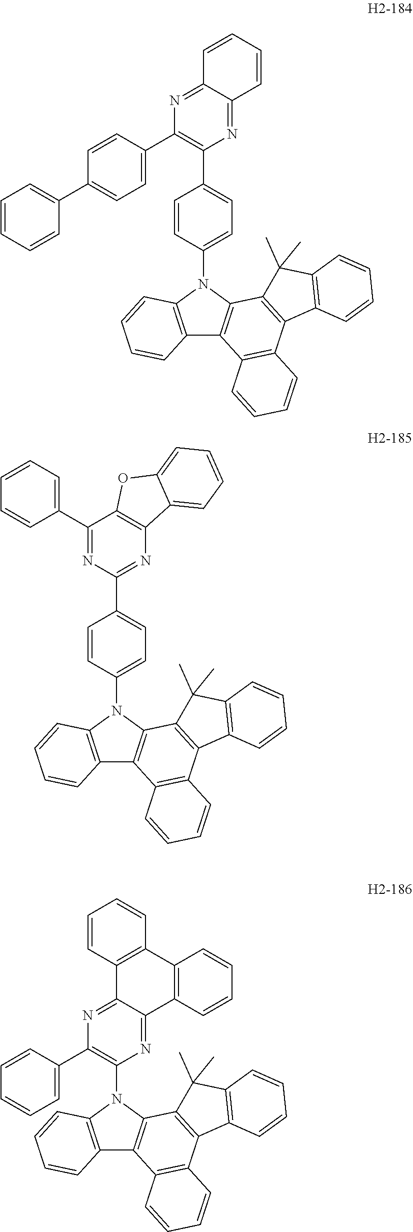 [SMILES: CC1(C)c2ccccc2-c2c1c1c(c3ccccc23)c2ccccc2n1-c1ccc(-c2nc3ccccc3nc2-c2ccc(-c3ccccc3)cc2)cc1 ; CC1(C)c2ccccc2-c2c1c1c(c3ccccc23)c2ccccc2n1-c1ccc(-c2nc(-c3ccccc3)c3oc4ccccc4c3n2)cc1 ; CC1(C)c2ccccc2-c2c1c1c(c3ccccc23)c2ccccc2n1-c1nc2c3ccccc3c3ccccc3c2nc1-c1ccccc1]

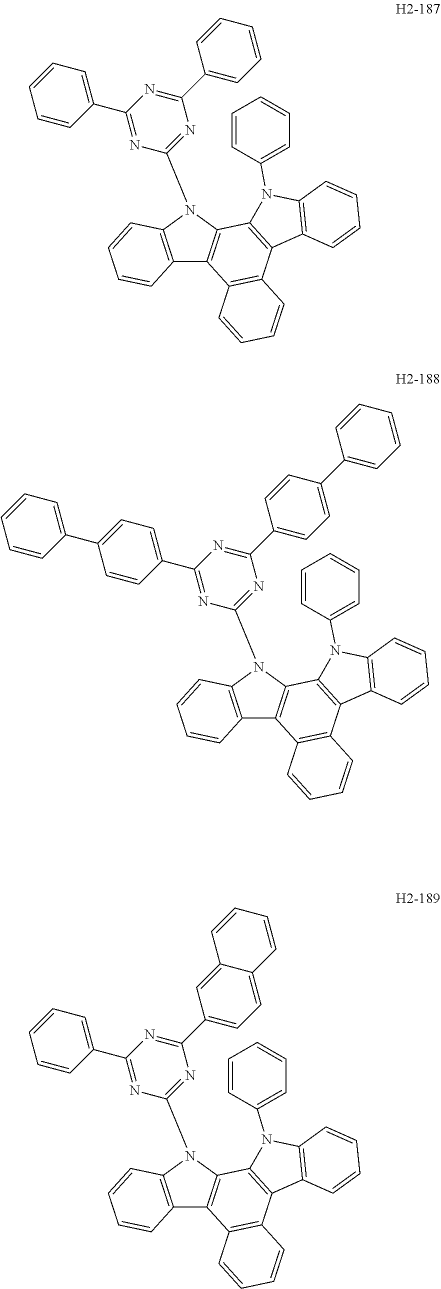 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2ccc(-c3nc(-c4ccc(-c5ccccc5)cc4)nc(-n4c5ccccc5c5c6ccccc6c6c7ccccc7n(-c7ccccc7)c6c54)n3)cc2)cc1 ; c1ccc(-c2nc(-c3ccc4ccccc4c3)nc(-n3c4ccccc4c4c5ccccc5c5c6ccccc6n(-c6ccccc6)c5c43)n2)cc1]

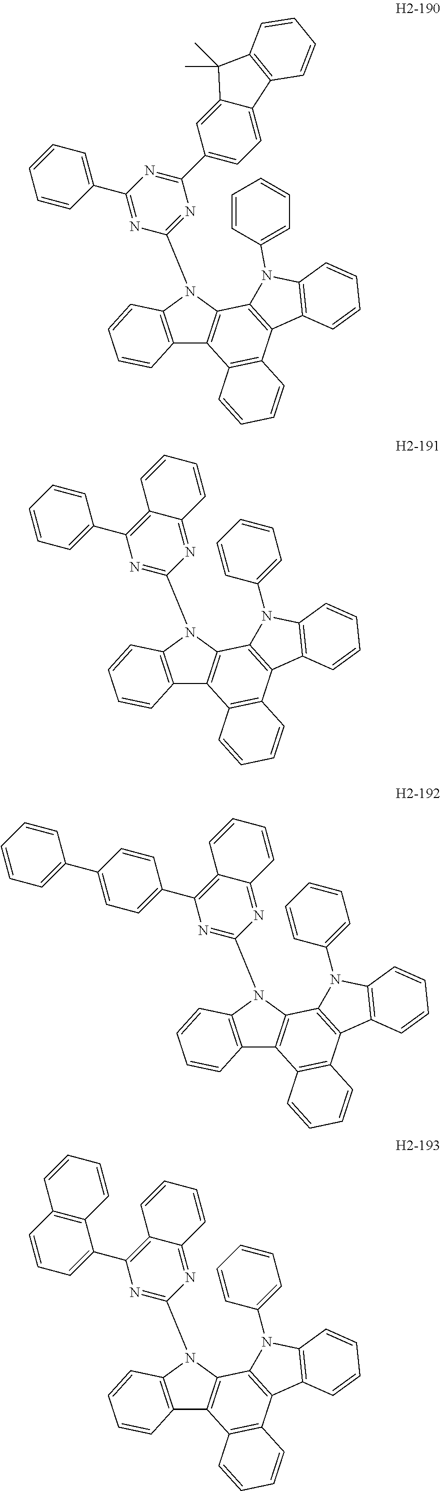 [SMILES: CC1(C)c2ccccc2-c2ccc(-c3nc(-c4ccccc4)nc(-n4c5ccccc5c5c6ccccc6c6c7ccccc7n(-c7ccccc7)c6c54)n3)cc21 ; c1ccc(-c2nc(-n3c4ccccc4c4c5ccccc5c5c6ccccc6n(-c6ccccc6)c5c43)nc3ccccc23)cc1 ; c1ccc(-c2ccc(-c3nc(-n4c5ccccc5c5c6ccccc6c6c7ccccc7n(-c7ccccc7)c6c54)nc4ccccc34)cc2)cc1 ; c1ccc(-n2c3ccccc3c3c4ccccc4c4c5ccccc5n(-c5nc(-c6cccc7ccccc67)c6ccccc6n5)c4c32)cc1]

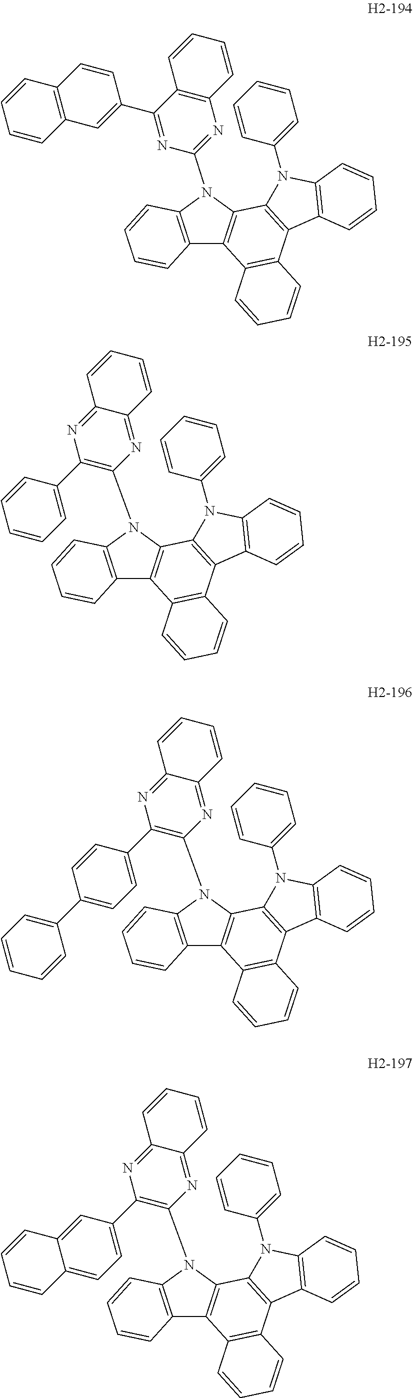 [SMILES: c1ccc(-n2c3ccccc3c3c4ccccc4c4c5ccccc5n(-c5nc(-c6ccc7ccccc7c6)c6ccccc6n5)c4c32)cc1 ; c1ccc(-c2nc3ccccc3nc2-n2c3ccccc3c3c4ccccc4c4c5ccccc5n(-c5ccccc5)c4c32)cc1 ; c1ccc(-c2ccc(-c3nc4ccccc4nc3-n3c4ccccc4c4c5ccccc5c5c6ccccc6n(-c6ccccc6)c5c43)cc2)cc1 ; c1ccc(-n2c3ccccc3c3c4ccccc4c4c5ccccc5n(-c5nc6ccccc6nc5-c5ccc6ccccc6c5)c4c32)cc1]

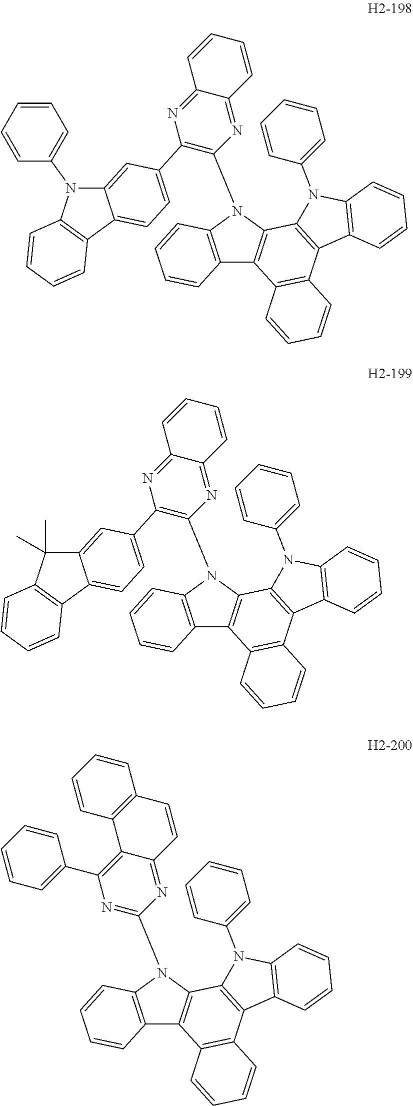 [SMILES: c1ccc(-n2c3ccccc3c3ccc(-c4nc5ccccc5nc4-n4c5ccccc5c5c6ccccc6c6c7ccccc7n(-c7ccccc7)c6c54)cc32)cc1 ; CC1(C)c2ccccc2-c2ccc(-c3nc4ccccc4nc3-n3c4ccccc4c4c5ccccc5c5c6ccccc6n(-c6ccccc6)c5c43)cc21 ; c1ccc(-c2nc(-n3c4ccccc4c4c5ccccc5c5c6ccccc6n(-c6ccccc6)c5c43)nc3ccc4ccccc4c23)cc1]

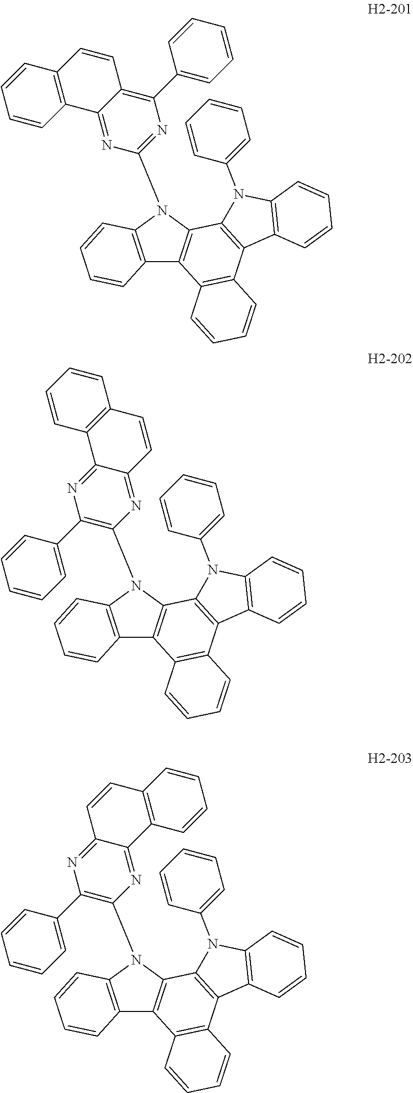 [SMILES: c1ccc(-c2nc(-n3c4ccccc4c4c5ccccc5c5c6ccccc6n(-c6ccccc6)c5c43)nc3c2ccc2ccccc23)cc1 ; c1ccc(-c2nc3c(ccc4ccccc43)nc2-n2c3ccccc3c3c4ccccc4c4c5ccccc5n(-c5ccccc5)c4c32)cc1 ; c1ccc(-c2nc3ccc4ccccc4c3nc2-n2c3ccccc3c3c4ccccc4c4c5ccccc5n(-c5ccccc5)c4c32)cc1]

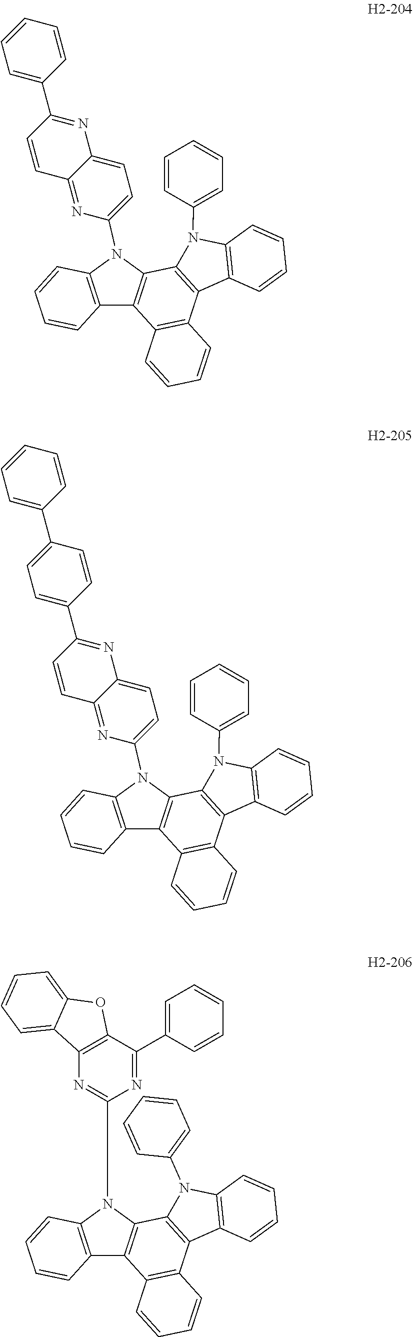 [SMILES: c1ccc(-c2ccc3nc(-n4c5ccccc5c5c6ccccc6c6c7ccccc7n(-c7ccccc7)c6c54)ccc3n2)cc1 ; c1ccc(-c2ccc(-c3ccc4nc(-n5c6ccccc6c6c7ccccc7c7c8ccccc8n(-c8ccccc8)c7c65)ccc4n3)cc2)cc1 ; c1ccc(-c2nc(-n3c4ccccc4c4c5ccccc5c5c6ccccc6n(-c6ccccc6)c5c43)nc3c2oc2ccccc23)cc1]

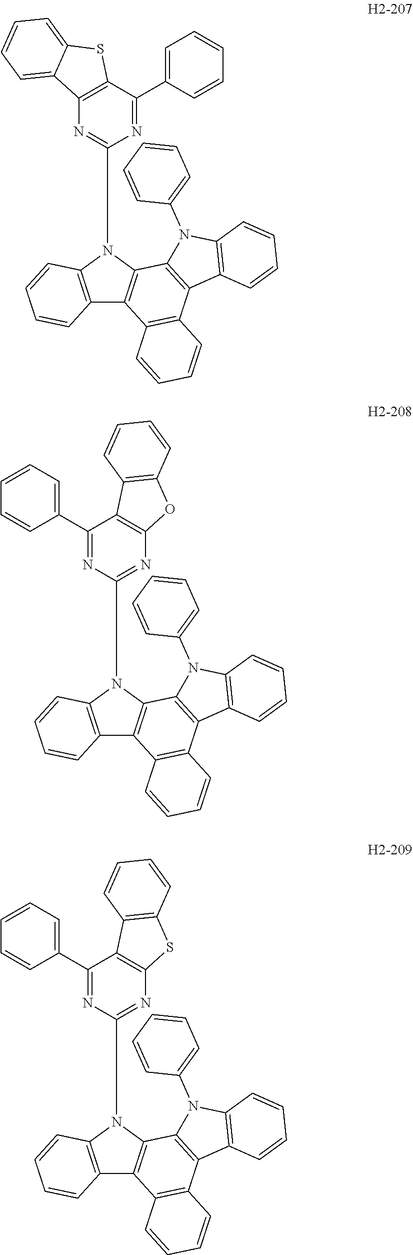

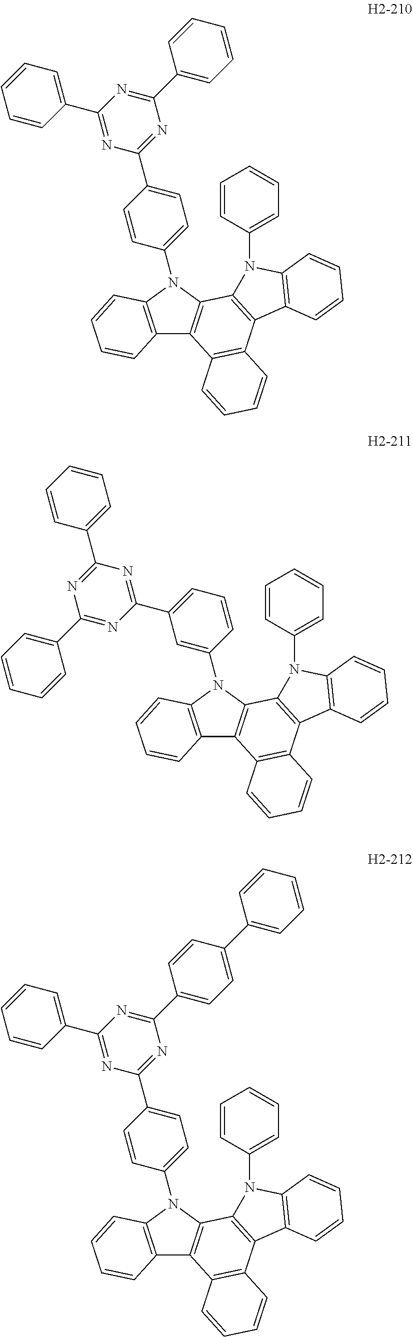 [SMILES: c1ccc(-c2nc(-c3ccccc3)nc(-c3ccc(-n4c5ccccc5c5c6ccccc6c6c7ccccc7n(-c7ccccc7)c6c54)cc3)n2)cc1 ; c1ccc(-c2nc(-c3ccccc3)nc(-c3cccc(-n4c5ccccc5c5c6ccccc6c6c7ccccc7n(-c7ccccc7)c6c54)c3)n2)cc1 ; c1ccc(-c2ccc(-c3nc(-c4ccccc4)nc(-c4ccc(-n5c6ccccc6c6c7ccccc7c7c8ccccc8n(-c8ccccc8)c7c65)cc4)n3)cc2)cc1]

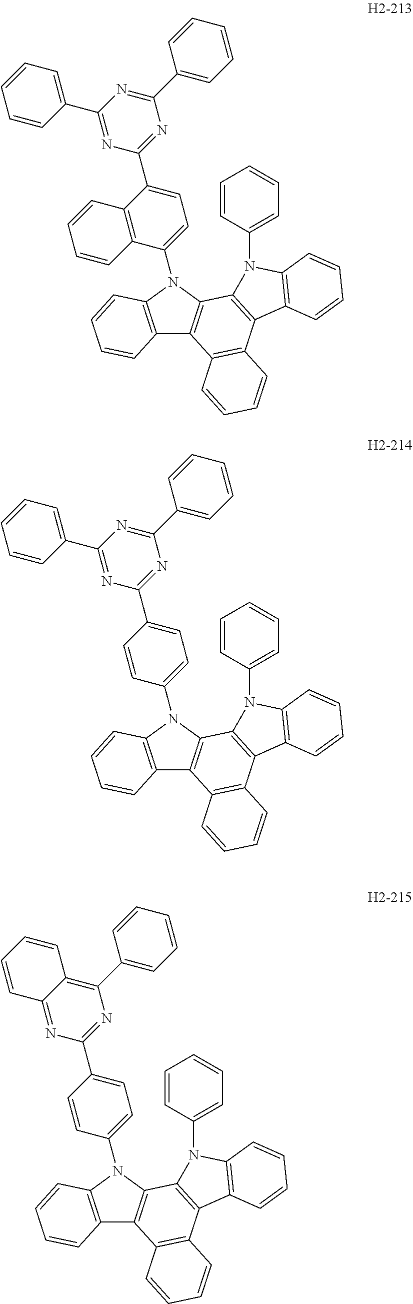 [SMILES: c1ccc(-c2nc(-c3ccccc3)nc(-c3ccc(-n4c5ccccc5c5c6ccccc6c6c7ccccc7n(-c7ccccc7)c6c54)c4ccccc34)n2)cc1 ; c1ccc(-c2nc(-c3ccccc3)nc(-c3ccc(-n4c5ccccc5c5c6ccccc6c6c7ccccc7n(-c7ccccc7)c6c54)cc3)n2)cc1 ; c1ccc(-c2nc(-c3ccc(-n4c5ccccc5c5c6ccccc6c6c7ccccc7n(-c7ccccc7)c6c54)cc3)nc3ccccc23)cc1]

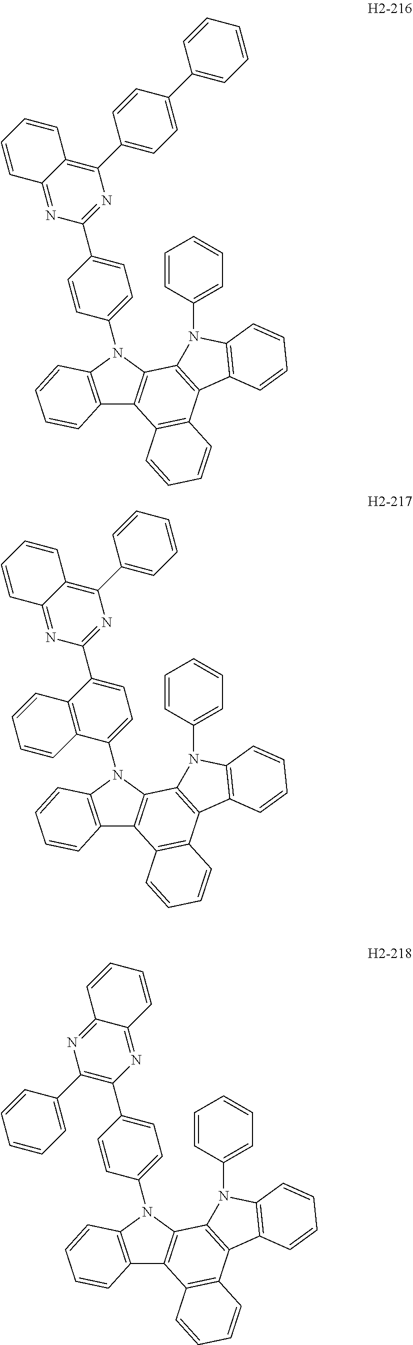

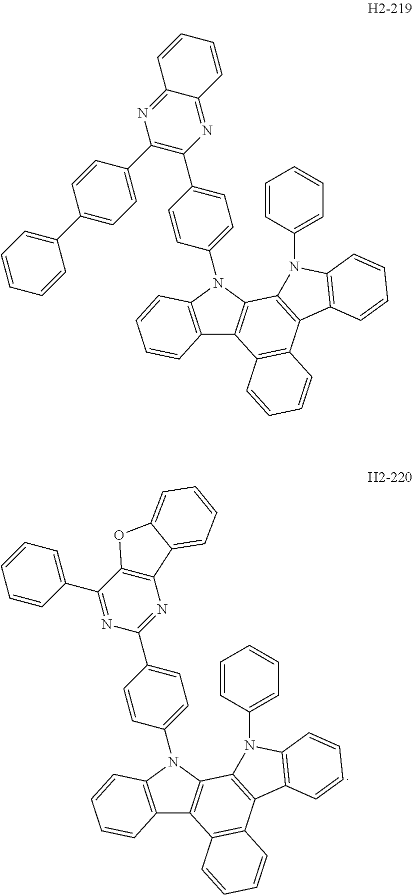 [SMILES: c1ccc(-c2ccc(-c3nc4ccccc4nc3-c3ccc(-n4c5ccccc5c5c6ccccc6c6c7ccccc7n(-c7ccccc7)c6c54)cc3)cc2)cc1 ; c1ccc(-c2nc(-c3ccc(-n4c5ccccc5c5c6ccccc6c6c7ccccc7n(-c7ccccc7)c6c54)cc3)nc3c2oc2ccccc23)cc1]

The compounds represented by formulas 1 and 2 according to the present disclosure can be prepared by a synthetic method known to a person skilled in the art. For example, the compound represented by formula 1 can be prepared by referring to Korean Patent Application Laying-Open Nos. 2013-0106255 (published on Sep. 27, 2013), 2014-0108637 (published on Sep. 12, 2014), 2014-0068883 (published on Jun. 9, 2014), etc., and the compound represented by formula 2 can be prepared by referring to Korean Patent Application Laying-Open No. 2015-0032447 (published on Mar. 26, 2015), etc., but is not limited thereto.

The present disclosure provides a mixture comprising a combination of the compound represented by formula 1 and the compound represented by formula 2. The mixture may be used as an organic electroluminescent material.

The organic electroluminescent device according to the present disclosure comprises an anode, a cathode, and at least one organic layer between the anode and the cathode. The organic layer may comprise a plurality of organic electroluminescent materials in which the compound represented by formula 1 is included as a first organic electroluminescent material, and the compound represented by formula 2 is included as a second organic electroluminescent material. In one embodiment of the present disclosure, the organic electroluminescent device according to the present disclosure comprises an anode, a cathode, and at least one light-emitting layer between the anode and the cathode, in which the light-emitting layer may comprise the compound represented by formula 1 and the compound represented by formula 2.

The organic electroluminescent device according to the present disclosure comprises an anode, a cathode, and at least one organic layer between the anode and the cathode, in which the organic layer comprises a light-emitting layer. The light-emitting layer comprises a host and a phosphorescent dopant. The host comprises a plurality of host materials, in which at least a first host compound of the plural host materials is represented by formula 1, and a second host compound is represented by formula 2.

The light-emitting layer is a layer from which light is emitted, and can be a single layer or a multi-layer of which two or more layers are stacked. In the plurality of host materials according to the present disclosure, the first and second host materials may both be comprised in one layer or may be respectively comprised in different light-emitting layers. In the light-emitting layer, it is preferable that the doping concentration of the dopant compound with respect to the host compound is less than 20 wt %.

The organic layer may comprise a light-emitting layer, and may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer.

In the organic electroluminescent device according to the present disclosure, the weight ratio of the first host compound to the second host compound is in the range of 1:99 to 99:1. The weight ratio is preferably about 10:90 to about 90:10, more preferably about 30:70 to about 70:30, even more preferably about 40:60 to 60:40, and still more preferably about 50:50.

The dopant comprised in the organic electroluminescent device according to the present disclosure may be at least one fluorescent or phosphorescent dopant, and preferably at least one phosphorescent dopant. The phosphorescent dopant material applied to the organic electroluminescent device of the present disclosure is not particularly limited, but may be preferably selected from metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device according to the present disclosure may include the compound represented by the following formula 101, but is not limited thereto.

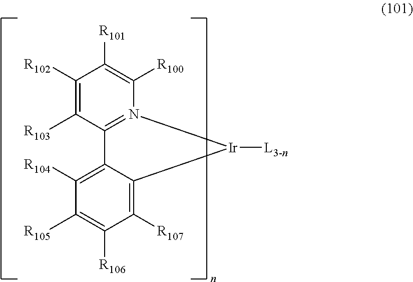

In formula 101, L is selected from the following structures:

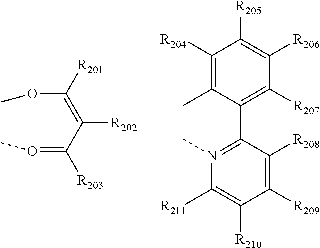

R₁₀₀ to R₁₀₃ each independently represent hydrogen, deuterium, a halogen, a (C1-C30)alkyl unsubstituted or substituted with a halogen, a substituted or unsubstituted (C3-C30)cycloalkyl, a substituted or unsubstituted (C6-C30)aryl, a cyano, a substituted or unsubstituted (C3-C30)heteroaryl, or a substituted or unsubstituted (C1-C30)alkoxy; or adjacent substituents of R₁₀₀ to R₁₀₃ may be linked to each other to form a substituted or unsubstituted (3- to 30-membered) ring, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent hydrogen, deuterium, a halogen, a (C1-C30)alkyl unsubstituted or substituted with a halogen, a substituted or unsubstituted (C3-C30)cycloalkyl, a substituted or unsubstituted (C6-C30)aryl, a substituted or unsubstituted (C3-C30)heteroaryl, a cyano, or a substituted or unsubstituted (C1-C30)alkoxy; or adjacent substituents of R₁₀₄ to R₁₀₇ may be linked to each other to form a substituted or unsubstituted (3- to 30-membered) ring, e.g., a substituted or unsubstituted naphthyl, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₂₀₁ to R₂₁₁, each independently, represent hydrogen, deuterium, a halogen, a (C1-C30)alkyl unsubstituted or substituted with a halogen, a substituted or unsubstituted (C3-C30)cycloalkyl, or a substituted or unsubstituted (C6-C30)aryl; or adjacent substituents of R₂₀₁ to R₂₁₁ may be linked to each other to form a substituted or unsubstituted (3- to 30-membered) ring; and

n represents an integer of 1 to 3.

Specifically, the dopant material includes the following compounds, but is not limited thereto:

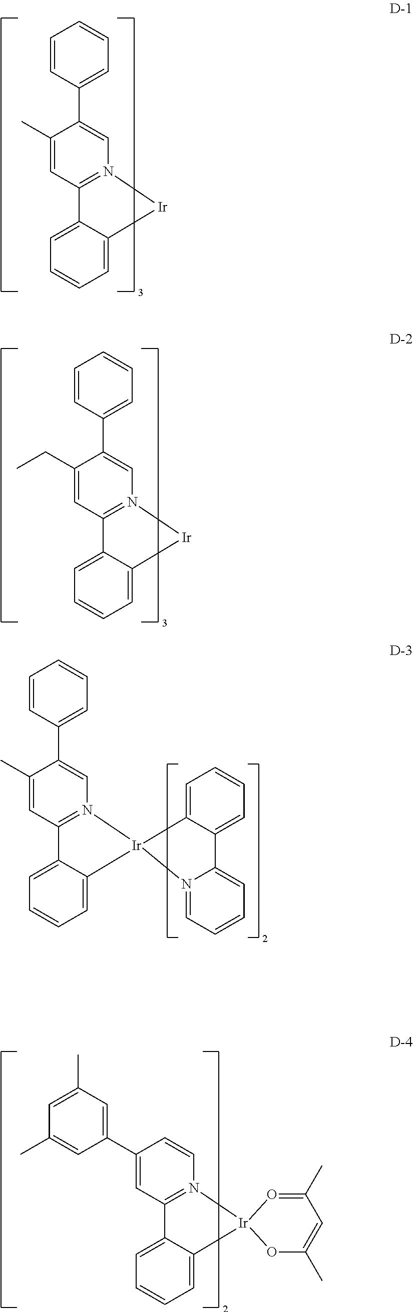

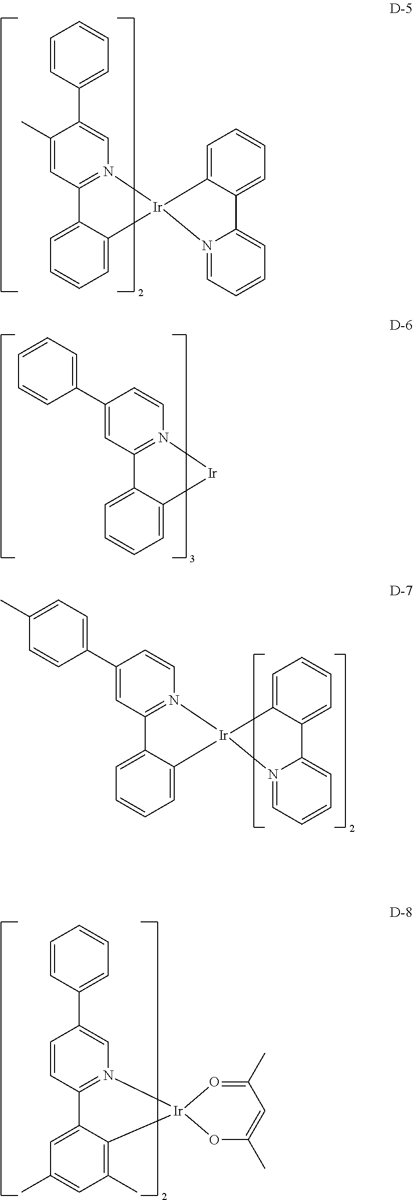

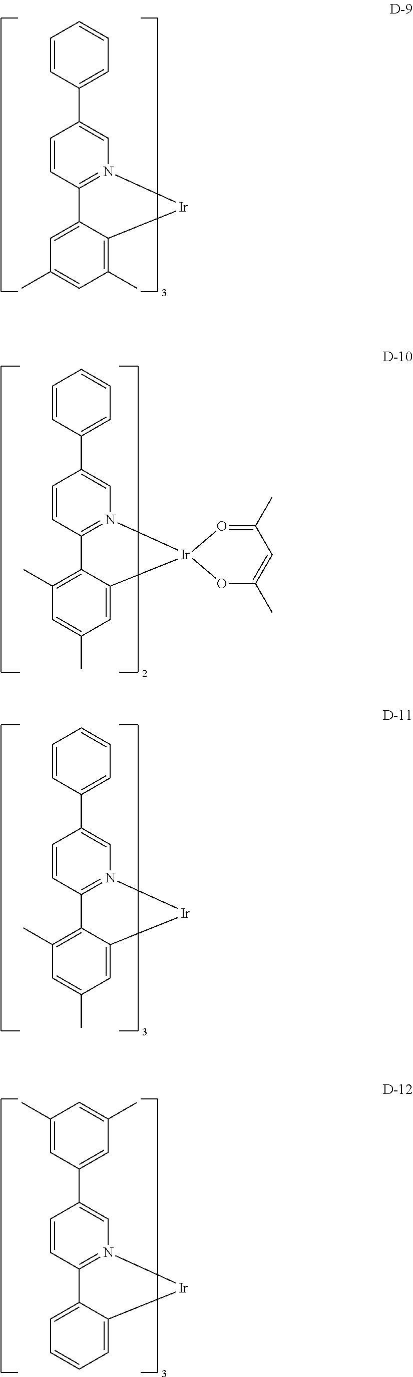

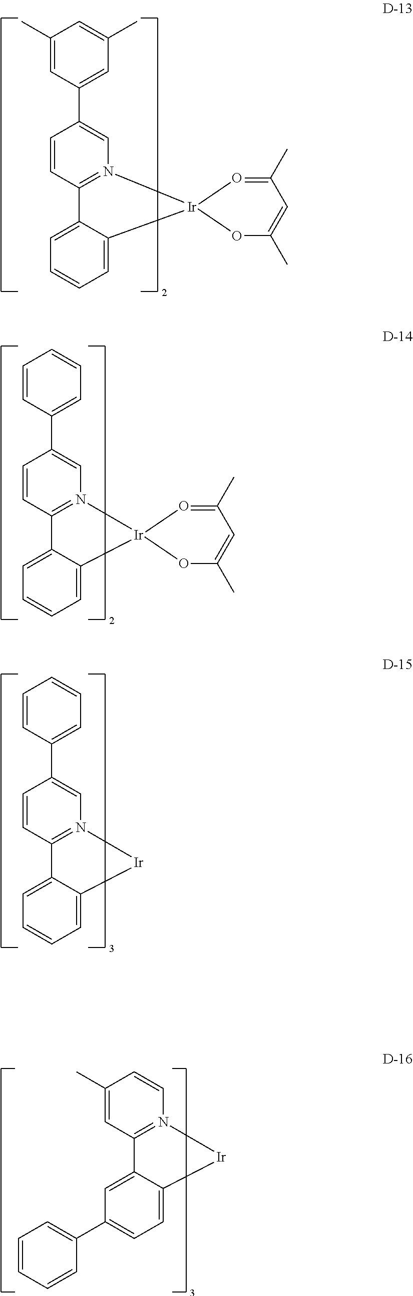

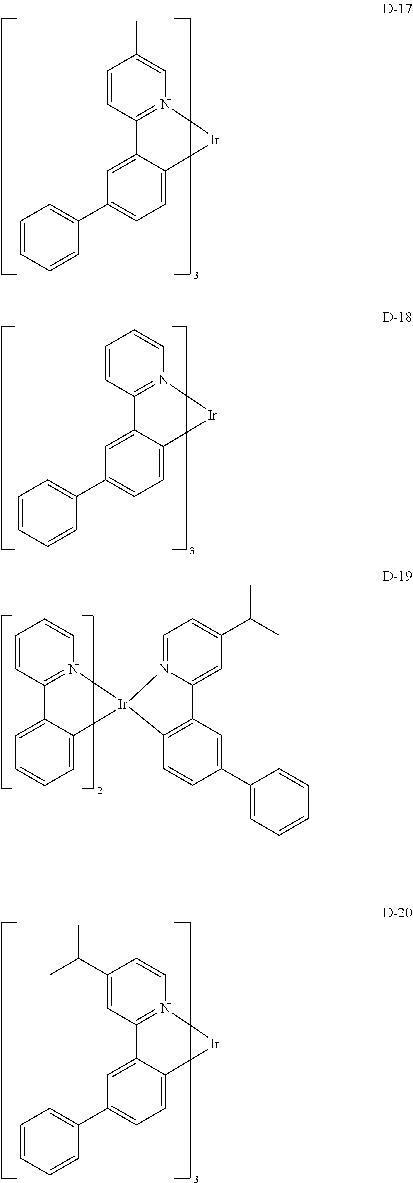

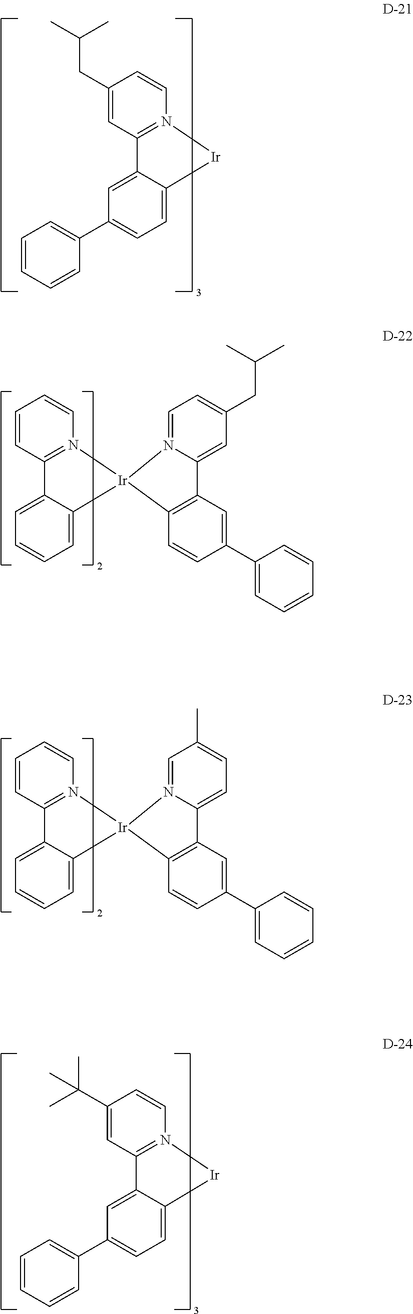

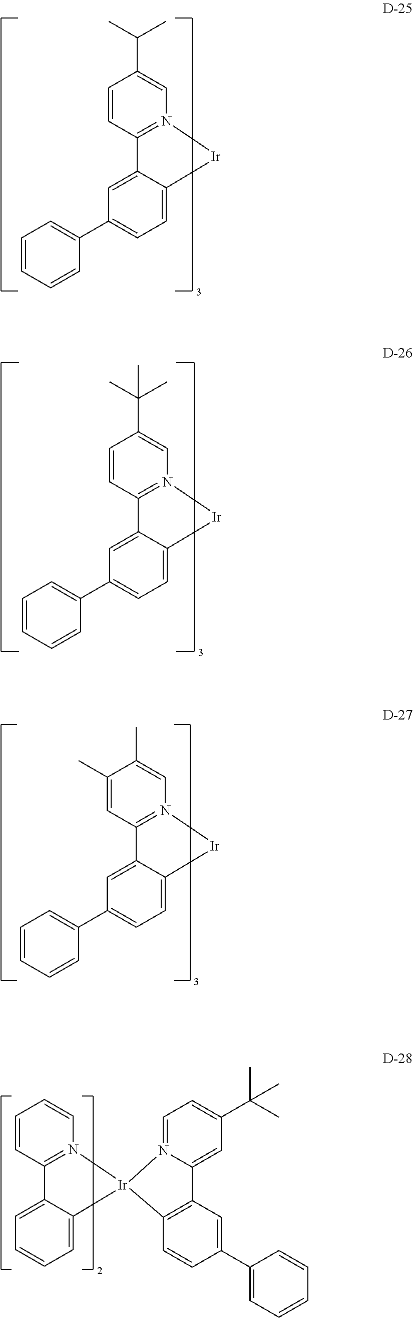

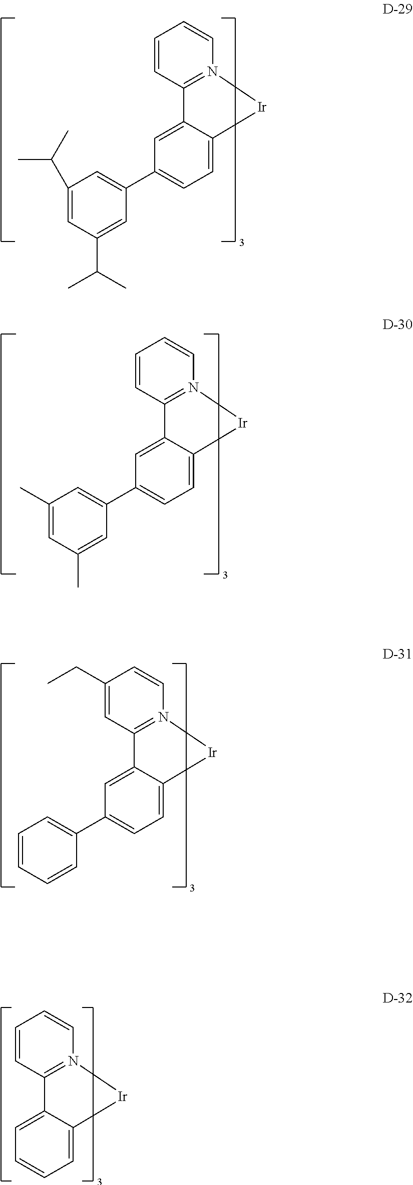

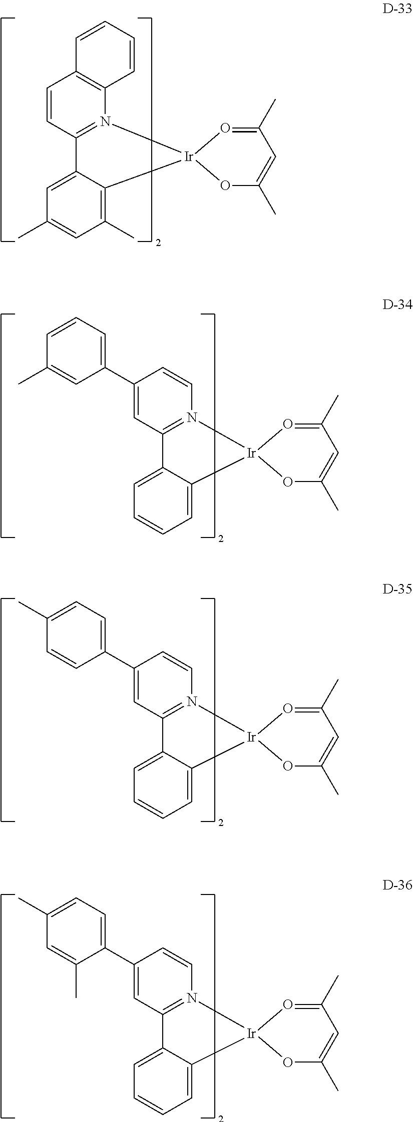

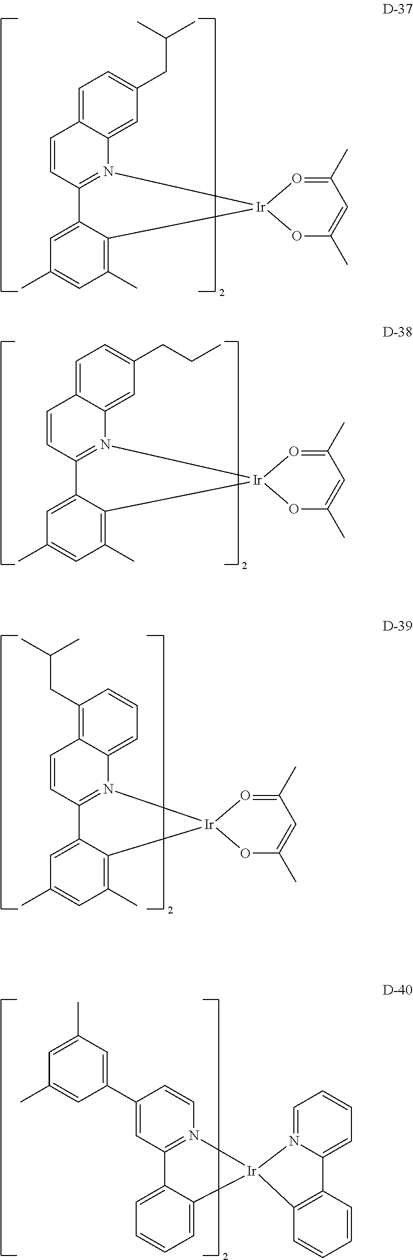

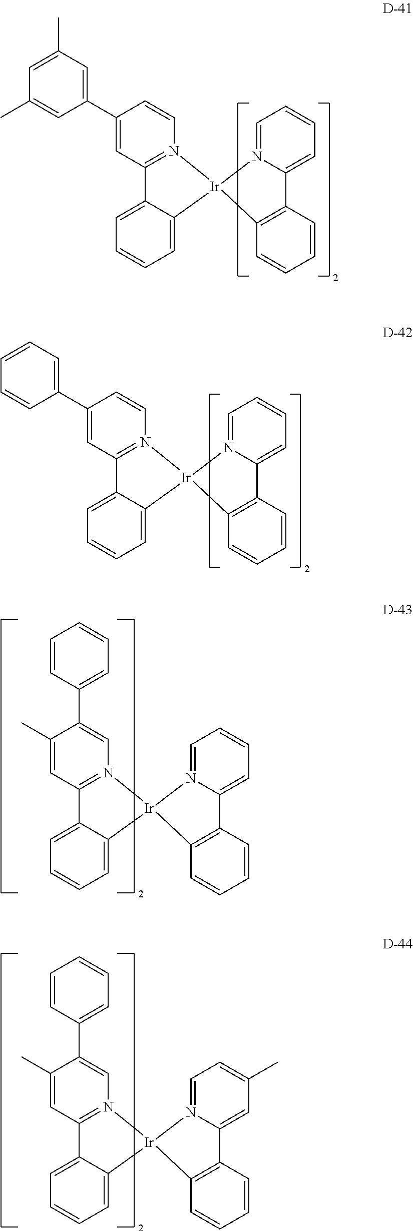

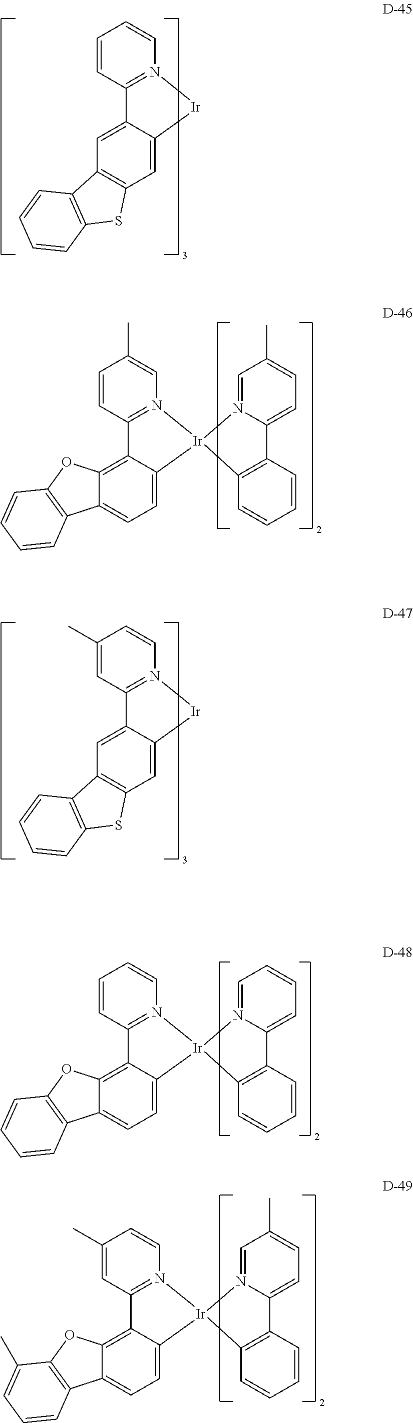

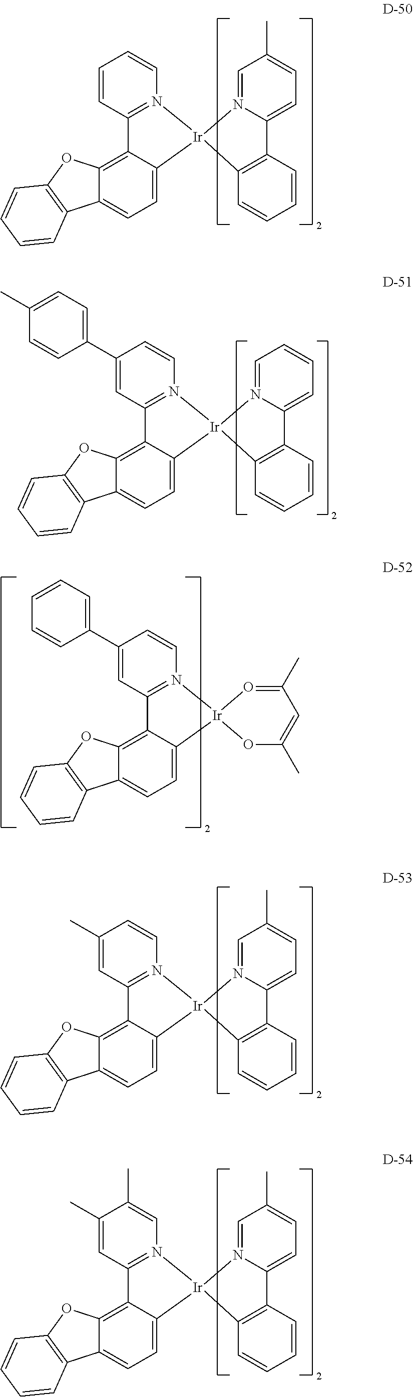

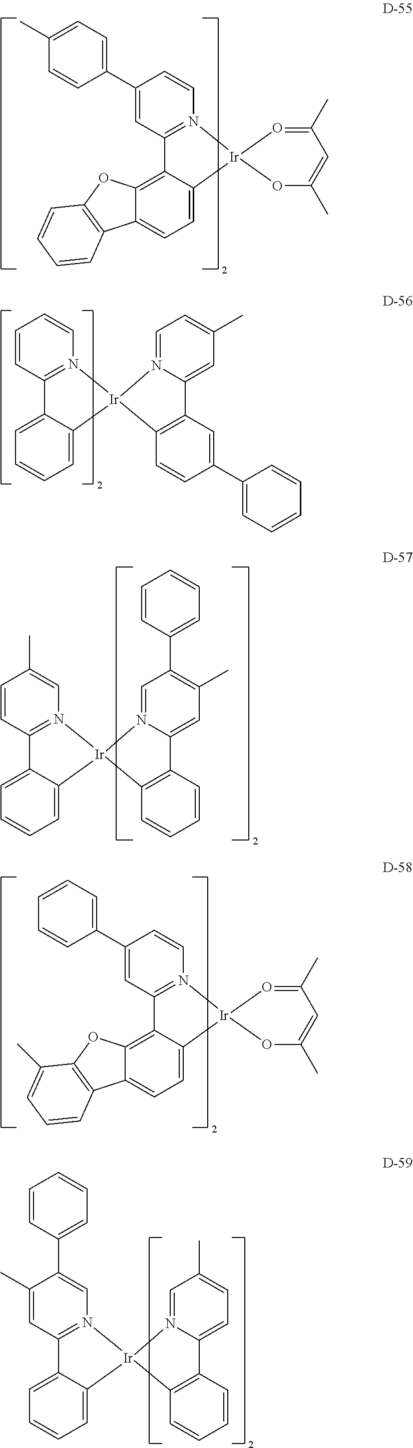

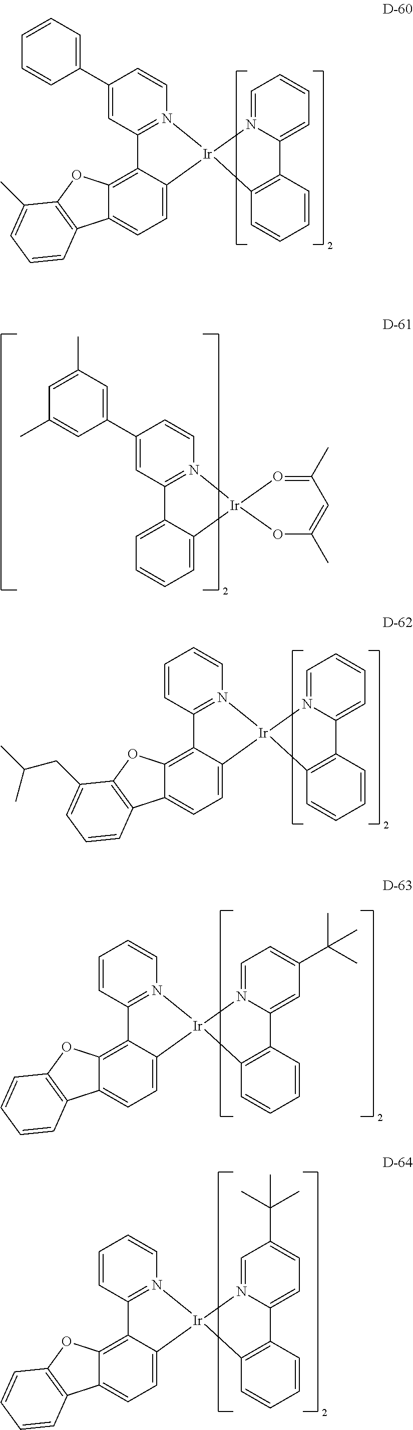

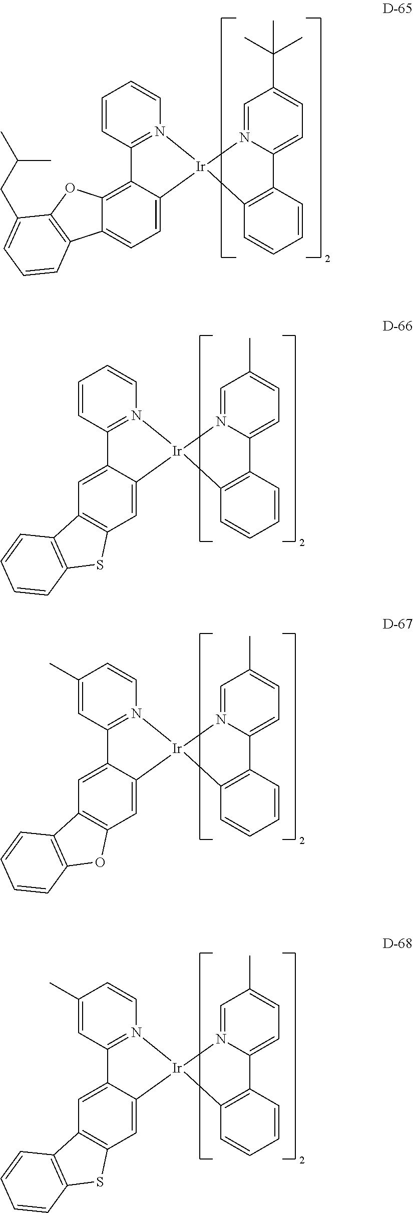

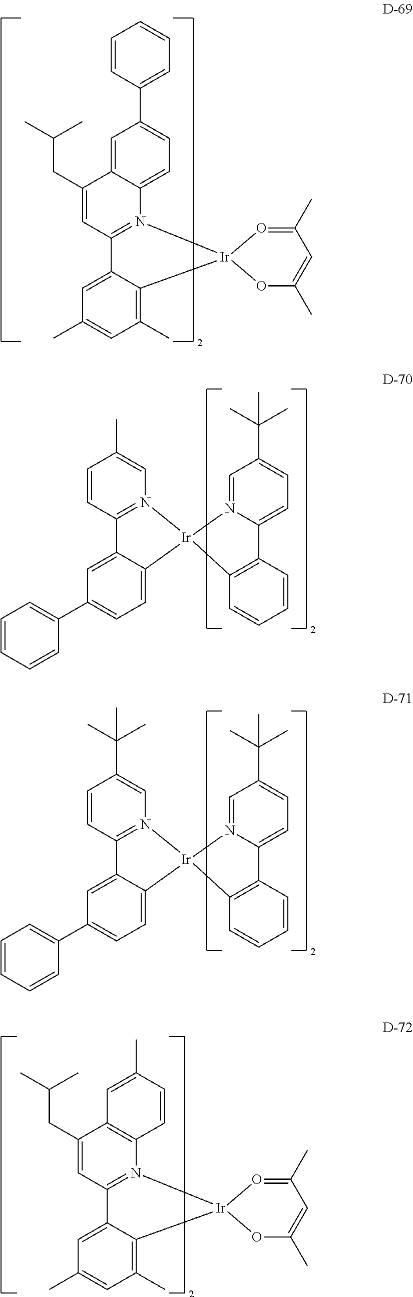

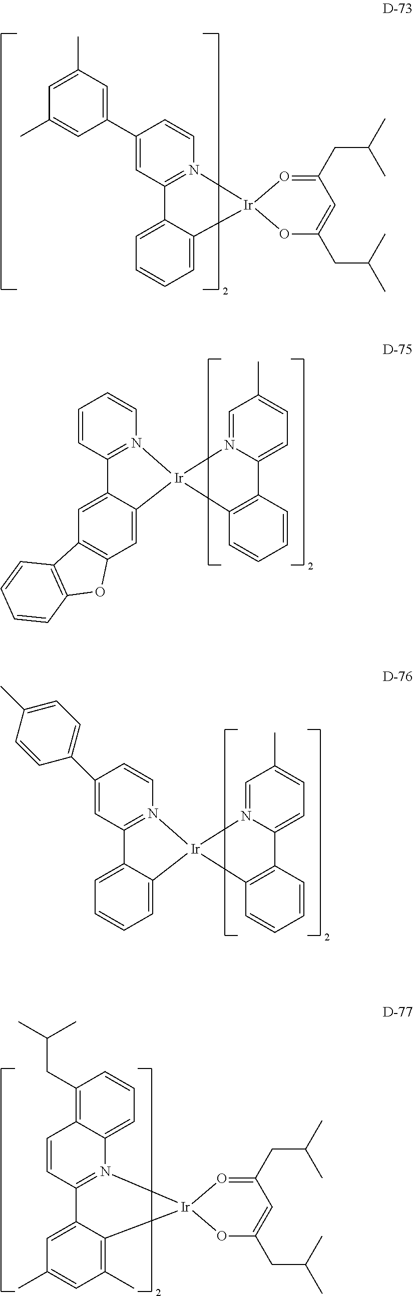

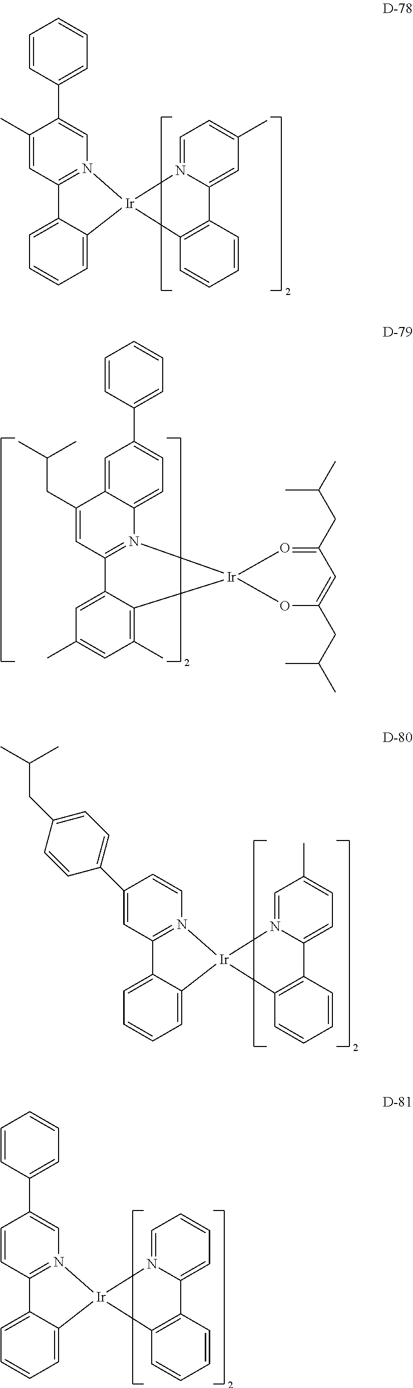

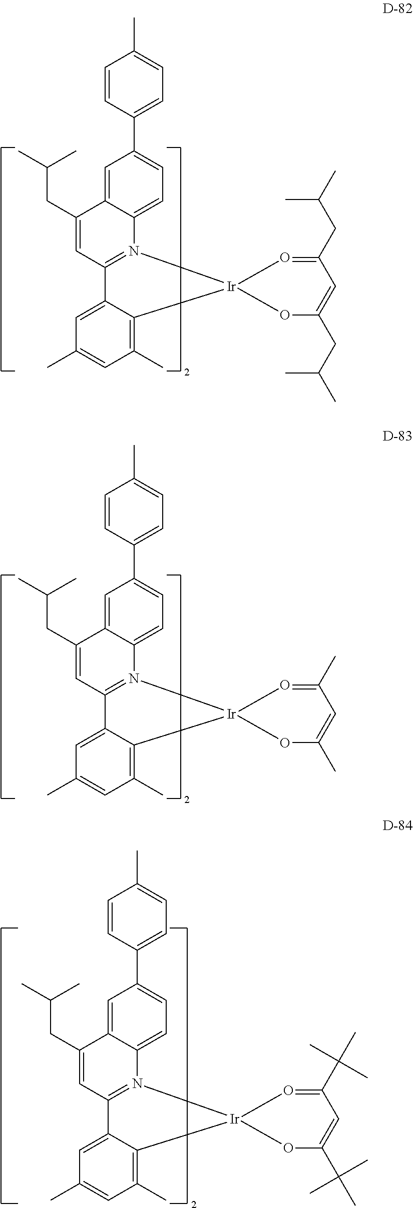

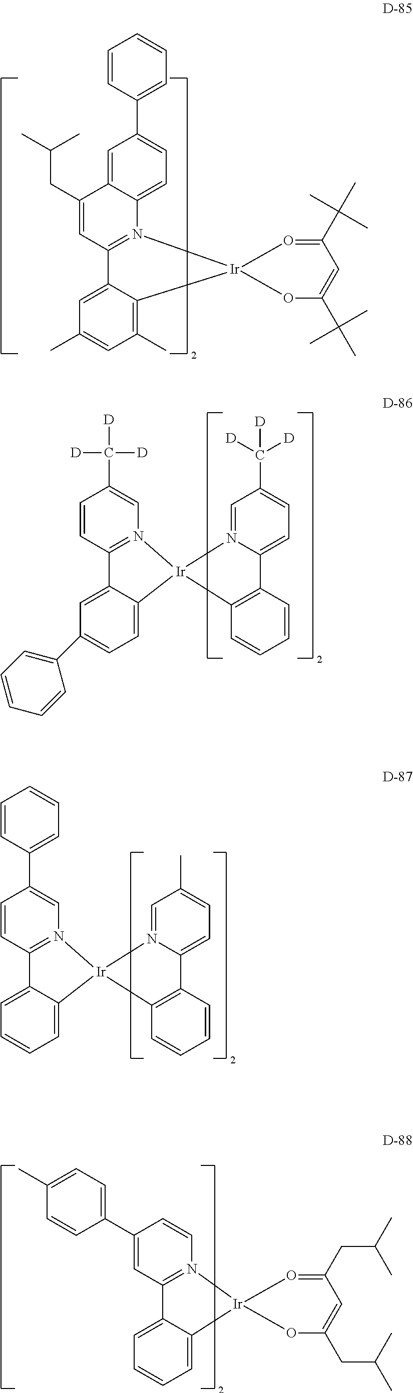

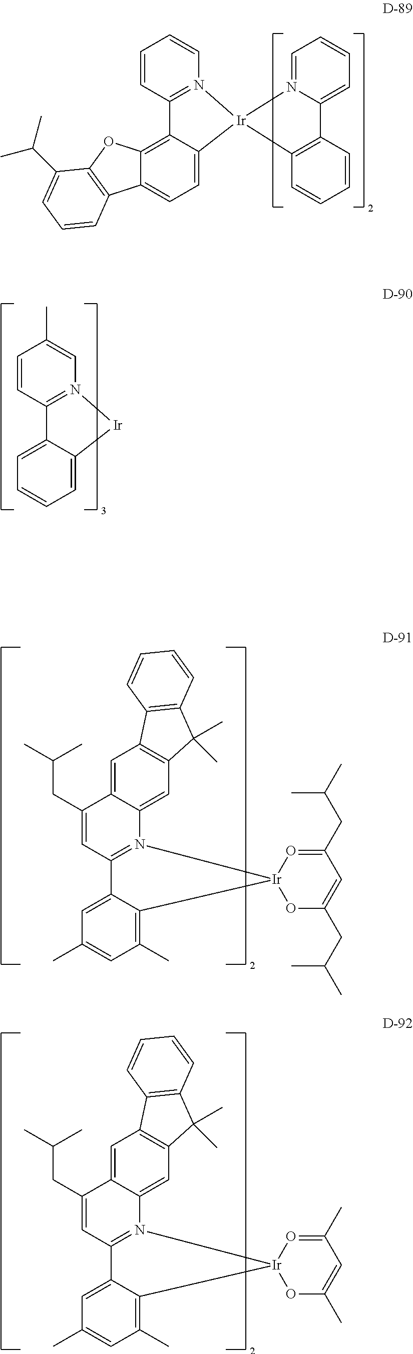

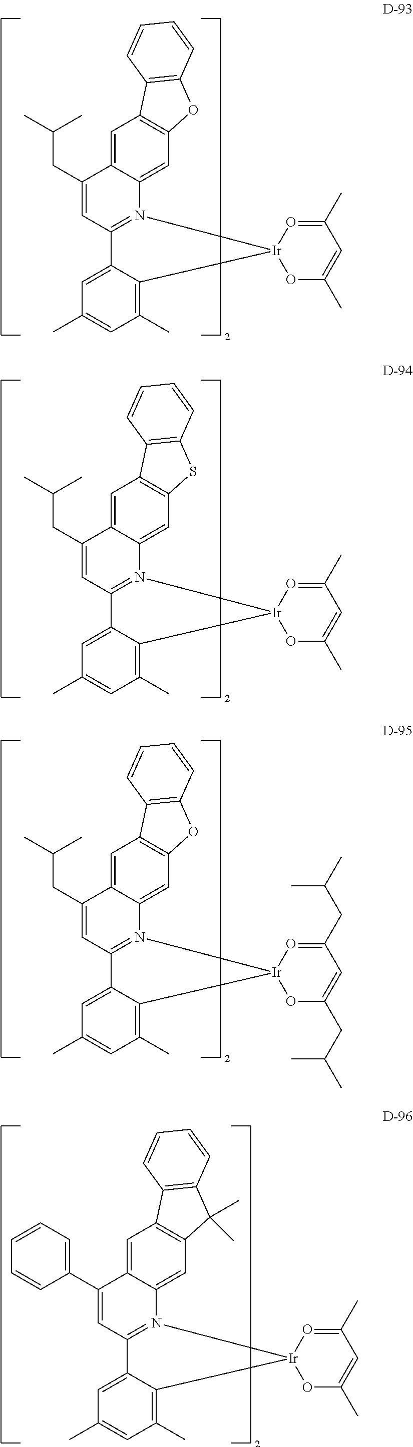

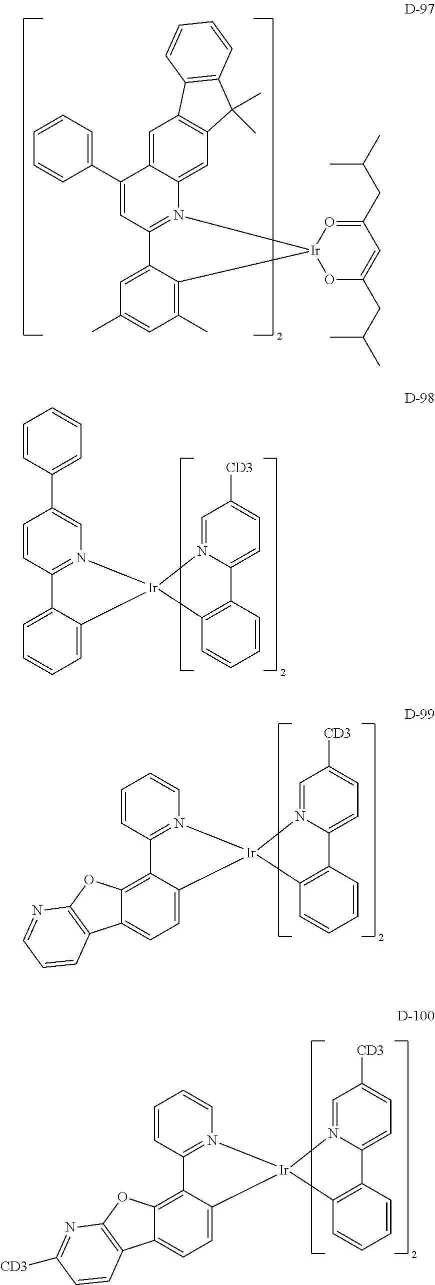

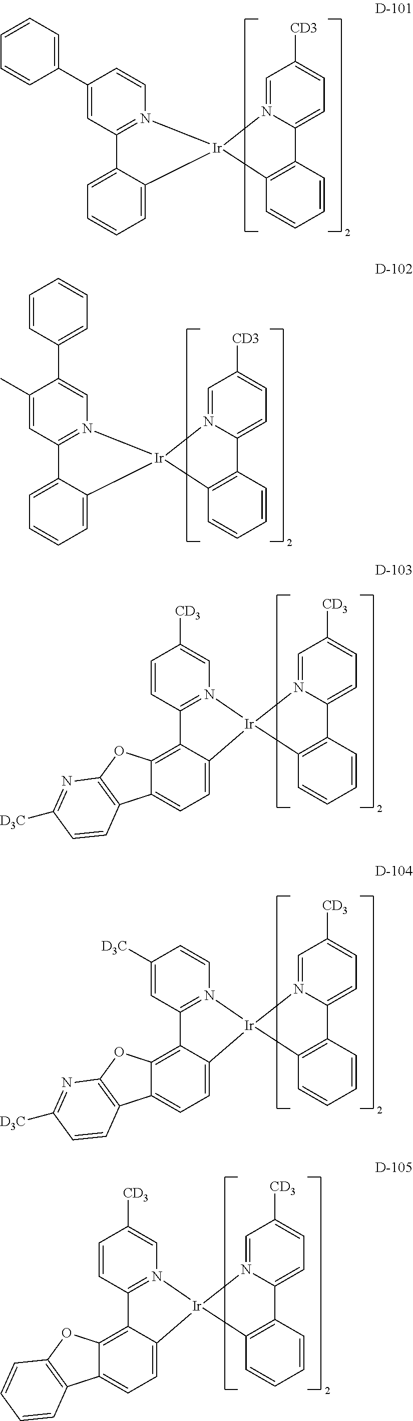

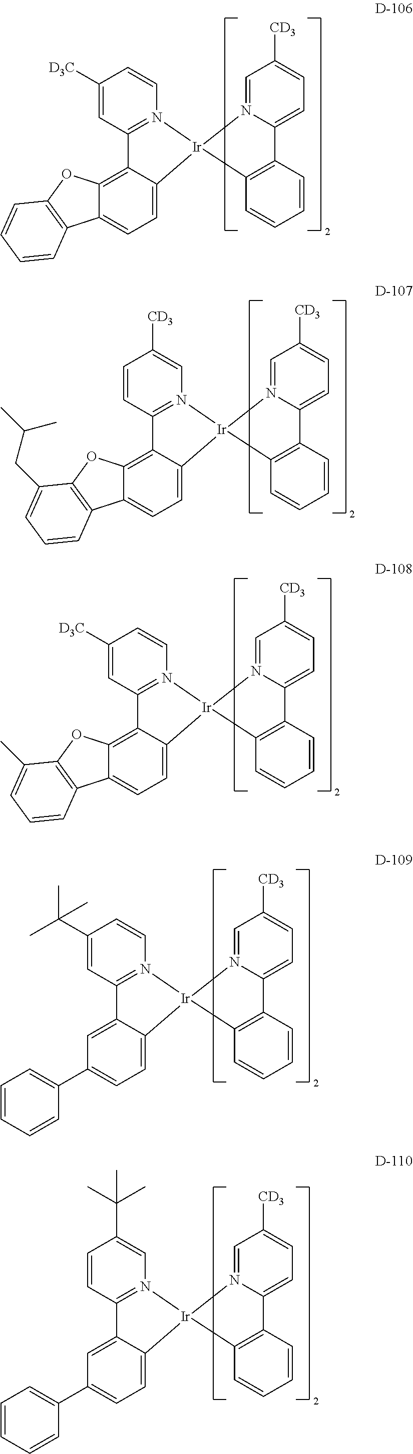

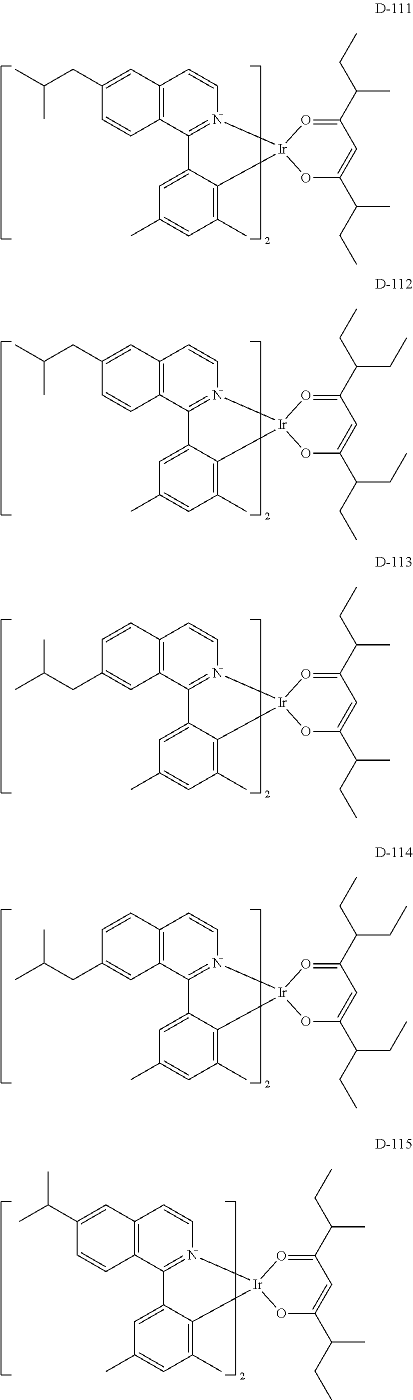

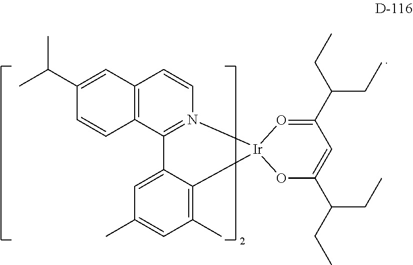

The organic electroluminescent device according to the present disclosure may further comprise at least one compound selected from the group consisting of arylamine-based compounds and styrylarylamine-based compounds in the organic layer.

In addition, in the organic electroluminescent device according to the present disclosure, the organic layer may further comprise at least one metal selected from the group consisting of metals of Group 1, metals of Group 2, transition metals of the 4th period, transition metals of the 5th period, lanthanides and organic metals of d-transition elements of the Periodic Table, or at least one complex compound comprising said metal.

In the organic electroluminescent device according to the present disclosure, at least one layer (hereinafter, “a surface layer”) selected from a chalcogenide layer, a metal halide layer and a metal oxide layer is preferably placed on an inner surface(s) of at least one of a pair of electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiO_{X}(1≤X≤2), AlO_{X}(1≤X≤1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Between the anode and the light-emitting layer, a hole injection layer, a hole transport layer, an electron blocking layer, or a combination thereof can be used. Multiple hole injection layers can be used in order to lower the hole injection barrier (or hole injection voltage) from the anode to the hole transport layer or the electron blocking layer. Two compounds can be simultaneously used in each layer. The hole transport layer or the electron blocking layer can also be formed of multi-layers.

Between the light-emitting layer and the cathode, an electron buffer layer, a hole blocking layer, an electron transport layer, an electron injection layer, or a combination thereof can be used. Multiple electron buffer layers can be used in order to control the injection of the electrons and enhance the interfacial characteristics between the light-emitting layer and the electron injection layer. Two compounds can be simultaneously used in each layer. The hole blocking layer or the electron transport layer can also be formed of multi-layers, and each layer can comprise two or more compounds.

In addition, in the organic electroluminescent device according to the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to produce an electroluminescent device having two or more light-emitting layers and emitting white light.

In order to form each layer of the organic electroluminescent device of the present disclosure, dry film-forming methods such as vacuum evaporation, sputtering, plasma and ion plating methods, or wet film-forming methods such as ink jet printing, nozzle printing, slot coating, spin coating, dip coating, and flow coating methods can be used.

When using a solvent in a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

In addition, the first and the second host compounds of the present disclosure may be film-formed in the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials.

By using the organic electroluminescent device of the present disclosure, a display system or a lighting system can be produced.

Hereinafter, the luminescent properties of the organic electroluminescent device comprising the plurality of host materials of the present disclosure will be explained in detail with reference to the following examples.

### Comparative Example 1: Production of a Red Light-Emitting OLED not According to the Present Disclosure

An OLED not according to the present disclosure was produced as follows: A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. Next, the ITO substrate was mounted on a substrate holder of a vacuum vapor deposition apparatus. Compound HI-1 was introduced into a cell of the vacuum vapor deposition apparatus, and the pressure in the chamber of the apparatus was then controlled to 10⁻⁷ torr. Thereafter, an electric current was applied to the cell to evaporate the introduced material, thereby forming a first hole injection layer having a thickness of 80 nm on the ITO substrate. Compound HI-2 was then introduced into another cell of the vacuum vapor deposition apparatus, and an electric current was applied to the cell to evaporate the introduced material, thereby forming a second hole injection layer having a thickness of 5 nm on the first hole injection layer. Compound HT-1 was introduced into another cell of the vacuum vapor deposition apparatus. Thereafter, an electric current was applied to the cell to evaporate the introduced material, thereby forming a first hole transport layer having a thickness of 10 nm on the second hole injection layer. Compound HT-2 was then introduced into another cell of the vacuum vapor deposition apparatus, and an electric current was applied to the cell to evaporate the introduced material, thereby forming a second hole transport layer having a thickness of 60 nm on the first hole transport layer. After forming the hole injection layers and the hole transport layers, a light-emitting layer was then deposited as follows. Compound H2-1 as a host was introduced into one cell of the vacuum vapor deposition apparatus and compound D-39 as a dopant was introduced into another cell of the apparatus. The two materials were evaporated at a different rate and the dopant was deposited in a doping amount of 3 wt %, based on the total weight of the host and dopant, to form a light-emitting layer having a thickness of 40 nm on the second hole transport layer. Next, compound ET-1 and compound EI-1 were evaporated in a weight ratio of 50:50 as electron transport materials to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited by another vacuum vapor deposition apparatus on the electron injection layer. Thus, an OLED was produced. All the materials used for producing the OLED were purified by vacuum sublimation at 10⁻⁶ torr.

### Device Examples 1 to 5: Production of a Red Light-Emitting OLED According to the Present Disclosure

In Device Examples 1 to 5, OLEDs were produced in the same manner as in Comparative Example 1, except that the first and second host compounds shown in Table 1 below as hosts were introduced into two cells of the vacuum vapor deposition apparatus and compound D-39 was introduced into another cell of the apparatus, the two host materials were evaporated at a rate of 1:1 and the dopant material was simultaneously deposited at a different rate in a doping amount of 3 wt %, based on the total weight of the host and dopant, to form a light-emitting layer having a thickness of 40 nm on the second hole transport layer.

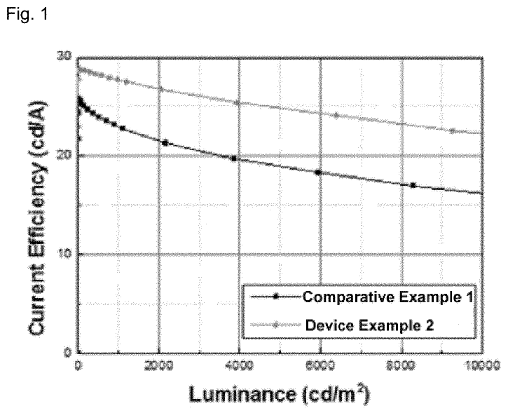

The driving voltage, luminous efficiency, light-emitting color, and the time taken for luminance to decrease from 100% to 95% at a luminance of 5,000 nit (lifespan; T95) of the OLEDs produced in Comparative Example 1 and Device Examples 1 to 5 are provided in Table 1 below. In addition, FIG. 1 illustrates current efficiency versus luminance of the OLEDs produced in Comparative Example 1 and Device Example 2.

TABLE 1

| | | | Luminous | Light- | |
|---|---|---|---|---|---|
| | First | Second | Efficiency | Emitting | Lifespan |
| | Host | Host | (cd/A) | Color | (T95, hr) |
| Comparative | — | H2-1 | 19.0 | Red | 116.4 |
| Example 1 | | | | | |
| Device | H1-7 | H2-1 | 23.7 | Red | 467.3 |
| Example 1 | | | | | |
| Device | H1-1 | H2-1 | 24.9 | Red | 411.4 |
| Example 2 | | | | | |
| Device | H1-127 | H2-1 | 21.1 | Red | 275.4 |
| Example 3 | | | | | |
| Device | H1-179 | H2-1 | 24.1 | Red | 232 |
| Example 4 | | | | | |
| Device | H1-181 | H2-1 | 23.7 | Red | 241 |
| Example 5 | | | | | |

From Device Examples 1 to 3, it is confirmed that the plurality of host materials of the present disclosure may be used to improve luminous efficiency and lifespan characteristic, while maintaining the driving voltage at a similar level or reducing the driving voltage. In particular, as in FIG. 1, the combination of the host materials significantly improves roll-off compared to the comparative example using a single host material.

[Characteristic Analysis]

In order to support the theory of the combination of host compounds mentioned herein, a Hole Only Device (HOD) and an Electron Only Device (EOD) were produced to compare the current characteristic of the combination of the first and second host compounds used in the present disclosure versus only the second host compound. The device structures of the HOD and EOD are as follows.

Hole Only Device (HOD) Example

An ITO substrate was mounted on a substrate holder of a vacuum vapor deposition apparatus. Compound HI-1 was introduced into a cell of the vacuum vapor deposition apparatus, and then the pressure in the chamber of the apparatus was controlled to 10⁻⁷ torr. Thereafter, an electric current was applied to the cell to evaporate the above-introduced material, thereby forming a hole injection layer having a thickness of 10 nm on the ITO substrate. Compound HT-1 was then introduced into another cell of the vacuum vapor deposition apparatus and was evaporated by applying an electric current to the cell, thereby forming a first hole transport layer having a thickness of 10 nm on the hole injection layer. Next, compound HT-2 was introduced into another cell of the vacuum vapor deposition apparatus and was evaporated by applying an electric current to the cell, thereby forming a second hole transport layer having a thickness of 10 nm on the first hole transport layer. After forming the hole injection layer and the hole transport layers, a light-emitting layer was formed thereon as follows: Compound H2-1 was introduced into one cell of the vacuum vapor deposition apparatus as a host, and compound D-39 was introduced into another cell as a dopant. The two materials were evaporated at a different rate and the dopant was deposited in a doping amount of 3 wt % based on the total amount of the host and dopant to form a light-emitting layer having a thickness of 30 nm on the second hole transport layer. Compound HT-1 was then introduced into one cell of the vacuum vapor deposition apparatus and evaporated to form an electron blocking layer having a thickness of 20 nm on the light-emitting layer. Next, an Al cathode having a thickness of 80 nm was deposited on the electron blocking layer by another vacuum vapor deposition apparatus. Thus, an OLED was produced. HOD was produced in the same manner, except that, in the case of a mixture of a first host compound and a second host compound, the first host compound (H1-7) and the second host compound (H2-1) were introduced into two cells of the vacuum vapor deposition apparatus and compound D-39 was introduced into another cell of the apparatus, the two host materials were evaporated at a rate of 1:1 and the dopant material was simultaneously deposited at a different rate in a doping amount of 3 wt %, based on the total weight of the host and dopant, to form a light-emitting layer having a thickness of 30 nm. Voltages at the current density of 10 mA/cm² and 100 mA/cm² are shown in Table 2 below.

TABLE 2

| Material for Light- | Voltage (V) | Voltage (V) |
|---|---|---|
| Emitting Layer | (10 mA/cm²) | (100 mA/cm²) |
| H2-1 | 9.6 | 12.2 |
| H1-7:H2-1 | 4.7 | 7.0 |

### Electron Only Device (EOD) Example

4,6-bis(3,5-di(pyridin-4-yl)phenyl)-2-methylpyrimidine (B4PyMPM) was introduced into one cell of a vacuum vapor deposition apparatus, and a current was applied to the cell to evaporate, thereby forming a hole blocking layer (HBL) having a thickness of 10 nm on ITO. Next, compound H2-1 was introduced into one cell of the vacuum vapor deposition apparatus as a host, and compound D-39 was introduced into another cell as a dopant. The two materials were evaporated at a different rate and the dopant was deposited in a doping amount of 2 wt % based on the total amount of the host and the dopant to form a light-emitting layer having a thickness of 40 nm on the hole blocking layer. Compound ET-1 and lithium quinolate were introduced into one cell and another cell of the vacuum vapor deposition apparatus, respectively, and the two materials were evaporated at the same rate and doped in a doping amount of 50 wt % to form an electron transport layer having a thickness of 30 nm on the light-emitting layer. After depositing lithium quinolate as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr. EOD was produced in the same manner, except that, in the case of a mixture of a first host compound and a second host compound, the first host compound (H1-7) and the second host compound (H2-1) were introduced into two cells of the vacuum vapor deposition apparatus and compound D-39 was introduced into another cell of the apparatus, the two host materials were evaporated at a rate of 1:1 and the dopant material was simultaneously deposited in a different rate in a doping amount of 3 wt %, based on the total weight of the host and dopant, to form a light-emitting layer having a thickness of 40 nm. Voltages at the current density of 10 mA/cm² and 100 mA/cm² are shown in Table 3 below.

TABLE 3

| Material for Light- | Voltage (V) | Voltage (V) |
|---|---|---|
| Emitting Layer | (10 mA/cm²) | (100 mA/cm²) |
| H2-1 | 3.0 | 4.3 |
| H1-7:H2-1 | 3.3 | 4.9 |

As can be seen from Table 2 above, according to the HOD Example, the device comprising a light-emitting layer of only compound H2-1 showed relatively high driving voltage characteristic compared to the device comprising the combination of compound H1-7 (the first host compound) and compound H2-1 (the second host compound), and thus it shows a hole injection blocking characteristic. Meanwhile, it is confirmed that the combination of compound H1-7 (the first host compound) and compound H2-1 (the second host compound) showed significantly improved hole current characteristic due to compound H1-7 (the first host compound). In addition, as can be seen from Table 3 above, according to the EOD Example, the device comprising a light-emitting layer of only compound H2-1 showed relatively low driving voltage characteristic compared to the device comprising the combination of Compound H1-7 (the first host compound) and compound H2-1 (the second host compound). Meanwhile, it is confirmed that the combination of compound H1-7 (the first host compound) and compound H2-1 (the second host compound) showed slightly reduced electron current characteristic due to compound H1-7 (the first host compound). As a result, the combination of the first host compound and the second host compound according to the present disclosure showed relatively good charge balance characteristic by relatively improving hole current characteristic and slightly reducing electron current characteristic.

The compounds used in the Comparative Example and Device Examples are shown in Table 4 below.

TABLE 4

| | | |
|---|---|---|
| Hole Injection Layer/ Hole Transport Layer | 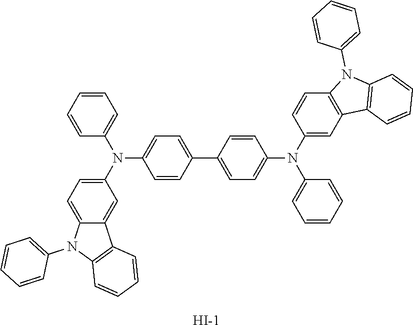 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] | 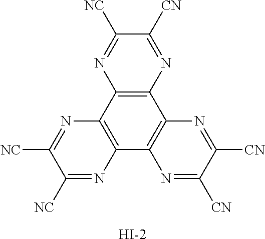 |
| | 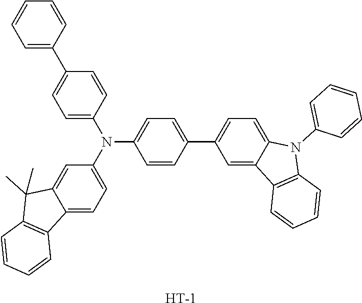 | 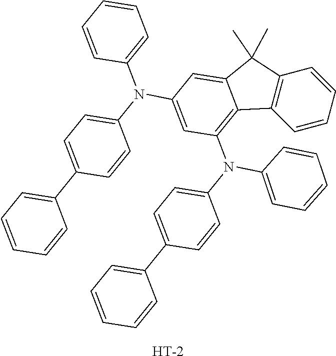 |

| | | | |
|---|---|---|---|
| Light- Emitting Layer | 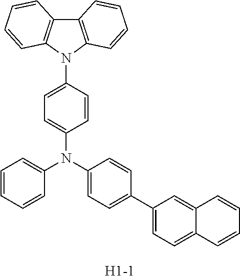 [SMILES: c1ccc(N(c2ccc(-c3ccc4ccccc4c3)cc2)c2ccc(-n3c4ccccc4c4ccccc43)cc2)cc1] | 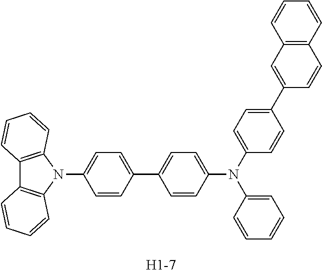 [SMILES: c1ccc(N(c2ccc(-c3ccc(-n4c5ccccc5c5ccccc54)cc3)cc2)c2ccc(-c3ccc4ccccc4c3)cc2)cc1] | 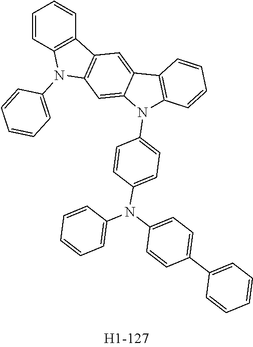 [SMILES: c1ccc(-c2ccc(N(c3ccccc3)c3ccc(-n4c5ccccc5c5cc6c7ccccc7n(-c7ccccc7)c6cc54)cc3)cc2)cc1] |

| | | |
|---|---|---|
| | 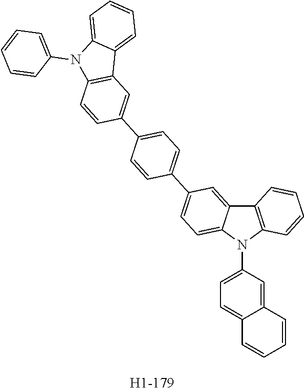 [SMILES: c1ccc(-n2c3ccccc3c3cc(-c4ccc(-c5ccc6c(c5)c5ccccc5n6-c5ccc6ccccc6c5)cc4)ccc32)cc1] | 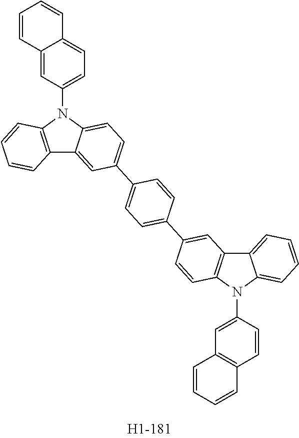 [SMILES: c1ccc2cc(-n3c4ccccc4c4cc(-c5ccc(-c6ccc7c(c6)c6ccccc6n7-c6ccc7ccccc7c6)cc5)ccc43)ccc2c1] |
| | 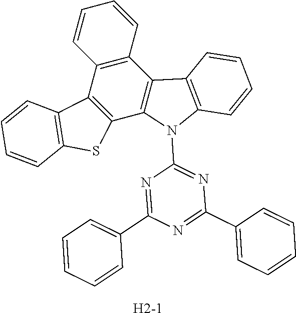 | 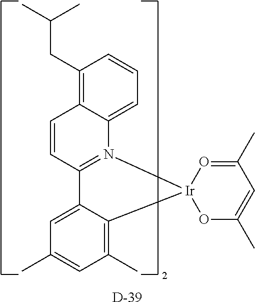 |
| Hole Blocking Layer/ Electron Transport Layer/ Electron Injection Layer | 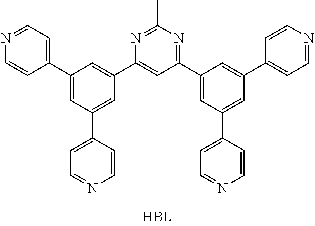 [SMILES: Cc1nc(-c2cc(-c3ccncc3)cc(-c3ccncc3)c2)cc(-c2cc(-c3ccncc3)cc(-c3ccncc3)c2)n1] | 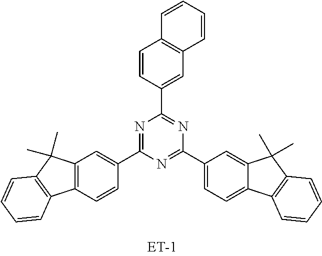 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 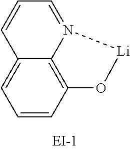 | |

1. A plurality of host materials comprising at least one first host compound and at least one second host compound, wherein the first host compound is represented by the following formula 1, and the second host compound is represented by the following formula 2: 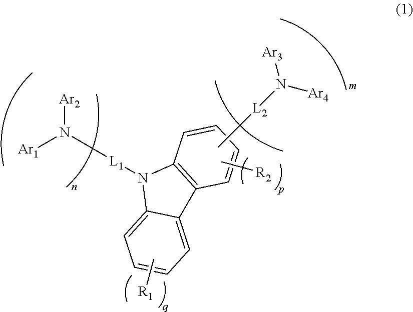
wherein
Ar₁ to Ar₄ each independently represent a substituted or unsubstituted (C6-C30)aryl; or Ar₁ and Ar₂, and Ar₃ and Ar₄ may be linked to each other to form a substituted or unsubstituted (3- to 30-membered) ring;
L₁ represents a single bond, or a substituted or unsubstituted (C6-C30)aryl(ene);
L₂ represents a single bond, or a substituted or unsubstituted (C6-C30)arylene;
if Ar₁ or Ar₂ represents a substituted or unsubstituted (C6-C30)aryl, and L₁ represents a substituted or unsubstituted (C6-C30)arylene, Ar₁ or Ar₂ and L₁ may be linked via a single bond to form a substituted or unsubstituted (3- to 30-membered) ring;
if Ar₃ or Ar₄ represents a substituted or unsubstituted (C6-C30)aryl, and L₂ represents a substituted or unsubstituted (C6-C30)arylene, Ar₃ or Ar₄ and L₂ may be linked via a single bond to form a substituted or unsubstituted (3- to 30-membered) ring;
R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring;
m and n each independently represent an integer of 0 to 2, with a proviso that at least one of m and n is 1 or more; and
p and q each independently represent an integer of 1 to 4, in which if p and q represent an integer of 2 or more, each of R₁ and R₂ may be the same or different; 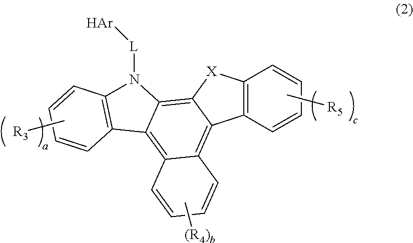
wherein
X represents —NR₁₁—, —CR₁₂R₁₃—, —O—, or —S—;
HAr represents a substituted or unsubstituted (3- to 30-membered)heteroaryl;
L represents a single bond, a substituted or unsubstituted (C6-C30)arylene, or a substituted or unsubstituted (3- to 30-membered)heteroarylene;
R₁₁ to R₁₃ each independently represent a substituted or unsubstituted (C1-C30)alkyl, a substituted or unsubstituted (C6-C30)aryl, or a substituted or unsubstituted (3- to 30-membered)heteroaryl;
R₃ to R₅ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a substituted or unsubstituted (3- to 30-membered) ring; and
a, b, and c each independently represent an integer of 1 to 4, in which if a, b, and c represent an integer of 2 or more, each of R₃, R₄, and R₅ may be the same or different.

2. The plurality of host materials according to claim 1, wherein formula 1 is represented by the following formula 1-1 or 1-2: 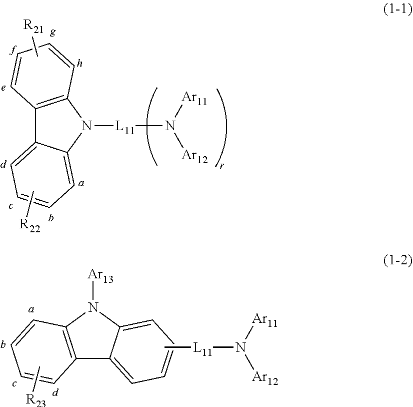
wherein
Ar₁₁ to Ar₁₃ each independently represent a substituted or unsubstituted (C6-C30)aryl; or Ar₁₁ and Ar₁₂ may be linked to each other to form a substituted or unsubstituted (3- to 30-membered) ring;
L₁₁ represents a single bond, or a substituted or unsubstituted (C6-C30)arylene;
if Ar₁₁ or Ar₁₂ represents a substituted or unsubstituted (C6-C30)aryl, and L₁₁ represents a substituted or unsubstituted (C6-C30)arylene, Ar₁₁ or Ar₁₂ and L₁₁ may be linked via a single bond to form a substituted or unsubstituted (3- to 30-membered) ring;
at least one of a and b, b and c, c and d, e and f, f and g, or g and h in formulas 1-1 and 1-2 and two * positions of the following formula 1-a, 1-b, or 1-c may be fused to form a ring: 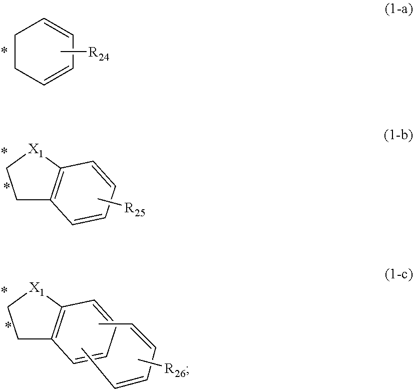
X₁ represents NR₃₁, O, S, or CR₃₂R₃₃;
R₃₁ to R₃₃ each independently represent a substituted or unsubstituted (C1-C30)alkyl, a substituted or unsubstituted (C6-C30)aryl, or a substituted or unsubstituted (3- to 30-membered)heteroaryl;
R₂₁ to R₂₆ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; and
r represents 1 or 2.

3. The plurality of host materials according to claim 1, wherein HAr in formula 2 represents a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, a substituted or unsubstituted pyridazinyl, a substituted or unsubstituted benzoimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted quinoxalinyl, a substituted or unsubstituted naphthyridyl, a substituted or unsubstituted phenanthridinyl, a substituted or unsubstituted benzofuranopyrimidinyl, a substituted or unsubstituted benzothiophenopyrimidinyl, a substituted or unsubstituted benzoquinazolinyl, a substituted or unsubstituted benzoquinoxalinyl, a substituted or unsubstituted pyridoquinoxalinyl, a substituted or unsubstituted pyrazinoquinoxalinyl, a substituted or unsubstituted dibenzoquinoxalinyl, or a substituted or unsubstituted pyridobenzoquinoxalinyl.

4. The plurality of host materials according to claim 1, wherein the substituents of the substituted alkyl, the substituted alkoxy, the substituted cycloalkyl, the substituted aryl(ene), the substituted heteroaryl(ene), the substituted trialkylsilyl, the substituted triarylsilyl, the substituted dialkylarylsilyl, the substituted alkyldiarylsilyl, the substituted mono- or di-alkylamino, the substituted mono- or di-arylamino, the substituted alkylarylamino, and the substituted ring in Ar₁ to Ar₄, HAr, R₁ to R₅, R₁₁ to R₁₃, L, L₁, and L₂ each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (3- to 7-membered)heterocycloalkyl, a (C6-C30)aryloxy, a (C6-C30)arylthio, a (3- to 30-membered)heteroaryl unsubstituted or substituted with a (C6-C30)aryl, a (C6-C30)aryl unsubstituted or substituted with a (3- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

5. The plurality of host materials according to claim 1, wherein the compound represented by formula 1 is selected from the following compounds:

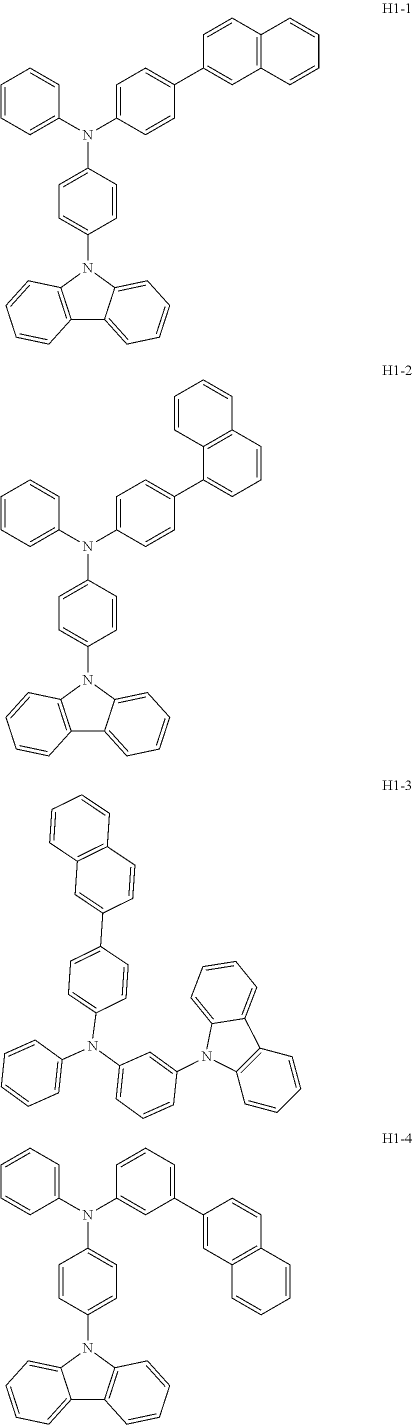

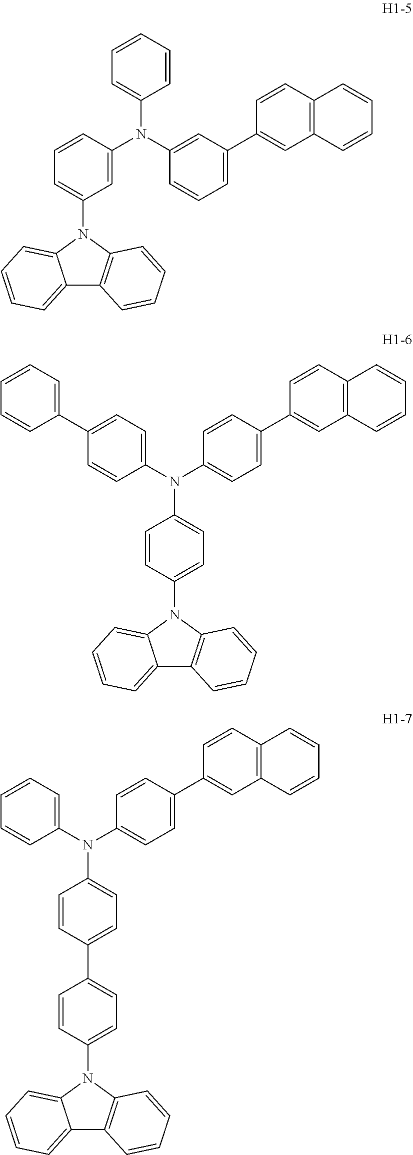

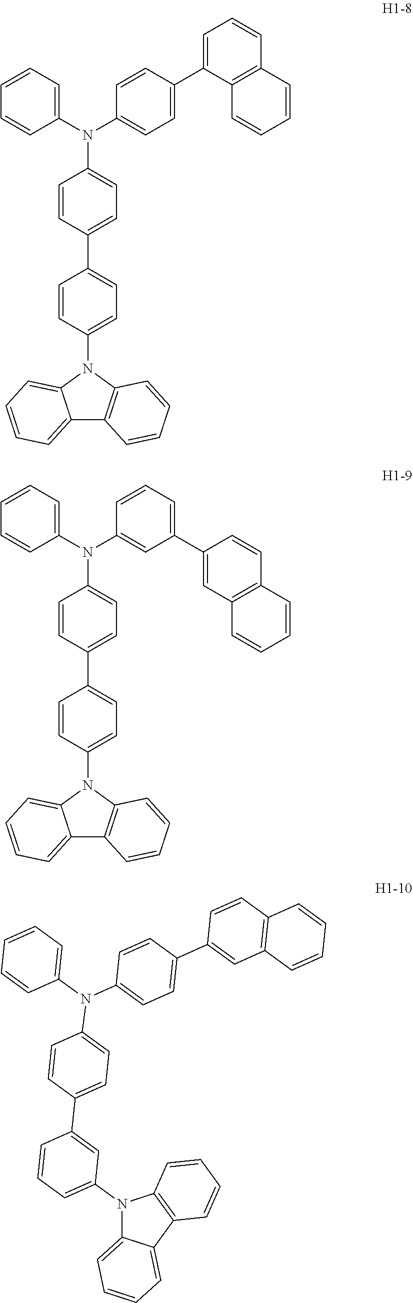

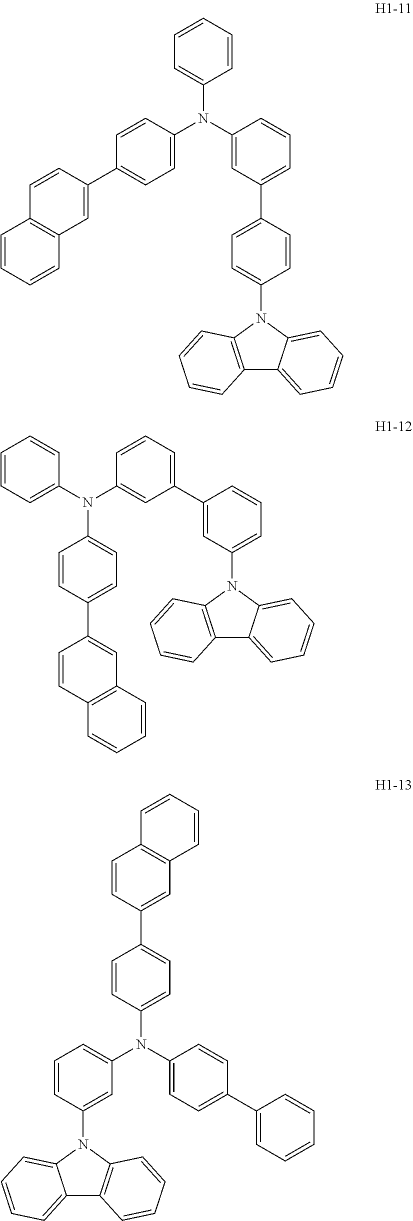 [SMILES: c1ccc(N(c2ccc(-c3ccc4ccccc4c3)cc2)c2cccc(-c3ccc(-n4c5ccccc5c5ccccc54)cc3)c2)cc1 ; c1ccc(N(c2ccc(-c3ccc4ccccc4c3)cc2)c2cccc(-c3cccc(-n4c5ccccc5c5ccccc54)c3)c2)cc1 ; c1ccc(-c2ccc(N(c3ccc(-c4ccc5ccccc5c4)cc3)c3cccc(-n4c5ccccc5c5ccccc54)c3)cc2)cc1]

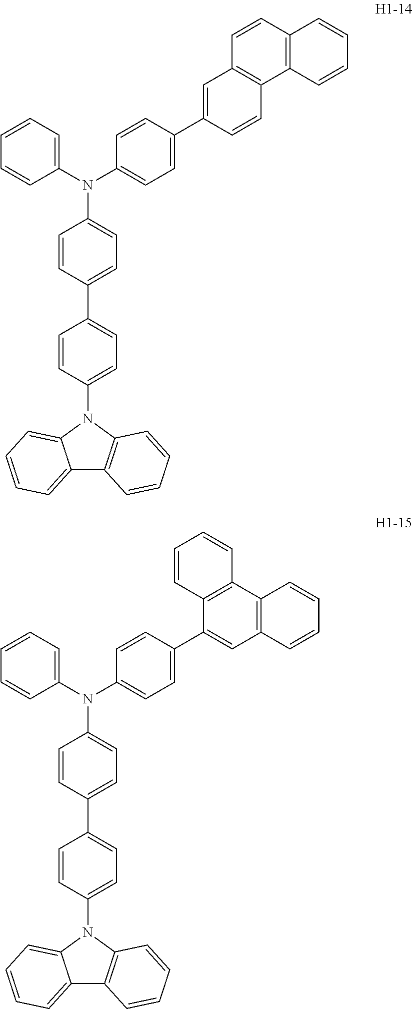 [SMILES: c1ccc(N(c2ccc(-c3ccc(-n4c5ccccc5c5ccccc54)cc3)cc2)c2ccc(-c3ccc4c(ccc5ccccc54)c3)cc2)cc1 ; c1ccc(N(c2ccc(-c3ccc(-n4c5ccccc5c5ccccc54)cc3)cc2)c2ccc(-c3cc4ccccc4c4ccccc34)cc2)cc1]

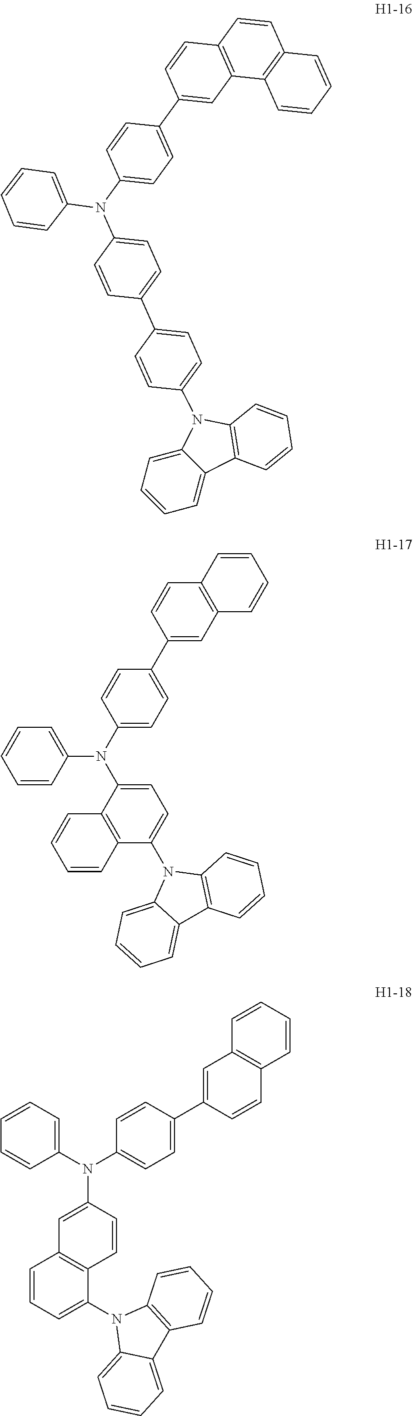 [SMILES: c1ccc(N(c2ccc(-c3ccc(-n4c5ccccc5c5ccccc54)cc3)cc2)c2ccc(-c3ccc4ccc5ccccc5c4c3)cc2)cc1 ; c1ccc(N(c2ccc(-c3ccc4ccccc4c3)cc2)c2ccc(-n3c4ccccc4c4ccccc43)c3ccccc23)cc1 ; c1ccc(N(c2ccc(-c3ccc4ccccc4c3)cc2)c2ccc3c(-n4c5ccccc5c5ccccc54)cccc3c2)cc1]

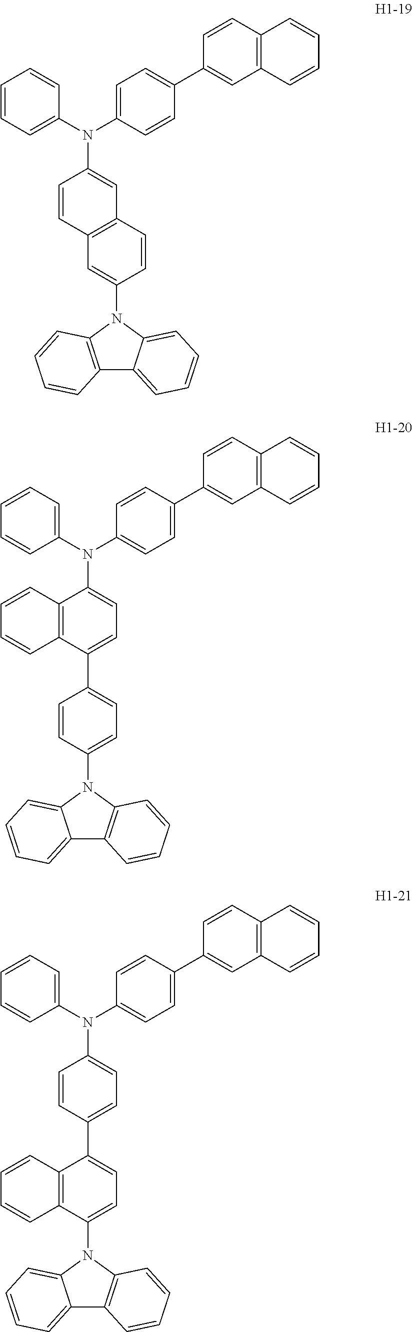 [SMILES: c1ccc(N(c2ccc(-c3ccc4ccccc4c3)cc2)c2ccc3cc(-n4c5ccccc5c5ccccc54)ccc3c2)cc1 ; c1ccc(N(c2ccc(-c3ccc4ccccc4c3)cc2)c2ccc(-c3ccc(-n4c5ccccc5c5ccccc54)cc3)c3ccccc23)cc1 ; c1ccc(N(c2ccc(-c3ccc4ccccc4c3)cc2)c2ccc(-c3ccc(-n4c5ccccc5c5ccccc54)c4ccccc34)cc2)cc1]

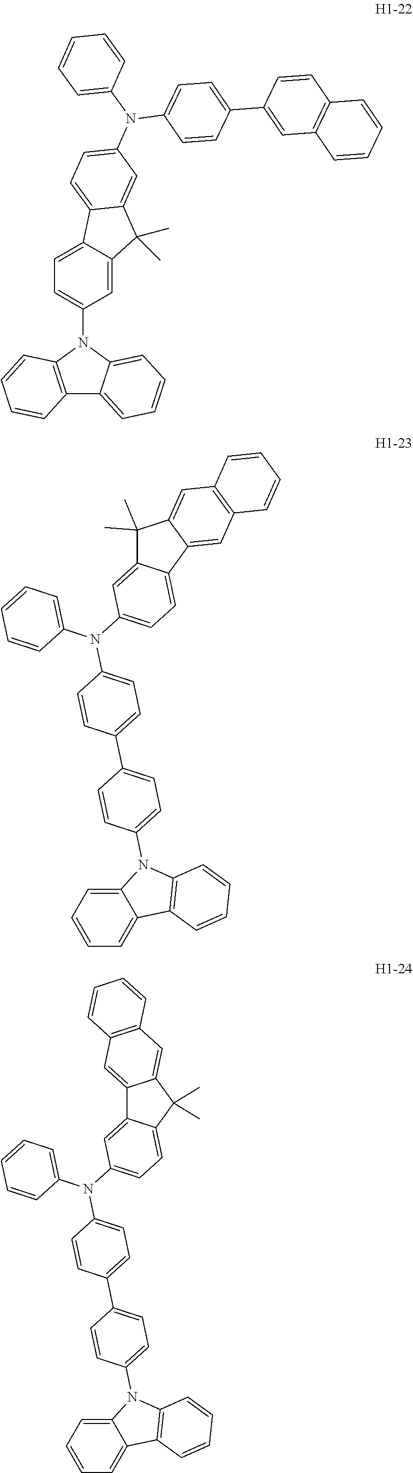

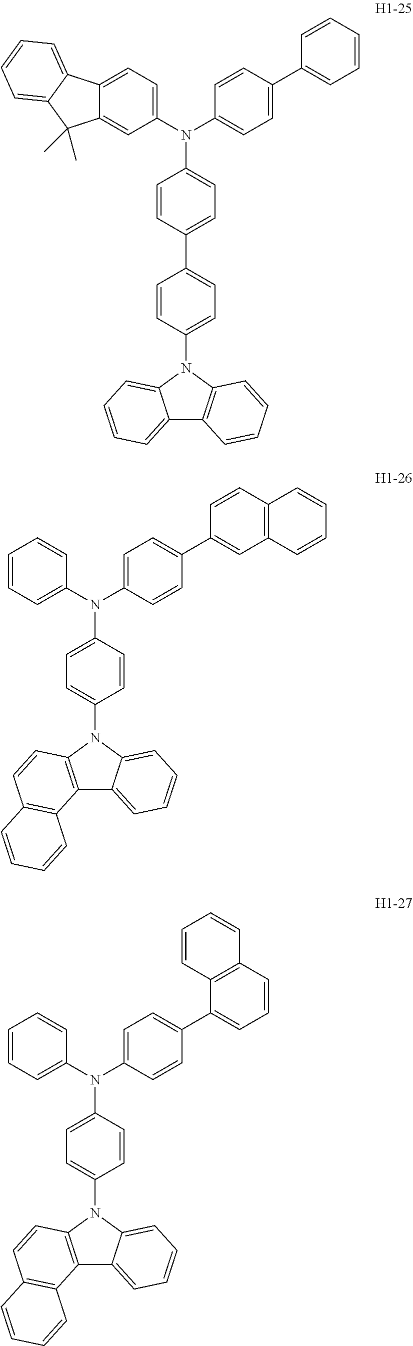

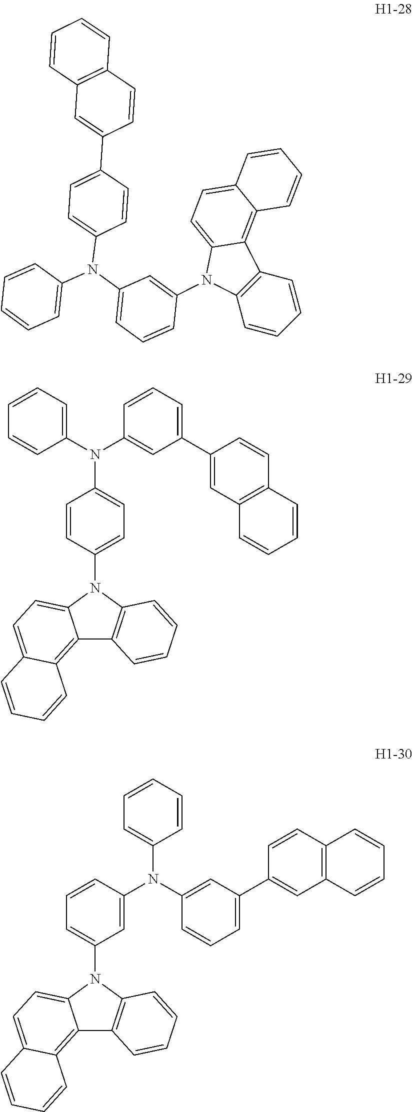 [SMILES: c1ccc(N(c2ccc(-c3ccc4ccccc4c3)cc2)c2cccc(-n3c4ccccc4c4c5ccccc5ccc43)c2)cc1 ; c1ccc(N(c2ccc(-n3c4ccccc4c4c5ccccc5ccc43)cc2)c2cccc(-c3ccc4ccccc4c3)c2)cc1 ; c1ccc(N(c2cccc(-c3ccc4ccccc4c3)c2)c2cccc(-n3c4ccccc4c4c5ccccc5ccc43)c2)cc1]

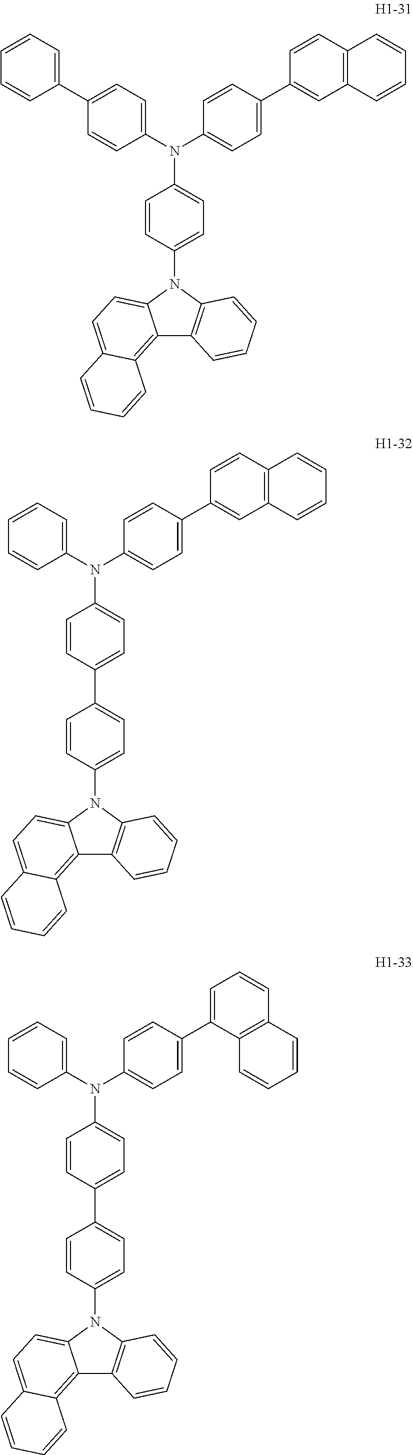 [SMILES: c1ccc(-c2ccc(N(c3ccc(-c4ccc5ccccc5c4)cc3)c3ccc(-n4c5ccccc5c5c6ccccc6ccc54)cc3)cc2)cc1 ; c1ccc(N(c2ccc(-c3ccc(-n4c5ccccc5c5c6ccccc6ccc54)cc3)cc2)c2ccc(-c3ccc4ccccc4c3)cc2)cc1 ; c1ccc(N(c2ccc(-c3ccc(-n4c5ccccc5c5c6ccccc6ccc54)cc3)cc2)c2ccc(-c3cccc4ccccc34)cc2)cc1]

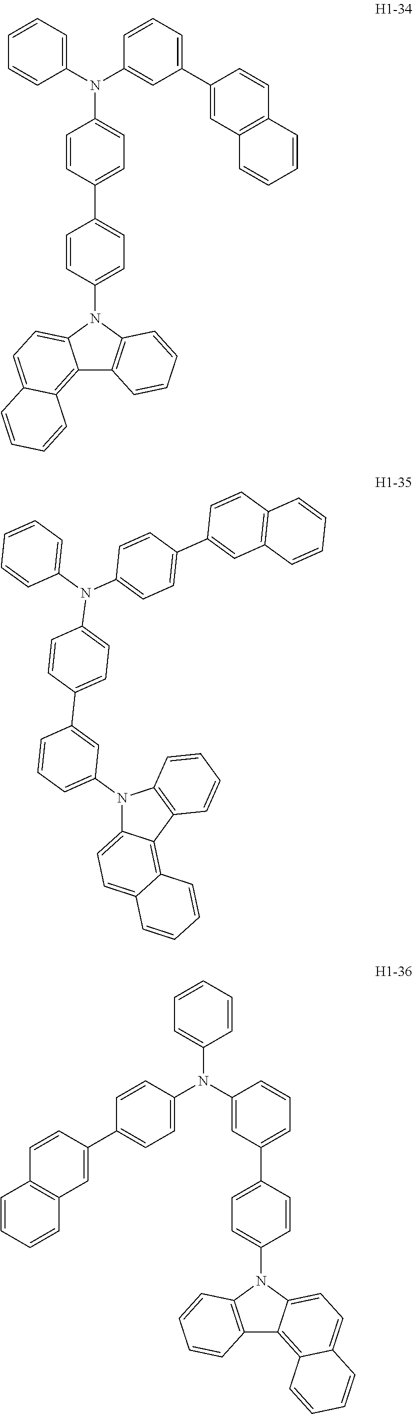 [SMILES: c1ccc(N(c2ccc(-c3ccc(-n4c5ccccc5c5c6ccccc6ccc54)cc3)cc2)c2cccc(-c3ccc4ccccc4c3)c2)cc1 ; c1ccc(N(c2ccc(-c3cccc(-n4c5ccccc5c5c6ccccc6ccc54)c3)cc2)c2ccc(-c3ccc4ccccc4c3)cc2)cc1 ; c1ccc(N(c2ccc(-c3ccc4ccccc4c3)cc2)c2cccc(-c3ccc(-n4c5ccccc5c5c6ccccc6ccc54)cc3)c2)cc1]

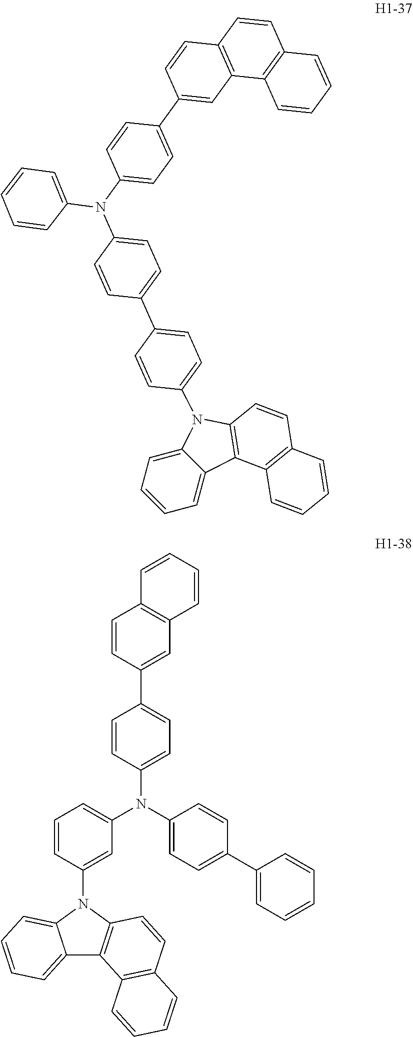

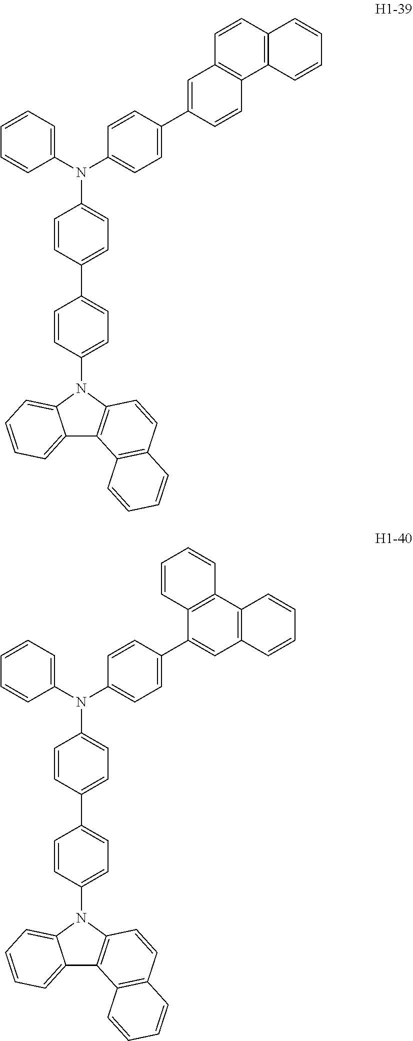 [SMILES: c1ccc(N(c2ccc(-c3ccc(-n4c5ccccc5c5c6ccccc6ccc54)cc3)cc2)c2ccc(-c3ccc4c(ccc5ccccc54)c3)cc2)cc1 ; c1ccc(N(c2ccc(-c3ccc(-n4c5ccccc5c5c6ccccc6ccc54)cc3)cc2)c2ccc(-c3cc4ccccc4c4ccccc34)cc2)cc1]

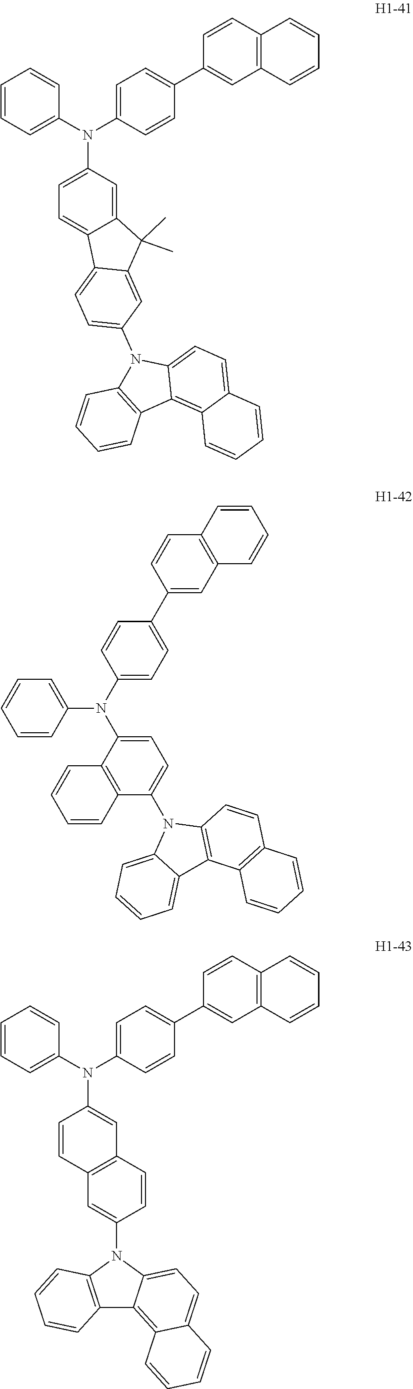

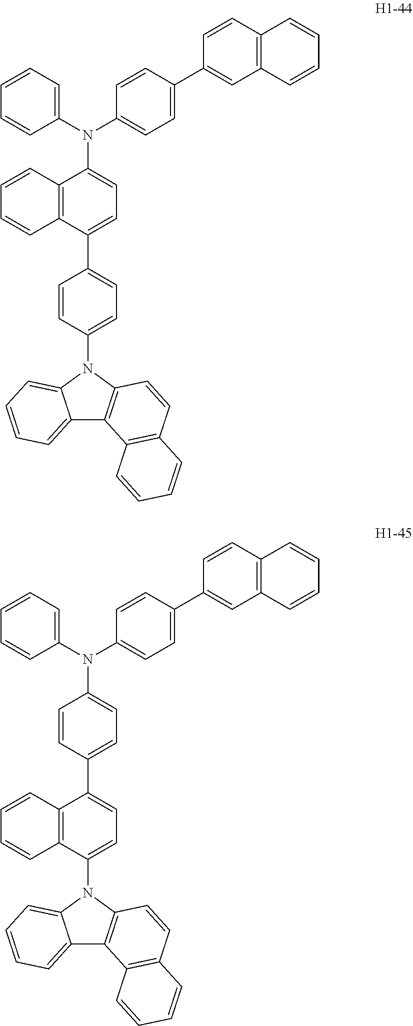 [SMILES: c1ccc(N(c2ccc(-c3ccc4ccccc4c3)cc2)c2ccc(-c3ccc(-n4c5ccccc5c5c6ccccc6ccc54)cc3)c3ccccc23)cc1 ; c1ccc(N(c2ccc(-c3ccc4ccccc4c3)cc2)c2ccc(-c3ccc(-n4c5ccccc5c5c6ccccc6ccc54)c4ccccc34)cc2)cc1]

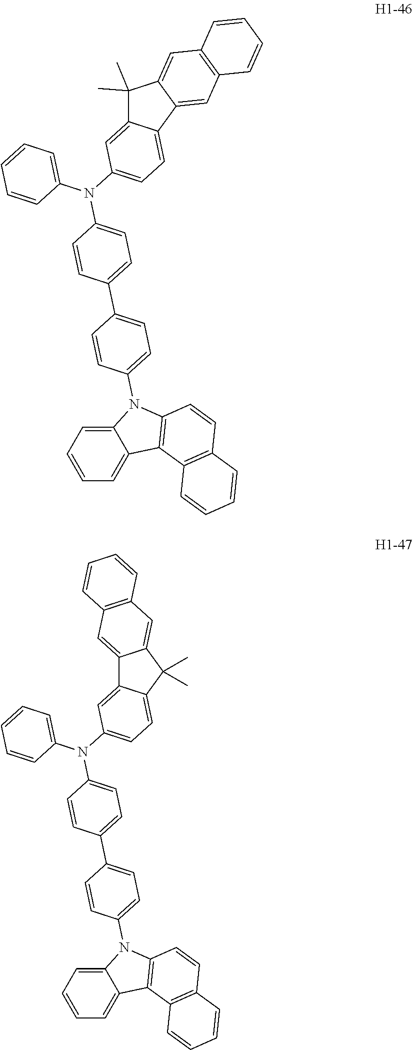 [SMILES: CC1(C)c2cc(N(c3ccccc3)c3ccc(-c4ccc(-n5c6ccccc6c6c7ccccc7ccc65)cc4)cc3)ccc2-c2cc3ccccc3cc21 ; CC1(C)c2ccc(N(c3ccccc3)c3ccc(-c4ccc(-n5c6ccccc6c6c7ccccc7ccc65)cc4)cc3)cc2-c2cc3ccccc3cc21]

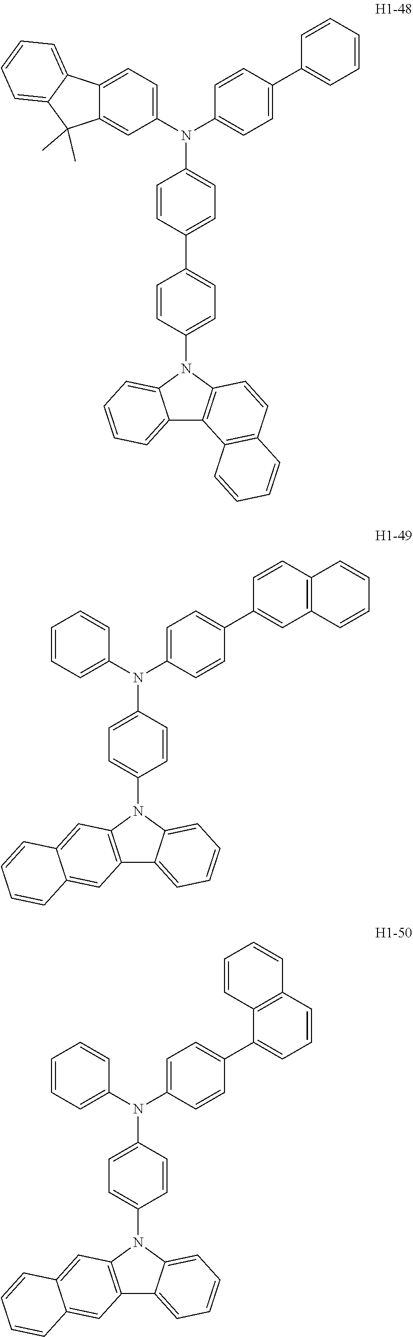 [SMILES: CC1(C)c2ccccc2-c2ccc(N(c3ccc(-c4ccccc4)cc3)c3ccc(-c4ccc(-n5c6ccccc6c6c7ccccc7ccc65)cc4)cc3)cc21 ; c1ccc(N(c2ccc(-c3ccc4ccccc4c3)cc2)c2ccc(-n3c4ccccc4c4cc5ccccc5cc43)cc2)cc1 ; c1ccc(N(c2ccc(-c3cccc4ccccc34)cc2)c2ccc(-n3c4ccccc4c4cc5ccccc5cc43)cc2)cc1]

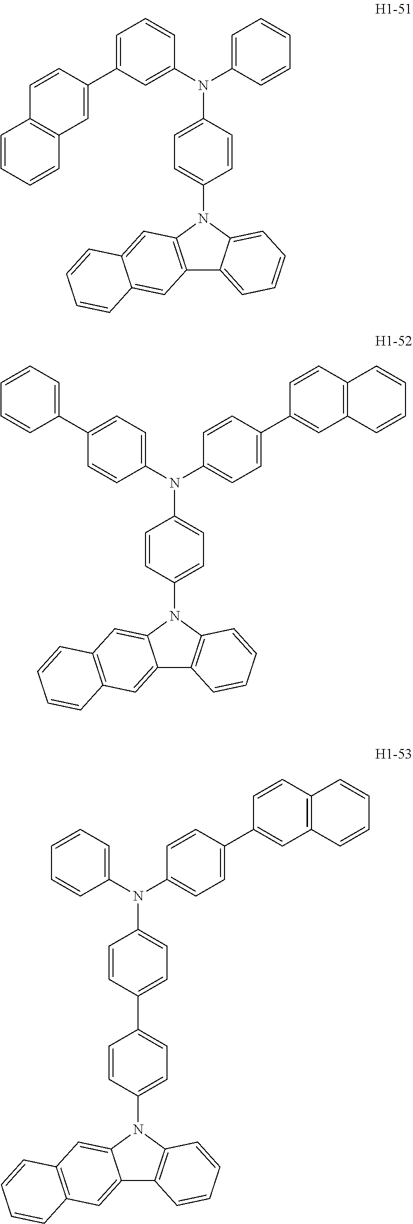 [SMILES: c1ccc(N(c2ccc(-n3c4ccccc4c4cc5ccccc5cc43)cc2)c2cccc(-c3ccc4ccccc4c3)c2)cc1 ; c1ccc(-c2ccc(N(c3ccc(-c4ccc5ccccc5c4)cc3)c3ccc(-n4c5ccccc5c5cc6ccccc6cc54)cc3)cc2)cc1 ; c1ccc(N(c2ccc(-c3ccc(-n4c5ccccc5c5cc6ccccc6cc54)cc3)cc2)c2ccc(-c3ccc4ccccc4c3)cc2)cc1]

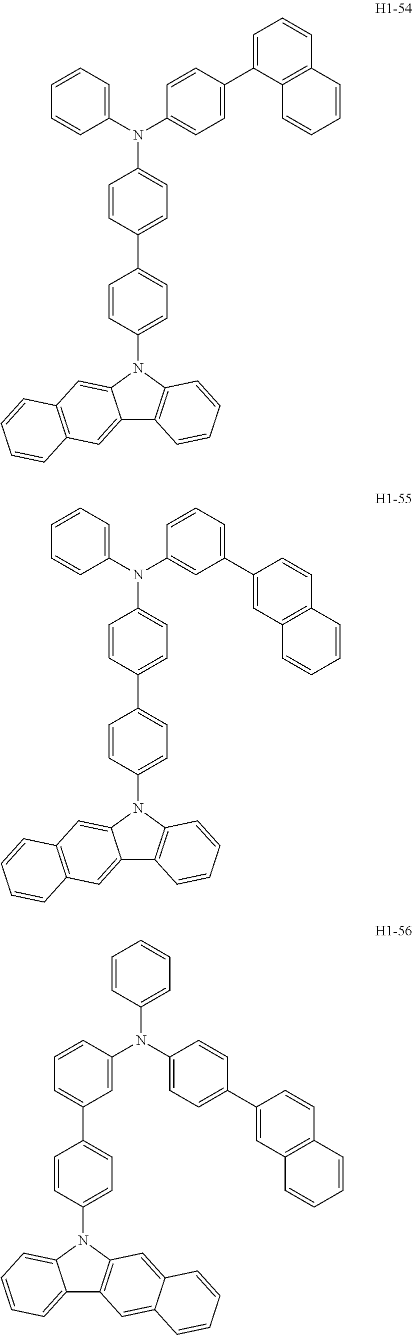 [SMILES: c1ccc(N(c2ccc(-c3ccc(-n4c5ccccc5c5cc6ccccc6cc54)cc3)cc2)c2ccc(-c3cccc4ccccc34)cc2)cc1 ; c1ccc(N(c2ccc(-c3ccc(-n4c5ccccc5c5cc6ccccc6cc54)cc3)cc2)c2cccc(-c3ccc4ccccc4c3)c2)cc1 ; c1ccc(N(c2ccc(-c3ccc4ccccc4c3)cc2)c2cccc(-c3ccc(-n4c5ccccc5c5cc6ccccc6cc54)cc3)c2)cc1]

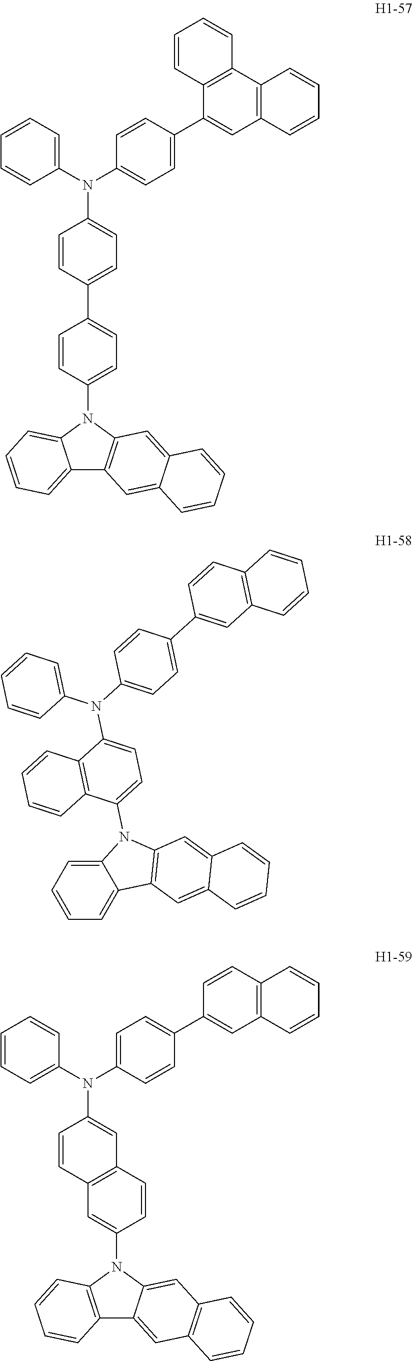 [SMILES: c1ccc(N(c2ccc(-c3ccc(-n4c5ccccc5c5cc6ccccc6cc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccccc4c4cc5ccccc5cc43)c3ccccc23)cc1 ; c1ccc(N(c2ccc(-c3ccc4ccccc4c3)cc2)c2ccc3cc(-n4c5ccccc5c5cc6ccccc6cc54)ccc3c2)cc1]

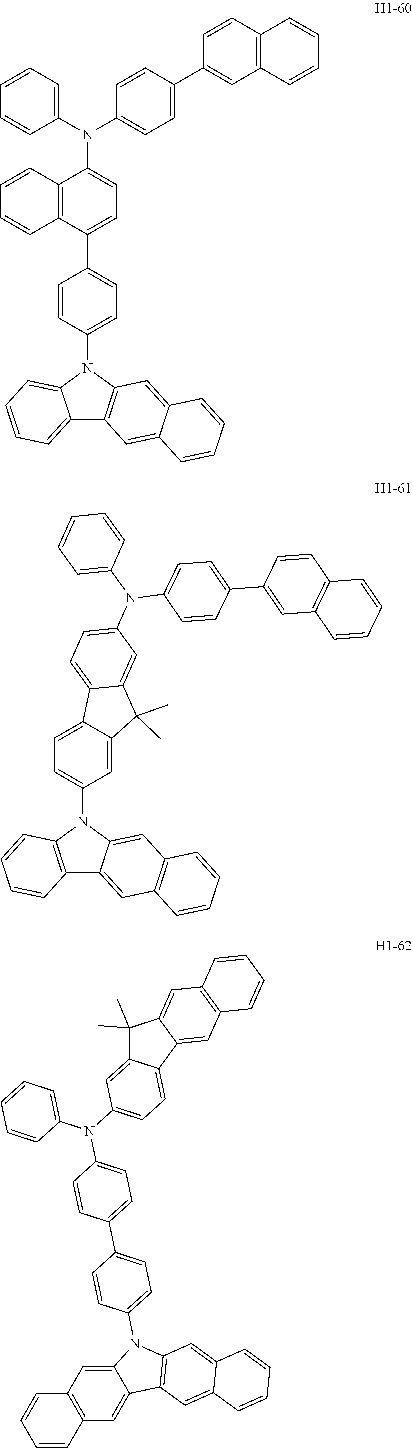 [SMILES: c1ccc(N(c2ccc(-c3ccc4ccccc4c3)cc2)c2ccc(-c3ccc(-n4c5ccccc5c5cc6ccccc6cc54)cc3)c3ccccc23)cc1 ; CC1(C)c2cc(N(c3ccccc3)c3ccc(-c4ccc5ccccc5c4)cc3)ccc2-c2ccc(-n3c4ccccc4c4cc5ccccc5cc43)cc21 ; CC1(C)c2cc(N(c3ccccc3)c3ccc(-c4ccc(-n5c6cc7ccccc7cc6c6cc7ccccc7cc65)cc4)cc3)ccc2-c2cc3ccccc3cc21]

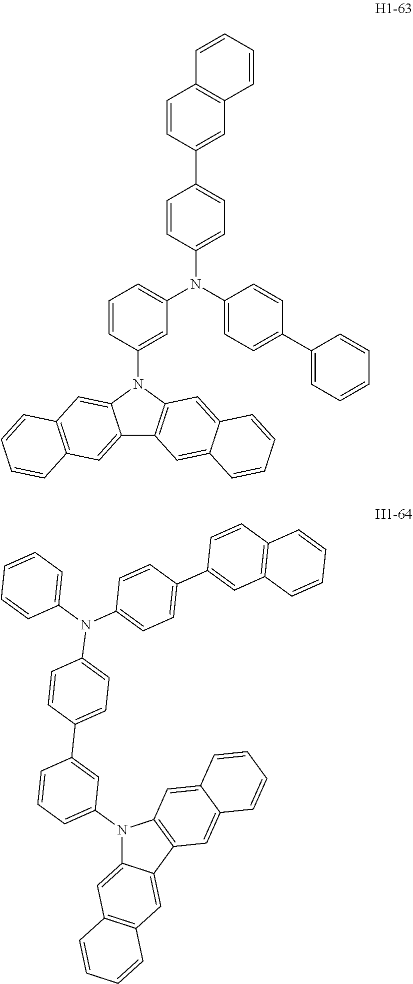 [SMILES: c1ccc(-c2ccc(N(c3ccc(-c4ccc5ccccc5c4)cc3)c3cccc(-n4c5cc6ccccc6cc5c5cc6ccccc6cc54)c3)cc2)cc1 ; c1ccc(N(c2ccc(-c3cccc(-n4c5cc6ccccc6cc5c5cc6ccccc6cc54)c3)cc2)c2ccc(-c3ccc4ccccc4c3)cc2)cc1]

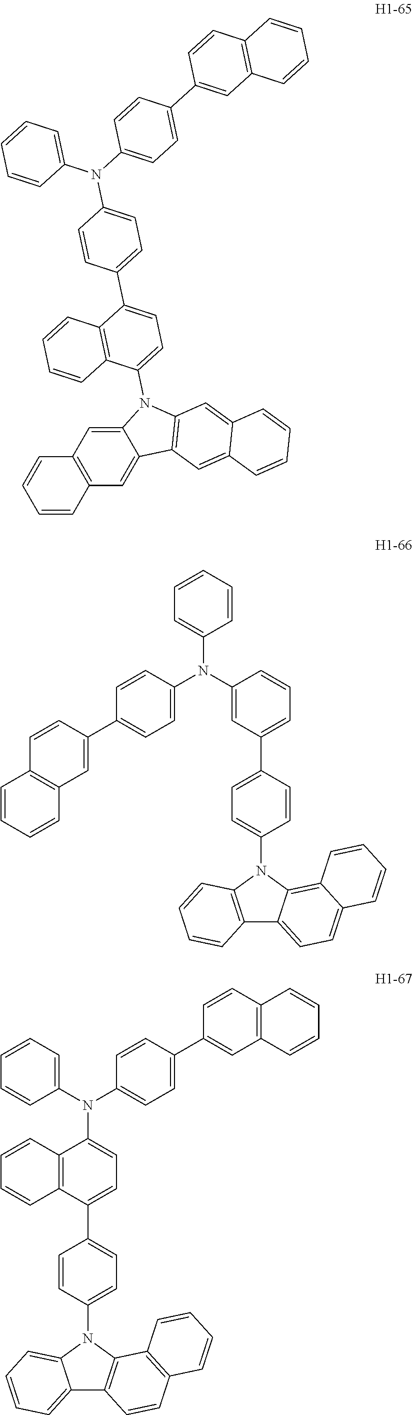

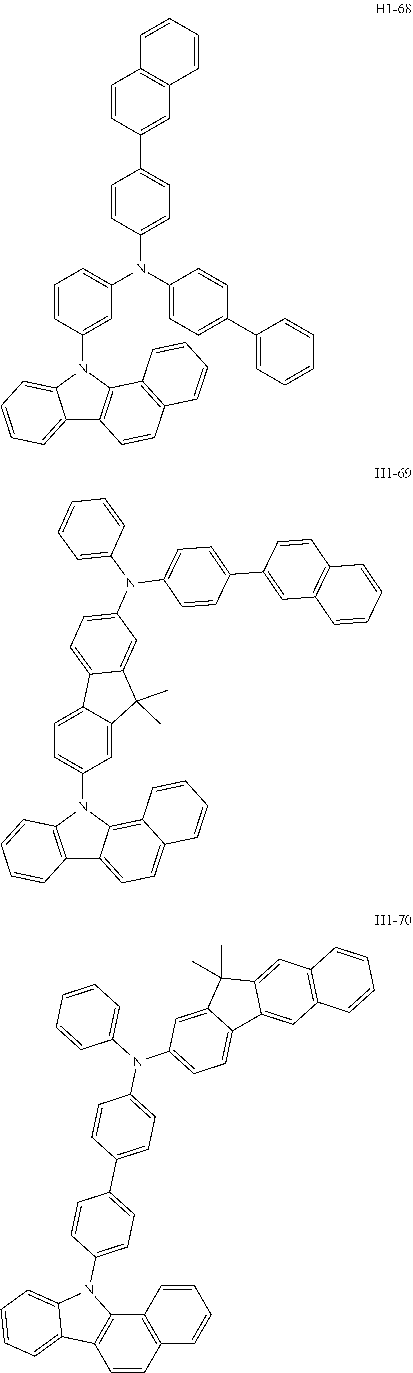 [SMILES: c1ccc(-c2ccc(N(c3ccc(-c4ccc5ccccc5c4)cc3)c3cccc(-n4c5ccccc5c5ccc6ccccc6c54)c3)cc2)cc1 ; CC1(C)c2cc(N(c3ccccc3)c3ccc(-c4ccc5ccccc5c4)cc3)ccc2-c2ccc(-n3c4ccccc4c4ccc5ccccc5c43)cc21 ; CC1(C)c2cc(N(c3ccccc3)c3ccc(-c4ccc(-n5c6ccccc6c6ccc7ccccc7c65)cc4)cc3)ccc2-c2cc3ccccc3cc21]

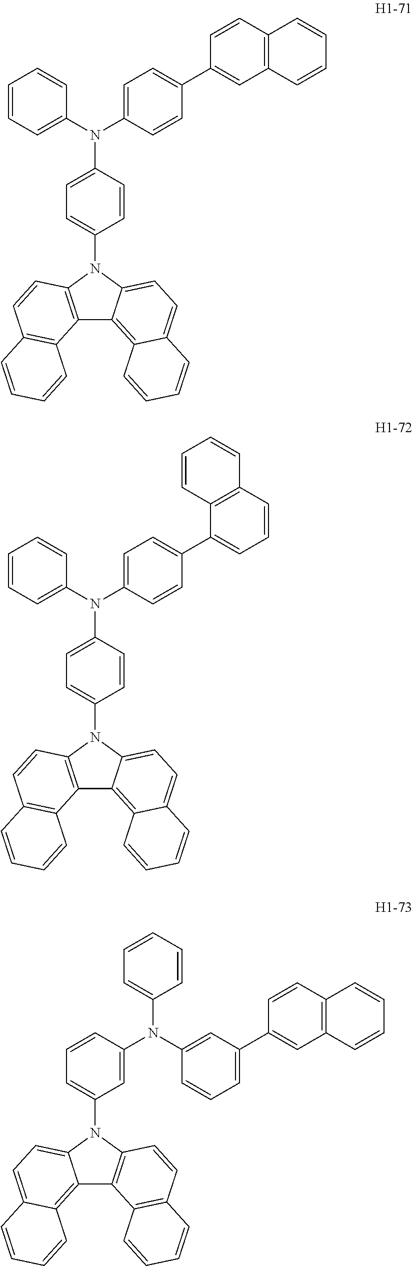 [SMILES: c1ccc(N(c2ccc(-c3ccc4ccccc4c3)cc2)c2ccc(-n3c4ccc5ccccc5c4c4c5ccccc5ccc43)cc2)cc1 ; c1ccc(N(c2ccc(-c3cccc4ccccc34)cc2)c2ccc(-n3c4ccc5ccccc5c4c4c5ccccc5ccc43)cc2)cc1 ; c1ccc(N(c2cccc(-c3ccc4ccccc4c3)c2)c2cccc(-n3c4ccc5ccccc5c4c4c5ccccc5ccc43)c2)cc1]

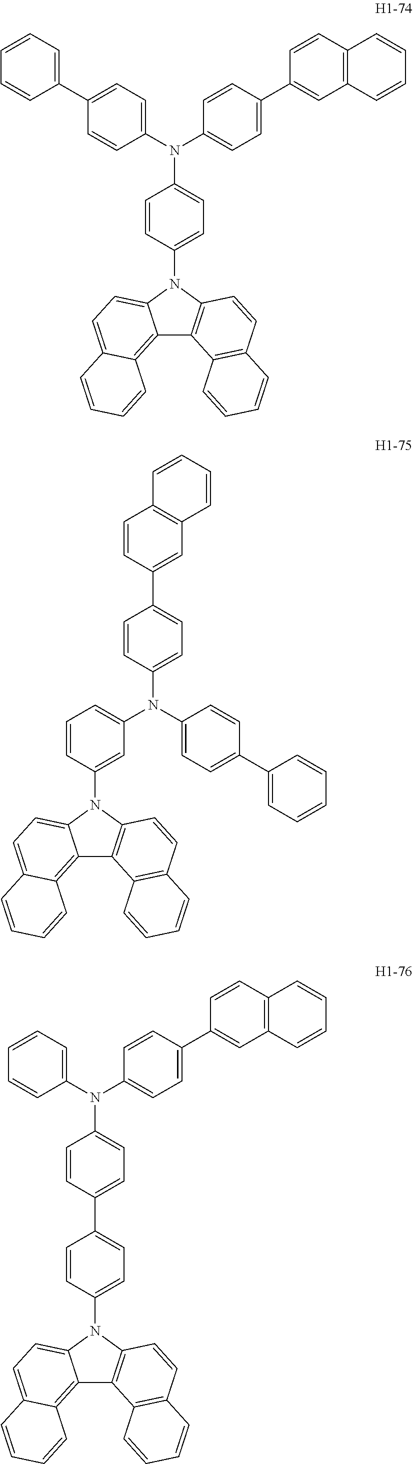 [SMILES: c1ccc(-c2ccc(N(c3ccc(-c4ccc5ccccc5c4)cc3)c3ccc(-n4c5ccc6ccccc6c5c5c6ccccc6ccc54)cc3)cc2)cc1 ; c1ccc(-c2ccc(N(c3ccc(-c4ccc5ccccc5c4)cc3)c3cccc(-n4c5ccc6ccccc6c5c5c6ccccc6ccc54)c3)cc2)cc1 ; c1ccc(N(c2ccc(-c3ccc(-n4c5ccc6ccccc6c5c5c6ccccc6ccc54)cc3)cc2)c2ccc(-c3ccc4ccccc4c3)cc2)cc1]

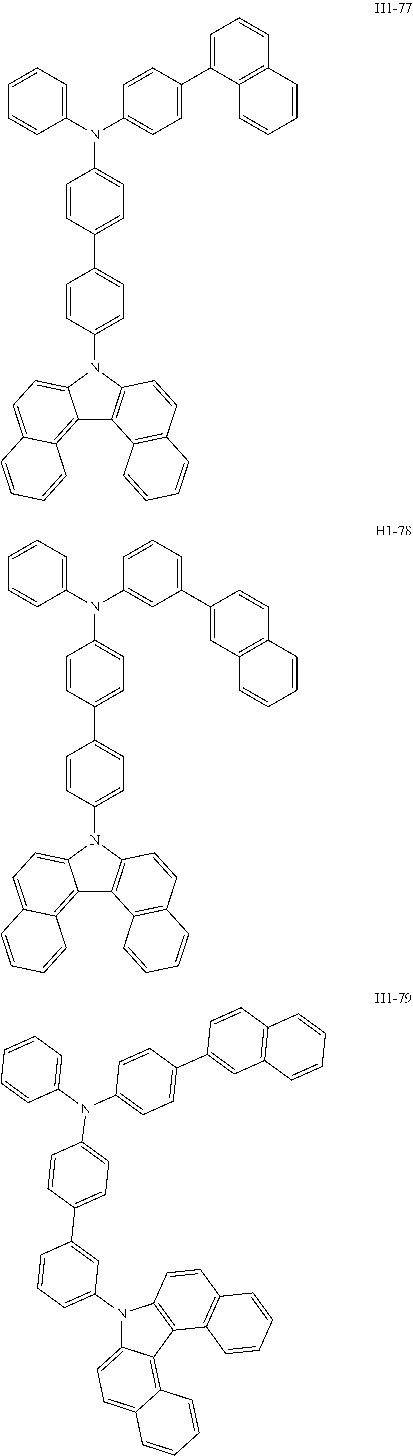

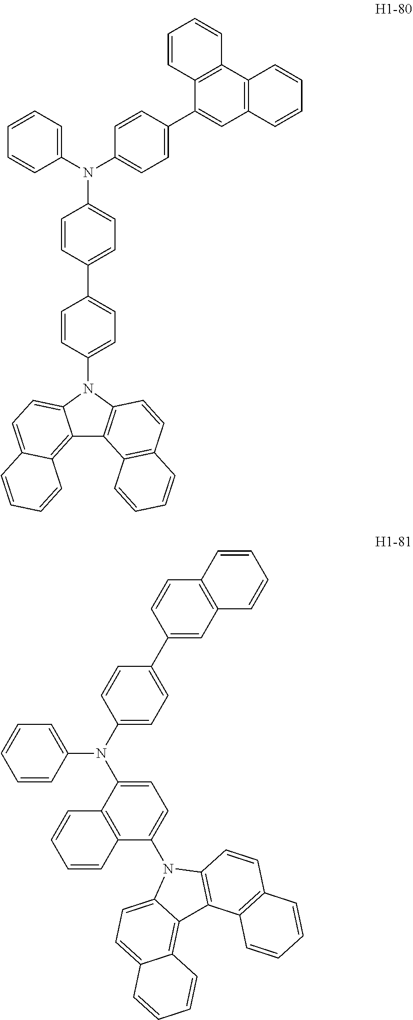 [SMILES: c1ccc(N(c2ccc(-c3ccc(-n4c5ccc6ccccc6c5c5c6ccccc6ccc54)cc3)cc2)c2ccc(-c3cc4ccccc4c4ccccc34)cc2)cc1 ; c1ccc(N(c2ccc(-c3ccc4ccccc4c3)cc2)c2ccc(-n3c4ccc5ccccc5c4c4c5ccccc5ccc43)c3ccccc23)cc1]

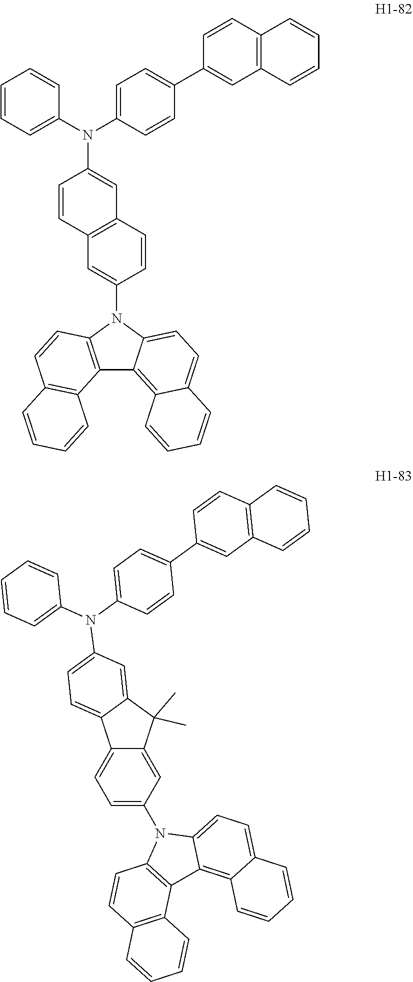 [SMILES: c1ccc(N(c2ccc(-c3ccc4ccccc4c3)cc2)c2ccc3cc(-n4c5ccc6ccccc6c5c5c6ccccc6ccc54)ccc3c2)cc1 ; CC1(C)c2cc(N(c3ccccc3)c3ccc(-c4ccc5ccccc5c4)cc3)ccc2-c2ccc(-n3c4ccc5ccccc5c4c4c5ccccc5ccc43)cc21]

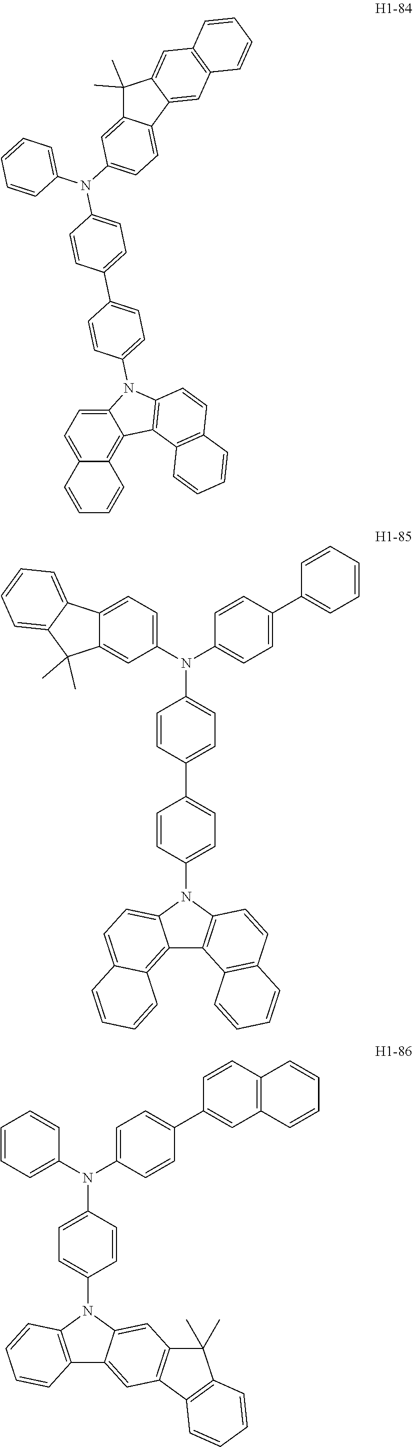

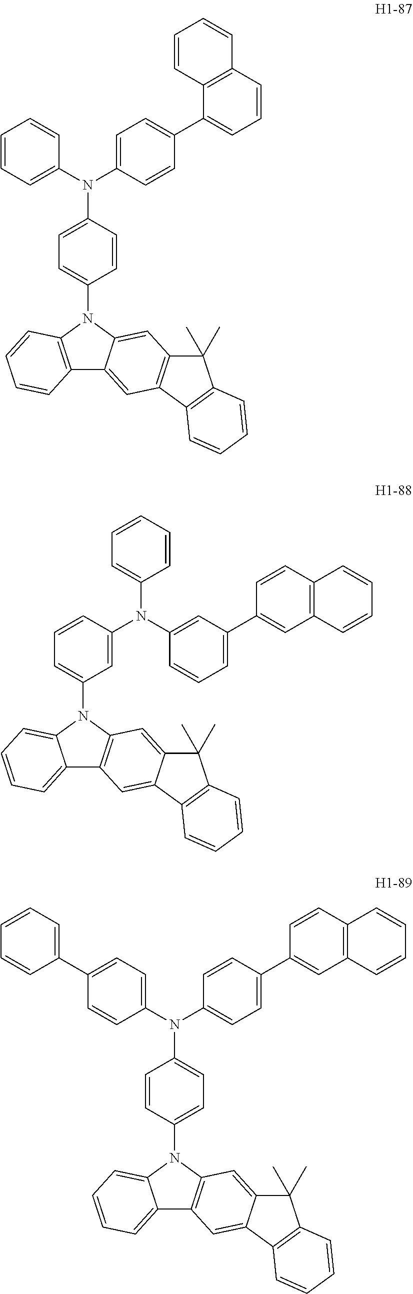 [SMILES: CC1(C)c2ccccc2-c2cc3c4ccccc4n(-c4ccc(N(c5ccccc5)c5ccc(-c6cccc7ccccc67)cc5)cc4)c3cc21 ; CC1(C)c2ccccc2-c2cc3c4ccccc4n(-c4cccc(N(c5ccccc5)c5cccc(-c6ccc7ccccc7c6)c5)c4)c3cc21 ; CC1(C)c2ccccc2-c2cc3c4ccccc4n(-c4ccc(N(c5ccc(-c6ccccc6)cc5)c5ccc(-c6ccc7ccccc7c6)cc5)cc4)c3cc21]

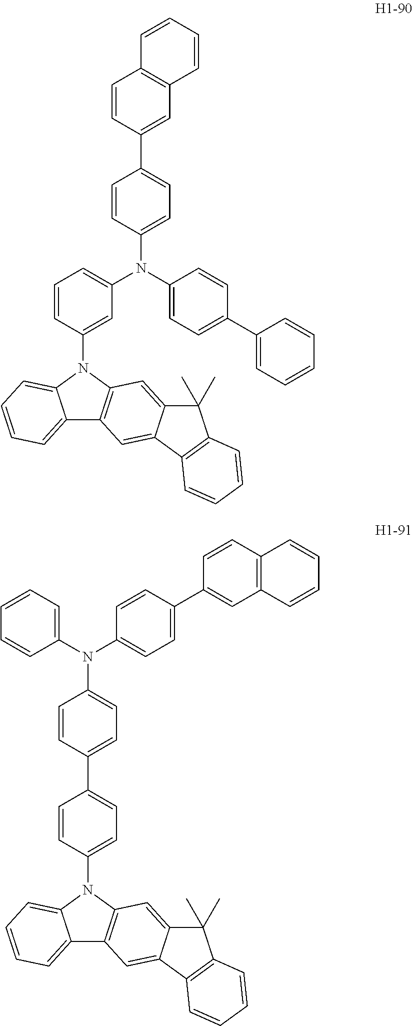

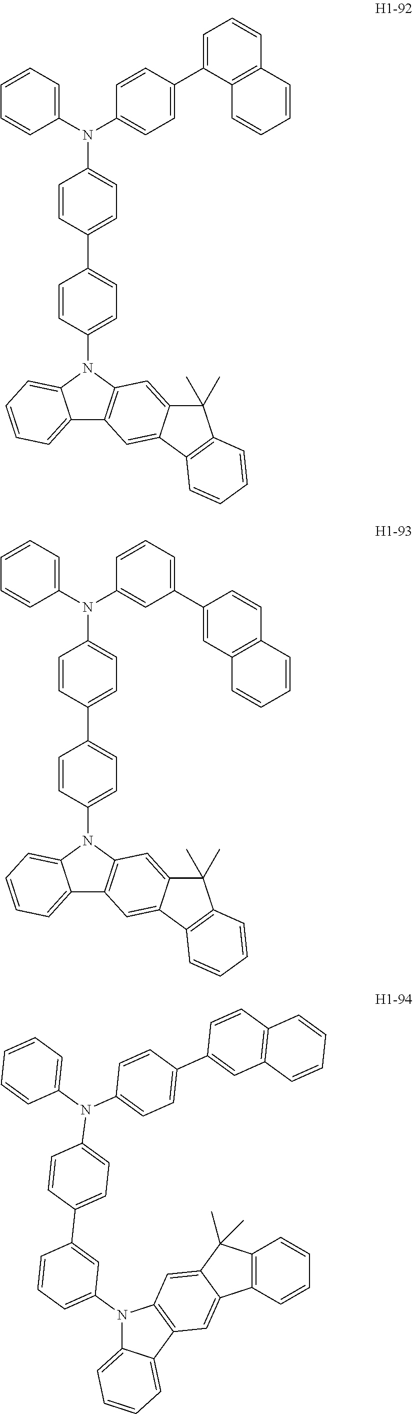

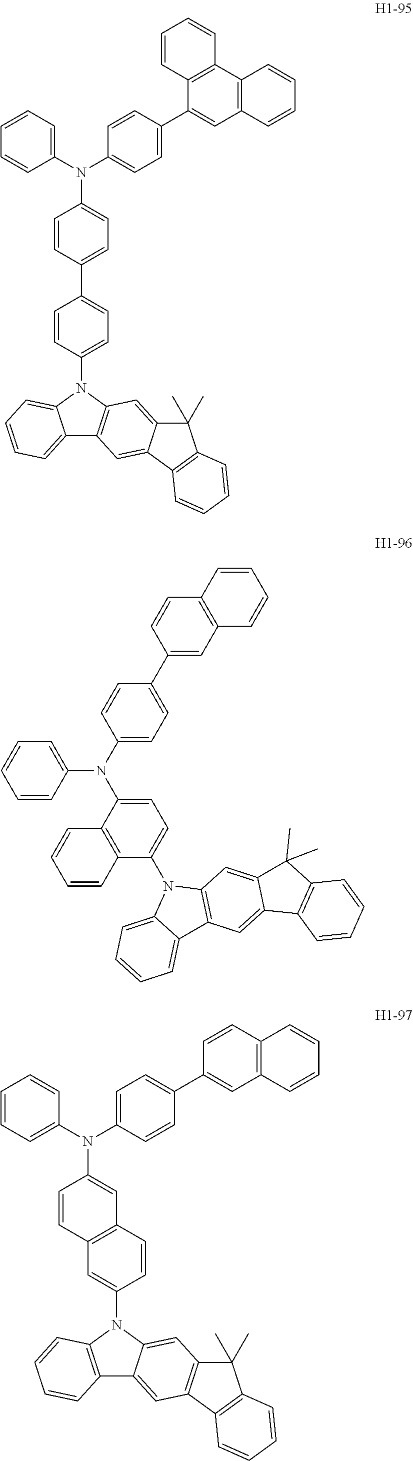 [SMILES: CC1(C)c2ccccc2-c2cc3c4ccccc4n(-c4ccc(-c5ccc(N(c6ccccc6)c6ccc(-c7cc8ccccc8c8ccccc78)cc6)cc5)cc4)c3cc21 ; CC1(C)c2ccccc2-c2cc3c4ccccc4n(-c4ccc(N(c5ccccc5)c5ccc(-c6ccc7ccccc7c6)cc5)c5ccccc45)c3cc21 ; CC1(C)c2ccccc2-c2cc3c4ccccc4n(-c4ccc5cc(N(c6ccccc6)c6ccc(-c7ccc8ccccc8c7)cc6)ccc5c4)c3cc21]

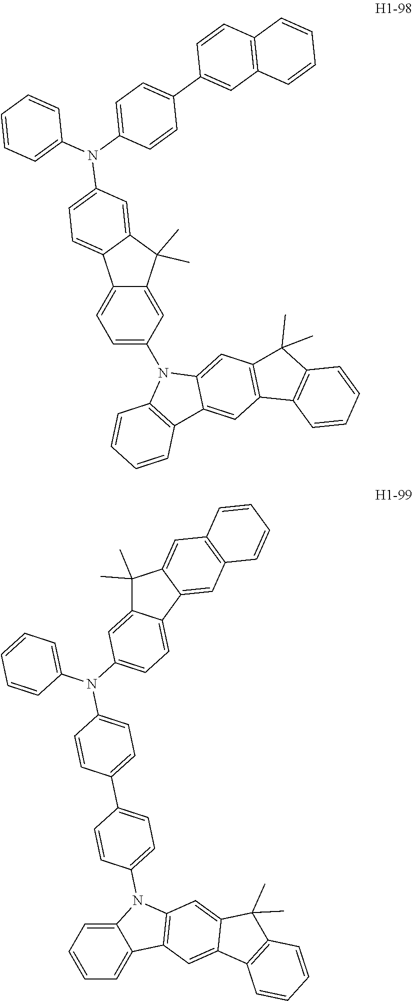 [SMILES: CC1(C)c2cc(N(c3ccccc3)c3ccc(-c4ccc5ccccc5c4)cc3)ccc2-c2ccc(-n3c4ccccc4c4cc5c(cc43)C(C)(C)c3ccccc3-5)cc21 ; CC1(C)c2cc(N(c3ccccc3)c3ccc(-c4ccc(-n5c6ccccc6c6cc7c(cc65)C(C)(C)c5ccccc5-7)cc4)cc3)ccc2-c2cc3ccccc3cc21]

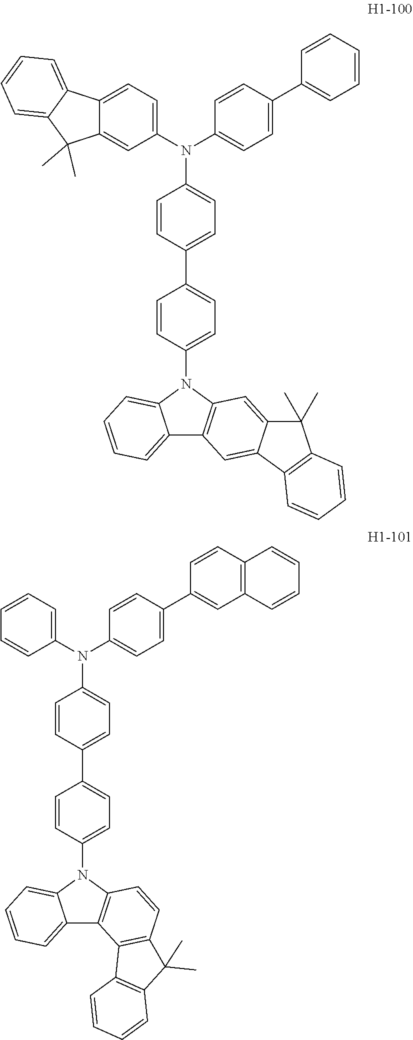

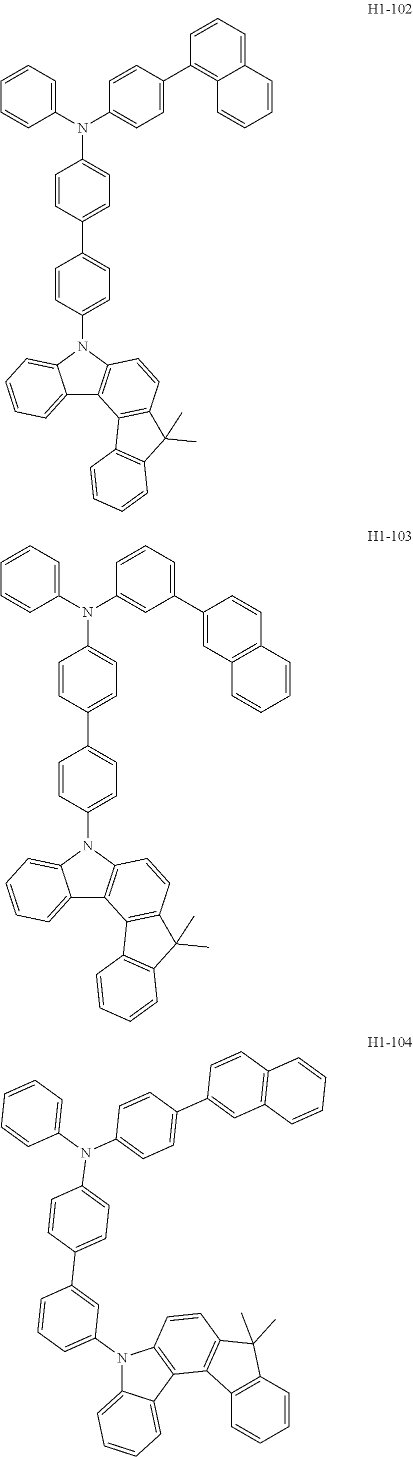

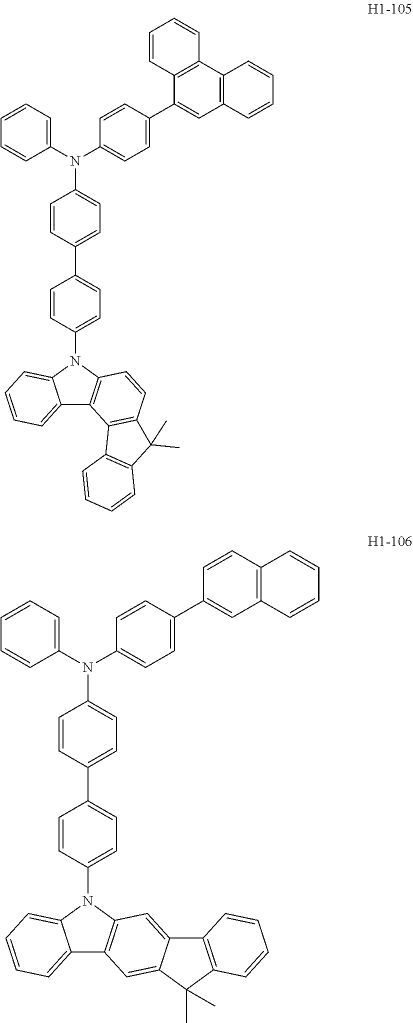 [SMILES: CC1(C)c2ccccc2-c2c1ccc1c2c2ccccc2n1-c1ccc(-c2ccc(N(c3ccccc3)c3ccc(-c4cc5ccccc5c5ccccc45)cc3)cc2)cc1 ; CC1(C)c2ccccc2-c2cc3c(cc21)c1ccccc1n3-c1ccc(-c2ccc(N(c3ccccc3)c3ccc(-c4ccc5ccccc5c4)cc3)cc2)cc1]

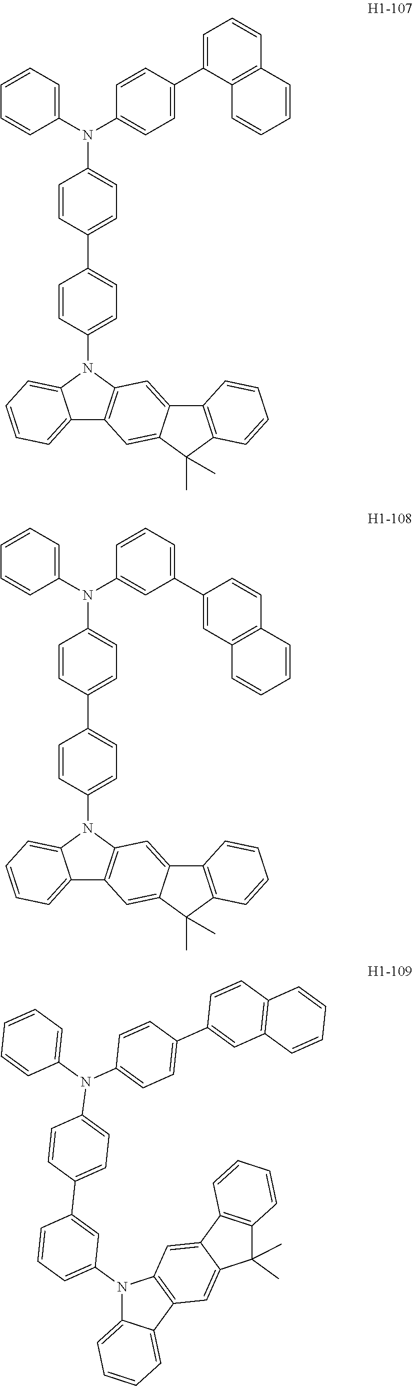 [SMILES: CC1(C)c2ccccc2-c2cc3c(cc21)c1ccccc1n3-c1ccc(-c2ccc(N(c3ccccc3)c3ccc(-c4cccc5ccccc45)cc3)cc2)cc1 ; CC1(C)c2ccccc2-c2cc3c(cc21)c1ccccc1n3-c1ccc(-c2ccc(N(c3ccccc3)c3cccc(-c4ccc5ccccc5c4)c3)cc2)cc1 ; CC1(C)c2ccccc2-c2cc3c(cc21)c1ccccc1n3-c1cccc(-c2ccc(N(c3ccccc3)c3ccc(-c4ccc5ccccc5c4)cc3)cc2)c1]

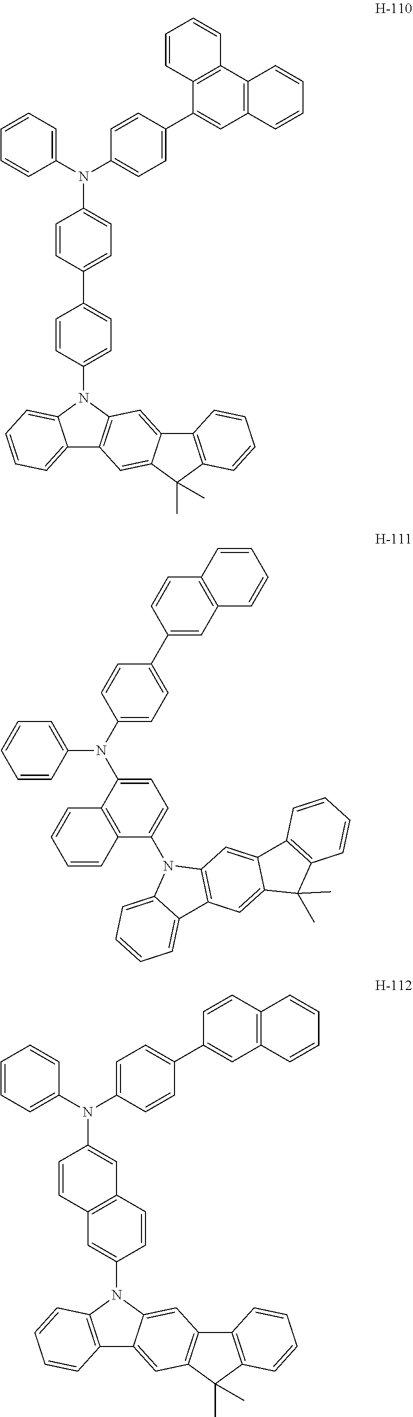 [SMILES: CC1(C)c2ccccc2-c2cc3c(cc21)c1ccccc1n3-c1ccc(-c2ccc(N(c3ccccc3)c3ccc(-c4cc5ccccc5c5ccccc45)cc3)cc2)cc1 ; CC1(C)c2ccccc2-c2cc3c(cc21)c1ccccc1n3-c1ccc(N(c2ccccc2)c2ccc(-c3ccc4ccccc4c3)cc2)c2ccccc12 ; CC1(C)c2ccccc2-c2cc3c(cc21)c1ccccc1n3-c1ccc2cc(N(c3ccccc3)c3ccc(-c4ccc5ccccc5c4)cc3)ccc2c1]

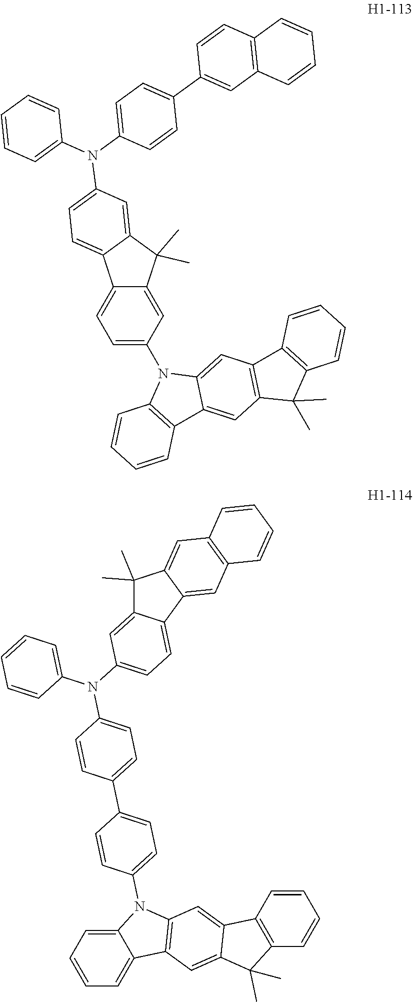 [SMILES: CC1(C)c2cc(N(c3ccccc3)c3ccc(-c4ccc5ccccc5c4)cc3)ccc2-c2ccc(-n3c4ccccc4c4cc5c(cc43)-c3ccccc3C5(C)C)cc21 ; CC1(C)c2cc(N(c3ccccc3)c3ccc(-c4ccc(-n5c6ccccc6c6cc7c(cc65)-c5ccccc5C7(C)C)cc4)cc3)ccc2-c2cc3ccccc3cc21]

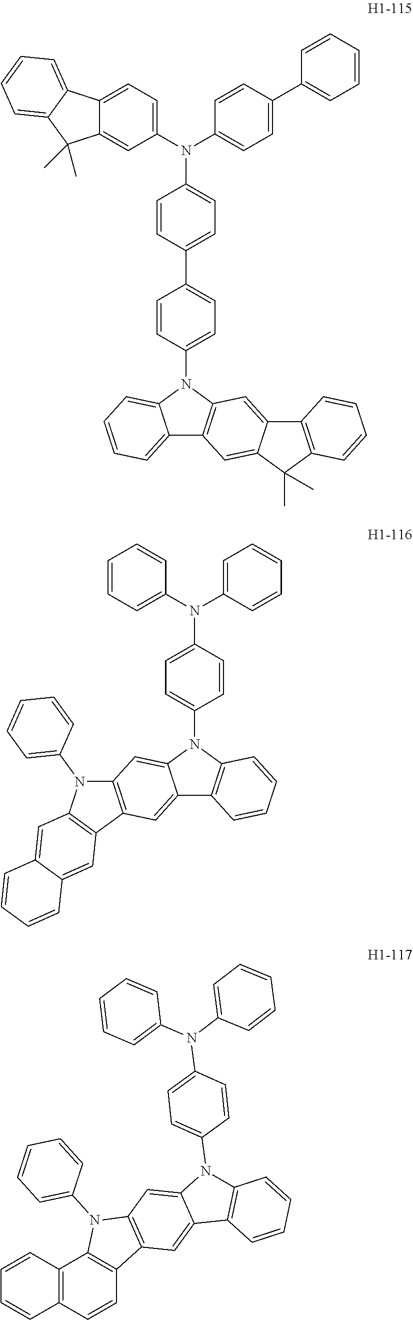 [SMILES: CC1(C)c2ccccc2-c2ccc(N(c3ccc(-c4ccccc4)cc3)c3ccc(-c4ccc(-n5c6ccccc6c6cc7c(cc65)-c5ccccc5C7(C)C)cc4)cc3)cc21 ; c1ccc(N(c2ccccc2)c2ccc(-n3c4ccccc4c4cc5c6cc7ccccc7cc6n(-c6ccccc6)c5cc43)cc2)cc1 ; c1ccc(N(c2ccccc2)c2ccc(-n3c4ccccc4c4cc5c6ccc7ccccc7c6n(-c6ccccc6)c5cc43)cc2)cc1]

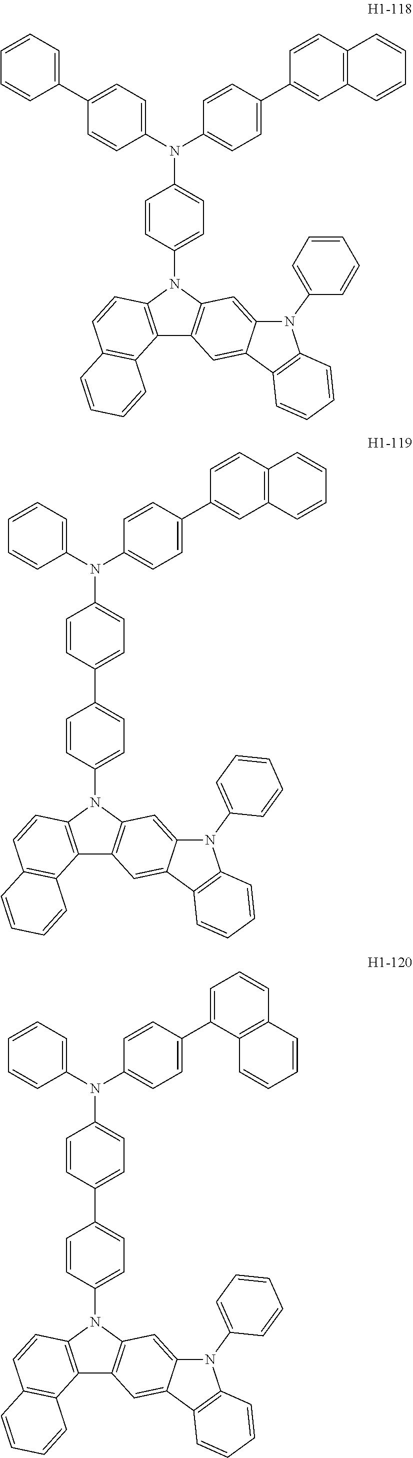

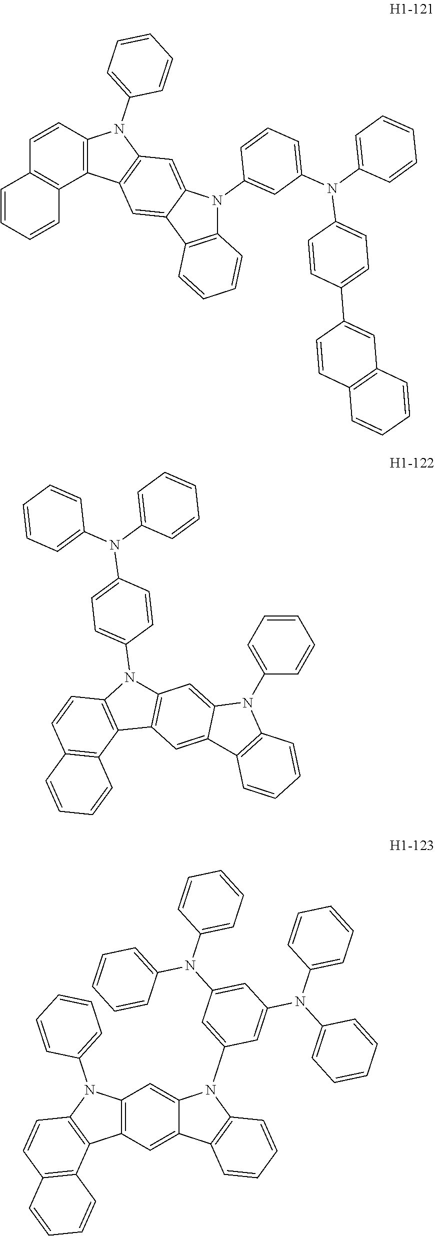 [SMILES: c1ccc(N(c2ccc(-c3ccc4ccccc4c3)cc2)c2cccc(-n3c4ccccc4c4cc5c6c7ccccc7ccc6n(-c6ccccc6)c5cc43)c2)cc1 ; c1ccc(N(c2ccccc2)c2ccc(-n3c4cc5c(cc4c4c6ccccc6ccc43)c3ccccc3n5-c3ccccc3)cc2)cc1 ; c1ccc(N(c2ccccc2)c2cc(N(c3ccccc3)c3ccccc3)cc(-n3c4ccccc4c4cc5c6c7ccccc7ccc6n(-c6ccccc6)c5cc43)c2)cc1]

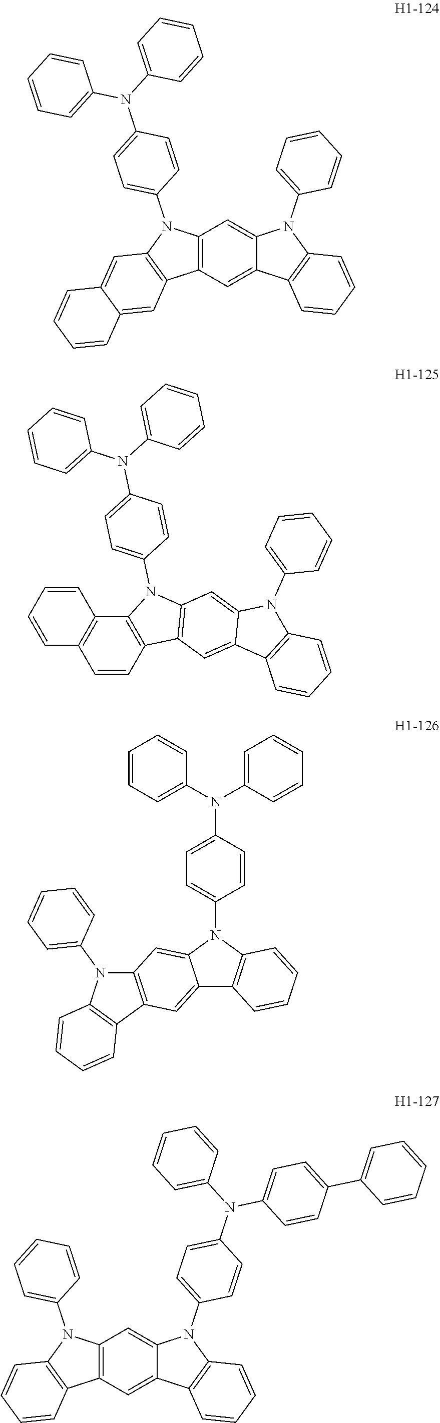

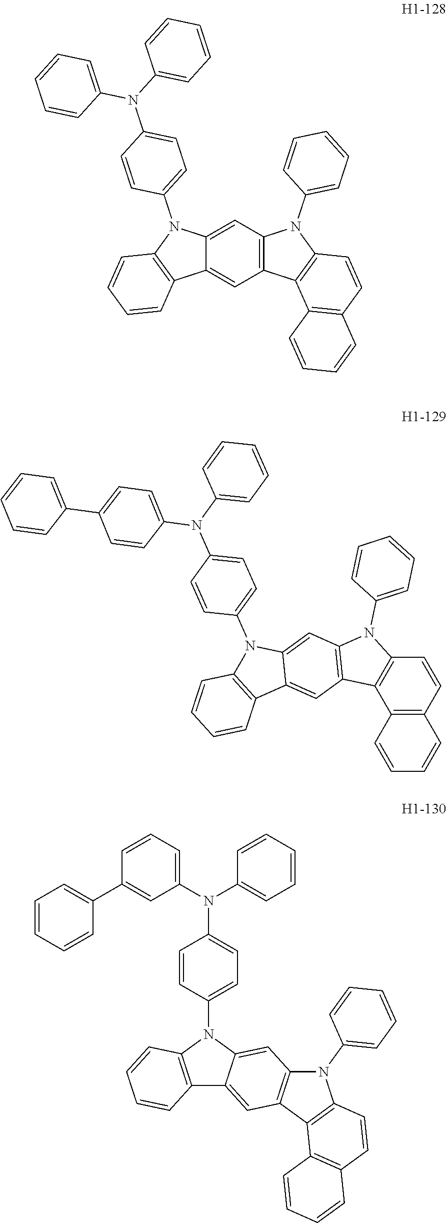 [SMILES: c1ccc(N(c2ccccc2)c2ccc(-n3c4ccccc4c4cc5c6c7ccccc7ccc6n(-c6ccccc6)c5cc43)cc2)cc1 ; c1ccc(-c2ccc(N(c3ccccc3)c3ccc(-n4c5ccccc5c5cc6c7c8ccccc8ccc7n(-c7ccccc7)c6cc54)cc3)cc2)cc1 ; c1ccc(-c2cccc(N(c3ccccc3)c3ccc(-n4c5ccccc5c5cc6c7c8ccccc8ccc7n(-c7ccccc7)c6cc54)cc3)c2)cc1]

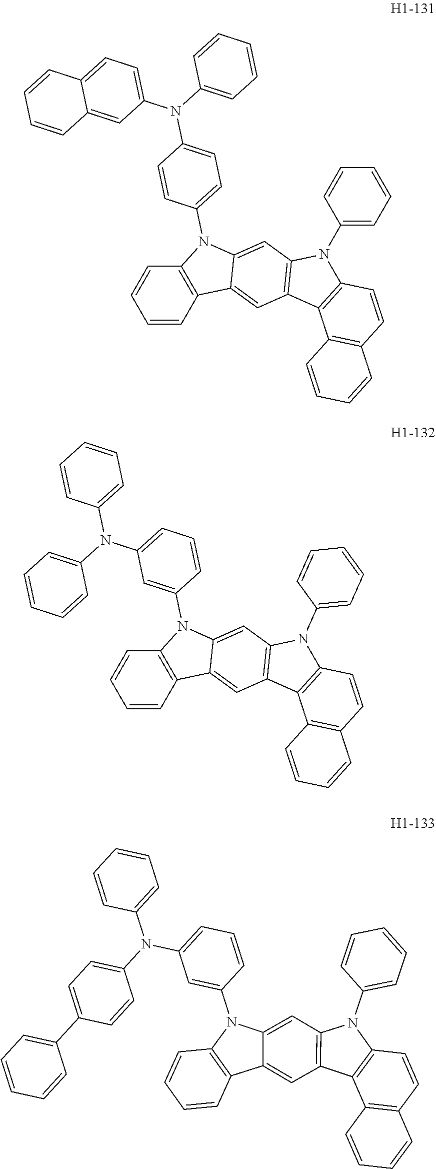 [SMILES: c1ccc(N(c2ccc(-n3c4ccccc4c4cc5c6c7ccccc7ccc6n(-c6ccccc6)c5cc43)cc2)c2ccc3ccccc3c2)cc1 ; c1ccc(N(c2ccccc2)c2cccc(-n3c4ccccc4c4cc5c6c7ccccc7ccc6n(-c6ccccc6)c5cc43)c2)cc1 ; c1ccc(-c2ccc(N(c3ccccc3)c3cccc(-n4c5ccccc5c5cc6c7c8ccccc8ccc7n(-c7ccccc7)c6cc54)c3)cc2)cc1]

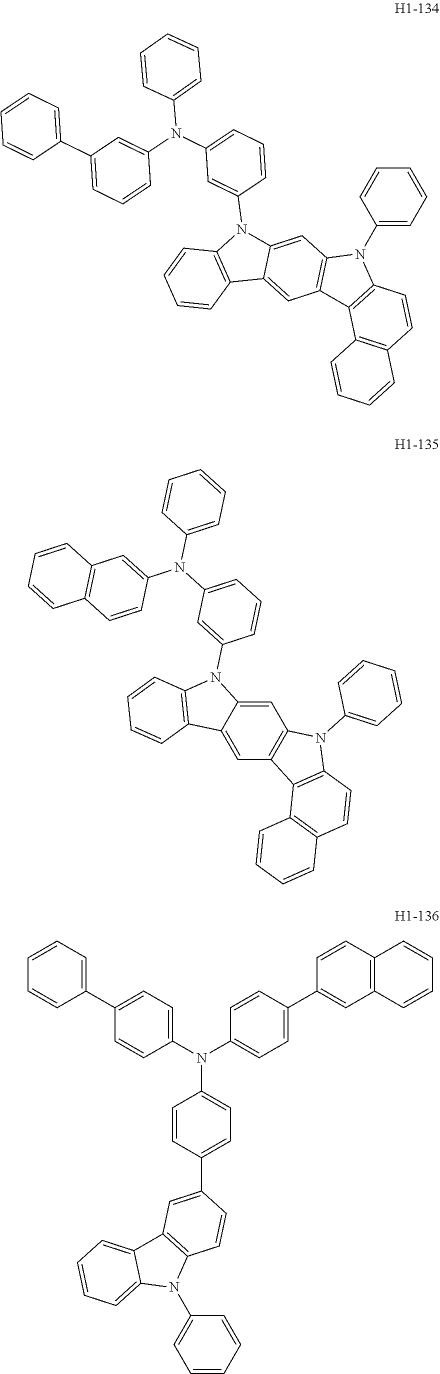 [SMILES: c1ccc(-c2cccc(N(c3ccccc3)c3cccc(-n4c5ccccc5c5cc6c7c8ccccc8ccc7n(-c7ccccc7)c6cc54)c3)c2)cc1 ; c1ccc(N(c2cccc(-n3c4ccccc4c4cc5c6c7ccccc7ccc6n(-c6ccccc6)c5cc43)c2)c2ccc3ccccc3c2)cc1 ; c1ccc(-c2ccc(N(c3ccc(-c4ccc5ccccc5c4)cc3)c3ccc(-c4ccc5c(c4)c4ccccc4n5-c4ccccc4)cc3)cc2)cc1]

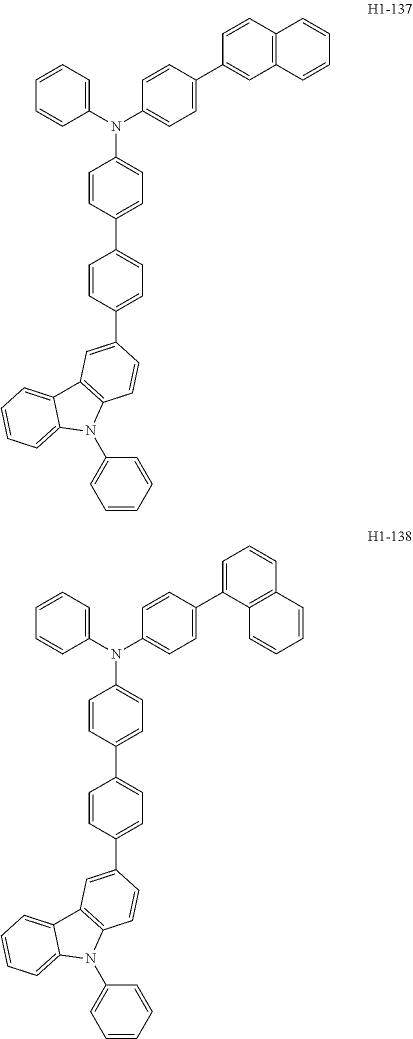 [SMILES: c1ccc(N(c2ccc(-c3ccc(-c4ccc5c(c4)c4ccccc4n5-c4ccccc4)cc3)cc2)c2ccc(-c3ccc4ccccc4c3)cc2)cc1 ; c1ccc(N(c2ccc(-c3ccc(-c4ccc5c(c4)c4ccccc4n5-c4ccccc4)cc3)cc2)c2ccc(-c3cccc4ccccc34)cc2)cc1]

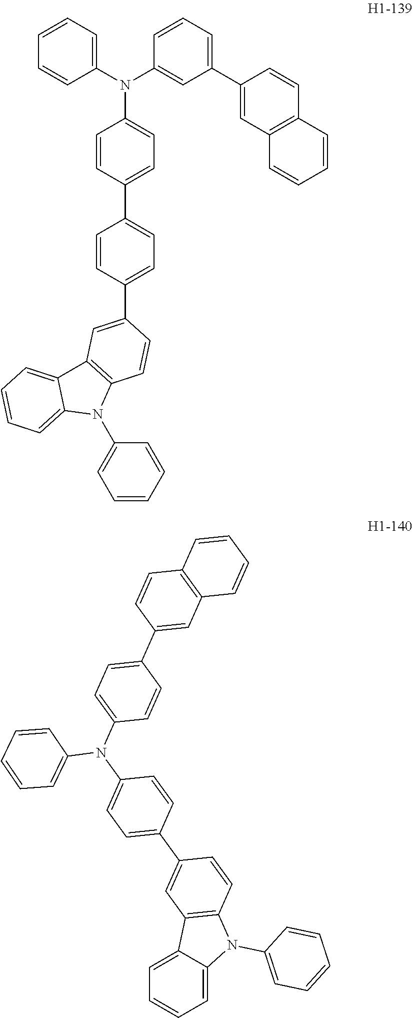 [SMILES: c1ccc(N(c2ccc(-c3ccc(-c4ccc5c(c4)c4ccccc4n5-c4ccccc4)cc3)cc2)c2cccc(-c3ccc4ccccc4c3)c2)cc1 ; c1ccc(N(c2ccc(-c3ccc4ccccc4c3)cc2)c2ccc(-c3ccc4c(c3)c3ccccc3n4-c3ccccc3)cc2)cc1]

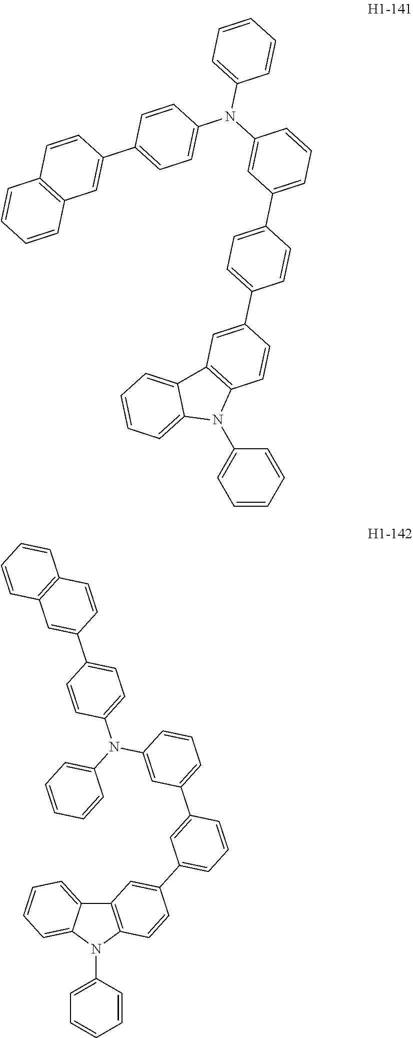 [SMILES: c1ccc(N(c2ccc(-c3ccc4ccccc4c3)cc2)c2cccc(-c3ccc(-c4ccc5c(c4)c4ccccc4n5-c4ccccc4)cc3)c2)cc1 ; c1ccc(N(c2ccc(-c3ccc4ccccc4c3)cc2)c2cccc(-c3cccc(-c4ccc5c(c4)c4ccccc4n5-c4ccccc4)c3)c2)cc1]

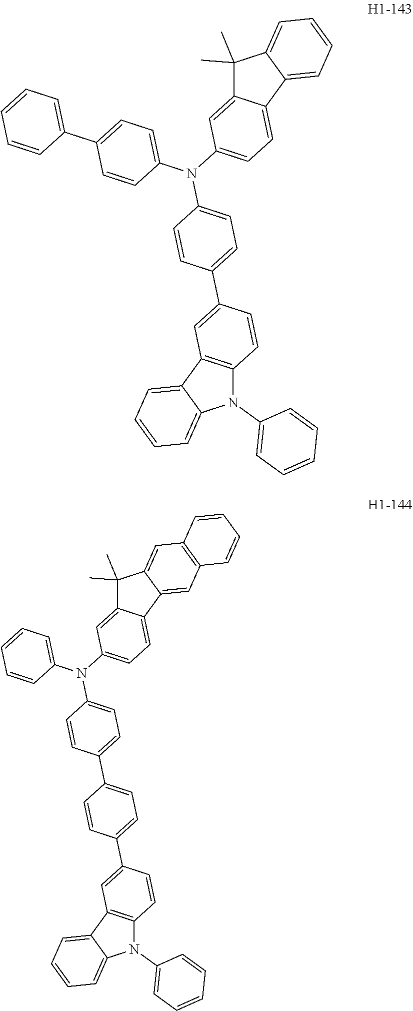 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2cc(N(c3ccccc3)c3ccc(-c4ccc(-c5ccc6c(c5)c5ccccc5n6-c5ccccc5)cc4)cc3)ccc2-c2cc3ccccc3cc21]

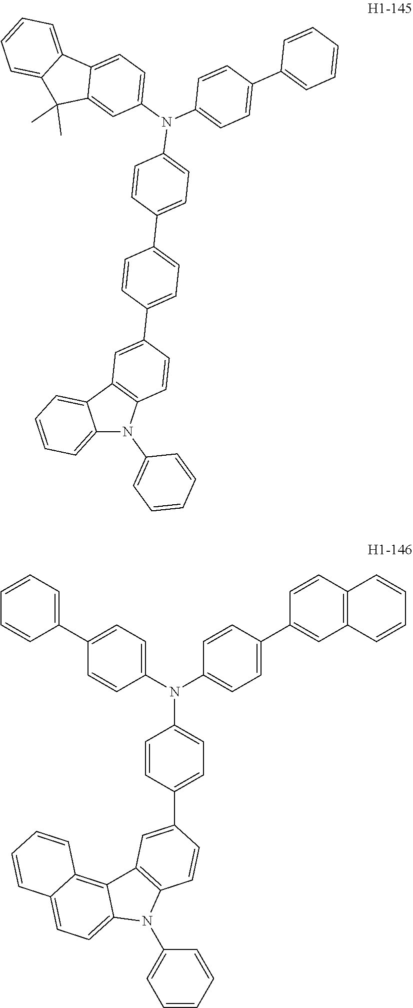 [SMILES: CC1(C)c2ccccc2-c2ccc(N(c3ccc(-c4ccccc4)cc3)c3ccc(-c4ccc(-c5ccc6c(c5)c5ccccc5n6-c5ccccc5)cc4)cc3)cc21 ; c1ccc(-c2ccc(N(c3ccc(-c4ccc5ccccc5c4)cc3)c3ccc(-c4ccc5c(c4)c4c6ccccc6ccc4n5-c4ccccc4)cc3)cc2)cc1]

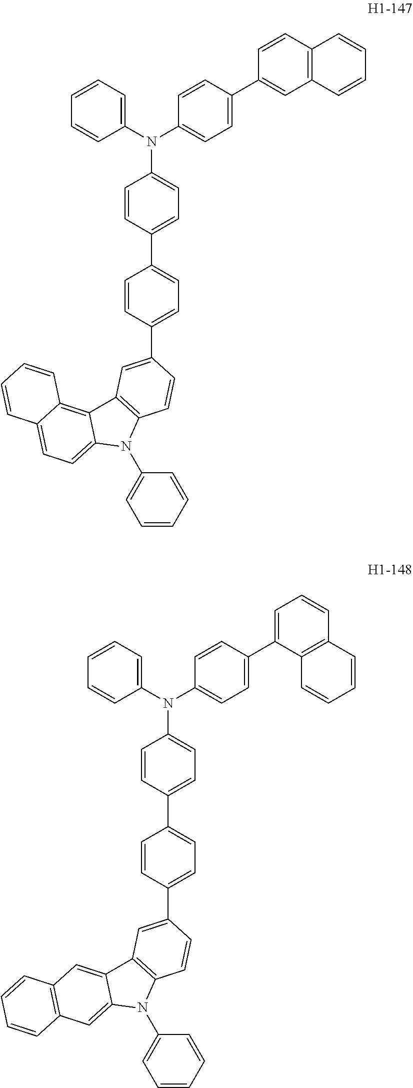 [SMILES: c1ccc(N(c2ccc(-c3ccc(-c4ccc5c(c4)c4c6ccccc6ccc4n5-c4ccccc4)cc3)cc2)c2ccc(-c3ccc4ccccc4c3)cc2)cc1 ; c1ccc(N(c2ccc(-c3ccc(-c4ccc5c(c4)c4cc6ccccc6cc4n5-c4ccccc4)cc3)cc2)c2ccc(-c3cccc4ccccc34)cc2)cc1]

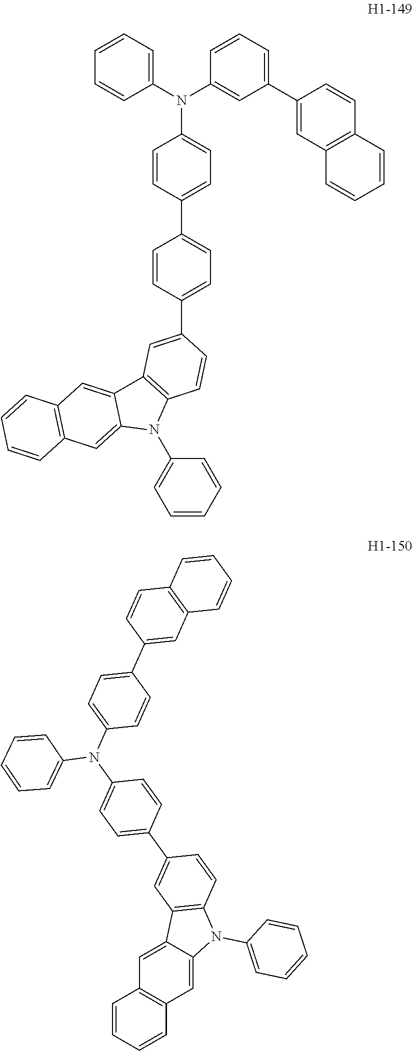

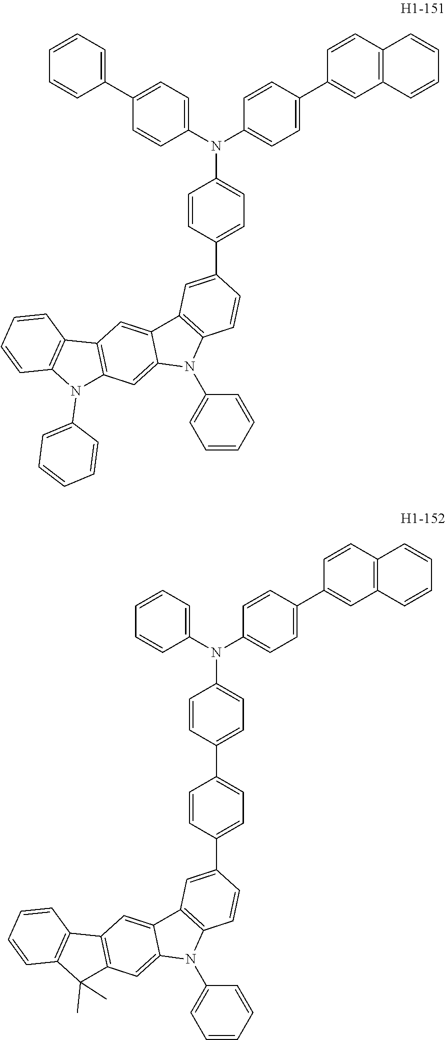 [SMILES: c1ccc(-c2ccc(N(c3ccc(-c4ccc5ccccc5c4)cc3)c3ccc(-c4ccc5c(c4)c4cc6c7ccccc7n(-c7ccccc7)c6cc4n5-c4ccccc4)cc3)cc2)cc1 ; CC1(C)c2ccccc2-c2cc3c4cc(-c5ccc(-c6ccc(N(c7ccccc7)c7ccc(-c8ccc9ccccc9c8)cc7)cc6)cc5)ccc4n(-c4ccccc4)c3cc21]

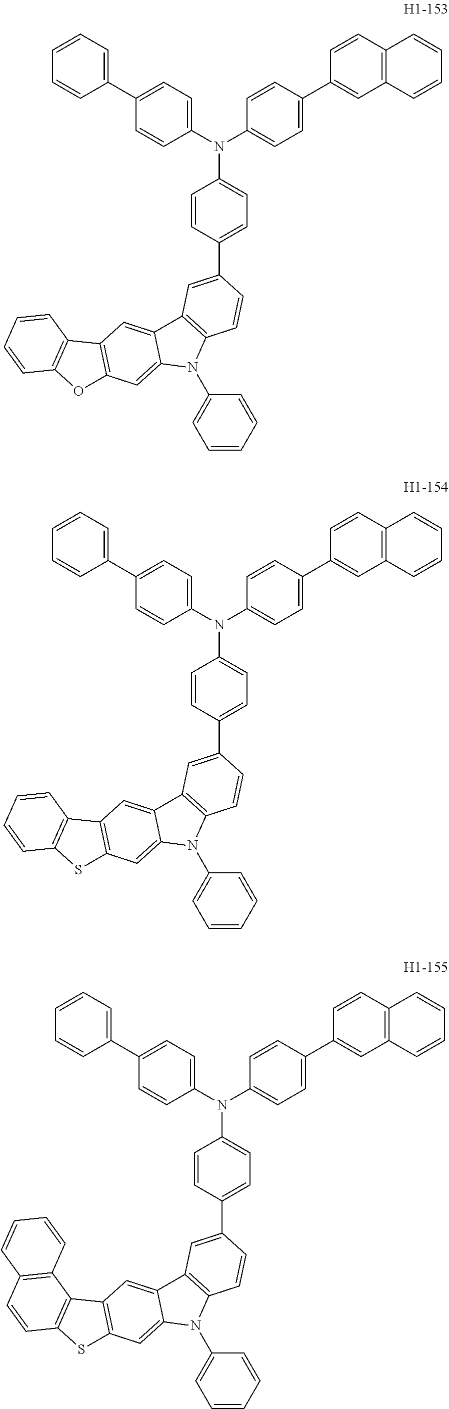 [SMILES: c1ccc(-c2ccc(N(c3ccc(-c4ccc5ccccc5c4)cc3)c3ccc(-c4ccc5c(c4)c4cc6c(cc4n5-c4ccccc4)oc4ccccc46)cc3)cc2)cc1 ; c1ccc(-c2ccc(N(c3ccc(-c4ccc5ccccc5c4)cc3)c3ccc(-c4ccc5c(c4)c4cc6c(cc4n5-c4ccccc4)sc4ccccc46)cc3)cc2)cc1 ; c1ccc(-c2ccc(N(c3ccc(-c4ccc5ccccc5c4)cc3)c3ccc(-c4ccc5c(c4)c4cc6c(cc4n5-c4ccccc4)sc4ccc5ccccc5c46)cc3)cc2)cc1]

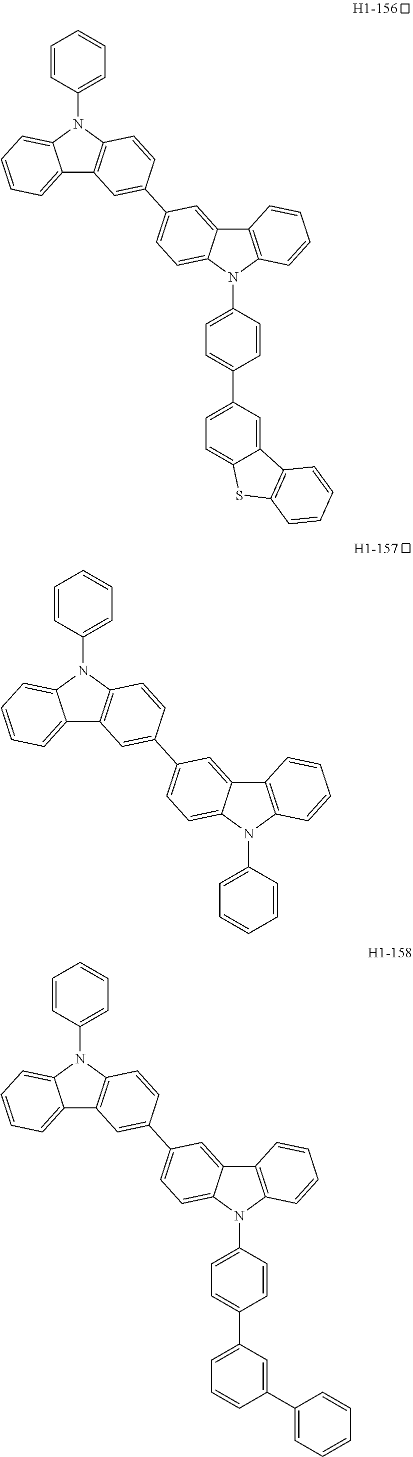 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccccc4)ccc32)cc1 ; c1ccc(-c2cccc(-c3ccc(-n4c5ccccc5c5cc(-c6ccc7c(c6)c6ccccc6n7-c6ccccc6)ccc54)cc3)c2)cc1]

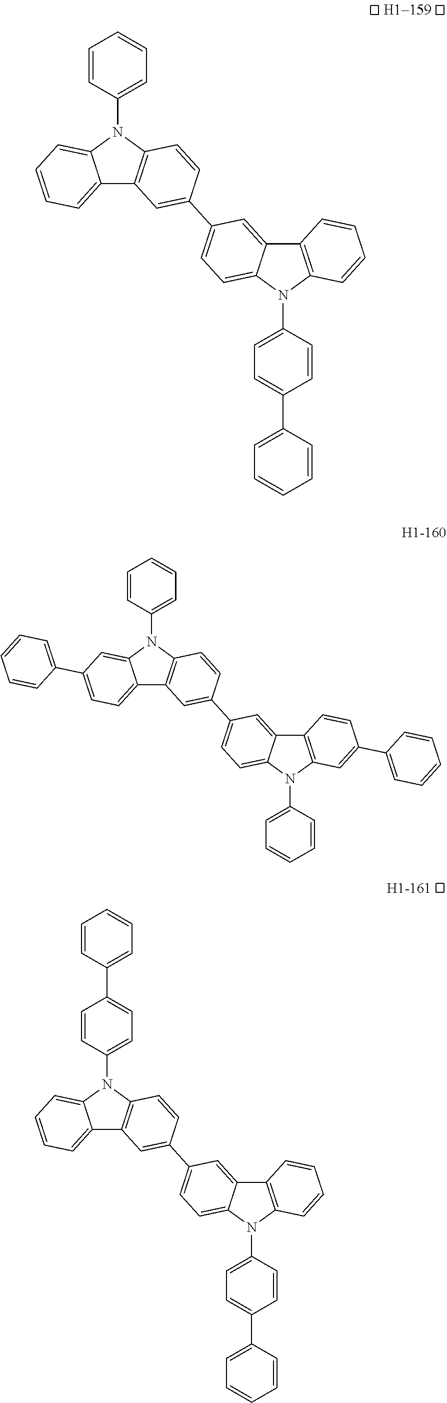 [SMILES: c1ccc(-c2ccc(-n3c4ccccc4c4cc(-c5ccc6c(c5)c5ccccc5n6-c5ccccc5)ccc43)cc2)cc1 ; c1ccc(-c2ccc3c4cc(-c5ccc6c(c5)c5ccc(-c7ccccc7)cc5n6-c5ccccc5)ccc4n(-c4ccccc4)c3c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

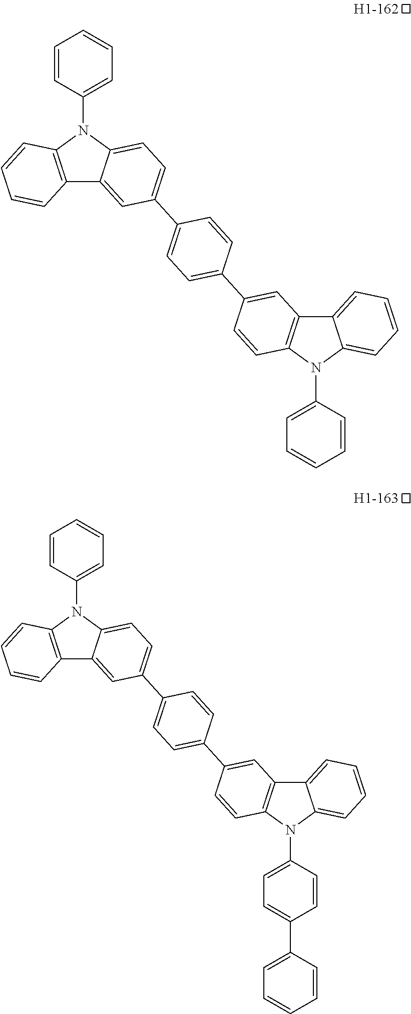 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

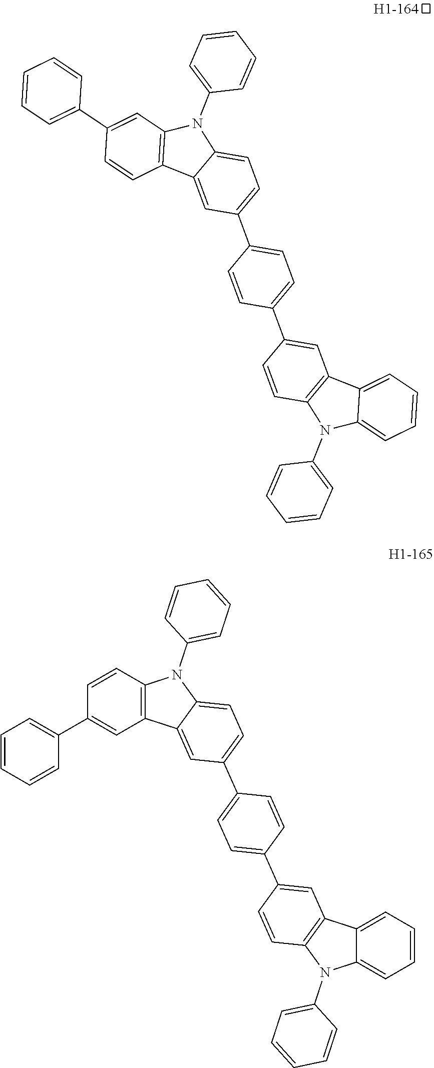 [SMILES: c1ccc(-c2ccc3c4cc(-c5ccc(-c6ccc7c(c6)c6ccccc6n7-c6ccccc6)cc5)ccc4n(-c4ccccc4)c3c2)cc1 ; c1ccc(-c2ccc3c(c2)c2cc(-c4ccc(-c5ccc6c(c5)c5ccccc5n6-c5ccccc5)cc4)ccc2n3-c2ccccc2)cc1]

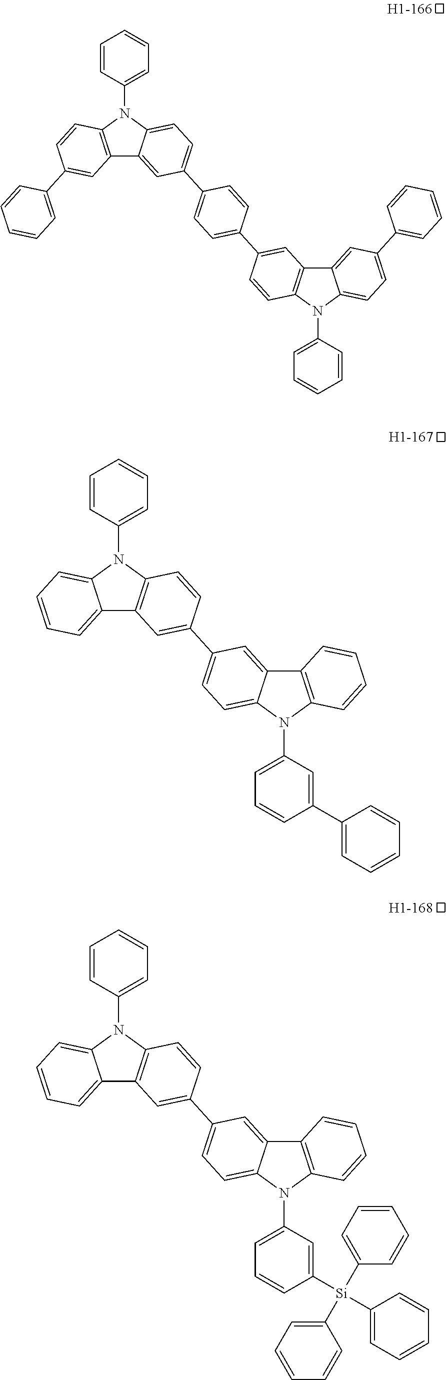 [SMILES: c1ccc(-c2ccc3c(c2)c2cc(-c4ccc(-c5ccc6c(c5)c5cc(-c7ccccc7)ccc5n6-c5ccccc5)cc4)ccc2n3-c2ccccc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

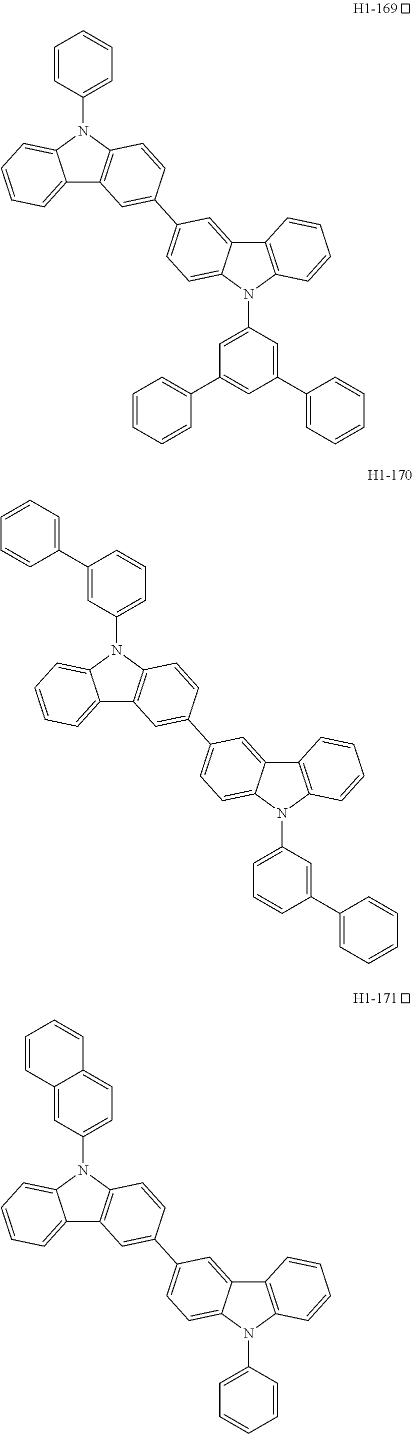 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c(c4)c4ccccc4n5-c4ccc5ccccc5c4)ccc32)cc1]

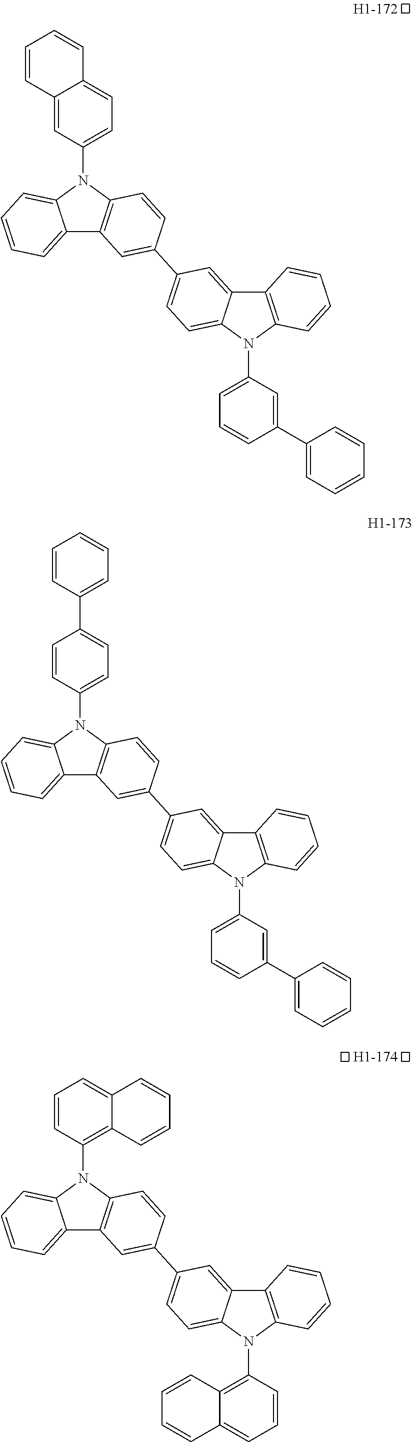 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-n3c4ccccc4c4cc(-c5ccc6c(c5)c5ccccc5n6-c5cccc6ccccc56)ccc43)cccc2c1]

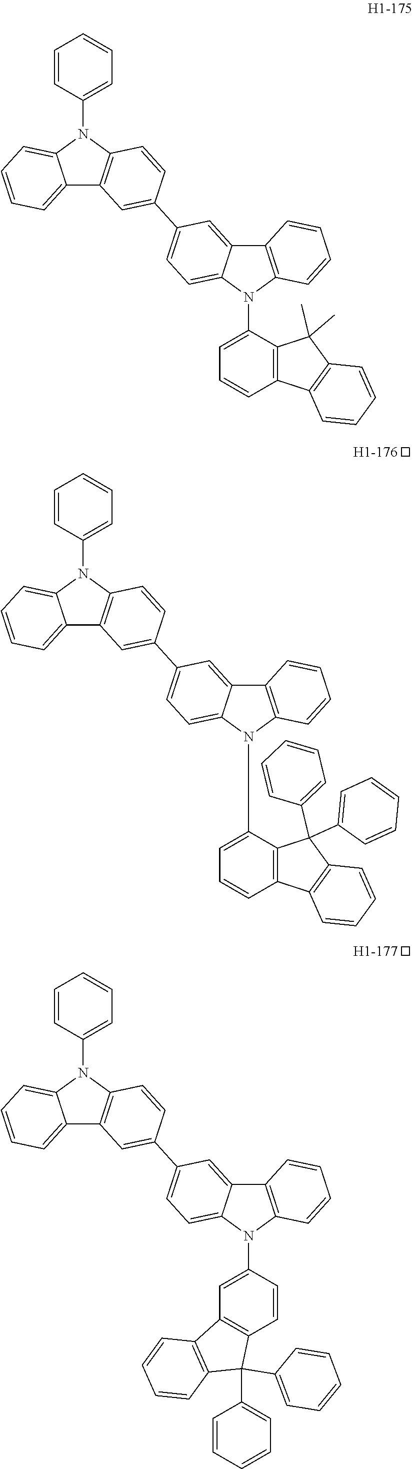 [SMILES: CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1]

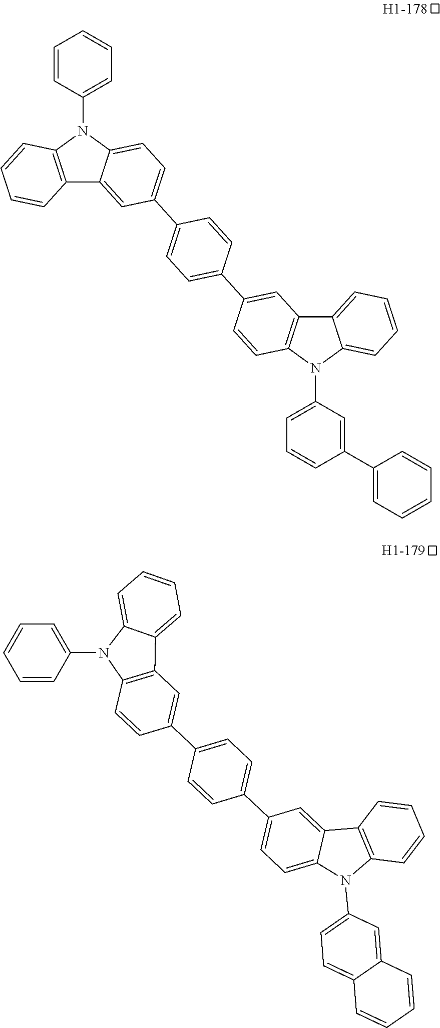 [SMILES: c1ccc(-c2cccc(-n3c4ccccc4c4cc(-c5ccc(-c6ccc7c(c6)c6ccccc6n7-c6ccccc6)cc5)ccc43)c2)cc1 ; c1ccc(-n2c3ccccc3c3cc(-c4ccc(-c5ccc6c(c5)c5ccccc5n6-c5ccc6ccccc6c5)cc4)ccc32)cc1]

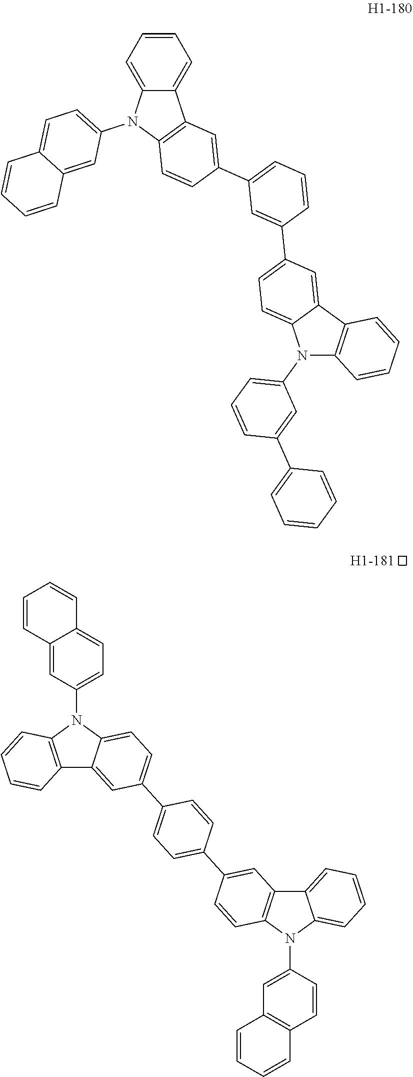 [SMILES: c1ccc(-c2cccc(-n3c4ccccc4c4cc(-c5cccc(-c6ccc7c(c6)c6ccccc6n7-c6ccc7ccccc7c6)c5)ccc43)c2)cc1 ; c1ccc2cc(-n3c4ccccc4c4cc(-c5ccc(-c6ccc7c(c6)c6ccccc6n7-c6ccc7ccccc7c6)cc5)ccc43)ccc2c1]

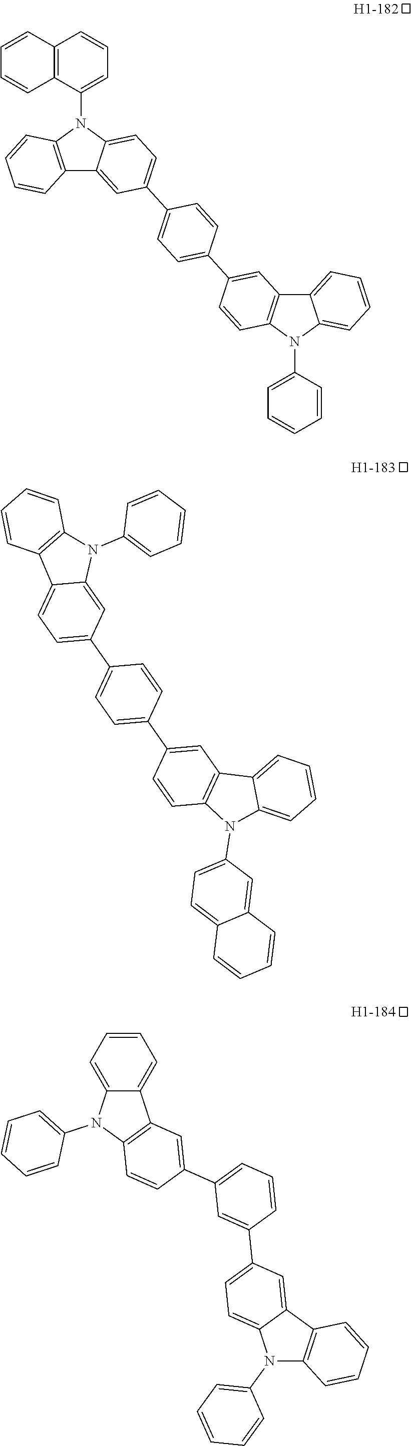 [SMILES: c1ccc(-n2c3ccccc3c3cc(-c4ccc(-c5ccc6c(c5)c5ccccc5n6-c5cccc6ccccc56)cc4)ccc32)cc1 ; c1ccc(-n2c3ccccc3c3ccc(-c4ccc(-c5ccc6c(c5)c5ccccc5n6-c5ccc6ccccc6c5)cc4)cc32)cc1 ; c1ccc(-n2c3ccccc3c3cc(-c4cccc(-c5ccc6c(c5)c5ccccc5n6-c5ccccc5)c4)ccc32)cc1]

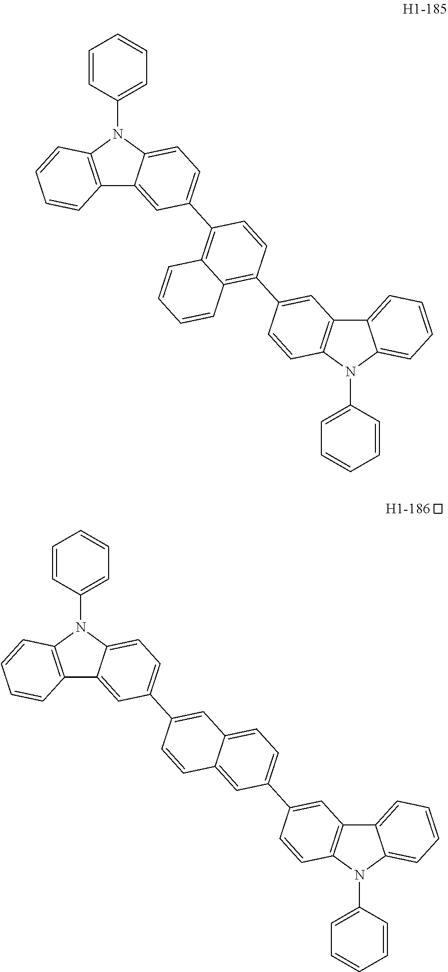 [SMILES: c1ccc(-n2c3ccccc3c3cc(-c4ccc(-c5ccc6c(c5)c5ccccc5n6-c5ccccc5)c5ccccc45)ccc32)cc1 ; c1ccc(-n2c3ccccc3c3cc(-c4ccc5cc(-c6ccc7c(c6)c6ccccc6n7-c6ccccc6)ccc5c4)ccc32)cc1]

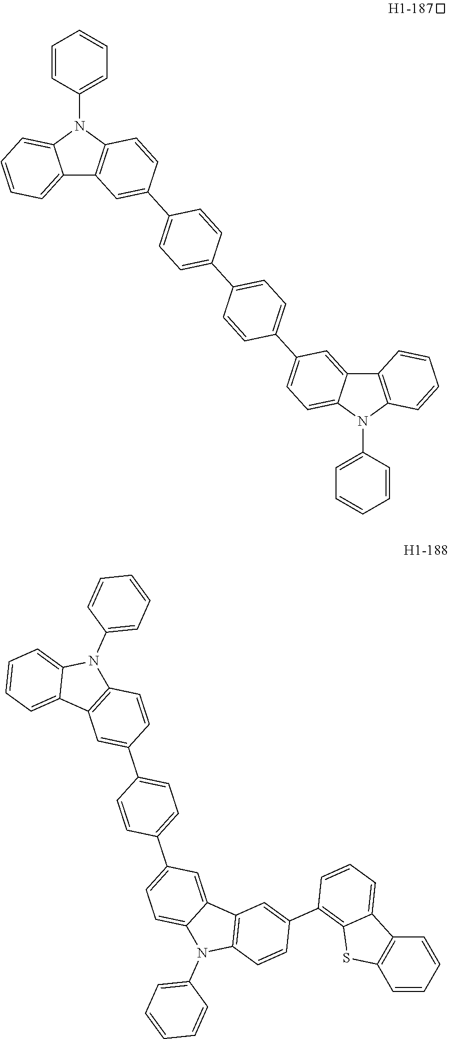 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5cc(-c7cccc8c7sc7ccccc78)ccc5n6-c5ccccc5)cc4)ccc32)cc1]

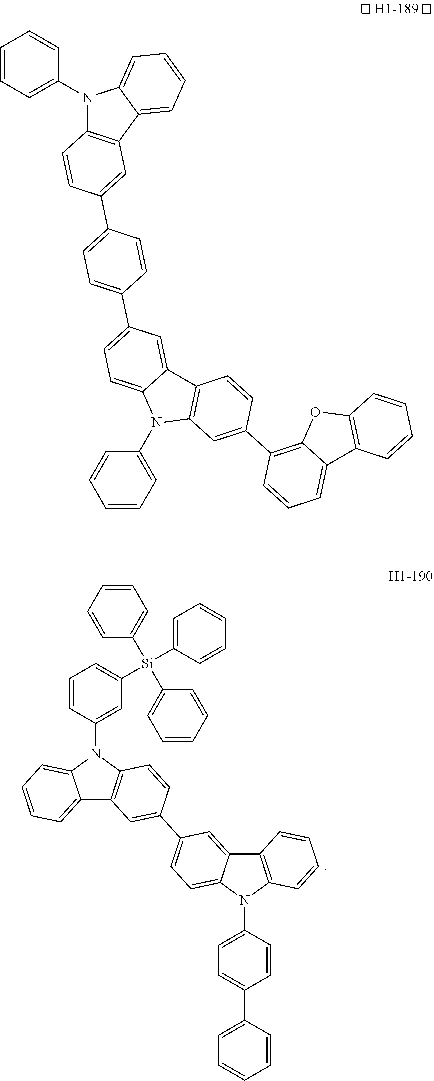 [SMILES: c1ccc(-n2c3ccccc3c3cc(-c4ccc(-c5ccc6c(c5)c5ccc(-c7cccc8c7oc7ccccc78)cc5n6-c5ccccc5)cc4)ccc32)cc1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1]

6. The plurality of host materials according to claim 1, wherein the compound represented by formula 2 is selected from the following compounds:

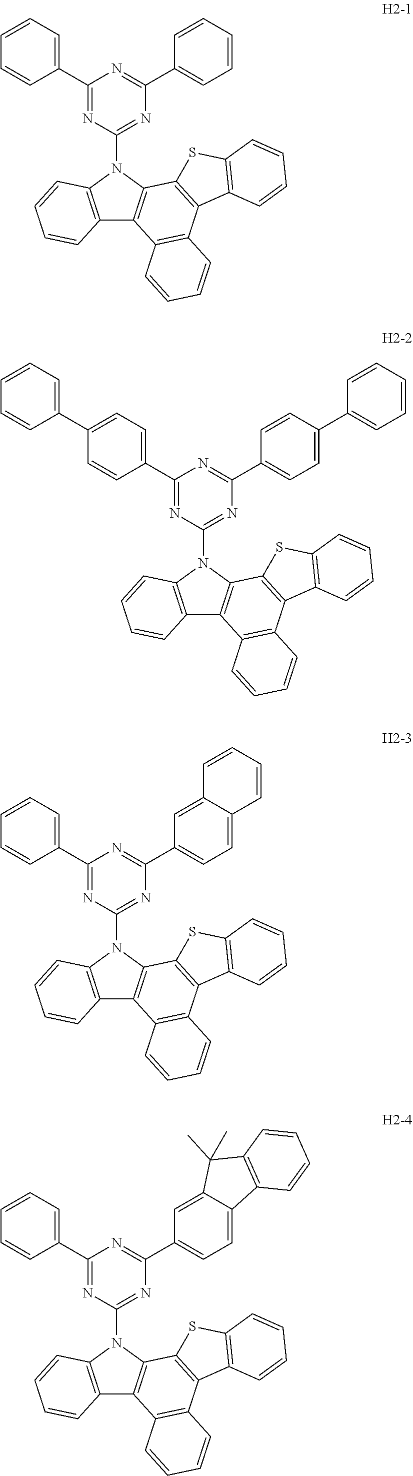 [SMILES: c1ccc(-c2nc(-c3ccccc3)nc(-n3c4ccccc4c4c5ccccc5c5c6ccccc6sc5c43)n2)cc1 ; c1ccc(-c2ccc(-c3nc(-c4ccc(-c5ccccc5)cc4)nc(-n4c5ccccc5c5c6ccccc6c6c7ccccc7sc6c54)n3)cc2)cc1 ; c1ccc(-c2nc(-c3ccc4ccccc4c3)nc(-n3c4ccccc4c4c5ccccc5c5c6ccccc6sc5c43)n2)cc1 ; CC1(C)c2ccccc2-c2ccc(-c3nc(-c4ccccc4)nc(-n4c5ccccc5c5c6ccccc6c6c7ccccc7sc6c54)n3)cc21]

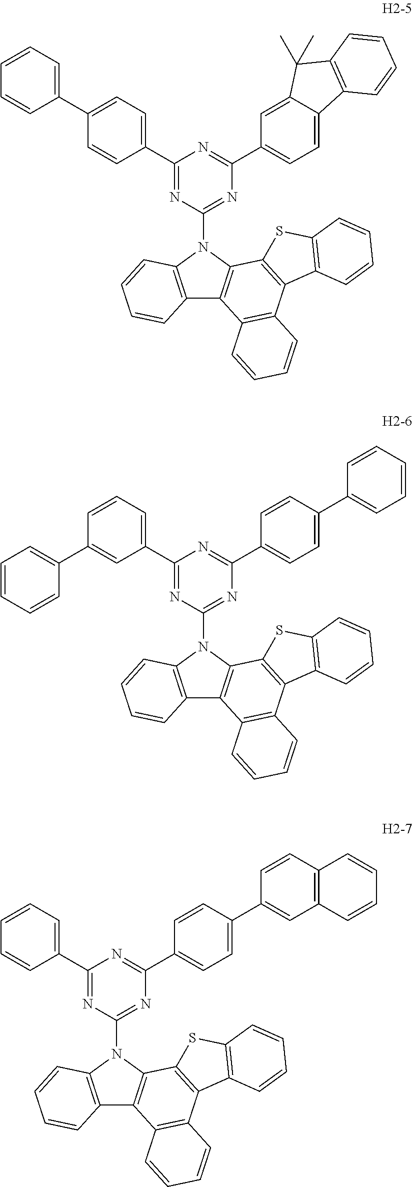 [SMILES: CC1(C)c2ccccc2-c2ccc(-c3nc(-c4ccc(-c5ccccc5)cc4)nc(-n4c5ccccc5c5c6ccccc6c6c7ccccc7sc6c54)n3)cc21 ; c1ccc(-c2ccc(-c3nc(-c4cccc(-c5ccccc5)c4)nc(-n4c5ccccc5c5c6ccccc6c6c7ccccc7sc6c54)n3)cc2)cc1 ; c1ccc(-c2nc(-c3ccc(-c4ccc5ccccc5c4)cc3)nc(-n3c4ccccc4c4c5ccccc5c5c6ccccc6sc5c43)n2)cc1]

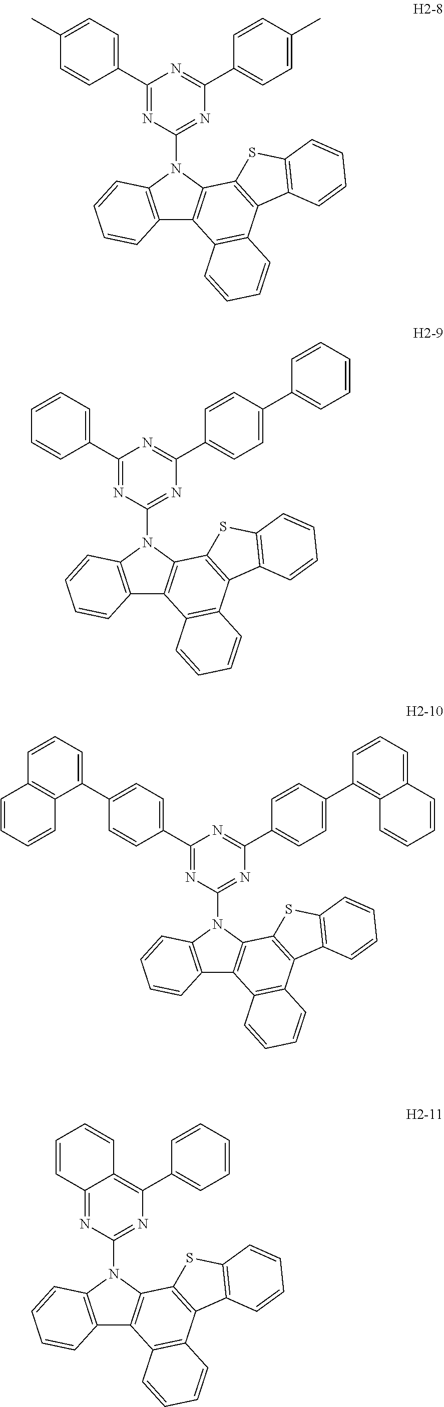 [SMILES: Cc1ccc(-c2nc(-c3ccc(C)cc3)nc(-n3c4ccccc4c4c5ccccc5c5c6ccccc6sc5c43)n2)cc1 ; c1ccc(-c2ccc(-c3nc(-c4ccccc4)nc(-n4c5ccccc5c5c6ccccc6c6c7ccccc7sc6c54)n3)cc2)cc1 ; c1ccc2c(-c3ccc(-c4nc(-c5ccc(-c6cccc7ccccc67)cc5)nc(-n5c6ccccc6c6c7ccccc7c7c8ccccc8sc7c65)n4)cc3)cccc2c1 ; c1ccc(-c2nc(-n3c4ccccc4c4c5ccccc5c5c6ccccc6sc5c43)nc3ccccc23)cc1]

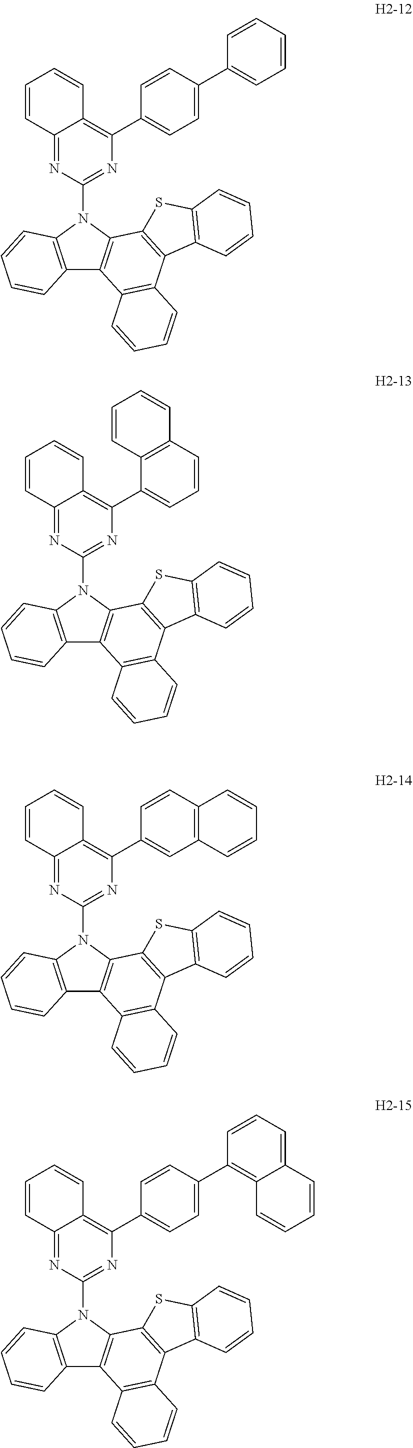

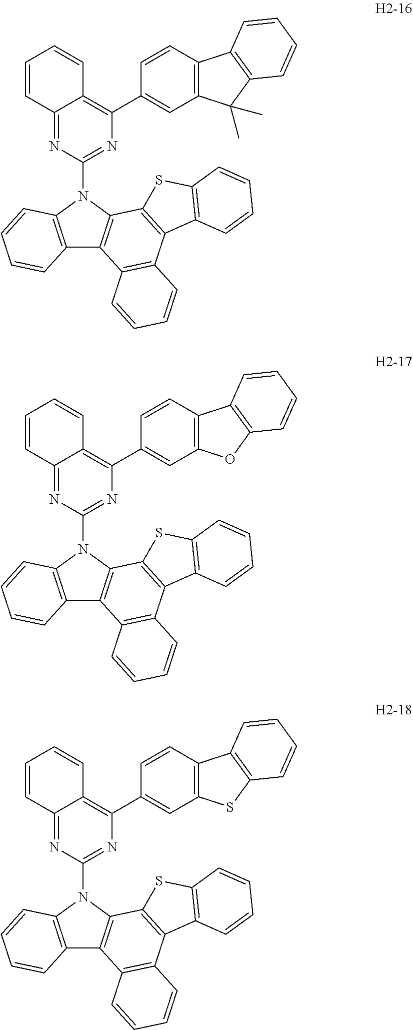 [SMILES: CC1(C)c2ccccc2-c2ccc(-c3nc(-n4c5ccccc5c5c6ccccc6c6c7ccccc7sc6c54)nc4ccccc34)cc21 ; c1ccc2c(-c3ccc4c(c3)oc3ccccc34)nc(-n3c4ccccc4c4c5ccccc5c5c6ccccc6sc5c43)nc2c1 ; c1ccc2c(-c3ccc4c(c3)sc3ccccc34)nc(-n3c4ccccc4c4c5ccccc5c5c6ccccc6sc5c43)nc2c1]

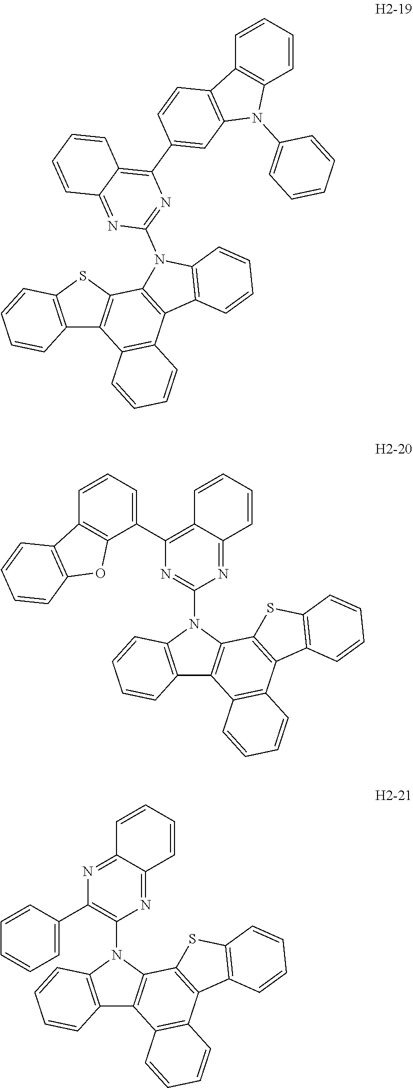 [SMILES: c1ccc(-n2c3ccccc3c3ccc(-c4nc(-n5c6ccccc6c6c7ccccc7c7c8ccccc8sc7c65)nc5ccccc45)cc32)cc1 ; c1ccc2c(-c3cccc4c3oc3ccccc34)nc(-n3c4ccccc4c4c5ccccc5c5c6ccccc6sc5c43)nc2c1 ; c1ccc(-c2nc3ccccc3nc2-n2c3ccccc3c3c4ccccc4c4c5ccccc5sc4c32)cc1]

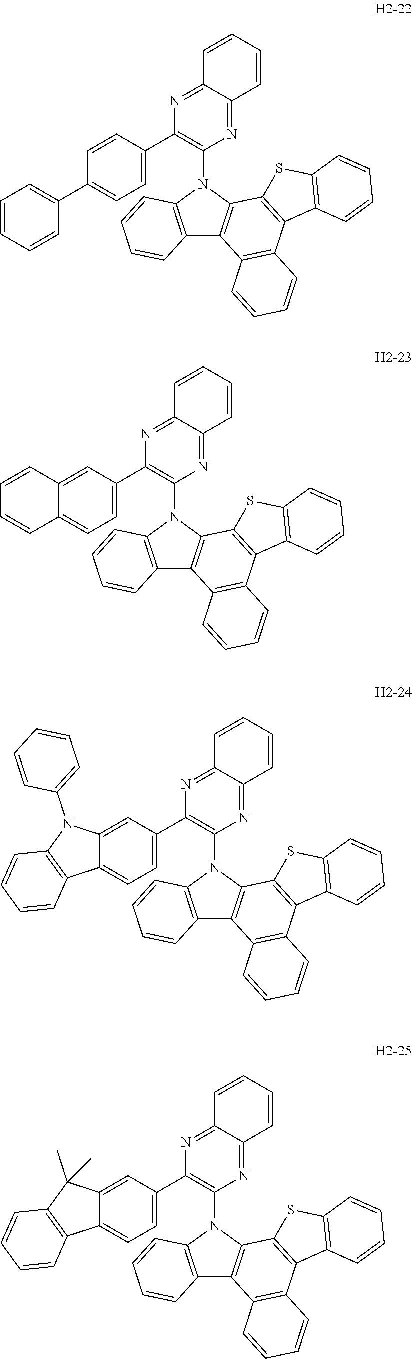 [SMILES: c1ccc(-c2ccc(-c3nc4ccccc4nc3-n3c4ccccc4c4c5ccccc5c5c6ccccc6sc5c43)cc2)cc1 ; c1ccc2cc(-c3nc4ccccc4nc3-n3c4ccccc4c4c5ccccc5c5c6ccccc6sc5c43)ccc2c1 ; c1ccc(-n2c3ccccc3c3ccc(-c4nc5ccccc5nc4-n4c5ccccc5c5c6ccccc6c6c7ccccc7sc6c54)cc32)cc1 ; CC1(C)c2ccccc2-c2ccc(-c3nc4ccccc4nc3-n3c4ccccc4c4c5ccccc5c5c6ccccc6sc5c43)cc21]

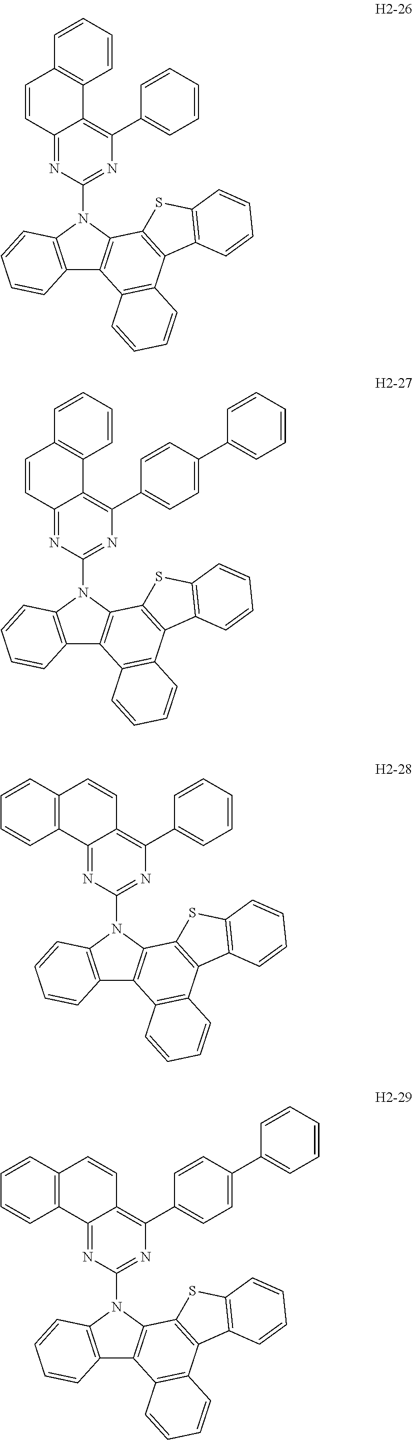 [SMILES: c1ccc(-c2nc(-n3c4ccccc4c4c5ccccc5c5c6ccccc6sc5c43)nc3ccc4ccccc4c23)cc1 ; c1ccc(-c2ccc(-c3nc(-n4c5ccccc5c5c6ccccc6c6c7ccccc7sc6c54)nc4ccc5ccccc5c34)cc2)cc1 ; c1ccc(-c2nc(-n3c4ccccc4c4c5ccccc5c5c6ccccc6sc5c43)nc3c2ccc2ccccc23)cc1 ; c1ccc(-c2ccc(-c3nc(-n4c5ccccc5c5c6ccccc6c6c7ccccc7sc6c54)nc4c3ccc3ccccc34)cc2)cc1]

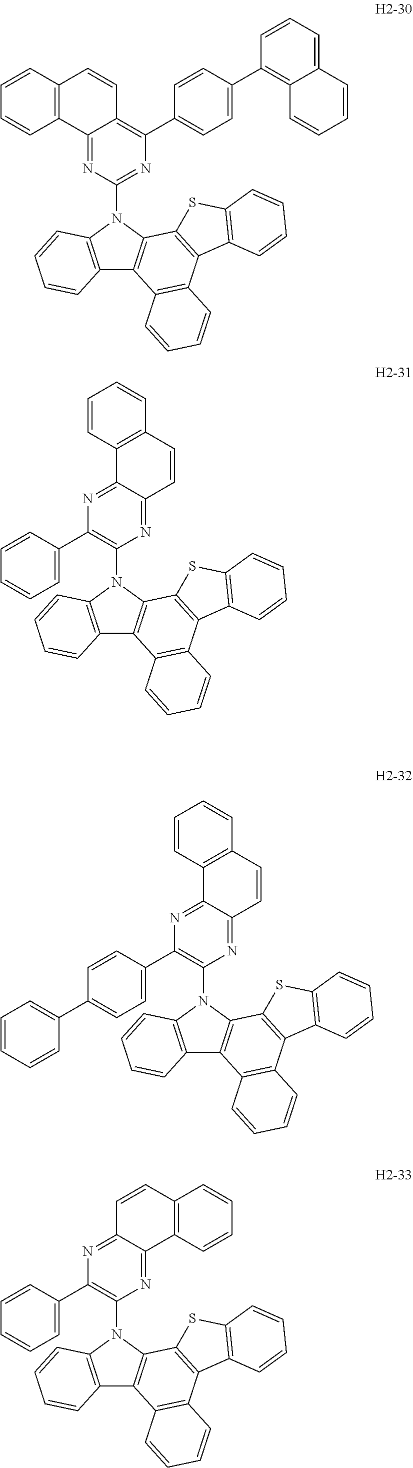 [SMILES: c1ccc2c(-c3ccc(-c4nc(-n5c6ccccc6c6c7ccccc7c7c8ccccc8sc7c65)nc5c4ccc4ccccc45)cc3)cccc2c1 ; c1ccc(-c2nc3c(ccc4ccccc43)nc2-n2c3ccccc3c3c4ccccc4c4c5ccccc5sc4c32)cc1 ; c1ccc(-c2ccc(-c3nc4c(ccc5ccccc54)nc3-n3c4ccccc4c4c5ccccc5c5c6ccccc6sc5c43)cc2)cc1 ; c1ccc(-c2nc3ccc4ccccc4c3nc2-n2c3ccccc3c3c4ccccc4c4c5ccccc5sc4c32)cc1]

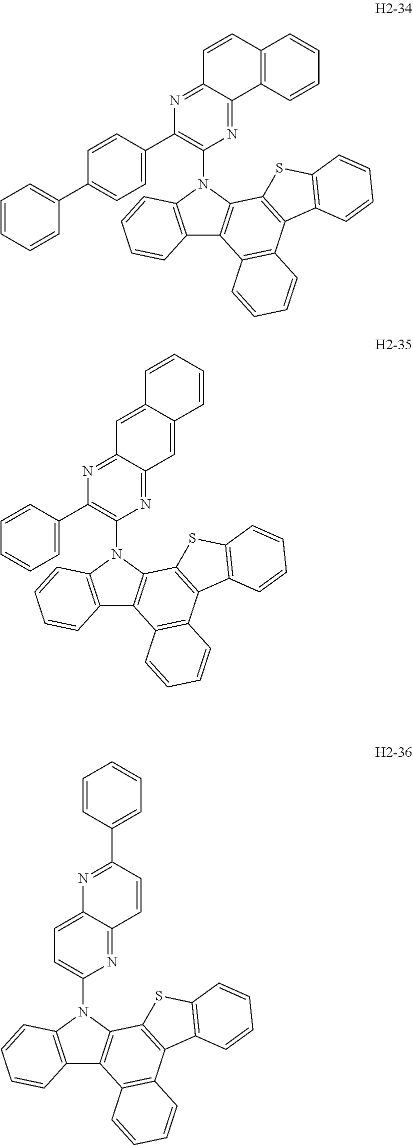 [SMILES: c1ccc(-c2ccc(-c3nc4ccc5ccccc5c4nc3-n3c4ccccc4c4c5ccccc5c5c6ccccc6sc5c43)cc2)cc1 ; c1ccc(-c2nc3cc4ccccc4cc3nc2-n2c3ccccc3c3c4ccccc4c4c5ccccc5sc4c32)cc1 ; c1ccc(-c2ccc3nc(-n4c5ccccc5c5c6ccccc6c6c7ccccc7sc6c54)ccc3n2)cc1]

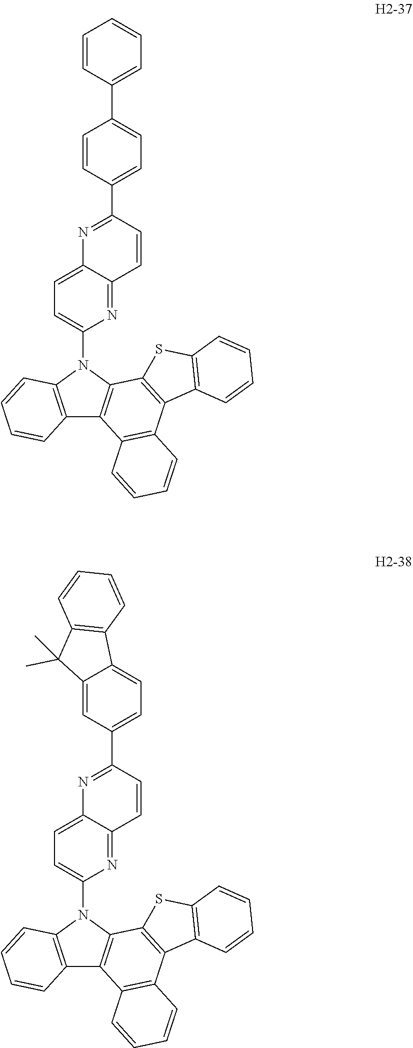 [SMILES: c1ccc(-c2ccc(-c3ccc4nc(-n5c6ccccc6c6c7ccccc7c7c8ccccc8sc7c65)ccc4n3)cc2)cc1 ; CC1(C)c2ccccc2-c2ccc(-c3ccc4nc(-n5c6ccccc6c6c7ccccc7c7c8ccccc8sc7c65)ccc4n3)cc21]

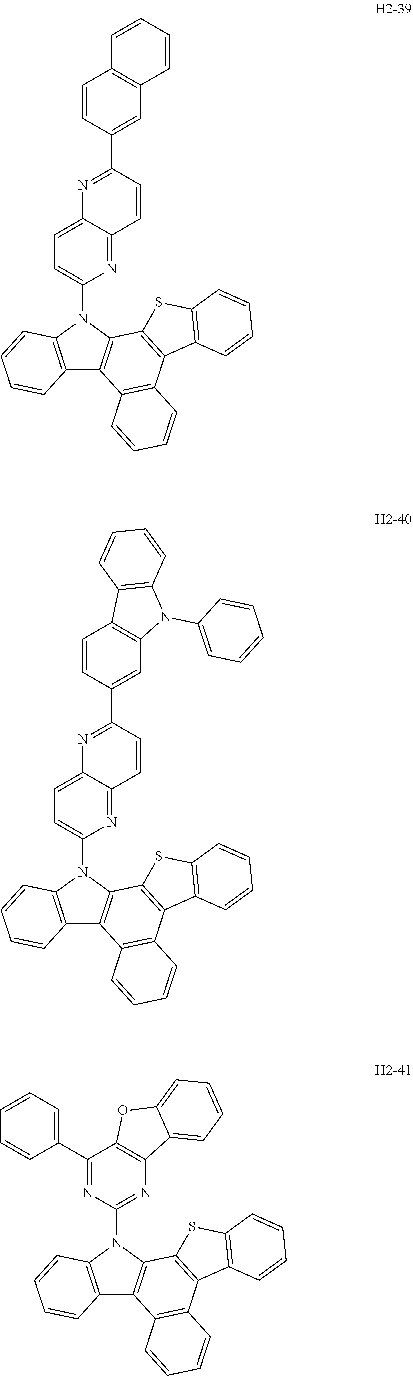 [SMILES: c1ccc2cc(-c3ccc4nc(-n5c6ccccc6c6c7ccccc7c7c8ccccc8sc7c65)ccc4n3)ccc2c1 ; c1ccc(-n2c3ccccc3c3ccc(-c4ccc5nc(-n6c7ccccc7c7c8ccccc8c8c9ccccc9sc8c76)ccc5n4)cc32)cc1 ; c1ccc(-c2nc(-n3c4ccccc4c4c5ccccc5c5c6ccccc6sc5c43)nc3c2oc2ccccc23)cc1]

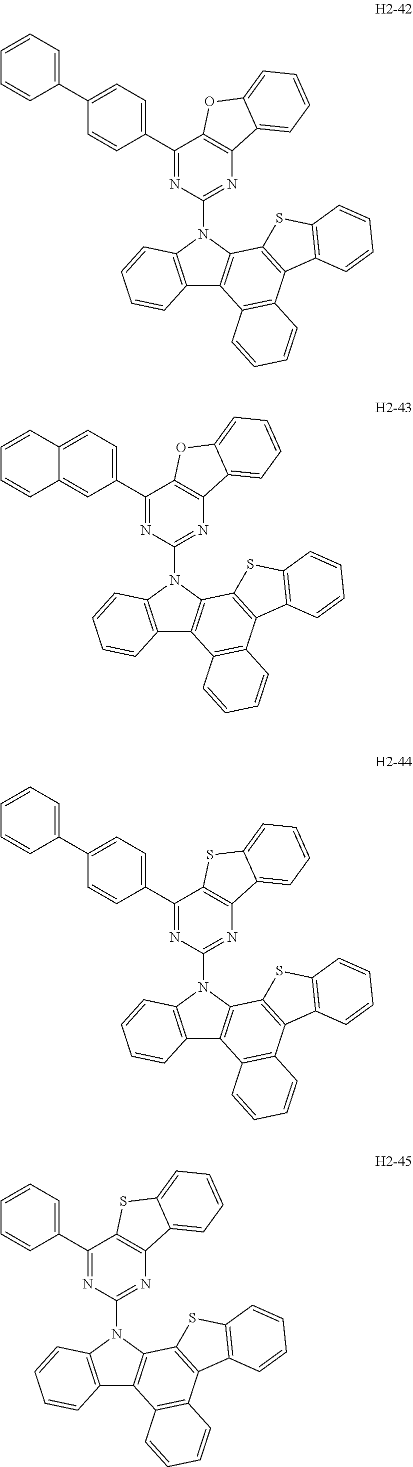 [SMILES: c1ccc(-c2ccc(-c3nc(-n4c5ccccc5c5c6ccccc6c6c7ccccc7sc6c54)nc4c3oc3ccccc34)cc2)cc1 ; c1ccc2cc(-c3nc(-n4c5ccccc5c5c6ccccc6c6c7ccccc7sc6c54)nc4c3oc3ccccc34)ccc2c1 ; c1ccc(-c2ccc(-c3nc(-n4c5ccccc5c5c6ccccc6c6c7ccccc7sc6c54)nc4c3sc3ccccc34)cc2)cc1 ; c1ccc(-c2nc(-n3c4ccccc4c4c5ccccc5c5c6ccccc6sc5c43)nc3c2sc2ccccc23)cc1]

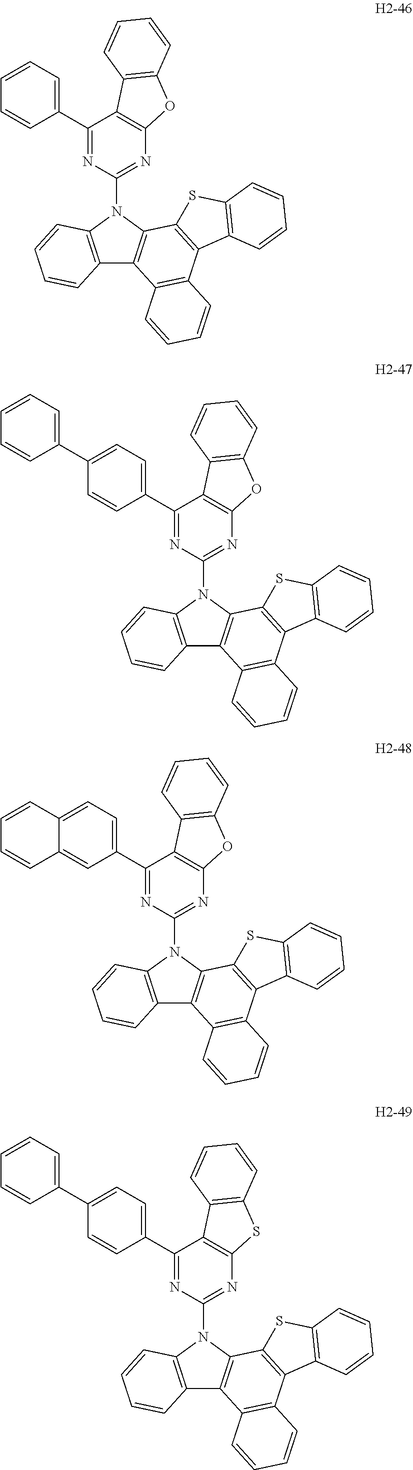 [SMILES: c1ccc(-c2nc(-n3c4ccccc4c4c5ccccc5c5c6ccccc6sc5c43)nc3oc4ccccc4c23)cc1 ; c1ccc(-c2ccc(-c3nc(-n4c5ccccc5c5c6ccccc6c6c7ccccc7sc6c54)nc4oc5ccccc5c34)cc2)cc1 ; c1ccc2cc(-c3nc(-n4c5ccccc5c5c6ccccc6c6c7ccccc7sc6c54)nc4oc5ccccc5c34)ccc2c1 ; c1ccc(-c2ccc(-c3nc(-n4c5ccccc5c5c6ccccc6c6c7ccccc7sc6c54)nc4sc5ccccc5c34)cc2)cc1]

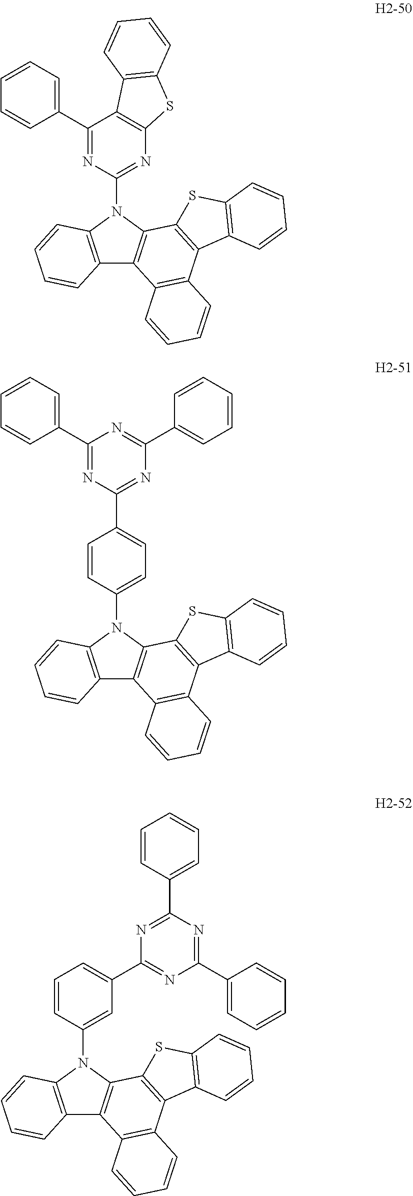

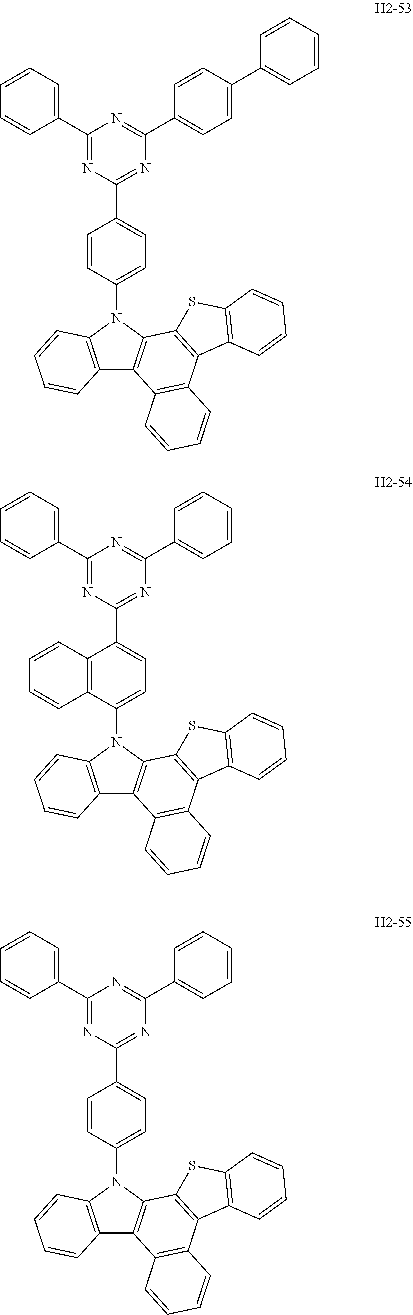 [SMILES: c1ccc(-c2ccc(-c3nc(-c4ccccc4)nc(-c4ccc(-n5c6ccccc6c6c7ccccc7c7c8ccccc8sc7c65)cc4)n3)cc2)cc1 ; c1ccc(-c2nc(-c3ccccc3)nc(-c3ccc(-n4c5ccccc5c5c6ccccc6c6c7ccccc7sc6c54)c4ccccc34)n2)cc1 ; c1ccc(-c2nc(-c3ccccc3)nc(-c3ccc(-n4c5ccccc5c5c6ccccc6c6c7ccccc7sc6c54)cc3)n2)cc1]

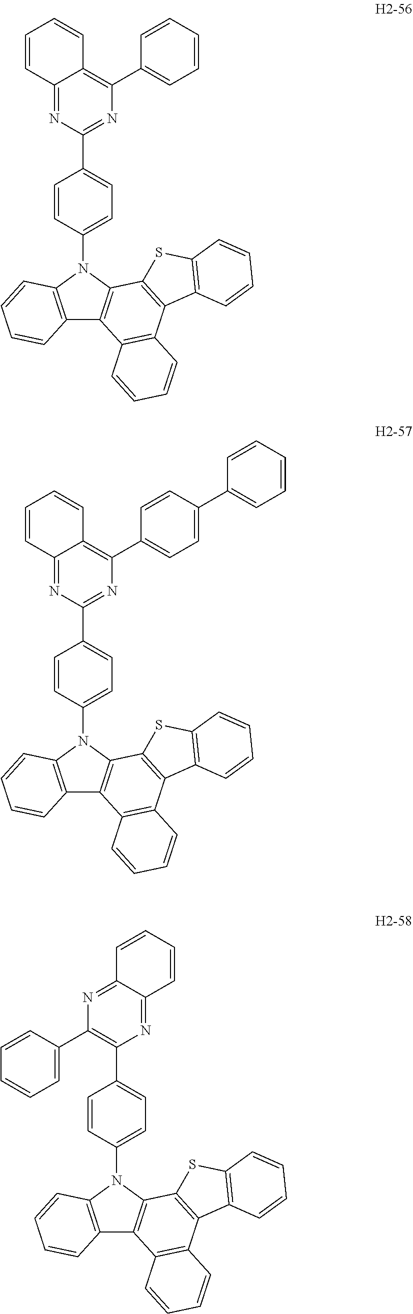 [SMILES: c1ccc(-c2nc(-c3ccc(-n4c5ccccc5c5c6ccccc6c6c7ccccc7sc6c54)cc3)nc3ccccc23)cc1 ; c1ccc(-c2ccc(-c3nc(-c4ccc(-n5c6ccccc6c6c7ccccc7c7c8ccccc8sc7c65)cc4)nc4ccccc34)cc2)cc1 ; c1ccc(-c2nc3ccccc3nc2-c2ccc(-n3c4ccccc4c4c5ccccc5c5c6ccccc6sc5c43)cc2)cc1]

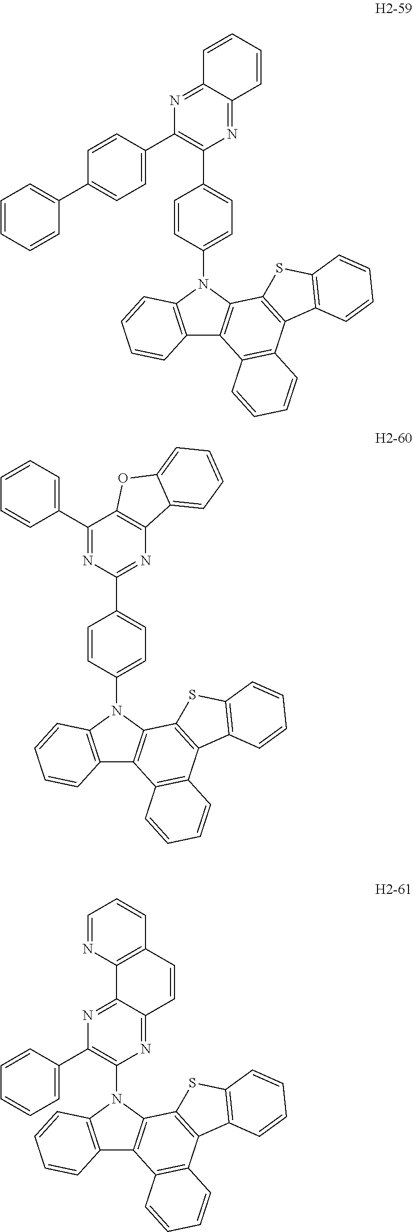 [SMILES: c1ccc(-c2ccc(-c3nc4ccccc4nc3-c3ccc(-n4c5ccccc5c5c6ccccc6c6c7ccccc7sc6c54)cc3)cc2)cc1 ; c1ccc(-c2nc(-c3ccc(-n4c5ccccc5c5c6ccccc6c6c7ccccc7sc6c54)cc3)nc3c2oc2ccccc23)cc1 ; c1ccc(-c2nc3c(ccc4cccnc43)nc2-n2c3ccccc3c3c4ccccc4c4c5ccccc5sc4c32)cc1]

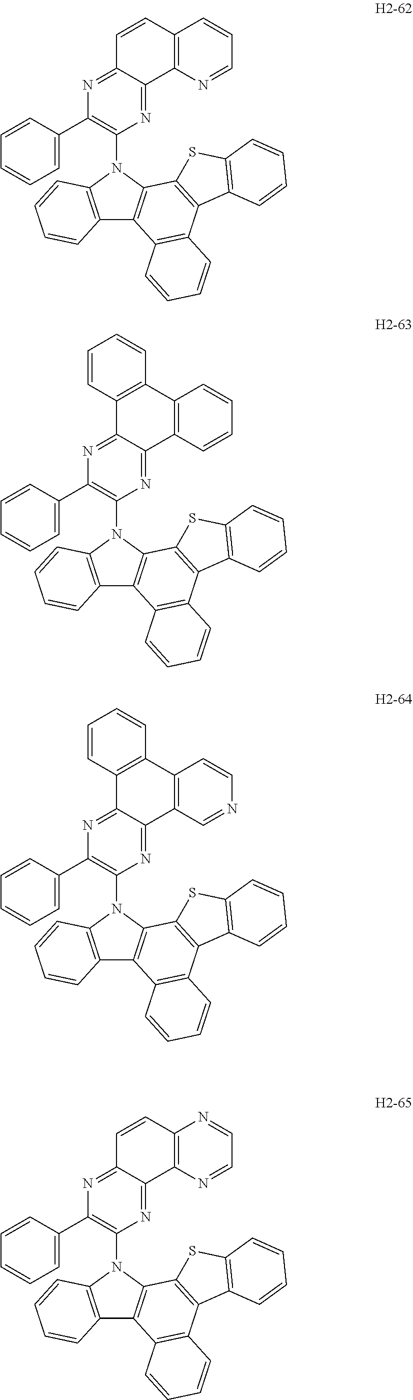 [SMILES: c1ccc(-c2nc3ccc4cccnc4c3nc2-n2c3ccccc3c3c4ccccc4c4c5ccccc5sc4c32)cc1 ; c1ccc(-c2nc3c4ccccc4c4ccccc4c3nc2-n2c3ccccc3c3c4ccccc4c4c5ccccc5sc4c32)cc1 ; c1ccc(-c2nc3c4ccccc4c4ccncc4c3nc2-n2c3ccccc3c3c4ccccc4c4c5ccccc5sc4c32)cc1 ; c1ccc(-c2nc3ccc4nccnc4c3nc2-n2c3ccccc3c3c4ccccc4c4c5ccccc5sc4c32)cc1]

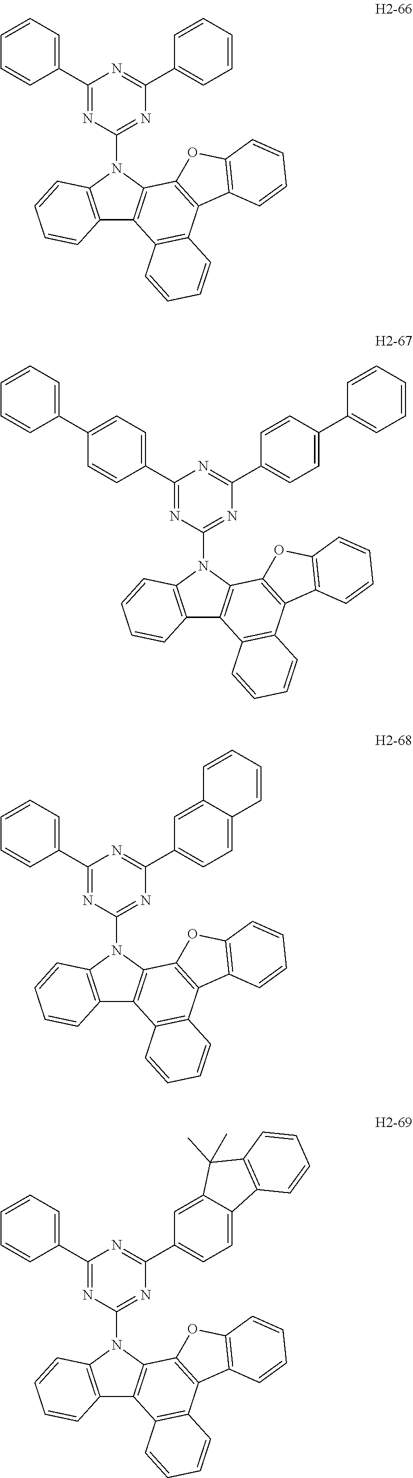 [SMILES: c1ccc(-c2nc(-c3ccccc3)nc(-n3c4ccccc4c4c5ccccc5c5c6ccccc6oc5c43)n2)cc1 ; c1ccc(-c2ccc(-c3nc(-c4ccc(-c5ccccc5)cc4)nc(-n4c5ccccc5c5c6ccccc6c6c7ccccc7oc6c54)n3)cc2)cc1 ; c1ccc(-c2nc(-c3ccc4ccccc4c3)nc(-n3c4ccccc4c4c5ccccc5c5c6ccccc6oc5c43)n2)cc1 ; CC1(C)c2ccccc2-c2ccc(-c3nc(-c4ccccc4)nc(-n4c5ccccc5c5c6ccccc6c6c7ccccc7oc6c54)n3)cc21]

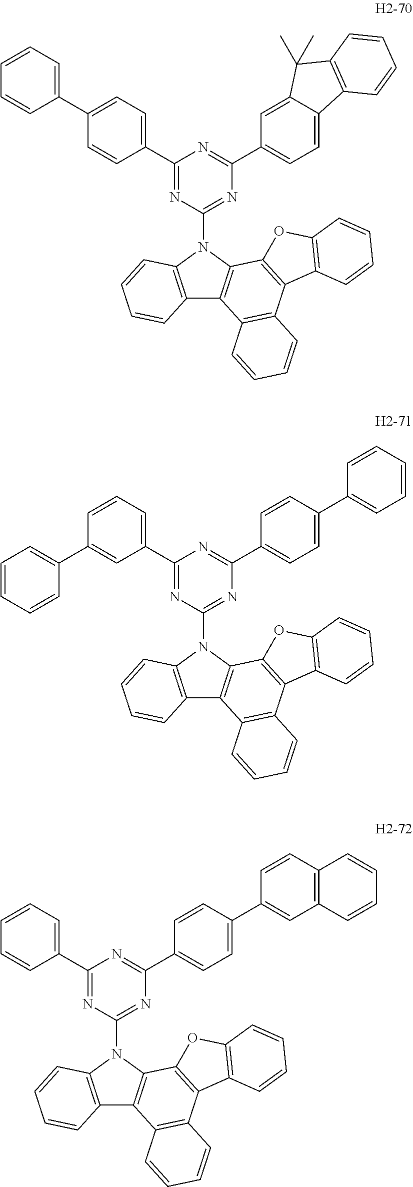 [SMILES: CC1(C)c2ccccc2-c2ccc(-c3nc(-c4ccc(-c5ccccc5)cc4)nc(-n4c5ccccc5c5c6ccccc6c6c7ccccc7oc6c54)n3)cc21 ; c1ccc(-c2ccc(-c3nc(-c4cccc(-c5ccccc5)c4)nc(-n4c5ccccc5c5c6ccccc6c6c7ccccc7oc6c54)n3)cc2)cc1 ; c1ccc(-c2nc(-c3ccc(-c4ccc5ccccc5c4)cc3)nc(-n3c4ccccc4c4c5ccccc5c5c6ccccc6oc5c43)n2)cc1]

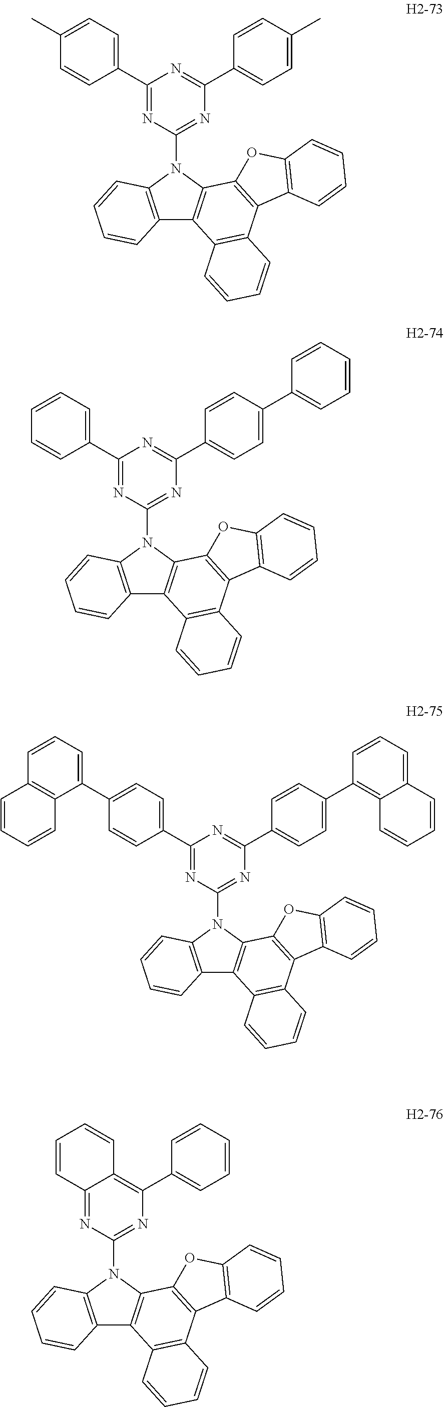 [SMILES: Cc1ccc(-c2nc(-c3ccc(C)cc3)nc(-n3c4ccccc4c4c5ccccc5c5c6ccccc6oc5c43)n2)cc1 ; c1ccc(-c2ccc(-c3nc(-c4ccccc4)nc(-n4c5ccccc5c5c6ccccc6c6c7ccccc7oc6c54)n3)cc2)cc1 ; c1ccc2c(-c3ccc(-c4nc(-c5ccc(-c6cccc7ccccc67)cc5)nc(-n5c6ccccc6c6c7ccccc7c7c8ccccc8oc7c65)n4)cc3)cccc2c1 ; c1ccc(-c2nc(-n3c4ccccc4c4c5ccccc5c5c6ccccc6oc5c43)nc3ccccc23)cc1]

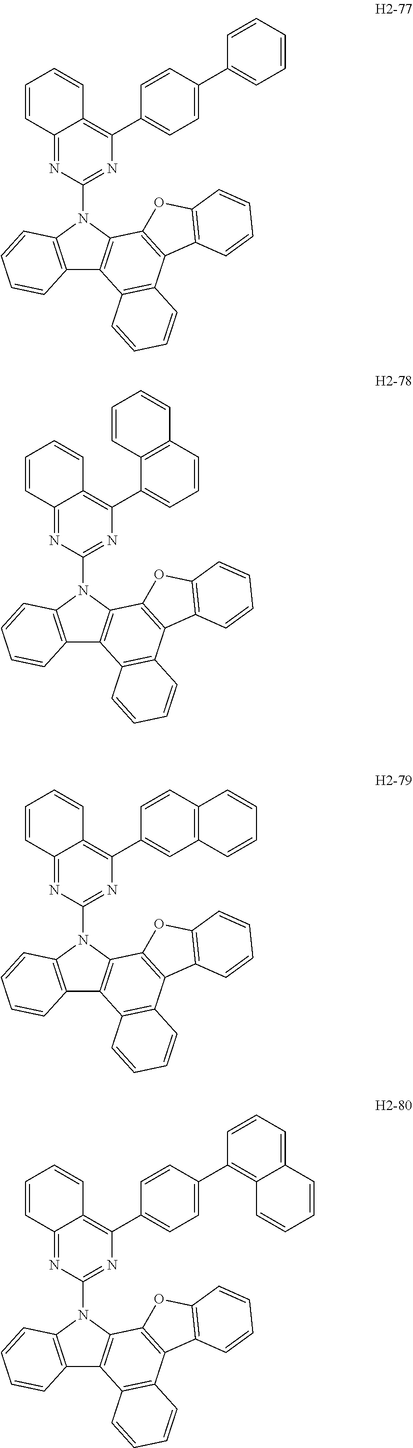

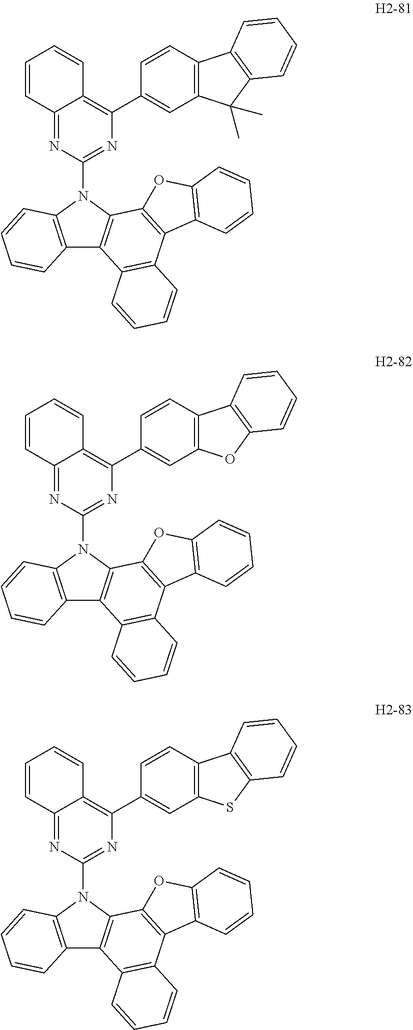 [SMILES: CC1(C)c2ccccc2-c2ccc(-c3nc(-n4c5ccccc5c5c6ccccc6c6c7ccccc7oc6c54)nc4ccccc34)cc21 ; c1ccc2c(-c3ccc4c(c3)oc3ccccc34)nc(-n3c4ccccc4c4c5ccccc5c5c6ccccc6oc5c43)nc2c1 ; c1ccc2c(-c3ccc4c(c3)sc3ccccc34)nc(-n3c4ccccc4c4c5ccccc5c5c6ccccc6oc5c43)nc2c1]

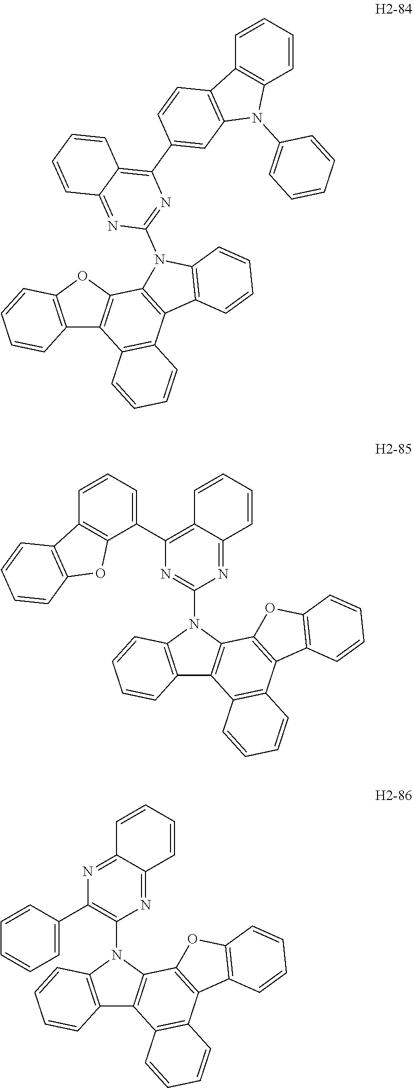 [SMILES: c1ccc(-n2c3ccccc3c3ccc(-c4nc(-n5c6ccccc6c6c7ccccc7c7c8ccccc8oc7c65)nc5ccccc45)cc32)cc1 ; c1ccc2c(-c3cccc4c3oc3ccccc34)nc(-n3c4ccccc4c4c5ccccc5c5c6ccccc6oc5c43)nc2c1 ; c1ccc(-c2nc3ccccc3nc2-n2c3ccccc3c3c4ccccc4c4c5ccccc5oc4c32)cc1]

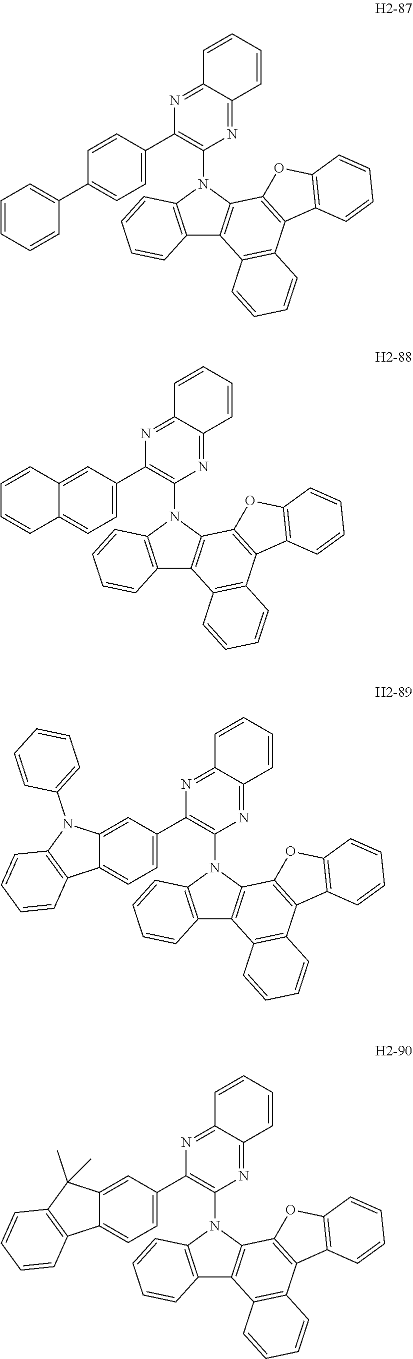 [SMILES: c1ccc(-c2ccc(-c3nc4ccccc4nc3-n3c4ccccc4c4c5ccccc5c5c6ccccc6oc5c43)cc2)cc1 ; c1ccc2cc(-c3nc4ccccc4nc3-n3c4ccccc4c4c5ccccc5c5c6ccccc6oc5c43)ccc2c1 ; c1ccc(-n2c3ccccc3c3ccc(-c4nc5ccccc5nc4-n4c5ccccc5c5c6ccccc6c6c7ccccc7oc6c54)cc32)cc1 ; CC1(C)c2ccccc2-c2ccc(-c3nc4ccccc4nc3-n3c4ccccc4c4c5ccccc5c5c6ccccc6oc5c43)cc21]

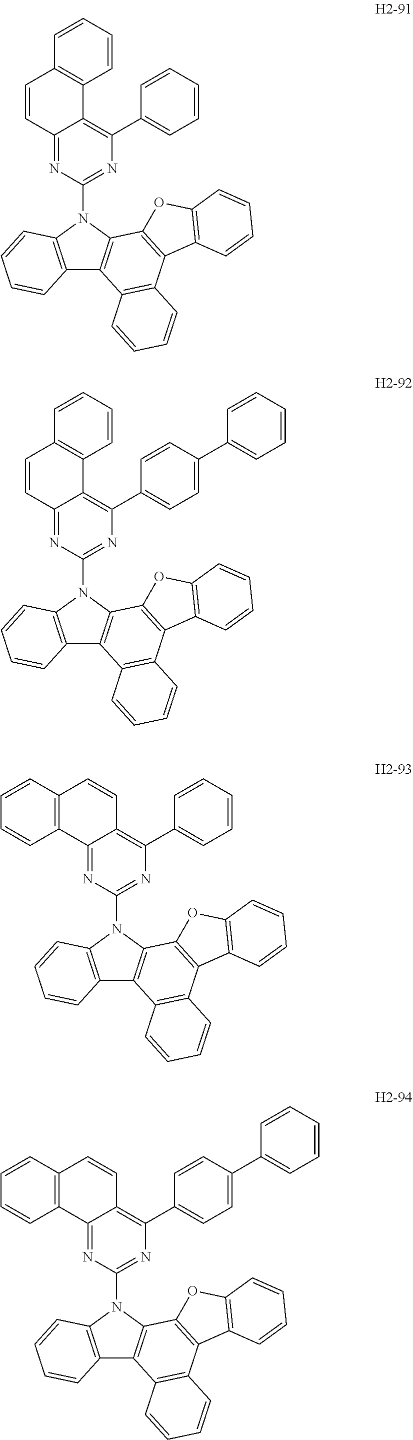 [SMILES: c1ccc(-c2nc(-n3c4ccccc4c4c5ccccc5c5c6ccccc6oc5c43)nc3ccc4ccccc4c23)cc1 ; c1ccc(-c2ccc(-c3nc(-n4c5ccccc5c5c6ccccc6c6c7ccccc7oc6c54)nc4ccc5ccccc5c34)cc2)cc1 ; c1ccc(-c2nc(-n3c4ccccc4c4c5ccccc5c5c6ccccc6oc5c43)nc3c2ccc2ccccc23)cc1 ; c1ccc(-c2ccc(-c3nc(-n4c5ccccc5c5c6ccccc6c6c7ccccc7oc6c54)nc4c3ccc3ccccc34)cc2)cc1]

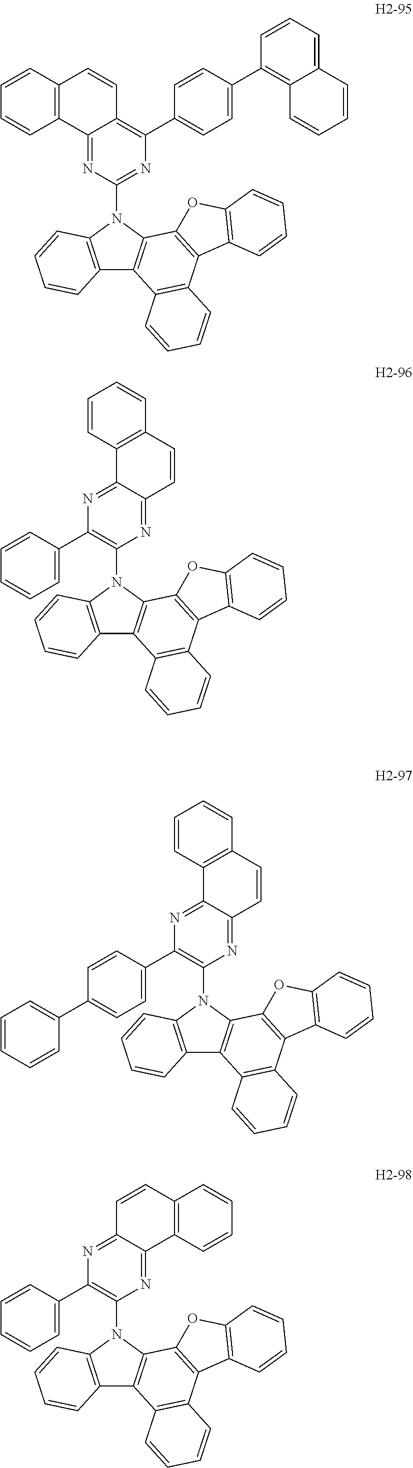 [SMILES: c1ccc2c(-c3ccc(-c4nc(-n5c6ccccc6c6c7ccccc7c7c8ccccc8oc7c65)nc5c4ccc4ccccc45)cc3)cccc2c1 ; c1ccc(-c2nc3c(ccc4ccccc43)nc2-n2c3ccccc3c3c4ccccc4c4c5ccccc5oc4c32)cc1 ; c1ccc(-c2ccc(-c3nc4c(ccc5ccccc54)nc3-n3c4ccccc4c4c5ccccc5c5c6ccccc6oc5c43)cc2)cc1 ; c1ccc(-c2nc3ccc4ccccc4c3nc2-n2c3ccccc3c3c4ccccc4c4c5ccccc5oc4c32)cc1]

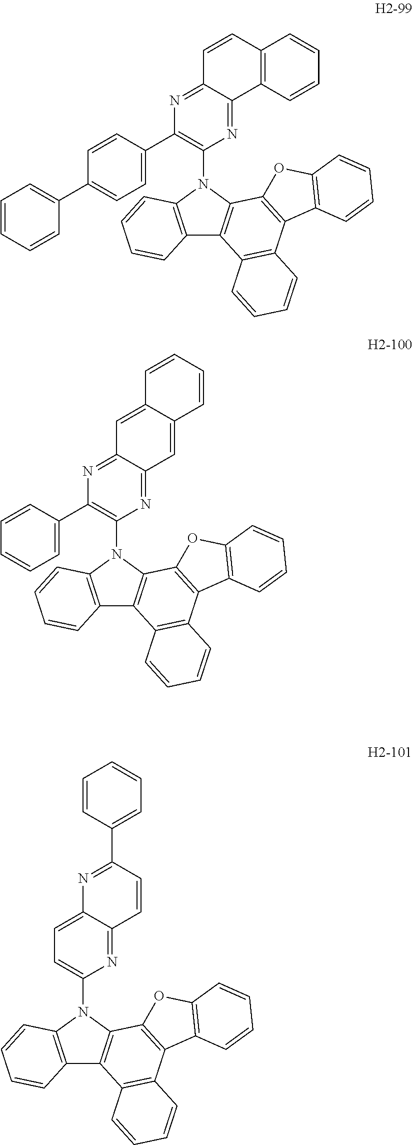 [SMILES: c1ccc(-c2ccc(-c3nc4ccc5ccccc5c4nc3-n3c4ccccc4c4c5ccccc5c5c6ccccc6oc5c43)cc2)cc1 ; c1ccc(-c2nc3cc4ccccc4cc3nc2-n2c3ccccc3c3c4ccccc4c4c5ccccc5oc4c32)cc1 ; c1ccc(-c2ccc3nc(-n4c5ccccc5c5c6ccccc6c6c7ccccc7oc6c54)ccc3n2)cc1]

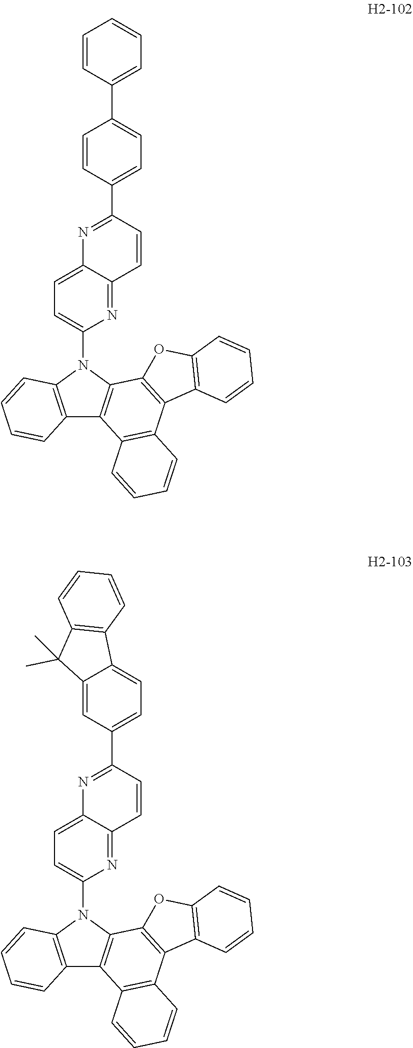 [SMILES: c1ccc(-c2ccc(-c3ccc4nc(-n5c6ccccc6c6c7ccccc7c7c8ccccc8oc7c65)ccc4n3)cc2)cc1 ; CC1(C)c2ccccc2-c2ccc(-c3ccc4nc(-n5c6ccccc6c6c7ccccc7c7c8ccccc8oc7c65)ccc4n3)cc21]

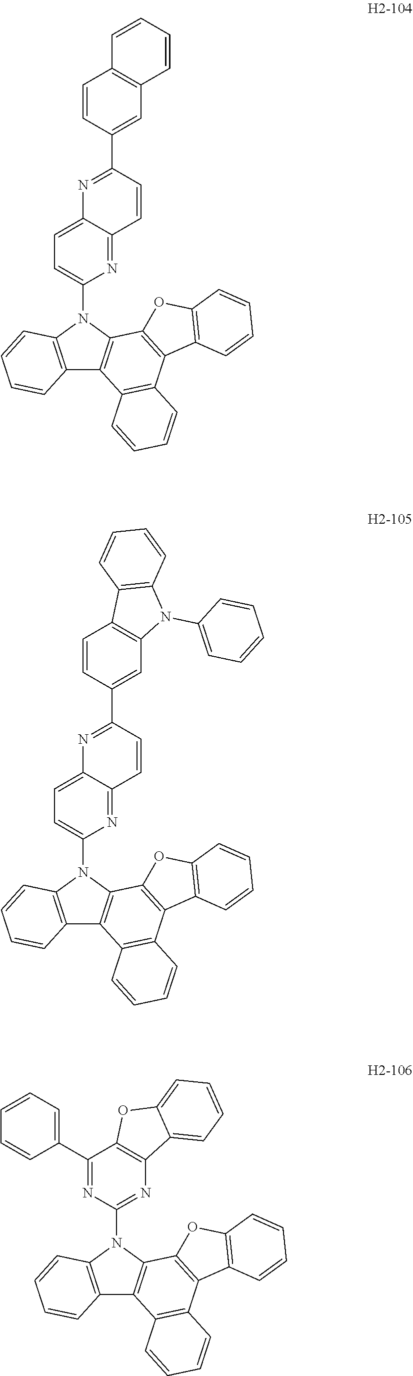 [SMILES: c1ccc2cc(-c3ccc4nc(-n5c6ccccc6c6c7ccccc7c7c8ccccc8oc7c65)ccc4n3)ccc2c1 ; c1ccc(-n2c3ccccc3c3ccc(-c4ccc5nc(-n6c7ccccc7c7c8ccccc8c8c9ccccc9oc8c76)ccc5n4)cc32)cc1 ; c1ccc(-c2nc(-n3c4ccccc4c4c5ccccc5c5c6ccccc6oc5c43)nc3c2oc2ccccc23)cc1]

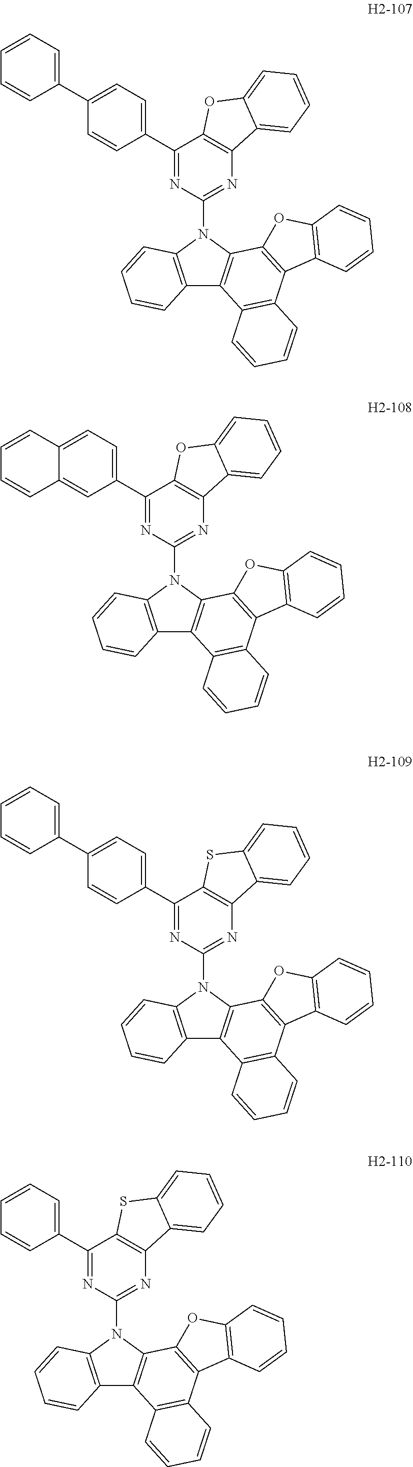 [SMILES: c1ccc(-c2ccc(-c3nc(-n4c5ccccc5c5c6ccccc6c6c7ccccc7oc6c54)nc4c3oc3ccccc34)cc2)cc1 ; c1ccc2cc(-c3nc(-n4c5ccccc5c5c6ccccc6c6c7ccccc7oc6c54)nc4c3oc3ccccc34)ccc2c1 ; c1ccc(-c2ccc(-c3nc(-n4c5ccccc5c5c6ccccc6c6c7ccccc7oc6c54)nc4c3sc3ccccc34)cc2)cc1 ; c1ccc(-c2nc(-n3c4ccccc4c4c5ccccc5c5c6ccccc6oc5c43)nc3c2sc2ccccc23)cc1]

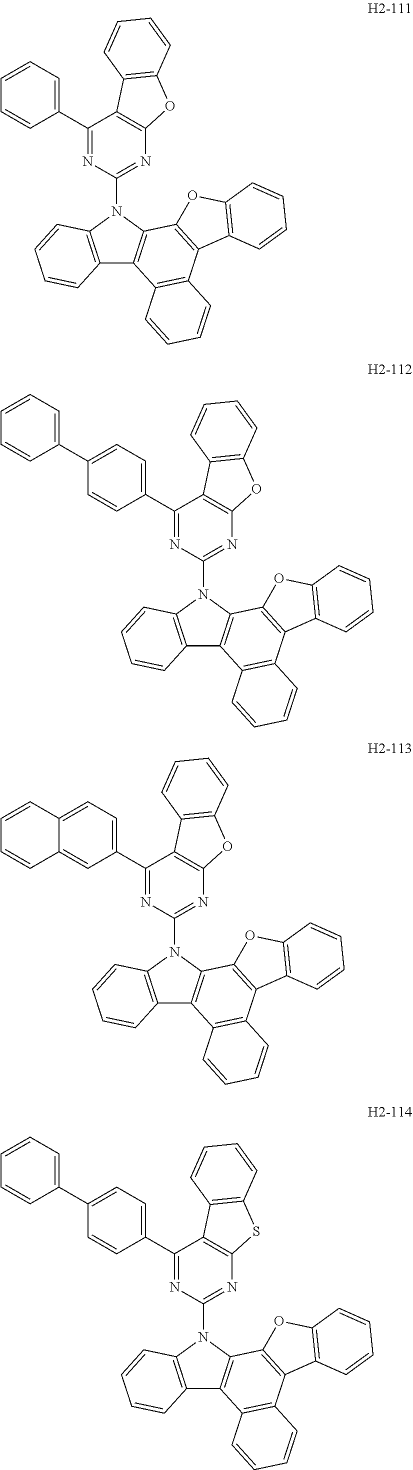 [SMILES: c1ccc(-c2nc(-n3c4ccccc4c4c5ccccc5c5c6ccccc6oc5c43)nc3oc4ccccc4c23)cc1 ; c1ccc(-c2ccc(-c3nc(-n4c5ccccc5c5c6ccccc6c6c7ccccc7oc6c54)nc4oc5ccccc5c34)cc2)cc1 ; c1ccc2cc(-c3nc(-n4c5ccccc5c5c6ccccc6c6c7ccccc7oc6c54)nc4oc5ccccc5c34)ccc2c1 ; c1ccc(-c2ccc(-c3nc(-n4c5ccccc5c5c6ccccc6c6c7ccccc7oc6c54)nc4sc5ccccc5c34)cc2)cc1]

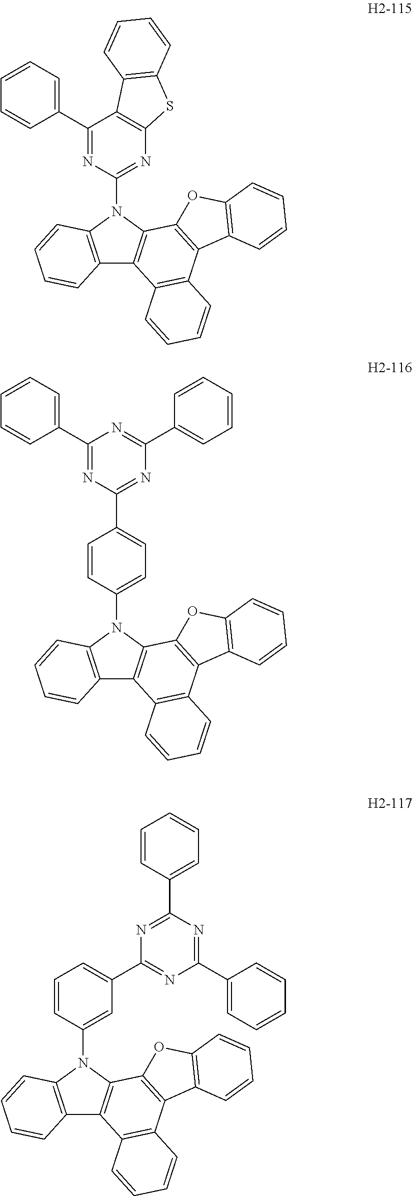

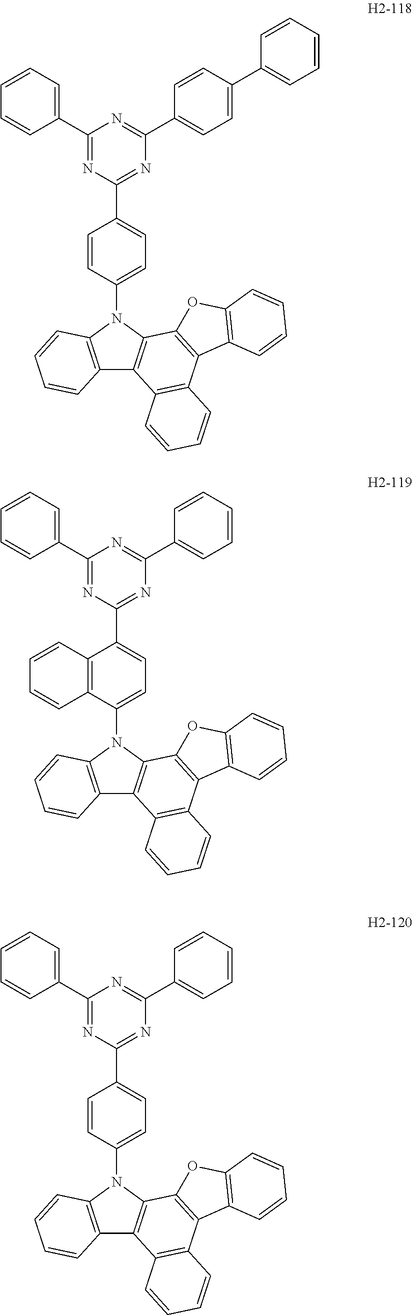 [SMILES: c1ccc(-c2ccc(-c3nc(-c4ccccc4)nc(-c4ccc(-n5c6ccccc6c6c7ccccc7c7c8ccccc8oc7c65)cc4)n3)cc2)cc1 ; c1ccc(-c2nc(-c3ccccc3)nc(-c3ccc(-n4c5ccccc5c5c6ccccc6c6c7ccccc7oc6c54)c4ccccc34)n2)cc1 ; c1ccc(-c2nc(-c3ccccc3)nc(-c3ccc(-n4c5ccccc5c5c6ccccc6c6c7ccccc7oc6c54)cc3)n2)cc1]

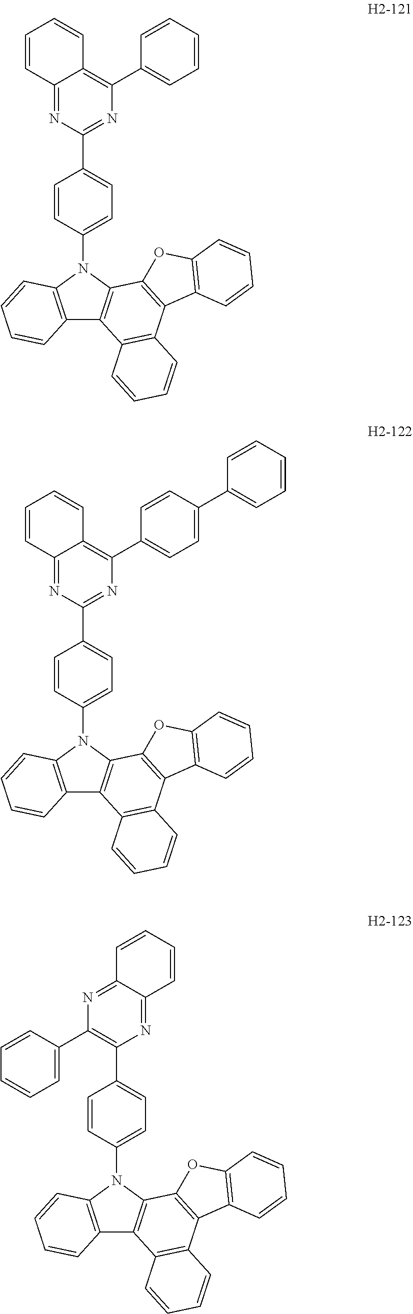 [SMILES: c1ccc(-c2nc(-c3ccc(-n4c5ccccc5c5c6ccccc6c6c7ccccc7oc6c54)cc3)nc3ccccc23)cc1 ; c1ccc(-c2ccc(-c3nc(-c4ccc(-n5c6ccccc6c6c7ccccc7c7c8ccccc8oc7c65)cc4)nc4ccccc34)cc2)cc1 ; c1ccc(-c2nc3ccccc3nc2-c2ccc(-n3c4ccccc4c4c5ccccc5c5c6ccccc6oc5c43)cc2)cc1]

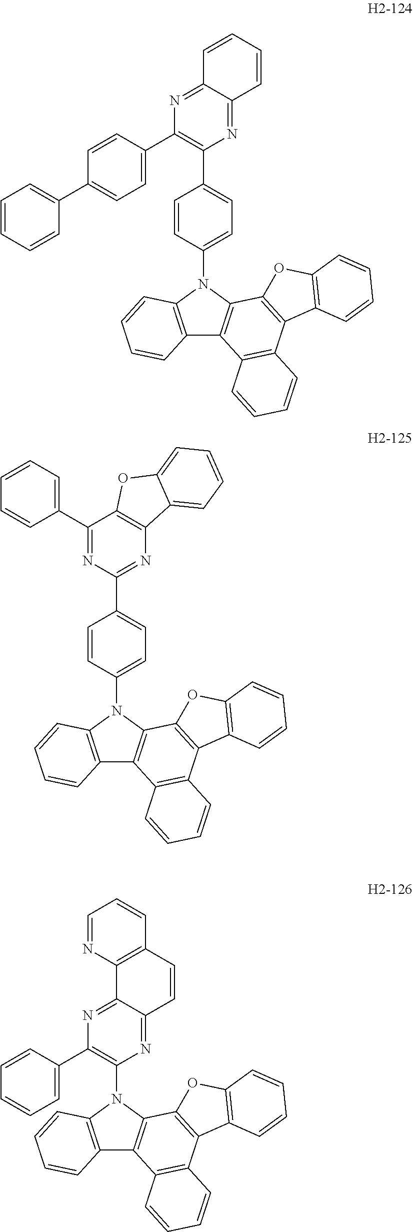 [SMILES: c1ccc(-c2ccc(-c3nc4ccccc4nc3-c3ccc(-n4c5ccccc5c5c6ccccc6c6c7ccccc7oc6c54)cc3)cc2)cc1 ; c1ccc(-c2nc(-c3ccc(-n4c5ccccc5c5c6ccccc6c6c7ccccc7oc6c54)cc3)nc3c2oc2ccccc23)cc1 ; c1ccc(-c2nc3c(ccc4cccnc43)nc2-n2c3ccccc3c3c4ccccc4c4c5ccccc5oc4c32)cc1]

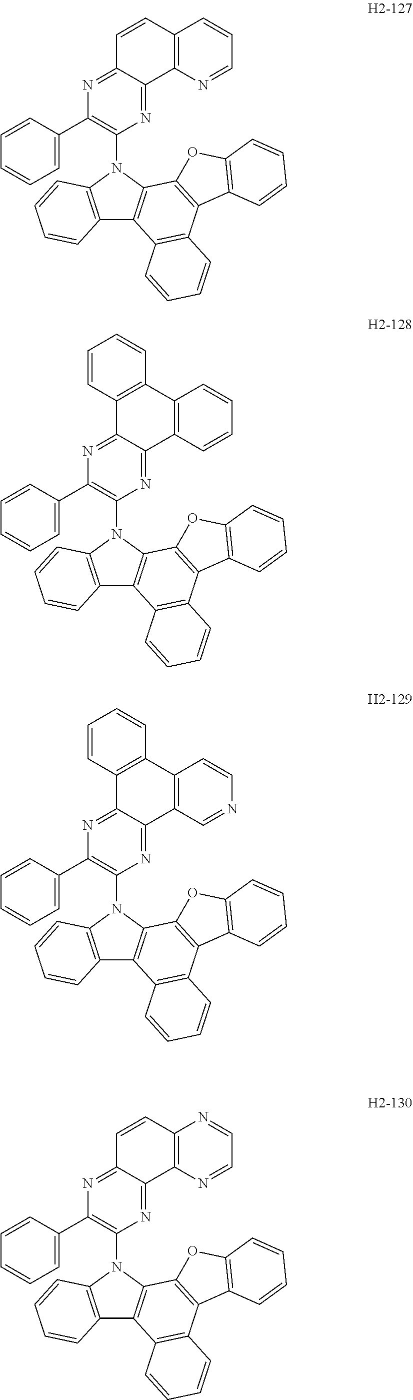 [SMILES: c1ccc(-c2nc3ccc4cccnc4c3nc2-n2c3ccccc3c3c4ccccc4c4c5ccccc5oc4c32)cc1 ; c1ccc(-c2nc3c4ccccc4c4ccccc4c3nc2-n2c3ccccc3c3c4ccccc4c4c5ccccc5oc4c32)cc1 ; c1ccc(-c2nc3c4ccccc4c4ccncc4c3nc2-n2c3ccccc3c3c4ccccc4c4c5ccccc5oc4c32)cc1 ; c1ccc(-c2nc3ccc4nccnc4c3nc2-n2c3ccccc3c3c4ccccc4c4c5ccccc5oc4c32)cc1]

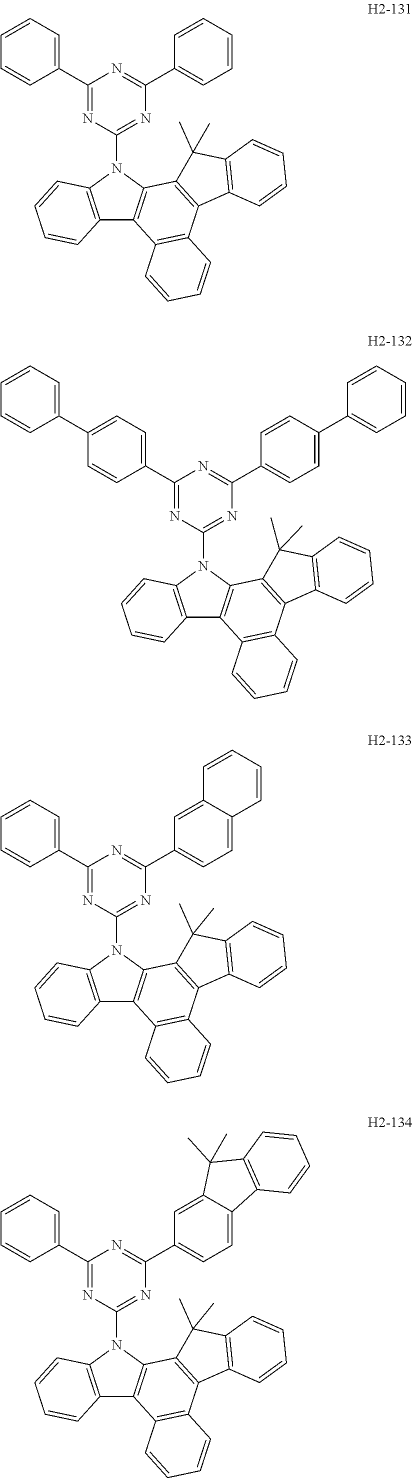 [SMILES: CC1(C)c2ccccc2-c2c1c1c(c3ccccc23)c2ccccc2n1-c1nc(-c2ccccc2)nc(-c2ccccc2)n1 ; CC1(C)c2ccccc2-c2c1c1c(c3ccccc23)c2ccccc2n1-c1nc(-c2ccc(-c3ccccc3)cc2)nc(-c2ccc(-c3ccccc3)cc2)n1 ; CC1(C)c2ccccc2-c2c1c1c(c3ccccc23)c2ccccc2n1-c1nc(-c2ccccc2)nc(-c2ccc3ccccc3c2)n1 ; CC1(C)c2ccccc2-c2ccc(-c3nc(-c4ccccc4)nc(-n4c5ccccc5c5c6ccccc6c6c(c54)C(C)(C)c4ccccc4-6)n3)cc21]

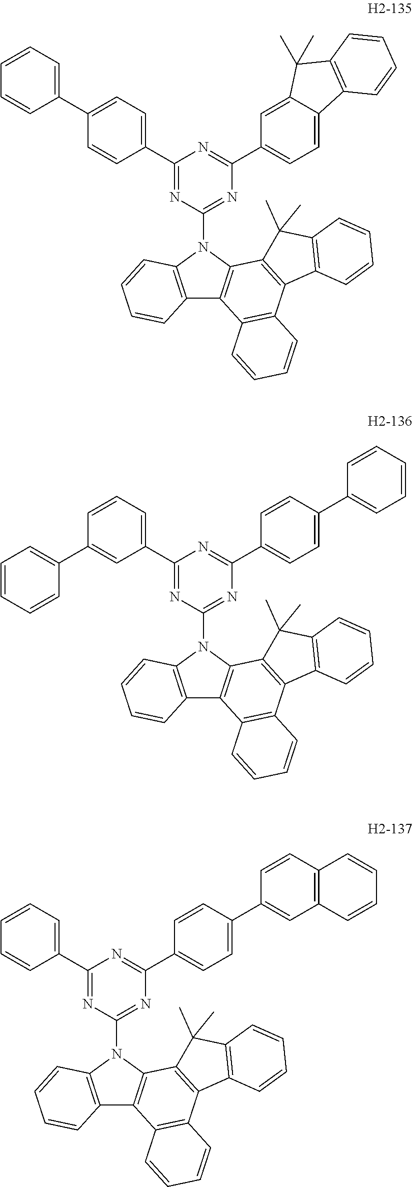 [SMILES: CC1(C)c2ccccc2-c2ccc(-c3nc(-c4ccc(-c5ccccc5)cc4)nc(-n4c5ccccc5c5c6ccccc6c6c(c54)C(C)(C)c4ccccc4-6)n3)cc21 ; CC1(C)c2ccccc2-c2c1c1c(c3ccccc23)c2ccccc2n1-c1nc(-c2ccc(-c3ccccc3)cc2)nc(-c2cccc(-c3ccccc3)c2)n1 ; CC1(C)c2ccccc2-c2c1c1c(c3ccccc23)c2ccccc2n1-c1nc(-c2ccccc2)nc(-c2ccc(-c3ccc4ccccc4c3)cc2)n1]

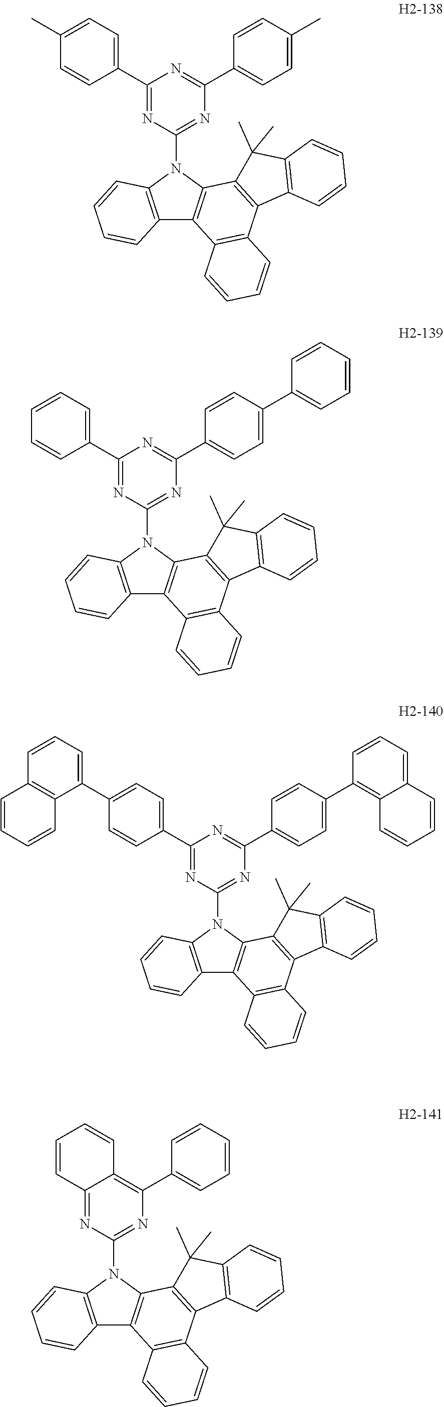 [SMILES: Cc1ccc(-c2nc(-c3ccc(C)cc3)nc(-n3c4ccccc4c4c5ccccc5c5c(c43)C(C)(C)c3ccccc3-5)n2)cc1 ; CC1(C)c2ccccc2-c2c1c1c(c3ccccc23)c2ccccc2n1-c1nc(-c2ccccc2)nc(-c2ccc(-c3ccccc3)cc2)n1 ; CC1(C)c2ccccc2-c2c1c1c(c3ccccc23)c2ccccc2n1-c1nc(-c2ccc(-c3cccc4ccccc34)cc2)nc(-c2ccc(-c3cccc4ccccc34)cc2)n1 ; CC1(C)c2ccccc2-c2c1c1c(c3ccccc23)c2ccccc2n1-c1nc(-c2ccccc2)c2ccccc2n1]

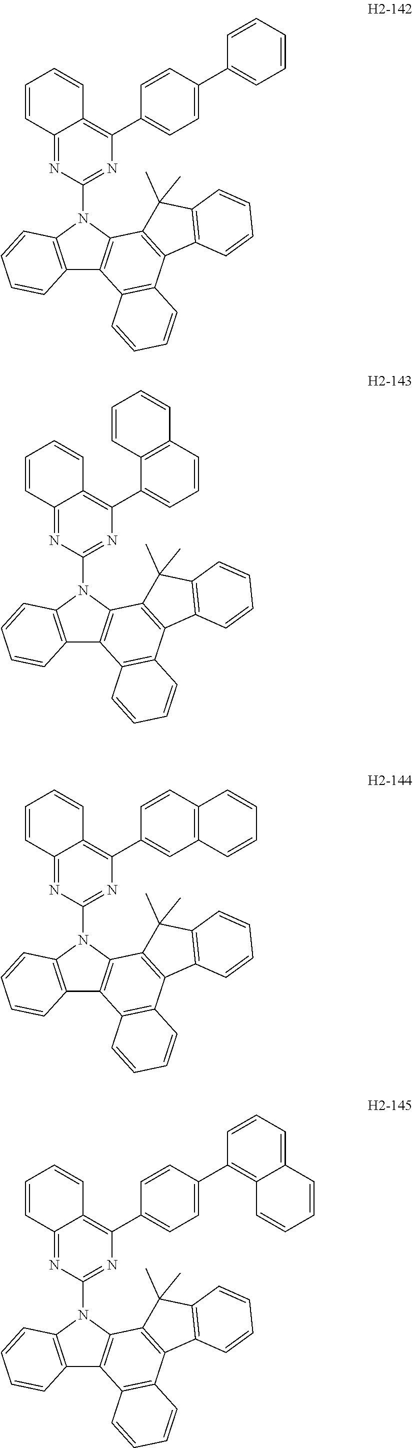

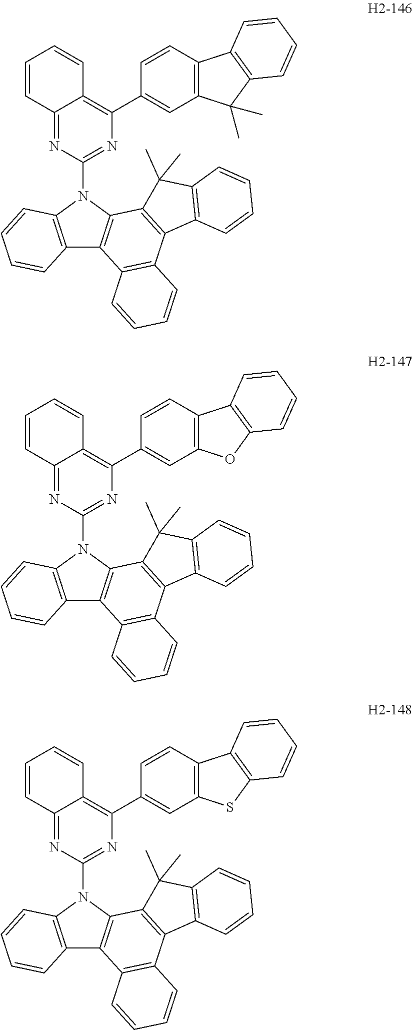 [SMILES: CC1(C)c2ccccc2-c2ccc(-c3nc(-n4c5ccccc5c5c6ccccc6c6c(c54)C(C)(C)c4ccccc4-6)nc4ccccc34)cc21 ; CC1(C)c2ccccc2-c2c1c1c(c3ccccc23)c2ccccc2n1-c1nc(-c2ccc3c(c2)oc2ccccc23)c2ccccc2n1 ; CC1(C)c2ccccc2-c2c1c1c(c3ccccc23)c2ccccc2n1-c1nc(-c2ccc3c(c2)sc2ccccc23)c2ccccc2n1]

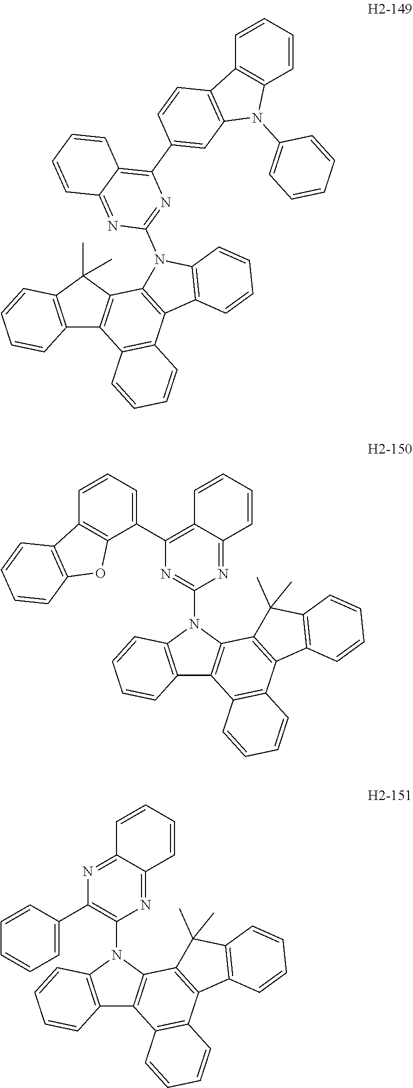 [SMILES: CC1(C)c2ccccc2-c2c1c1c(c3ccccc23)c2ccccc2n1-c1nc(-c2ccc3c4ccccc4n(-c4ccccc4)c3c2)c2ccccc2n1 ; CC1(C)c2ccccc2-c2c1c1c(c3ccccc23)c2ccccc2n1-c1nc(-c2cccc3c2oc2ccccc23)c2ccccc2n1 ; CC1(C)c2ccccc2-c2c1c1c(c3ccccc23)c2ccccc2n1-c1nc2ccccc2nc1-c1ccccc1]

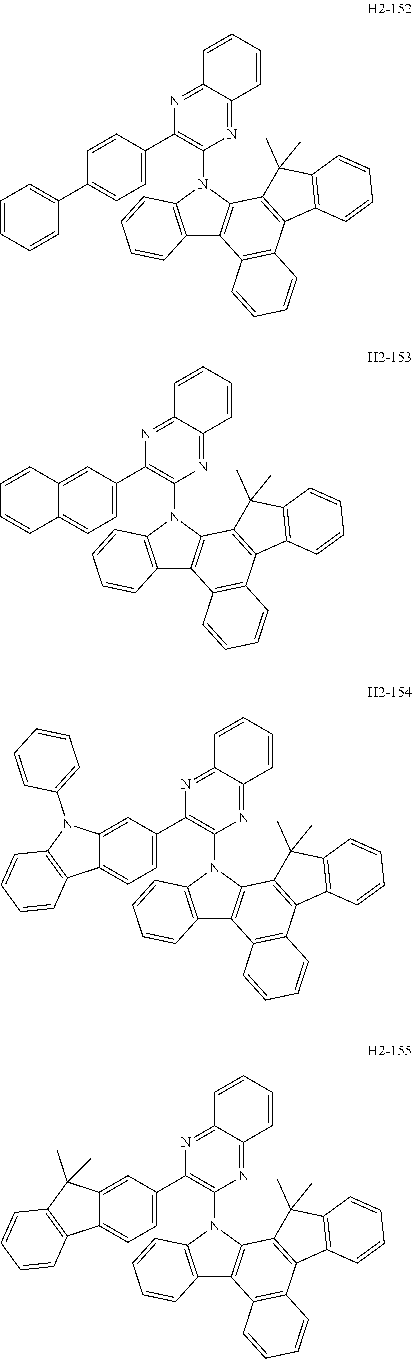 [SMILES: CC1(C)c2ccccc2-c2c1c1c(c3ccccc23)c2ccccc2n1-c1nc2ccccc2nc1-c1ccc(-c2ccccc2)cc1 ; CC1(C)c2ccccc2-c2c1c1c(c3ccccc23)c2ccccc2n1-c1nc2ccccc2nc1-c1ccc2ccccc2c1 ; CC1(C)c2ccccc2-c2c1c1c(c3ccccc23)c2ccccc2n1-c1nc2ccccc2nc1-c1ccc2c3ccccc3n(-c3ccccc3)c2c1 ; CC1(C)c2ccccc2-c2ccc(-c3nc4ccccc4nc3-n3c4ccccc4c4c5ccccc5c5c(c43)C(C)(C)c3ccccc3-5)cc21]

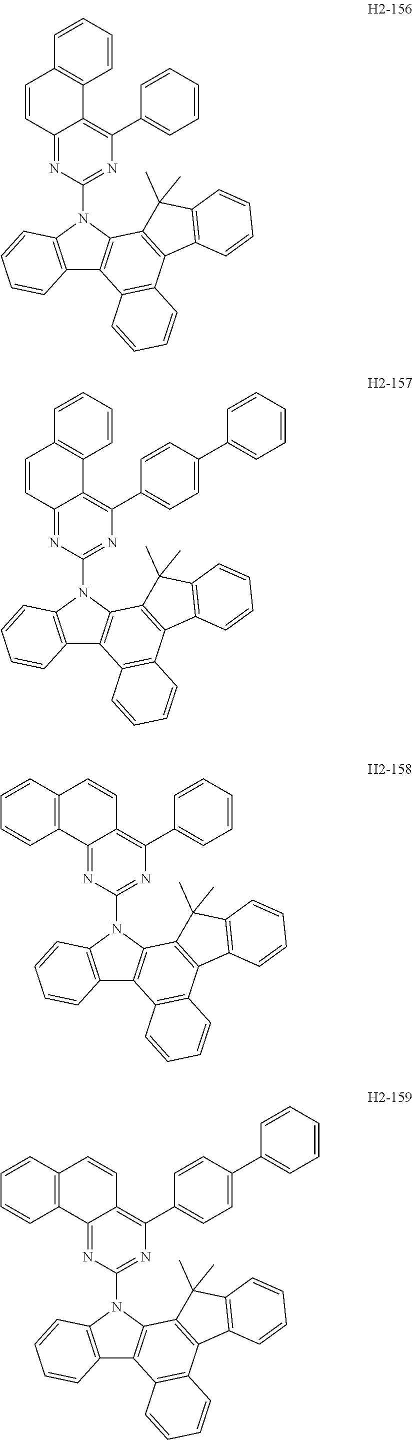 [SMILES: CC1(C)c2ccccc2-c2c1c1c(c3ccccc23)c2ccccc2n1-c1nc(-c2ccccc2)c2c(ccc3ccccc32)n1 ; CC1(C)c2ccccc2-c2c1c1c(c3ccccc23)c2ccccc2n1-c1nc(-c2ccc(-c3ccccc3)cc2)c2c(ccc3ccccc32)n1 ; CC1(C)c2ccccc2-c2c1c1c(c3ccccc23)c2ccccc2n1-c1nc(-c2ccccc2)c2ccc3ccccc3c2n1 ; CC1(C)c2ccccc2-c2c1c1c(c3ccccc23)c2ccccc2n1-c1nc(-c2ccc(-c3ccccc3)cc2)c2ccc3ccccc3c2n1]

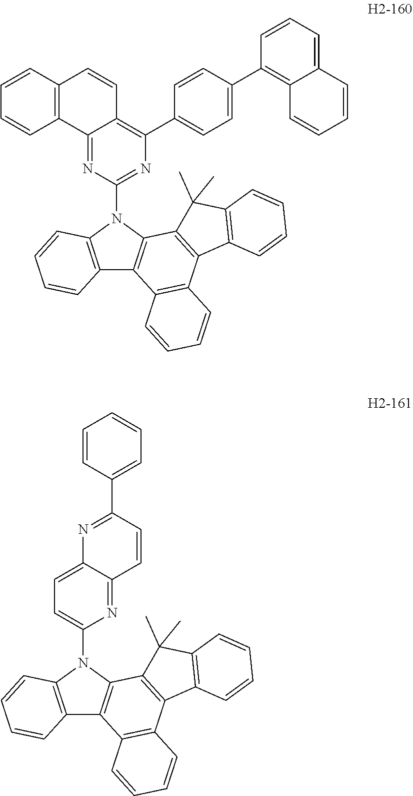 [SMILES: CC1(C)c2ccccc2-c2c1c1c(c3ccccc23)c2ccccc2n1-c1nc(-c2ccc(-c3cccc4ccccc34)cc2)c2ccc3ccccc3c2n1 ; CC1(C)c2ccccc2-c2c1c1c(c3ccccc23)c2ccccc2n1-c1ccc2nc(-c3ccccc3)ccc2n1]

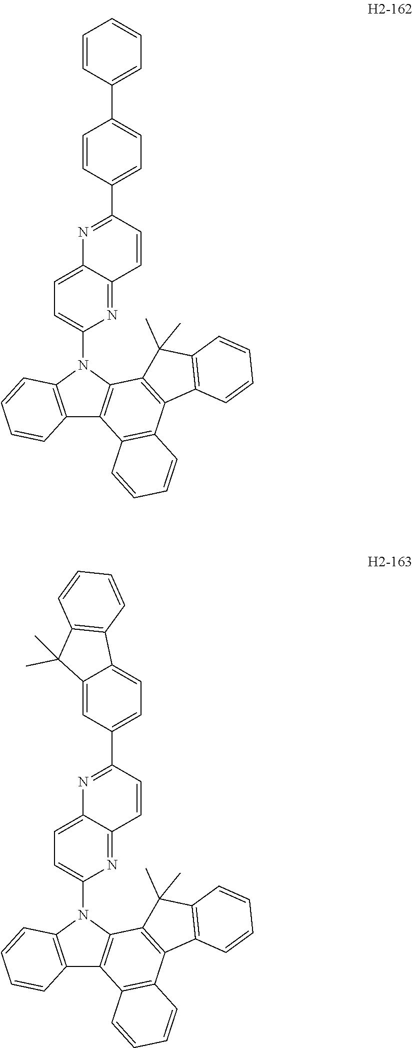 [SMILES: CC1(C)c2ccccc2-c2c1c1c(c3ccccc23)c2ccccc2n1-c1ccc2nc(-c3ccc(-c4ccccc4)cc3)ccc2n1 ; CC1(C)c2ccccc2-c2ccc(-c3ccc4nc(-n5c6ccccc6c6c7ccccc7c7c(c65)C(C)(C)c5ccccc5-7)ccc4n3)cc21]

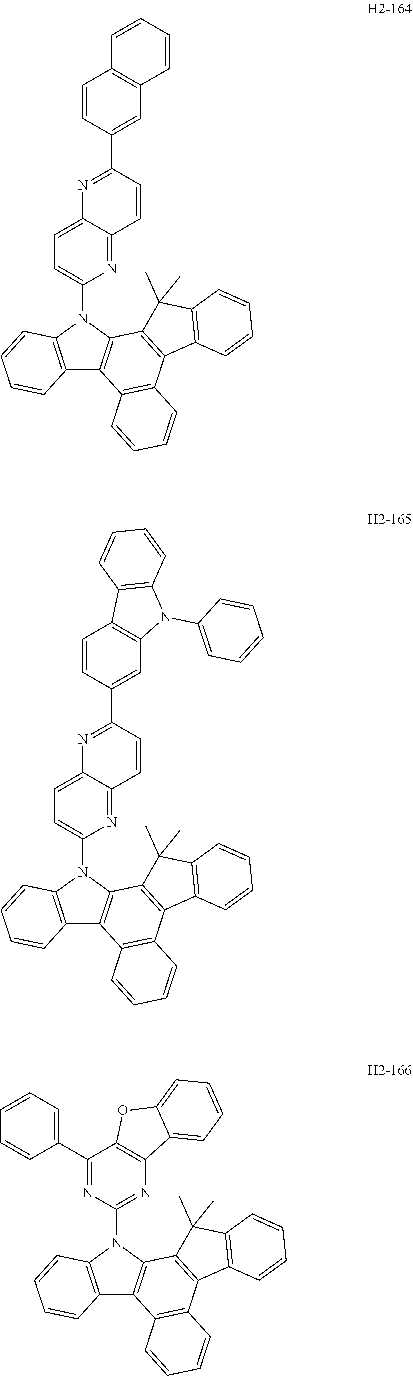 [SMILES: CC1(C)c2ccccc2-c2c1c1c(c3ccccc23)c2ccccc2n1-c1ccc2nc(-c3ccc4ccccc4c3)ccc2n1 ; CC1(C)c2ccccc2-c2c1c1c(c3ccccc23)c2ccccc2n1-c1ccc2nc(-c3ccc4c5ccccc5n(-c5ccccc5)c4c3)ccc2n1 ; CC1(C)c2ccccc2-c2c1c1c(c3ccccc23)c2ccccc2n1-c1nc(-c2ccccc2)c2oc3ccccc3c2n1]

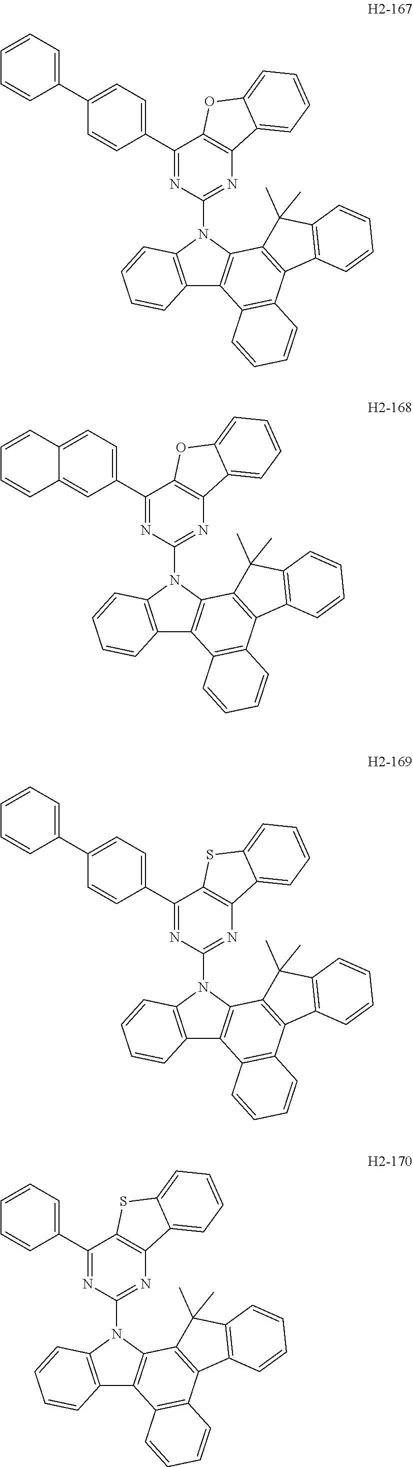 [SMILES: CC1(C)c2ccccc2-c2c1c1c(c3ccccc23)c2ccccc2n1-c1nc(-c2ccc(-c3ccccc3)cc2)c2oc3ccccc3c2n1 ; CC1(C)c2ccccc2-c2c1c1c(c3ccccc23)c2ccccc2n1-c1nc(-c2ccc3ccccc3c2)c2oc3ccccc3c2n1 ; CC1(C)c2ccccc2-c2c1c1c(c3ccccc23)c2ccccc2n1-c1nc(-c2ccc(-c3ccccc3)cc2)c2sc3ccccc3c2n1 ; CC1(C)c2ccccc2-c2c1c1c(c3ccccc23)c2ccccc2n1-c1nc(-c2ccccc2)c2sc3ccccc3c2n1]

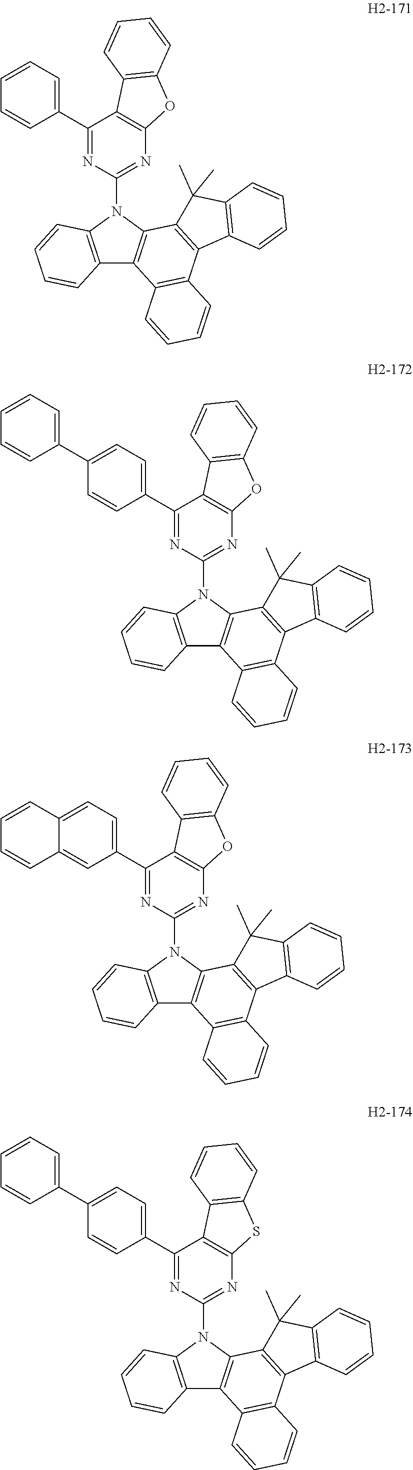 [SMILES: CC1(C)c2ccccc2-c2c1c1c(c3ccccc23)c2ccccc2n1-c1nc(-c2ccccc2)c2c(n1)oc1ccccc12 ; CC1(C)c2ccccc2-c2c1c1c(c3ccccc23)c2ccccc2n1-c1nc(-c2ccc(-c3ccccc3)cc2)c2c(n1)oc1ccccc12 ; CC1(C)c2ccccc2-c2c1c1c(c3ccccc23)c2ccccc2n1-c1nc(-c2ccc3ccccc3c2)c2c(n1)oc1ccccc12 ; CC1(C)c2ccccc2-c2c1c1c(c3ccccc23)c2ccccc2n1-c1nc(-c2ccc(-c3ccccc3)cc2)c2c(n1)sc1ccccc12]

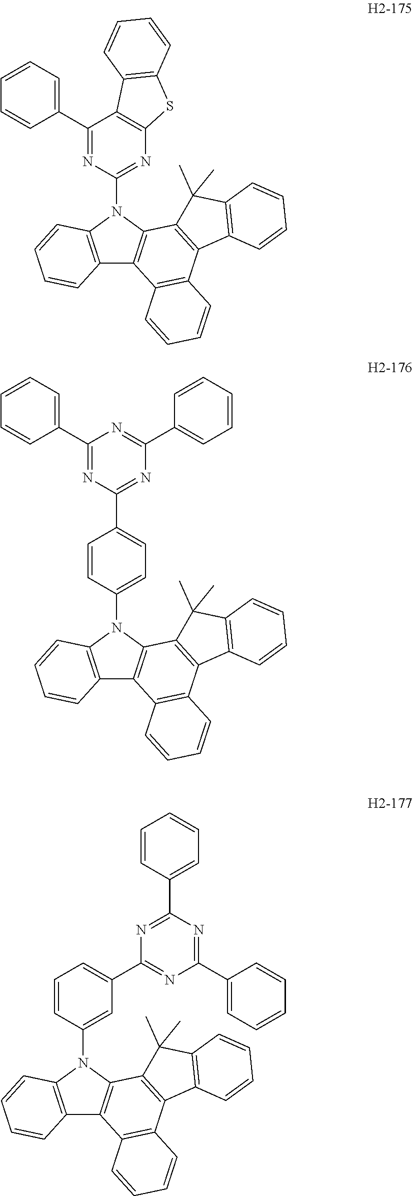

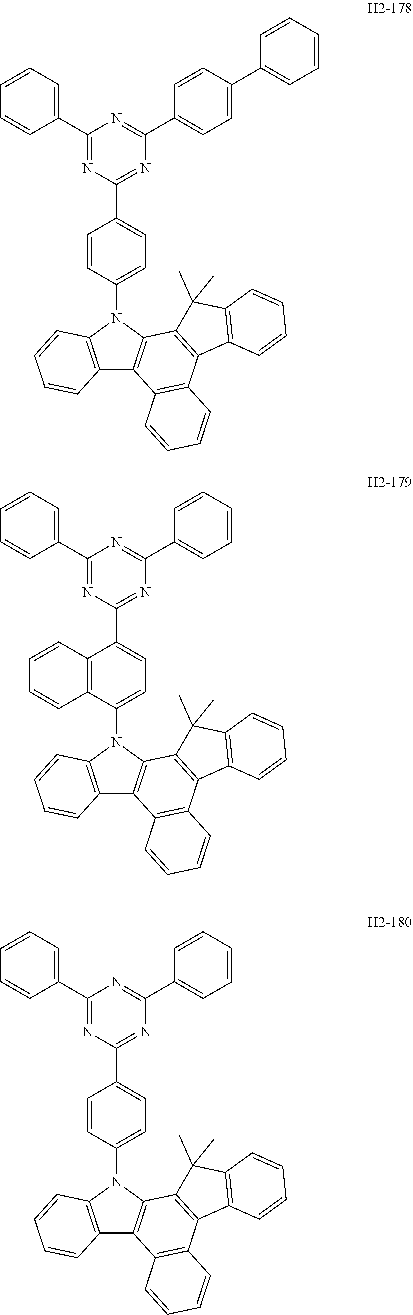 [SMILES: CC1(C)c2ccccc2-c2c1c1c(c3ccccc23)c2ccccc2n1-c1ccc(-c2nc(-c3ccccc3)nc(-c3ccc(-c4ccccc4)cc3)n2)cc1 ; CC1(C)c2ccccc2-c2c1c1c(c3ccccc23)c2ccccc2n1-c1ccc(-c2nc(-c3ccccc3)nc(-c3ccccc3)n2)c2ccccc12 ; CC1(C)c2ccccc2-c2c1c1c(c3ccccc23)c2ccccc2n1-c1ccc(-c2nc(-c3ccccc3)nc(-c3ccccc3)n2)cc1]

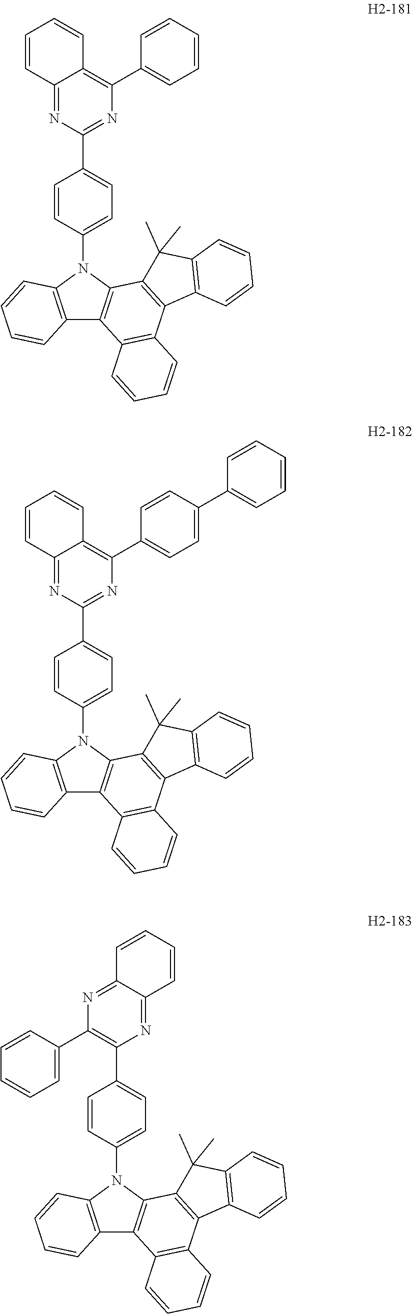 [SMILES: CC1(C)c2ccccc2-c2c1c1c(c3ccccc23)c2ccccc2n1-c1ccc(-c2nc(-c3ccccc3)c3ccccc3n2)cc1 ; CC1(C)c2ccccc2-c2c1c1c(c3ccccc23)c2ccccc2n1-c1ccc(-c2nc(-c3ccc(-c4ccccc4)cc3)c3ccccc3n2)cc1 ; CC1(C)c2ccccc2-c2c1c1c(c3ccccc23)c2ccccc2n1-c1ccc(-c2nc3ccccc3nc2-c2ccccc2)cc1]

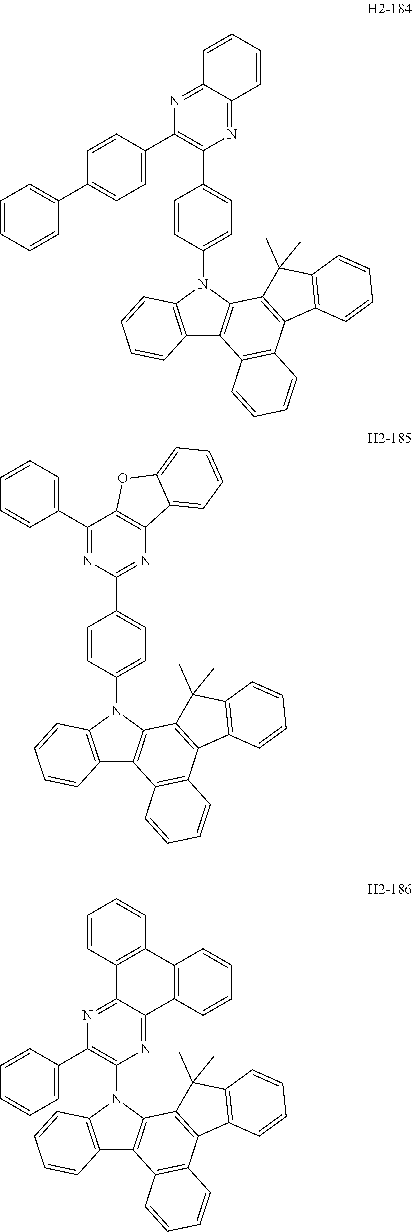 [SMILES: CC1(C)c2ccccc2-c2c1c1c(c3ccccc23)c2ccccc2n1-c1ccc(-c2nc3ccccc3nc2-c2ccc(-c3ccccc3)cc2)cc1 ; CC1(C)c2ccccc2-c2c1c1c(c3ccccc23)c2ccccc2n1-c1ccc(-c2nc(-c3ccccc3)c3oc4ccccc4c3n2)cc1 ; CC1(C)c2ccccc2-c2c1c1c(c3ccccc23)c2ccccc2n1-c1nc2c3ccccc3c3ccccc3c2nc1-c1ccccc1]

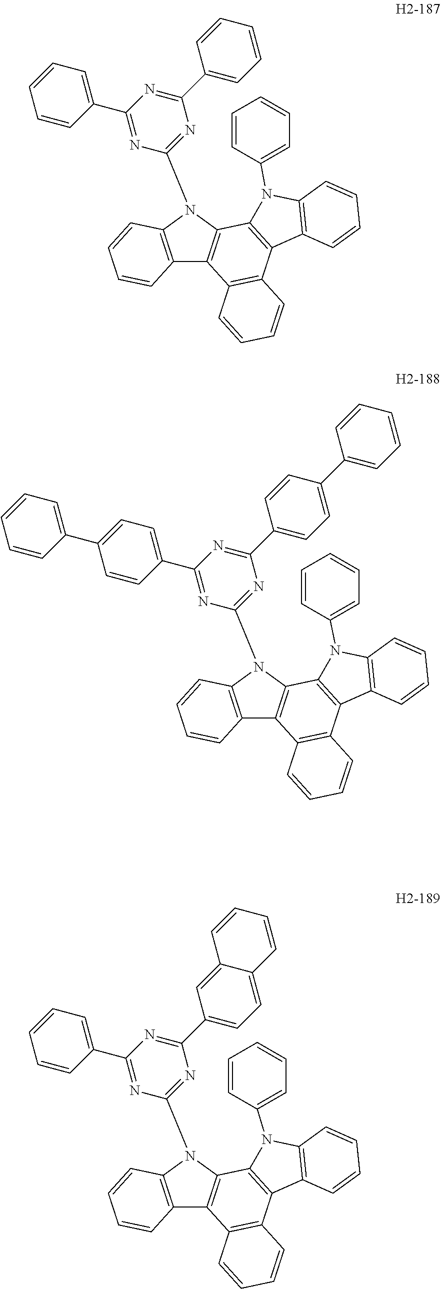 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2ccc(-c3nc(-c4ccc(-c5ccccc5)cc4)nc(-n4c5ccccc5c5c6ccccc6c6c7ccccc7n(-c7ccccc7)c6c54)n3)cc2)cc1 ; c1ccc(-c2nc(-c3ccc4ccccc4c3)nc(-n3c4ccccc4c4c5ccccc5c5c6ccccc6n(-c6ccccc6)c5c43)n2)cc1]

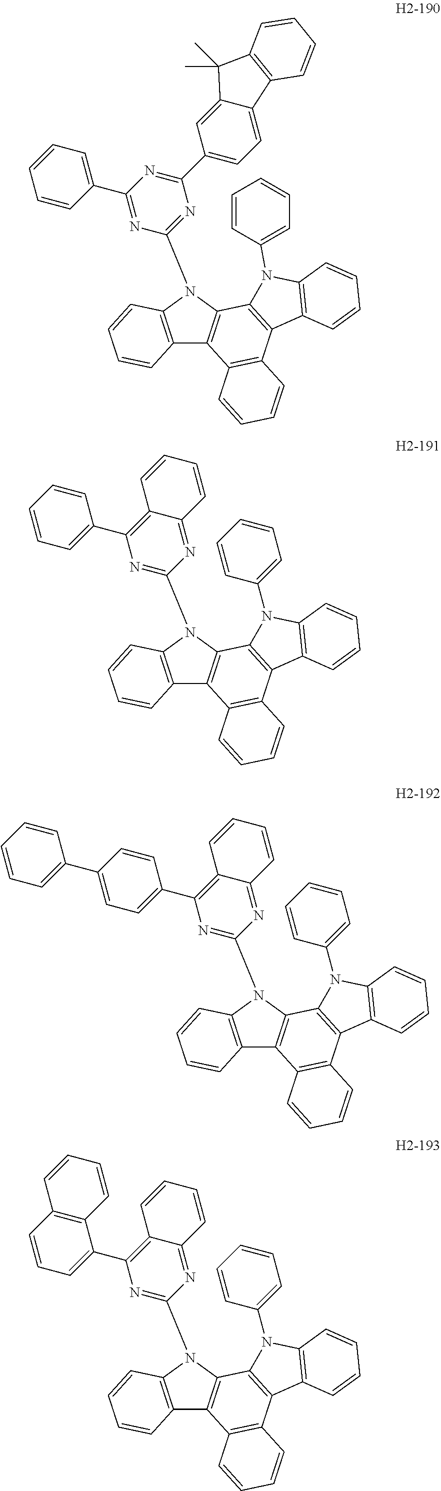 [SMILES: CC1(C)c2ccccc2-c2ccc(-c3nc(-c4ccccc4)nc(-n4c5ccccc5c5c6ccccc6c6c7ccccc7n(-c7ccccc7)c6c54)n3)cc21 ; c1ccc(-c2nc(-n3c4ccccc4c4c5ccccc5c5c6ccccc6n(-c6ccccc6)c5c43)nc3ccccc23)cc1 ; c1ccc(-c2ccc(-c3nc(-n4c5ccccc5c5c6ccccc6c6c7ccccc7n(-c7ccccc7)c6c54)nc4ccccc34)cc2)cc1 ; c1ccc(-n2c3ccccc3c3c4ccccc4c4c5ccccc5n(-c5nc(-c6cccc7ccccc67)c6ccccc6n5)c4c32)cc1]

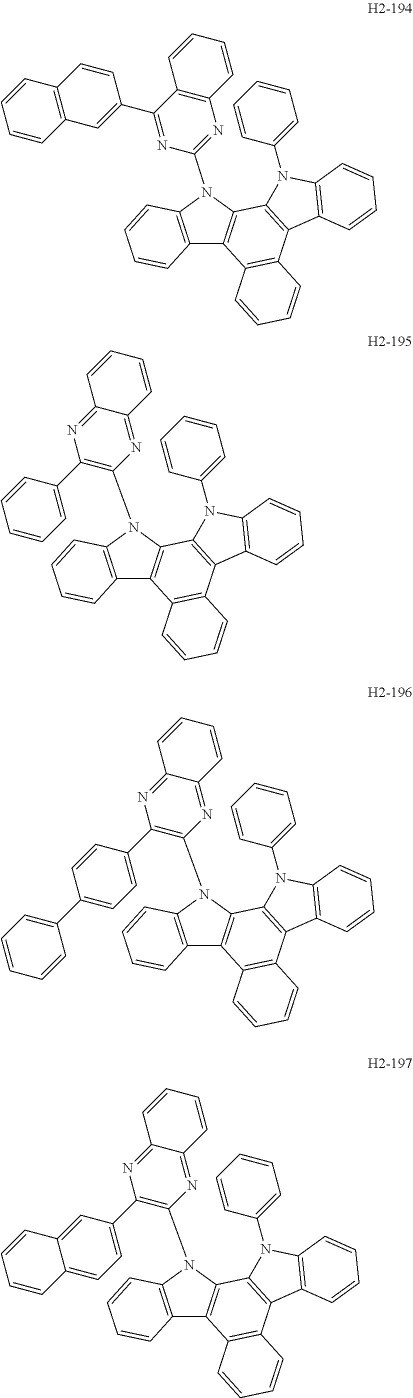 [SMILES: c1ccc(-n2c3ccccc3c3c4ccccc4c4c5ccccc5n(-c5nc(-c6ccc7ccccc7c6)c6ccccc6n5)c4c32)cc1 ; c1ccc(-c2nc3ccccc3nc2-n2c3ccccc3c3c4ccccc4c4c5ccccc5n(-c5ccccc5)c4c32)cc1 ; c1ccc(-c2ccc(-c3nc4ccccc4nc3-n3c4ccccc4c4c5ccccc5c5c6ccccc6n(-c6ccccc6)c5c43)cc2)cc1 ; c1ccc(-n2c3ccccc3c3c4ccccc4c4c5ccccc5n(-c5nc6ccccc6nc5-c5ccc6ccccc6c5)c4c32)cc1]

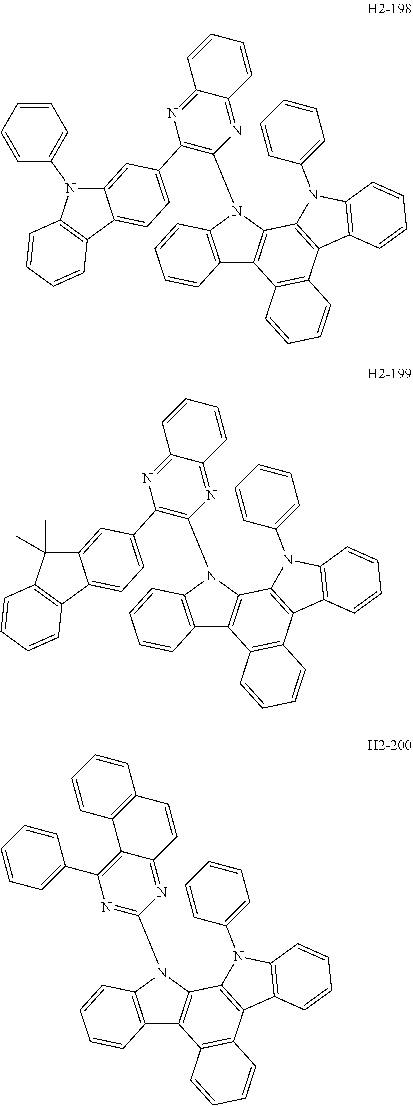 [SMILES: c1ccc(-n2c3ccccc3c3ccc(-c4nc5ccccc5nc4-n4c5ccccc5c5c6ccccc6c6c7ccccc7n(-c7ccccc7)c6c54)cc32)cc1 ; CC1(C)c2ccccc2-c2ccc(-c3nc4ccccc4nc3-n3c4ccccc4c4c5ccccc5c5c6ccccc6n(-c6ccccc6)c5c43)cc21 ; c1ccc(-c2nc(-n3c4ccccc4c4c5ccccc5c5c6ccccc6n(-c6ccccc6)c5c43)nc3ccc4ccccc4c23)cc1]

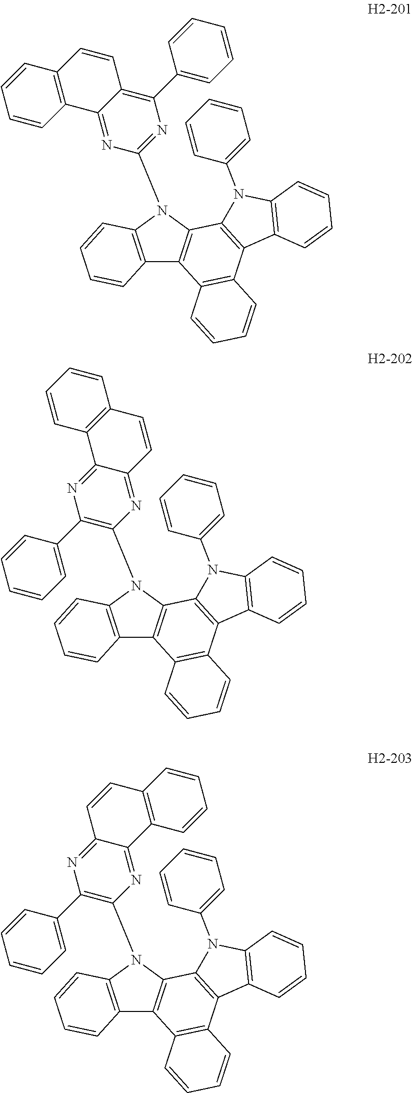 [SMILES: c1ccc(-c2nc(-n3c4ccccc4c4c5ccccc5c5c6ccccc6n(-c6ccccc6)c5c43)nc3c2ccc2ccccc23)cc1 ; c1ccc(-c2nc3c(ccc4ccccc43)nc2-n2c3ccccc3c3c4ccccc4c4c5ccccc5n(-c5ccccc5)c4c32)cc1 ; c1ccc(-c2nc3ccc4ccccc4c3nc2-n2c3ccccc3c3c4ccccc4c4c5ccccc5n(-c5ccccc5)c4c32)cc1]

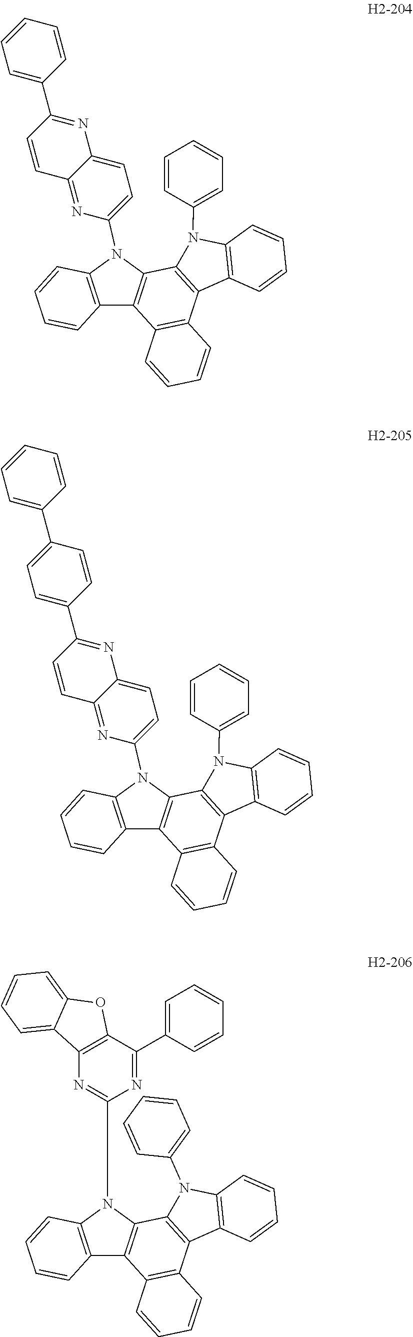 [SMILES: c1ccc(-c2ccc3nc(-n4c5ccccc5c5c6ccccc6c6c7ccccc7n(-c7ccccc7)c6c54)ccc3n2)cc1 ; c1ccc(-c2ccc(-c3ccc4nc(-n5c6ccccc6c6c7ccccc7c7c8ccccc8n(-c8ccccc8)c7c65)ccc4n3)cc2)cc1 ; c1ccc(-c2nc(-n3c4ccccc4c4c5ccccc5c5c6ccccc6n(-c6ccccc6)c5c43)nc3c2oc2ccccc23)cc1]

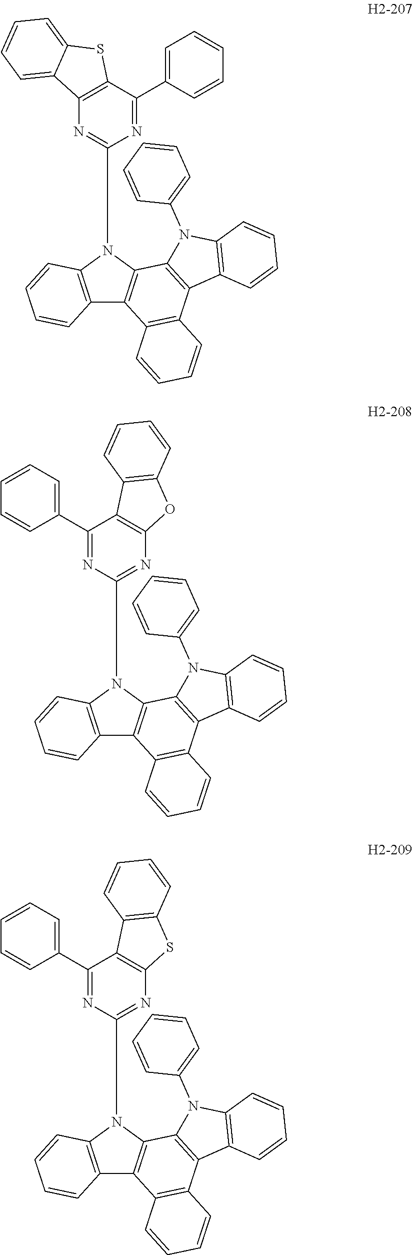

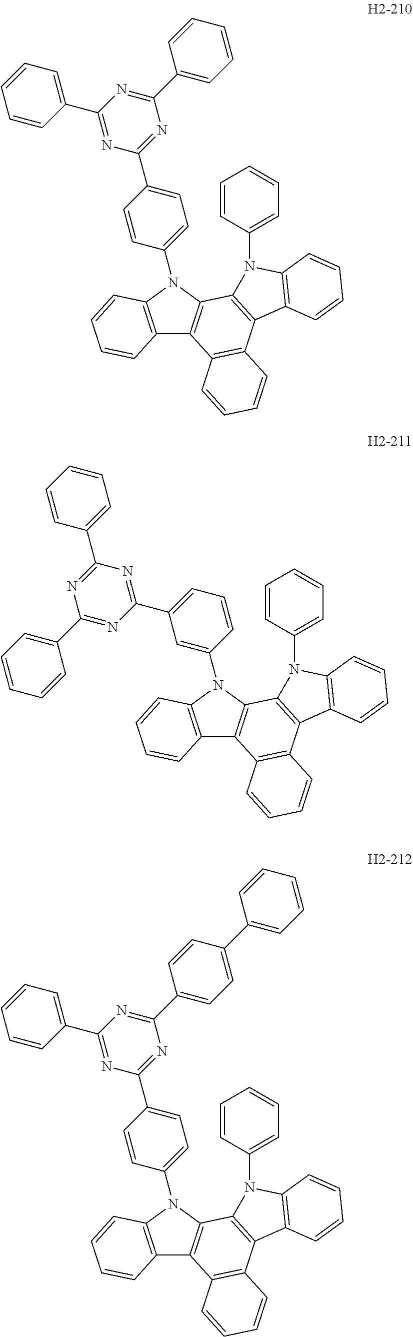 [SMILES: c1ccc(-c2nc(-c3ccccc3)nc(-c3ccc(-n4c5ccccc5c5c6ccccc6c6c7ccccc7n(-c7ccccc7)c6c54)cc3)n2)cc1 ; c1ccc(-c2nc(-c3ccccc3)nc(-c3cccc(-n4c5ccccc5c5c6ccccc6c6c7ccccc7n(-c7ccccc7)c6c54)c3)n2)cc1 ; c1ccc(-c2ccc(-c3nc(-c4ccccc4)nc(-c4ccc(-n5c6ccccc6c6c7ccccc7c7c8ccccc8n(-c8ccccc8)c7c65)cc4)n3)cc2)cc1]

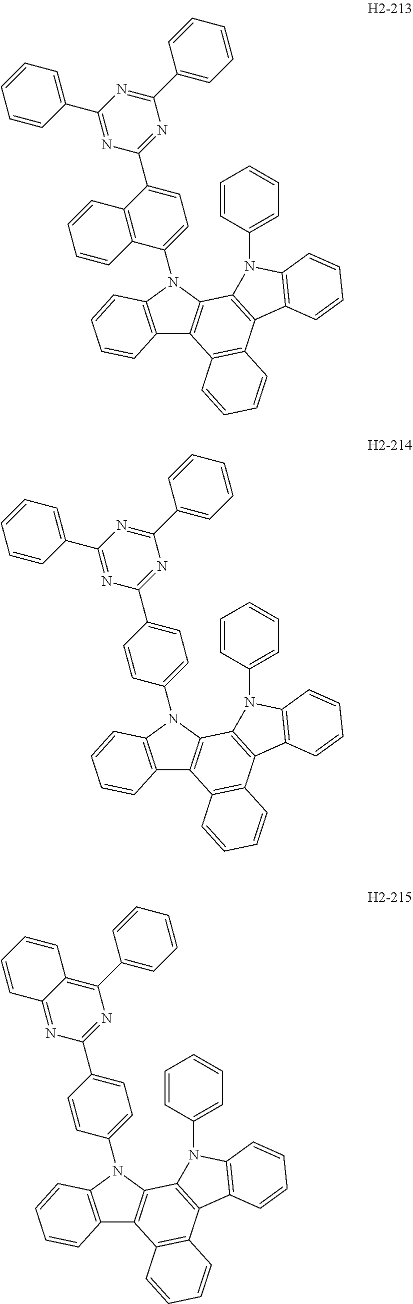 [SMILES: c1ccc(-c2nc(-c3ccccc3)nc(-c3ccc(-n4c5ccccc5c5c6ccccc6c6c7ccccc7n(-c7ccccc7)c6c54)c4ccccc34)n2)cc1 ; c1ccc(-c2nc(-c3ccccc3)nc(-c3ccc(-n4c5ccccc5c5c6ccccc6c6c7ccccc7n(-c7ccccc7)c6c54)cc3)n2)cc1 ; c1ccc(-c2nc(-c3ccc(-n4c5ccccc5c5c6ccccc6c6c7ccccc7n(-c7ccccc7)c6c54)cc3)nc3ccccc23)cc1]

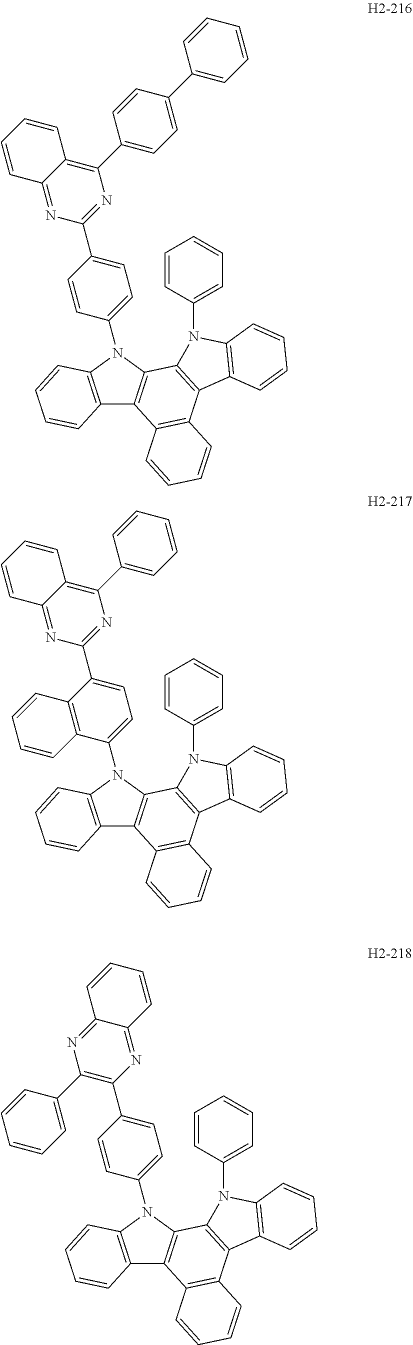

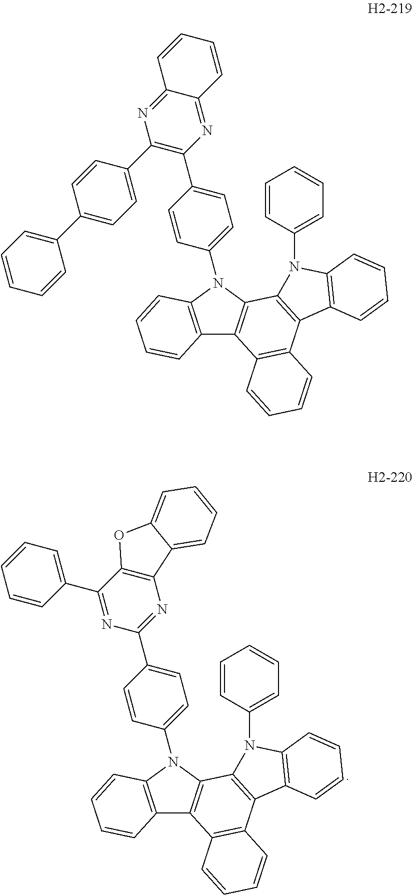 [SMILES: c1ccc(-c2ccc(-c3nc4ccccc4nc3-c3ccc(-n4c5ccccc5c5c6ccccc6c6c7ccccc7n(-c7ccccc7)c6c54)cc3)cc2)cc1 ; c1ccc(-c2nc(-c3ccc(-n4c5ccccc5c5c6ccccc6c6c7ccccc7n(-c7ccccc7)c6c54)cc3)nc3c2oc2ccccc23)cc1]

7. A mixture comprising a combination of the compound represented by formula 1 according to claim 1 and the compound represented by formula 2 according to claim 1.

8. An organic electroluminescent device comprising an anode, a cathode, and at least one light-emitting layer between the anode and the cathode, wherein the light-emitting layer comprises a host and a phosphorescent dopant, and the host comprises the plurality of host materials according to claim 1.
